(19) United States
(12) Patent Application Publication
(10) Pub. No.: US 2025/0382373 A1
(43) Pub. Date: Dec. 18, 2025

(54) ANTI-EGFR/MET ANTIBODIES AND USES THEREOF

(71) Applicant: Biocytogen Pharmaceuticals (Beijing) Co., Ltd., Beijing (CN)

(72) Inventors: Yanfei Han, Beijing (CN); Chengzhang Shang, Beijing (CN); Baihong Liu, Beijing (CN); Yi Yang, Beijing (CN); Yuelei Shen, Beijing (CN)

(21) Appl. No.: 18/878,766

(22) PCT Filed: Jun. 29, 2023
(86) PCT No.: PCT/CN2023/103811

§ 371 Date: Dec. 24, 2024

(30) Foreign Application Priority Data
Jun. 29, 2022 (WO) PCT/CN2022/102231
Jul. 29, 2022 (WO) PCT/CN2022/108838
Sep. 2, 2022 (WO) PCT/CN2022/116678
Dec. 23, 2022 (WO) PCT/CN2022/141416

(51) Int. Cl. C07K 16/28; A61K 39/00; A61K 47/68; A61P 35/00; C07K 16/40

(52) U.S. Cl. CPC C07K 16/2863; A61K 47/68031; A61K 47/6879; A61P 35/00; C07K 16/40; C12Y 207/10001; A61K 2039/505; C07K 2317/24; C07K 2317/31; C07K 2317/33; C07K 2317/77; C07K 2317/92; C07K 2317/94

(57) ABSTRACT

Provided is antigen-binding protein constructs (e.g., bispecific antibodies, antigen-binding fragments thereof, or ADCs), wherein the antigen-binding protein constructs specifically bind to two different antigens (e.g., EGFR and MET).

### CLAIM OF PRIORITY

This application claims the benefit of PCT Application No. PCT/CN2022/102231, filed on Jun. 29, 2022, PCT Application No. PCT/CN2022/108838, filed on Jul. 29, 2022, PCT Application No. PCT/CN2022/116678, filed on Sep. 2, 2022, and PCT Application No. PCT/CN2022/141416, filed on Dec. 23, 2022. The entire contents of the foregoing are incorporated herein by reference.

### TECHNICAL FIELD

This disclosure relates to antigen-binding protein constructs (e.g., bispecific antibodies or antigen-binding fragments thereof).

### BACKGROUND

A bispecific antibody is an artificial protein that can simultaneously bind to two different types of antigens or two different epitopes. This dual specificity opens up a wide range of applications, including redirecting T cells to tumor cells, dual targeting of different disease mediators, and delivering payloads to targeted sites. The approval of catumaxomab (anti-EpCAM and anti-CD3) and blinatumomab (anti-CD19 and anti-CD3) has become a major milestone in the development of bispecific antibodies.

As bispecific antibodies have various applications, there is a need to continue to develop various therapeutics based on bispecific antibodies.

### SUMMARY

This disclosure relates to antigen-binding protein constructs, wherein the antigen-binding protein construct specifically bind to two different antigens (e.g., EGFR and MET). In some embodiments, the multispecific antibody (e.g., bispecific antibody) has identical light chain variable regions. In some embodiments, the multispecific antibody (e.g., bispecific antibody) has a common light chain. In some embodiments, the multispecific antibody (e.g., bispecific antibody) forms part of an antibody drug conjugate.

In one aspect, the disclosure is related to an antibody or antigen-binding fragment thereof that binds to EGFR (epidermal growth factor receptor) comprising: a heavy chain variable region (VH) comprising complementarity determining regions (CDRs) 1, 2, and 3, in some embodiments, the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, in some embodiments, the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence, in some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 4-6, respectively, and the selected VL CDRs 1, 2, 3 amino acid sequences are set forth in SEQ ID NOs: 1-3, respectively;
(2) the selected VH CDRs 1, 2, 3 amino acid sequences are set forth in SEQ ID NOs: 7-9, respectively, and the selected VL CDRs 1, 2, 3 amino acid sequences are set forth in SEQ ID NOs: 1-3, respectively;
(3) the selected VH CDRs 1, 2, 3 amino acid sequences are set forth in SEQ ID NOs: 10-12, respectively, and the selected VL CDRs 1, 2, 3 amino acid sequences are set forth in SEQ ID NOs: 1-3, respectively;
(4) the selected VH CDRs 1, 2, 3 amino acid sequences are set forth in SEQ ID NOs: 13-15, respectively, and the selected VL CDRs 1, 2, 3 amino acid sequences are set forth in SEQ ID NOs: 1-3, respectively;
(5) the selected VH CDRs 1, 2, 3 amino acid sequences are set forth in SEQ ID NOs: 16-18, respectively, and the selected VL CDRs 1, 2, 3 amino acid sequences are set forth in SEQ ID NOs: 1-3, respectively;
(6) the selected VH CDRs 1, 2, 3 amino acid sequences are set forth in SEQ ID NOs: 34-36, respectively, and the selected VL CDRs 1, 2, 3 amino acid sequences are set forth in SEQ ID NOs: 1-3, respectively;
(7) the selected VH CDRs 1, 2, 3 amino acid sequences are set forth in SEQ ID NOs: 37-39, respectively, and the selected VL CDRs 1, 2, 3 amino acid sequences are set forth in SEQ ID NOs: 1-3, respectively;
(8) the selected VH CDRs 1, 2, 3 amino acid sequences are set forth in SEQ ID NOs: 40-42, respectively, and the selected VL CDRs 1, 2, 3 amino acid sequences are set forth in SEQ ID NOs: 1-3, respectively;
(9) the selected VH CDRs 1, 2, 3 amino acid sequences are set forth in SEQ ID NOs: 43-45, respectively, and the selected VL CDRs 1, 2, 3 amino acid sequences are set forth in SEQ ID NOs: 1-3, respectively;
(10) the selected VH CDRs 1, 2, 3 amino acid sequences are set forth in SEQ ID NOs: 46-48, respectively, and the selected VL CDRs 1, 2, 3 amino acid sequences are set forth in SEQ ID NOs: 1-3, respectively;
(11) the selected VH CDRs 1, 2, 3 amino acid sequences are set forth in SEQ ID NOs: 82-84, respectively, and the selected VL CDRs 1, 2, 3 amino acid sequences are set forth in SEQ ID NOs: 1-3, respectively;
(12) the selected VH CDRs 1, 2, 3 amino acid sequences are set forth in SEQ ID NOs: 85-87, respectively, and the selected VL CDRs 1, 2, 3 amino acid sequences are set forth in SEQ ID NOs: 1-3, respectively;
(13) the selected VH CDRs 1, 2, 3 amino acid sequences are set forth in SEQ ID NOs: 88-90, respectively, and the selected VL CDRs 1, 2, 3 amino acid sequences are set forth in SEQ ID NOs: 1-3, respectively;
(14) the selected VH CDRs 1, 2, 3 amino acid sequences are set forth in SEQ ID NOs: 91-93, respectively, and the selected VL CDRs 1, 2, 3 amino acid sequences are set forth in SEQ ID NOs: 1-3, respectively;
(15) the selected VH CDRs 1, 2, 3 amino acid sequences are set forth in SEQ ID NOs: 94-96, respectively, and the selected VL CDRs 1, 2, 3 amino acid sequences are set forth in SEQ ID NOs: 1-3, respectively;
(16) the selected VH CDRs 1, 2, 3 amino acid sequences are set forth in SEQ ID NOs: 97-99, respectively, and the selected VL CDRs 1, 2, 3 amino acid sequences are set forth in SEQ ID NOs: 1-3, respectively;
(17) the selected VH CDRs 1, 2, 3 amino acid sequences are set forth in SEQ ID NOs: 100-102, respectively, and the selected VL CDRs 1, 2, 3 amino acid sequences are set forth in SEQ ID NOs: 1-3, respectively; and
(18) the selected VH CDRs 1, 2, 3 amino acid sequences are set forth in SEQ ID NOs: 103-105, respectively, and the selected VL CDRs 1, 2, 3 amino acid sequences are set forth in SEQ ID NOs: 1-3, respectively.

In some embodiments, the VH comprises CDRs 1, 2, 3 with the amino acid sequences set forth in SEQ ID NOs: 4-6, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 7-9, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 10-12, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 13-15, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 16-18, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 82-84, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 85-87, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 88-90, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 91-93, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 34-36, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 37-39, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 40-42, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 43-45, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 46-48, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 94-96, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 97-99, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 100-102, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 103-105, respectively, and the VL comprises CDRs 1, 2, 3 with the amino acid sequences set forth in SEQ ID NOs: 1-3, respectively, according to the Chothia numbering scheme.

In one aspect, the disclosure is related to an antibody or antigen-binding fragment thereof that binds to EGFR comprising a heavy chain variable region (VH) comprising an amino acid sequence that is at least 90% identical to a selected VH sequence, and a light chain variable region (VL) comprising an amino acid sequence that is at least 90% identical to a selected VL sequence, in some embodiments, the selected VH sequence and the selected VL sequence are one of the following:
(1) the selected VH sequence is SEQ ID NO:62, and the selected VL sequence is SEQ ID NO:61;
(2) the selected VH sequence is SEQ ID NO:63, and the selected VL sequence is SEQ ID NO:61;
(3) the selected VH sequence is SEQ ID NO:64, and the selected VL sequence is SEQ ID NO:61;
(4) the selected VH sequence is SEQ ID NO:65, and the selected VL sequence is SEQ ID NO:61;
(5) the selected VH sequence is SEQ ID NO:66, and the selected VL sequence is SEQ ID NO:61;
(6) the selected VH sequence is SEQ ID NO:106, and the selected VL sequence is SEQ ID NO:61;
(7) the selected VH sequence is SEQ ID NO:107, and the selected VL sequence is SEQ ID NO:61;
(8) the selected VH sequence is SEQ ID NO:108, and the selected VL sequence is SEQ ID NO:61; and
(9) the selected VH sequence is SEQ ID NO:109, and the selected VL sequence is SEQ ID NO:61.

In some embodiments, the VH comprises the sequence of SEQ ID NO: 62 and the VL comprises the sequence of SEQ ID NO: 61. In some embodiments, the VH comprises the sequence of SEQ ID NO: 63 and the VL comprises the sequence of SEQ ID NO: 61. In some embodiments, the VH comprises the sequence of SEQ ID NO: 64 and the VL comprises the sequence of SEQ ID NO: 61. In some embodiments, the VH comprises the sequence of SEQ ID NO: 65 and the VL comprises the sequence of SEQ ID NO: 61. In some embodiments, the VH comprises the sequence of SEQ ID NO: 66 and the VL comprises the sequence of SEQ ID NO: 61. In some embodiments, the VH comprises the sequence of SEQ ID NO: 106 and the VL comprises the sequence of SEQ ID NO: 61. In some embodiments, the VH comprises the sequence of SEQ ID NO: 107 and the VL comprises the sequence of SEQ ID NO: 61. In some embodiments, the VH comprises the sequence of SEQ ID NO: 108 and the VL comprises the sequence of SEQ ID NO: 61. In some embodiments, the VH comprises the sequence of SEQ ID NO: 109 and the VL comprises the sequence of SEQ ID NO: 61.

In one aspect, the disclosure is related to an antibody or antigen-binding fragment thereof that binds to EGFR comprising a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, in some embodiments, the selected VH sequence and the selected VL sequence are one of the following:
(1) the selected VH sequence is SEQ ID NO:62, and the selected VL sequence is SEQ ID NO:61;
(2) the selected VH sequence is SEQ ID NO:63, and the selected VL sequence is SEQ ID NO:61;
(3) the selected VH sequence is SEQ ID NO:64, and the selected VL sequence is SEQ ID NO:61;
(4) the selected VH sequence is SEQ ID NO:65, and the selected VL sequence is SEQ ID NO:61;
(5) the selected VH sequence is SEQ ID NO:66, and the selected VL sequence is SEQ ID NO:61;
(6) the selected VH sequence is SEQ ID NO:106, and the selected VL sequence is SEQ ID NO:61;
(7) the selected VH sequence is SEQ ID NO:107, and the selected VL sequence is SEQ ID NO:61;
(8) the selected VH sequence is SEQ ID NO:108, and the selected VL sequence is SEQ ID NO:61; and
(9) the selected VH sequence is SEQ ID NO:109, and the selected VL sequence is SEQ ID NO:61.

In some embodiments, the antibody or antigen-binding fragment thereof specifically binds to human or monkey EGFR. In some embodiments, the antibody or antigen-binding fragment thereof is a human or humanized antibody or antigen-binding fragment thereof. In some embodiments, the antibody or antigen-binding fragment is a single-chain variable fragment (scFV). In some embodiments, the antibody or antigen-binding fragment thereof is a multispecific antibody (e.g., a bispecific antibody) or an antigen-binding fragment thereof. In some embodiments, the antibody or antigen-binding fragment thereof further specifically binds to MET. In one aspect, the disclosure is related to an antibody or antigen-binding fragment thereof that cross-competes with the antibody or antigen-binding fragment thereof as described herein.

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 4-6, respectively, and in some embodiments, the VH, when paired with a light chain variable region (VL) comprising the amino acid sequence set forth in SEQ ID NO: 61, binds to EGFR;
(2) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and in some embodiments, the VL, when paired with a heavy chain variable region (VH) comprising the amino acid sequence set forth in SEQ ID NO: 62, binds to EGFR;
(3) an immunoglobulin heavy chain or a fragment thereof comprising a VH comprising CDRs 1, 2, and 3 comprising the amino acid sequences set forth in SEQ ID NOs: 7-9, respectively, and in some embodiments, the VH, when paired with a VL comprising the amino acid sequence set forth in SEQ ID NO: 61, binds to EGFR;
(4) an immunoglobulin light chain or a fragment thereof comprising a VL comprising CDRs 1, 2, and 3 comprising the amino acid sequences set forth in SEQ ID NOs: 1-3, respectively, and in some embodiments, the VL, when paired with a VH comprising the amino acid sequence set forth in SEQ ID NO: 63, binds to EGFR;
(5) an immunoglobulin heavy chain or a fragment thereof comprising a VH comprising CDRs 1, 2, and 3 comprising the amino acid sequences set forth in SEQ ID NOs: 10-12, respectively, and in some embodiments, the VH, when paired with a VL comprising the amino acid sequence set forth in SEQ ID NO: 61, binds to EGFR;
(6) an immunoglobulin light chain or a fragment thereof comprising a VL comprising CDRs 1, 2, and 3 comprising the amino acid sequences set forth in SEQ ID NOs: 1-3, respectively, and in some embodiments, the VL, when paired with a VH comprising the amino acid sequence set forth in SEQ ID NO: 64, binds to EGFR;
(7) an immunoglobulin heavy chain or a fragment thereof comprising a VH comprising CDRs 1, 2, and 3 comprising the amino acid sequences set forth in SEQ ID NOs: 13-15, respectively, and in some embodiments, the VH, when paired with a VL comprising the amino acid sequence set forth in SEQ ID NO: 61, binds to EGFR
(8) an immunoglobulin light chain or a fragment thereof comprising a VL comprising CDRs 1, 2, and 3 comprising the amino acid sequences set forth in SEQ ID NOs: 1-3, respectively, and in some embodiments, the VL, when paired with a VH comprising the amino acid sequence set forth in SEQ ID NO: 65, binds to EGFR;
(9) an immunoglobulin heavy chain or a fragment thereof comprising a VH comprising CDRs 1, 2, and 3 comprising the amino acid sequences set forth in SEQ ID NOs: 16-18, respectively, and in some embodiments, the VH, when paired with a VL comprising the amino acid sequence set forth in SEQ ID NO: 61, binds to EGFR;
(10) an immunoglobulin light chain or a fragment thereof comprising a VL comprising CDRs 1, 2, and 3 comprising the amino acid sequences set forth in SEQ ID NOs: 1-3, respectively, and in some embodiments, the VL, when paired with a VH comprising the amino acid sequence set forth in SEQ ID NO: 66, binds to EGFR;
(11) an immunoglobulin heavy chain or a fragment thereof comprising a VH comprising CDRs 1, 2, and 3 comprising the amino acid sequences set forth in SEQ ID NOs: 34-36, respectively, and in some embodiments, the VH, when paired with a VL comprising the amino acid sequence set forth in SEQ ID NO: 61, binds to EGFR;
(12) an immunoglobulin heavy chain or a fragment thereof comprising a VH comprising CDRs 1, 2, and 3 comprising the amino acid sequences set forth in SEQ ID NOs: 37-39, respectively, and in some embodiments, the VH, when paired with a VL comprising the amino acid sequence set forth in SEQ ID NO: 61, binds to EGFR;
(13) an immunoglobulin heavy chain or a fragment thereof comprising a VH comprising CDRs 1, 2, and 3 comprising the amino acid sequences set forth in SEQ ID NOs: 40-42, respectively, and in some embodiments, the VH, when paired with a VL comprising the amino acid sequence set forth in SEQ ID NO: 61, binds to EGFR;
(14) an immunoglobulin heavy chain or a fragment thereof comprising a VH comprising CDRs 1, 2, and 3 comprising the amino acid sequences set forth in SEQ ID NOs: 43-45, respectively, and in some embodiments, the VH, when paired with a VL comprising the amino acid sequence set forth in SEQ ID NO: 61, binds to EGFR;
(15) an immunoglobulin heavy chain or a fragment thereof comprising a VH comprising CDRs 1, 2, and 3 comprising the amino acid sequences set forth in SEQ ID NOs: 46-48, respectively, and in some embodiments, the VH, when paired with a VL comprising the amino acid sequence set forth in SEQ ID NO: 61, binds to EGFR;
(16) an immunoglobulin heavy chain or a fragment thereof comprising a VH comprising CDRs 1, 2, and 3 comprising the amino acid sequences set forth in SEQ ID NOs: 82-84, respectively, and wherein the VH, when paired with a VL comprising the amino acid sequence set forth in SEQ ID NO: 61, binds to EGFR;
(17) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 106, binds to EGFR;
(18) an immunoglobulin heavy chain or a fragment thereof comprising a VH comprising CDRs 1, 2, and 3 comprising the amino acid sequences set forth in SEQ ID NOs: 85-87, respectively, and wherein the VH, when paired with a VL comprising the amino acid sequence set forth in SEQ ID NO: 61, binds to EGFR;
(19) an immunoglobulin light chain or a fragment thereof comprising a VL comprising CDRS 1, 2, and 3 comprising the amino acid sequences set forth in SEQ ID NOs: 1-3, respectively, and wherein the VL, when paired with a VH comprising the amino acid sequence set forth in SEQ ID NO: 107, binds to EGFR;
(20) an immunoglobulin heavy chain or a fragment thereof comprising a VH comprising CDRs 1, 2, and 3 comprising the amino acid sequences set forth in SEQ ID NOs: 88-90, respectively, and wherein the VH, when paired with a VL comprising the amino acid sequence set forth in SEQ ID NO: 61, binds to EGFR;
(21) an immunoglobulin light chain or a fragment thereof comprising a VL comprising CDRS 1, 2, and 3 comprising the amino acid sequences set forth in SEQ ID NOs: 1-3, respectively, and wherein the VL, when paired with a VH comprising the amino acid sequence set forth in SEQ ID NO: 108, binds to EGFR;
(22) an immunoglobulin heavy chain or a fragment thereof comprising a VH comprising CDRs 1, 2, and 3 comprising the amino acid sequences set forth in SEQ ID NOs: 91-93, respectively, and wherein the VH, when paired with a VL comprising the amino acid sequence set forth in SEQ ID NO: 61, binds to EGFR;
(23) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 109, binds to EGFR;
(24) an immunoglobulin heavy chain or a fragment thereof comprising a VH comprising CDRs 1, 2, and 3 comprising the amino acid sequences set forth in SEQ ID NOs: 94-96, respectively, and wherein the VH, when paired with a VL comprising the amino acid sequence set forth in SEQ ID NO: 61, binds to EGFR;
(25) an immunoglobulin heavy chain or a fragment thereof comprising a VH comprising CDRs 1, 2, and 3 comprising the amino acid sequences set forth in SEQ ID NOs: 97-99, respectively, and wherein the VH, when paired with a VL comprising the amino acid sequence set forth in SEQ ID NO: 61, binds to EGFR;
(26) an immunoglobulin heavy chain or a fragment thereof comprising a VH comprising CDRs 1, 2, and 3 comprising the amino acid sequences set forth in SEQ ID NOs: 100-102, respectively, and wherein the VH, when paired with a VL comprising the amino acid sequence set forth in SEQ ID NO: 61, binds to EGFR; and/or
(27) an immunoglobulin heavy chain or a fragment thereof comprising a VH comprising CDRs 1, 2, and 3 comprising the amino acid sequences set forth in SEQ ID NOs: 103-105, respectively, and wherein the VH, when paired with a VL comprising the amino acid sequence set forth in SEQ ID NO: 61, binds to EGFR.

In some embodiments, the VH when paired with a VL specifically binds to human or monkey EGFR. In some embodiments, the immunoglobulin heavy chain or the fragment thereof is a human or humanized immunoglobulin heavy chain or a fragment thereof. In some embodiments, the nucleic acid encodes a single-chain variable fragment (scFv). In some embodiments, the nucleic acid is cDNA.

In one aspect, the disclosure is related to an antibody or antigen-binding fragment thereof that binds to MET (tyrosine-protein kinase Met) comprising: a heavy chain variable region (VH) comprising complementarity determining regions (CDRs) 1, 2, and 3, in some embodiments, the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, in some embodiments, the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence, in some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 19-21, respectively, and the selected VL CDRs 1, 2, 3 amino acid sequences are set forth in SEQ ID NOs: 1-3, respectively;
(2) the selected VH CDRs 1, 2, 3 amino acid sequences are set forth in SEQ ID NOs: 22-24, respectively, and the selected VL CDRs 1, 2, 3 amino acid sequences are set forth in SEQ ID NOs: 1-3, respectively;
(3) the selected VH CDRs 1, 2, 3 amino acid sequences are set forth in SEQ ID NOs: 25-27, respectively, and the selected VL CDRs 1, 2, 3 amino acid sequences are set forth in SEQ ID NOs: 1-3, respectively;
(4) the selected VH CDRs 1, 2, 3 amino acid sequences are set forth in SEQ ID NOs: 28-30, respectively, and the selected VL CDRs 1, 2, 3 amino acid sequences are set forth in SEQ ID NOs: 1-3, respectively;
(5) the selected VH CDRs 1, 2, 3 amino acid sequences are set forth in SEQ ID NOs: 49-51, respectively, and the selected VL CDRs 1, 2, 3 amino acid sequences are set forth in SEQ ID NOs: 1-3, respectively;
(6) the selected VH CDRs 1, 2, 3 amino acid sequences are set forth in SEQ ID NOs: 52-54, respectively, and the selected VL CDRs 1, 2, 3 amino acid sequences are set forth in SEQ ID NOs: 1-3, respectively;
(7) the selected VH CDRs 1, 2, 3 amino acid sequences are set forth in SEQ ID NOs: 55-57, respectively, and the selected VL CDRs 1, 2, 3 amino acid sequences are set forth in SEQ ID NOs: 1-3, respectively;
(8) the selected VH CDRs 1, 2, 3 amino acid sequences are set forth in SEQ ID NOs: 58-60, respectively, and the selected VL CDRs 1, 2, 3 amino acid sequences are set forth in SEQ ID NOs: 1-3, respectively;
(9) the selected VH CDRs 1, 2, 3 amino acid sequences are set forth in SEQ ID NOs: 112-114, respectively, and the selected VL CDRs 1, 2, 3 amino acid sequences are set forth in SEQ ID NOs: 1-3, respectively;
(10) the selected VH CDRs 1, 2, 3 amino acid sequences are set forth in SEQ ID NOs: 115-117, respectively, and the selected VL CDRs 1, 2, 3 amino acid sequences are set forth in SEQ ID NOs: 1-3, respectively;
(11) the selected VH CDRs 1, 2, 3 amino acid sequences are set forth in SEQ ID NOs: 118-120, respectively, and the selected VL CDRs 1, 2, 3 amino acid sequences are set forth in SEQ ID NOs: 1-3, respectively;
(12) the selected VH CDRs 1, 2, 3 amino acid sequences are set forth in SEQ ID NOs: 121-123, respectively, and the selected VL CDRs 1, 2, 3 amino acid sequences are set forth in SEQ ID NOs: 1-3, respectively;
(13) the selected VH CDRs 1, 2, 3 amino acid sequences are set forth in SEQ ID NOs: 124-126, respectively, and the selected VL CDRs 1, 2, 3 amino acid sequences are set forth in SEQ ID NOs: 1-3, respectively;
(14) the selected VH CDRs 1, 2, 3 amino acid sequences are set forth in SEQ ID NOs: 127-129, respectively, and the selected VL CDRs 1, 2, 3 amino acid sequences are set forth in SEQ ID NOs: 1-3, respectively;
(15) the selected VH CDRs 1, 2, 3 amino acid sequences are set forth in SEQ ID NOs: 130-132, respectively, and the selected VL CDRs 1, 2, 3 amino acid sequences are set forth in SEQ ID NOs: 1-3, respectively;
(16) the selected VH CDRs 1, 2, 3 amino acid sequences are set forth in SEQ ID NOs: 133-135, respectively, and the selected VL CDRs 1, 2, 3 amino acid sequences are set forth in SEQ ID NOs: 1-3, respectively;
(17) the selected VH CDRs 1, 2, 3 amino acid sequences are set forth in SEQ ID NOs: 136-138, respectively, and the selected VL CDRs 1, 2, 3 amino acid sequences are set forth in SEQ ID NOs: 1-3, respectively;
(18) the selected VH CDRs 1, 2, 3 amino acid sequences are set forth in SEQ ID NOs: 139-141, respectively, and the selected VL CDRs 1, 2, 3 amino acid sequences are set forth in SEQ ID NOs: 1-3, respectively;
(19) the selected VH CDRs 1, 2, 3 amino acid sequences are set forth in SEQ ID NOs: 142-144, respectively, and the selected VL CDRs 1, 2, 3 amino acid sequences are set forth in SEQ ID NOs: 1-3, respectively; and
(20) the selected VH CDRs 1, 2, 3 amino acid sequences are set forth in SEQ ID NOs: 145-147, respectively, and the selected VL CDRs 1, 2, 3 amino acid sequences are set forth in SEQ ID NOs: 1-3, respectively.

In some embodiments, the VH comprises CDRs 1, 2, 3 with the amino acid sequences set forth in SEQ ID NOs: 19-21, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 22-24, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 25-27, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 28-30, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 112-114, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 115-117, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 118-120, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 121-124, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 127-129, respectively, and the VL comprises CDRs 1, 2, 3 with the amino acid sequences set forth in SEQ ID NOs: 1-3, respectively, according to the Kabat numbering scheme. In some embodiments, the VH comprises CDRs 1, 2, 3 with the amino acid sequences set forth in SEQ ID NOs: 49-51, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 52-54, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 55-57, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 58-60, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 130-132, respectively, and the VL comprises CDRs 1, 2, 3 with the amino acid sequences set forth in SEQ ID NOs: 1-3, respectively, according to the Chothia numbering scheme. In some embodiments, the VII comprises CDRs 1, 2, 3 with the amino acid sequences set forth in SEQ ID NOs: 133-135, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 136-138, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 139-141, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 142-1144, respectively, and the VL comprises CDRs 1, 2, 3 with the amino acid sequences set forth in SEQ ID NOs: 1-3, respectively, according to the Chothia numbering scheme. In some embodiments, the VH comprises CDRs 1, 2, 3 with the amino acid sequences set forth in SEQ ID NOs: 145-147, respectively, and the VL comprises CDRs 1, 2, 3 with the amino acid sequences set forth in SEQ ID NOs: 1-3, respectively, according to the Chothia numbering scheme.

In one aspect, the disclosure is related to an antibody or antigen-binding fragment thereof that binds to MET comprising a heavy chain variable region (VH) comprising an amino acid sequence that is at least 90% identical to a selected VH sequence, and a light chain variable region (VL) comprising an amino acid sequence that is at least 90% identical to a selected VL sequence, in some embodiments, the selected VH sequence and the selected VL sequence are one of the following:
(1) the selected VH sequence is SEQ ID NO:67, and the selected VL sequence is SEQ ID NO:61;
(2) the selected VH sequence is SEQ ID NO:68, and the selected VL sequence is SEQ ID NO:61;
(3) the selected VH sequence is SEQ ID NO:69, and the selected VL sequence is SEQ ID NO:61;
(4) the selected VH sequence is SEQ ID NO:70, and the selected VL sequence is SEQ ID NO:61;
(5) the selected VH sequence is SEQ ID NO:148, and the selected VL sequence is SEQ ID NO:61;
(6) the selected VH sequence is SEQ ID NO:149, and the selected VL sequence is SEQ ID NO:61;
(7) the selected VH sequence is SEQ ID NO:150, and the selected VL sequence is SEQ ID NO:61;
(8) the selected VH sequence is SEQ ID NO:151, and the selected VL sequence is SEQ ID NO:61;
(9) the selected VH sequence is SEQ ID NO:152, and the selected VL sequence is SEQ ID NO:61; and
(10) the selected VH sequence is SEQ ID NO:153, and the selected VL sequence is SEQ ID NO:61.

In some embodiments, the VH comprises the sequence of SEQ ID NO: 67 and the VL comprises the sequence of SEQ ID NO: 61. In some embodiments, the VH comprises the sequence of SEQ ID NO: 68 and the VL comprises the sequence of SEQ ID NO: 61. In some embodiments, the VH comprises the sequence of SEQ ID NO: 69 and the VL comprises the sequence of SEQ ID NO: 61. In some embodiments, the VH comprises the sequence of SEQ ID NO: 70 and the VL comprises the sequence of SEQ ID NO: 61. In some embodiments, the VH comprises the sequence of SEQ ID NO: 148 and the VL comprises the sequence of SEQ ID NO: 61. In some embodiments, the VH comprises the sequence of SEQ ID NO:149 and the VL comprises the sequence of SEQ ID NO: 61. In some embodiments, the VH comprises the sequence of SEQ ID NO: 150 and the VL comprises the sequence of SEQ ID NO: 61. In some embodiments, the VH comprises the sequence of SEQ ID NO: 151 and the VL comprises the sequence of SEQ ID NO: 61. In some embodiments, the VH comprises the sequence of SEQ ID NO: 152 and the VL comprises the sequence of SEQ ID NO: 61. In some embodiments, the VH comprises the sequence of SEQ ID NO:153 and the VL comprises the sequence of SEQ ID NO: 61. In some embodiments, the VH comprises the sequence of SEQ ID NO: 70 and the VL comprises the sequence of SEQ ID NO: 61.

In one aspect, the disclosure is related to an antibody or antigen-binding fragment thereof that binds to MET comprising a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, in some embodiments, the selected VH sequence and the selected VL sequence are one of the following:
(1) the selected VH sequence is SEQ ID NO:67, and the selected VL sequence is SEQ ID NO:61;
(2) the selected VH sequence is SEQ ID NO:68, and the selected VL sequence is SEQ ID NO:61;
(3) the selected VH sequence is SEQ ID NO:69, and the selected VL sequence is SEQ ID NO:61;
(4) the selected VH sequence is SEQ ID NO:70, and the selected VL sequence is SEQ ID NO:61;
(5) the selected VH sequence is SEQ ID NO:148, and the selected VL sequence is SEQ ID NO:61;
(6) the selected VH sequence is SEQ ID NO:149, and the selected VL sequence is SEQ ID NO:61;
(7) the selected VH sequence is SEQ ID NO:150, and the selected VL sequence is SEQ ID NO:61;
(8) the selected VH sequence is SEQ ID NO:151, and the selected VL sequence is SEQ ID NO:61;
(9) the selected VH sequence is SEQ ID NO:152, and the selected VL sequence is SEQ ID NO:61; and
(10) the selected VH sequence is SEQ ID NO:153, and the selected VL sequence is SEQ ID NO:61.

In some embodiments, the antibody or antigen-binding fragment thereof specifically binds to human, monkey or dog MET. In some embodiments, the antibody or antigen-binding fragment is a human or humanized antibody or antigen-binding fragment thereof. In some embodiments, the antibody or antigen-binding fragment thereof is a single-chain variable fragment (scFV). In some embodiments, the antibody or antigen-binding fragment thereof is a multispecific antibody (e.g., a bispecific antibody) or an antigen-binding fragment thereof. In some embodiments, the antibody or antigen-binding fragment thereof further specifically binds to EGFR. In one aspect, the disclosure is related to an antibody or antigen-binding fragment thereof that cross-competes with the antibody or antigen-binding fragment thereof as described herein.

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 19-21, respectively, and in some embodiments, the VH, when paired with a light chain variable region (VL) comprising the amino acid sequence set forth in SEQ ID NO: 61, binds to MET;
(2) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and in some embodiments, the VL, when paired with a heavy chain variable region (VH) comprising the amino acid sequence set forth in SEQ ID NO: 67, binds to MET;
(3) an immunoglobulin heavy chain or a fragment thereof comprising a VH comprising CDRs 1, 2, and 3 comprising the amino acid sequences set forth in SEQ ID NOs: 22-24, respectively, and in some embodiments, the VH, when paired with a VL comprising the amino acid sequence set forth in SEQ ID NO: 61, binds to MET;
(4) an immunoglobulin light chain or a fragment thereof comprising a VL comprising CDRs 1, 2, and 3 comprising the amino acid sequences set forth in SEQ ID NOs: 1-3, respectively, and in some embodiments, the VL, when paired with a VH comprising the amino acid sequence set forth in SEQ ID NO: 68, binds to MET;
(5) an immunoglobulin heavy chain or a fragment thereof comprising a VH comprising CDRs 1, 2, and 3 comprising the amino acid sequences set forth in SEQ ID NOs: 25-27, respectively, and in some embodiments, the VH, when paired with a VL comprising the amino acid sequence set forth in SEQ ID NO: 61, binds to MET;
(6) an immunoglobulin light chain or a fragment thereof comprising a VL comprising CDRs 1, 2, and 3 comprising the amino acid sequences set forth in SEQ ID NOs: 1-3, respectively, and in some embodiments, the VL, when paired with a VH comprising the amino acid sequence set forth in SEQ ID NO: 69, binds to MET;
(7) an immunoglobulin heavy chain or a fragment thereof comprising a VH comprising CDRs 1, 2, and 3 comprising the amino acid sequences set forth in SEQ ID NOs: 28-30, respectively, and in some embodiments, the VH, when paired with a VL comprising the amino acid sequence set forth in SEQ ID NO: 61, binds to MET;
(8) an immunoglobulin light chain or a fragment thereof comprising a VL comprising CDRs 1, 2, and 3 comprising the amino acid sequences set forth in SEQ ID NOs: 1-3, respectively, and in some embodiments, the VL, when paired with a VH comprising the amino acid sequence set forth in SEQ ID NO: 70, binds to MET;
(9) an immunoglobulin heavy chain or a fragment thereof comprising a VH comprising CDRs 1, 2, and 3 comprising the amino acid sequences set forth in SEQ ID NOs: 49-51, respectively, and in some embodiments, the VH, when paired with a VL comprising the amino acid sequence set forth in SEQ ID NO: 61, binds to MET;
(10) an immunoglobulin heavy chain or a fragment thereof comprising a VH comprising CDRs 1, 2, and 3 comprising the amino acid sequences set forth in SEQ ID NOs: 52-54, respectively, and in some embodiments, the VH, when paired with a VL comprising the amino acid sequence set forth in SEQ ID NO: 61, binds to MET;
(11) an immunoglobulin heavy chain or a fragment thereof comprising a VH comprising CDRs 1, 2, and 3 comprising the amino acid sequences set forth in SEQ ID NOs: 55-57, respectively, and in some embodiments, the VH, when paired with a VL comprising the amino acid sequence set forth in SEQ ID NO: 61, binds to MET;
(12) an immunoglobulin heavy chain or a fragment thereof comprising a VH comprising CDRs 1, 2, and 3 comprising the amino acid sequences set forth in SEQ ID NOs: 58-60, respectively, and in some embodiments, the VH, when paired with a VL comprising the amino acid sequence set forth in SEQ ID NO: 61, binds to MET;
(13) an immunoglobulin heavy chain or a fragment thereof comprising a VH comprising CDRs 1, 2, and 3 comprising the amino acid sequences set forth in SEQ ID NOs: 112-114, respectively, and wherein the VH, when paired with a VL comprising the amino acid sequence set forth in SEQ ID NO: 61, binds to MET;
(14) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 148, binds to MET;
(15) an immunoglobulin heavy chain or a fragment thereof comprising a VH comprising CDRs 1, 2, and 3 comprising the amino acid sequences set forth in SEQ ID NOs: 115-117, respectively, and wherein the VH, when paired with a VL comprising the amino acid sequence set forth in SEQ ID NO: 61, binds to MET;
(16) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 149, binds to MET;
(17) an immunoglobulin heavy chain or a fragment thereof comprising a VH comprising CDRs 1, 2, and 3 comprising the amino acid sequences set forth in SEQ ID NOs: 118-120, respectively, and wherein the VH, when paired with a VL comprising the amino acid sequence set forth in SEQ ID NO: 61, binds to MET;
(18) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 150, binds to MET;
(19) an immunoglobulin heavy chain or a fragment thereof comprising a VH comprising CDRs 1, 2, and 3 comprising the amino acid sequences set forth in SEQ ID NOs: 121-123, respectively, and wherein the VH, when paired with a VL comprising the amino acid sequence set forth in SEQ ID NO: 61, binds to MET;
(20) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 151, binds to MET;
(21) an immunoglobulin heavy chain or a fragment thereof comprising a VH comprising CDRs 1, 2, and 3 comprising the amino acid sequences set forth in SEQ ID NOs: 124-126, respectively, and wherein the VH, when paired with a VL comprising the amino acid sequence set forth in SEQ ID NO: 61, binds to MET;
(22) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 152, binds to MET;
(23) an immunoglobulin heavy chain or a fragment thereof comprising a VH comprising CDRs 1, 2, and 3 comprising the amino acid sequences set forth in SEQ ID NOs: 127-129, respectively, and wherein the VH, when paired with a VL comprising the amino acid sequence set forth in SEQ ID NO: 61, binds to MET;
(24) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 153, binds to MET;
(25) an immunoglobulin heavy chain or a fragment thereof comprising a VH comprising CDRs 1, 2, and 3 comprising the amino acid sequences set forth in SEQ ID NOs: 130-132, respectively, and wherein the VH, when paired with a VL comprising the amino acid sequence set forth in SEQ ID NO: 61, binds to MET;
(26) an immunoglobulin heavy chain or a fragment thereof comprising a VH comprising CDRs 1, 2, and 3 comprising the amino acid sequences set forth in SEQ ID NOs: 133-135, respectively, and wherein the VH, when paired with a VL comprising the amino acid sequence set forth in SEQ ID NO: 61, binds to MET;
(27) an immunoglobulin heavy chain or a fragment thereof comprising a VH comprising CDRs 1, 2, and 3 comprising the amino acid sequences set forth in SEQ ID NOs: 136-138, respectively, and wherein the VH, when paired with a VL comprising the amino acid sequence set forth in SEQ ID NO: 61, binds to MET;
(28) an immunoglobulin heavy chain or a fragment thereof comprising a VH comprising CDRs 1, 2, and 3 comprising the amino acid sequences set forth in SEQ ID NOs: 139-141, respectively, and wherein the VH, when paired with a VL comprising the amino acid sequence set forth in SEQ ID NO: 61, binds to MET;
(29) an immunoglobulin heavy chain or a fragment thereof comprising a VH comprising CDRs 1, 2, and 3 comprising the amino acid sequences set forth in SEQ ID NOs: 142-144, respectively, and wherein the VH, when paired with a VL comprising the amino acid sequence set forth in SEQ ID NO: 61, binds to MET; and
(30) an immunoglobulin heavy chain or a fragment thereof comprising a VH comprising CDRs 1, 2, and 3 comprising the amino acid sequences set forth in SEQ ID NOs: 145-147, respectively, and wherein the VH, when paired with a VL comprising the amino acid sequence set forth in SEQ ID NO: 61, binds to MET.

In some embodiments, the VH when paired with a VL specifically binds to human, monkey or dog MET. In some embodiments, the immunoglobulin heavy chain or the fragment thereof is a human or humanized immunoglobulin heavy chain or a fragment thereof. In some embodiments, the nucleic acid encodes a single-chain variable fragment (scFv). In some embodiments, the nucleic acid is cDNA.

In one aspect, the disclosure is related to an antigen-binding protein construct, comprising: a first antigen-binding domain that specifically binds to EGFR; and a second antigen-binding domain that specially binds to MET. In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the first heavy chain variable region (VH1) comprises complementarity determining regions (CDRs) 1, 2, and 3, in some embodiments, the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and the first light chain variable region (VL1) comprises CDRs 1, 2, and 3, in some embodiments, the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence, in some embodiments, the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively;
(7) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively;
(8) the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively;
(9) the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively;
(10) the selected VH1 CDRs 1, 2, 3 amino acid sequences are set forth in SEQ ID NOs: 46-48, respectively, and the selected VL1 CDRs 1, 2, 3 amino acid sequences are set forth in SEQ ID NOs: 1-3, respectively;
(11) the selected VH1 CDRs 1, 2, 3 amino acid sequences are set forth in SEQ ID NOs: 82-84, respectively, and the selected VL1 CDRs 1, 2, 3 amino acid sequences are set forth in SEQ ID NOs: 1-3, respectively;
(12) the selected VH1 CDRs 1, 2, 3 amino acid sequences are set forth in SEQ ID NOs: 85-87, respectively, and the selected VL1 CDRs 1, 2, 3 amino acid sequences are set forth in SEQ ID NOs: 1-3, respectively;
(13) the selected VH1 CDRs 1, 2, 3 amino acid sequences are set forth in SEQ ID NOs: 88-90, respectively, and the selected VL1 CDRs 1, 2, 3 amino acid sequences are set forth in SEQ ID NOs: 1-3, respectively;
(14) the selected VH1 CDRs 1, 2, 3 amino acid sequences are set forth in SEQ ID NOs: 91-93, respectively, and the selected VL1 CDRs 1, 2, 3 amino acid sequences are set forth in SEQ ID NOs: 1-3, respectively;
(15) the selected VH1 CDRs 1, 2, 3 amino acid sequences are set forth in SEQ ID NOs: 94-96, respectively, and the selected VL1 CDRs 1, 2, 3 amino acid sequences are set forth in SEQ ID NOs: 1-3, respectively;
(16) the selected VH1 CDRs 1, 2, 3 amino acid sequences are set forth in SEQ ID NOs: 97-99, respectively, and the selected VL1 CDRs 1, 2, 3 amino acid sequences are set forth in SEQ ID NOs: 1-3, respectively;
(17) the selected VH1 CDRs 1, 2, 3 amino acid sequences are set forth in SEQ ID NOs: 100-102, respectively, and the selected VL1 CDRs 1, 2, 3 amino acid sequences are set forth in SEQ ID NOs: 1-3, respectively; and
(18) the selected VH1 CDRs 1, 2, 3 amino acid sequences are set forth in SEQ ID NOs: 103-105, respectively, and the selected VL1 CDRs 1, 2, 3 amino acid sequences are set forth in SEQ ID NOs: 1-3, respectively.

In some embodiments, the second heavy chain variable region (VH2) comprises CDRs 1, 2, and 3, in some embodiments, the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and the second light chain variable region (VL2) comprises CDRs 1, 2, and 3, in some embodiments, the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence, in some embodiments, the selected VH2 CDRS 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(2) the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(3) the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(4) the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(5) the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(6) the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(7) the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(8) the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(9) the selected VH2 CDRs 1, 2, 3 amino acid sequences are set forth in SEQ ID NOs: 112-114, respectively, and the selected VL2 CDRs 1, 2, 3 amino acid sequences are set forth in SEQ ID NOs: 1-3, respectively;
(10) the selected VH2 CDRs 1, 2, 3 amino acid sequences are set forth in SEQ ID NOs: 115-117, respectively, and the selected VL2 CDRs 1, 2, 3 amino acid sequences are set forth in SEQ ID NOs: 1-3, respectively;
(11) the selected VH2 CDRs 1, 2, 3 amino acid sequences are set forth in SEQ ID NOs: 118-120, respectively, and the selected VL2 CDRs 1, 2, 3 amino acid sequences are set forth in SEQ ID NOs: 1-3, respectively;
(12) the selected VH2 CDRs 1, 2, 3 amino acid sequences are set forth in SEQ ID NOs: 121-123, respectively, and the selected VL2 CDRs 1, 2, 3 amino acid sequences are set forth in SEQ ID NOs: 1-3, respectively;
(13) the selected VH2 CDRs 1, 2, 3 amino acid sequences are set forth in SEQ ID NOs: 124-126, respectively, and the selected VL2 CDRs 1, 2, 3 amino acid sequences are set forth in SEQ ID NOs: 1-3, respectively;
(14) the selected VH2 CDRs 1, 2, 3 amino acid sequences are set forth in SEQ ID NOs: 127-129, respectively, and the selected VL2 CDRs 1, 2, 3 amino acid sequences are set forth in SEQ ID NOs: 1-3, respectively;
(15) the selected VH2 CDRs 1, 2, 3 amino acid sequences are set forth in SEQ ID NOs: 130-132, respectively, and the selected VL2 CDRs 1, 2, 3 amino acid sequences are set forth in SEQ ID NOs: 1-3, respectively;
(16) the selected VH2 CDRs 1, 2, 3 amino acid sequences are set forth in SEQ ID NOs: 133-135, respectively, and the selected VL2 CDRs 1, 2, 3 amino acid sequences are set forth in SEQ ID NOs: 1-3, respectively;
(17) the selected VH2 CDRs 1, 2, 3 amino acid sequences are set forth in SEQ ID NOs: 136-138, respectively, and the selected VL2 CDRs 1, 2, 3 amino acid sequences are set forth in SEQ ID NOs: 1-3, respectively;
(18) the selected VH2 CDRs 1, 2, 3 amino acid sequences are set forth in SEQ ID NOs: 139-141, respectively, and the selected VL2 CDRs 1, 2, 3 amino acid sequences are set forth in SEQ ID NOs: 1-3, respectively;
(19) the selected VH2 CDRs 1, 2, 3 amino acid sequences are set forth in SEQ ID NOs: 142-144, respectively, and the selected VL2 CDRs 1, 2, 3 amino acid sequences are set forth in SEQ ID NOs: 1-3, respectively; and
(20) the selected VH2 CDRs 1, 2, 3 amino acid sequences are set forth in SEQ ID NOs: 145-147, respectively, and the selected VL2 CDRs 1, 2, 3 amino acid sequences are set forth in SEQ ID NOs: 1-3, respectively.

In some embodiments, the antigen-binding protein construct as described herein has one of the following features:
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(7) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(8) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(9) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(10) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(11) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(12) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(13) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(14) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(15) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(16) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(17) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(18) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(19) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(20) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(21) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(22) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(23) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(24) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(25) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(26) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(27) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 31-33, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(28) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(29) the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(30) the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(31) the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(32) the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(33) the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(34) the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(35) the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(36) the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(37) the selected VH1 CDRs 1, 2, 3 amino acid sequences are set forth in SEQ ID NOs: 46-48, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(38) the selected VH1 CDRs 1, 2, 3 amino acid sequences are set forth in SEQ ID NOs: 46-48, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(39) the selected VH1 CDRs 1, 2, 3 amino acid sequences are set forth in SEQ ID NOs: 46-48, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(40) the selected VH1 CDRs 1, 2, 3 amino acid sequences are set forth in SEQ ID NOs: 46-48, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(41) the selected VH1 CDRs 1, 2, 3 amino acid sequences are set forth in SEQ ID NOs: 82-84, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(42) the selected VH1 CDRs 1, 2, 3 amino acid sequences are set forth in SEQ ID NOs: 82-84, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(43) the selected VH1 CDRs 1, 2, 3 amino acid sequences are set forth in SEQ ID NOs: 82-84, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(44) the selected VH1 CDRs 1, 2, 3 amino acid sequences are set forth in SEQ ID NOs: 82-84, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(45) the selected VH1 CDRs 1, 2, 3 amino acid sequences are set forth in SEQ ID NOs: 85-87, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(46) the selected VH1 CDRs 1, 2, 3 amino acid sequences are set forth in SEQ ID NOs: 85-87, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(47) the selected VH1 CDRs 1, 2, 3 amino acid sequences are set forth in SEQ ID NOs: 85-87, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(48) the selected VH1 CDRs 1, 2, 3 amino acid sequences are set forth in SEQ ID NOs: 85-87, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(49) the selected VH1 CDRs 1, 2, 3 amino acid sequences are set forth in SEQ ID NOs: 88-90, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(50) the selected VH1 CDRs 1, 2, 3 amino acid sequences are set forth in SEQ ID NOs: 88-90, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(51) the selected VH1 CDRs 1, 2, 3 amino acid sequences are set forth in SEQ ID NOs: 88-90, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(52) the selected VH1 CDRs 1, 2, 3 amino acid sequences are set forth in SEQ ID NOs: 88-90, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(53) the selected VH1 CDRs 1, 2, 3 amino acid sequences are set forth in SEQ ID NOs: 91-93, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(54) the selected VH1 CDRs 1, 2, 3 amino acid sequences are set forth in SEQ ID NOs: 91-93, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(55) the selected VH1 CDRs 1, 2, 3 amino acid sequences are set forth in SEQ ID NOs: 91-93, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(56) the selected VH1 CDRs 1, 2, 3 amino acid sequences are set forth in SEQ ID NOs: 91-93, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(57) the selected VH1 CDRs 1, 2, 3 amino acid sequences are set forth in SEQ ID NOs: 94-96, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(58) the selected VH1 CDRs 1, 2, 3 amino acid sequences are set forth in SEQ ID NOs: 94-96, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(59) the selected VH1 CDRs 1, 2, 3 amino acid sequences are set forth in SEQ ID NOs: 94-96, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(60) the selected VH1 CDRs 1, 2, 3 amino acid sequences are set forth in SEQ ID NOs: 94-96, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(61) the selected VH1 CDRs 1, 2, 3 amino acid sequences are set forth in SEQ ID NOs: 97-99, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(62) the selected VH1 CDRs 1, 2, 3 amino acid sequences are set forth in SEQ ID NOs: 97-99, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(63) the selected VH1 CDRs 1, 2, 3 amino acid sequences are set forth in SEQ ID NOs: 97-99, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(64) the selected VH1 CDRs 1, 2, 3 amino acid sequences are set forth in SEQ ID NOs: 97-99, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRS 1, 2, 3 amino acid sequences are set forth in SEQ ID NOs: 1-3, respectively;
(65) the selected VH1 CDRs 1, 2, 3 amino acid sequences are set forth in SEQ ID NOs: 100-102, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(66) the selected VH1 CDRs 1, 2, 3 amino acid sequences are set forth in SEQ ID NOs: 100-102, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(67) the selected VH1 CDRs 1, 2, 3 amino acid sequences are set forth in SEQ ID NOs: 100-102, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(68) the selected VH1 CDRs 1, 2, 3 amino acid sequences are set forth in SEQ ID NOs: 100-102, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(69) the selected VH1 CDRs 1, 2, 3 amino acid sequences are set forth in SEQ ID NOs: 103-105, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(70) the selected VH1 CDRs 1, 2, 3 amino acid sequences are set forth in SEQ ID NOs: 103-105, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(71) the selected VH1 CDRs 1, 2, 3 amino acid sequences are set forth in SEQ ID NOs: 103-105, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively; and
(72) the selected VH1 CDRs 1, 2, 3 amino acid sequences are set forth in SEQ ID NOs: 103-105, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively.

In some embodiments, the first heavy chain variable region comprises a sequence that is at least 80%, 85%, 90%, or 95% identical to SEQ ID NO: 62, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 67, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 62, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 68, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 62, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 69, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 62, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 70, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 63, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 67, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 63, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 68, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 63, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 69, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 63, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 70, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 64, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 67, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 64, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 68, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 64, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 69, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 64, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 70, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 65, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 67, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 65, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 68, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 65, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 69, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 65, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 70, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 66, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 67, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 66, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 68, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 66, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 69, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 66, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 70, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 106, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 67, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 106, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 68, and the second light chain variable region comprises a sequence that is at least 80%, 85%, 90%, or 95% identical to SEQ ID NO: 61. In some embodiments, the first heavy chain variable region comprises a sequence that is at least 80%, 85%, 90%, or 95% identical to SEQ ID NO: 106, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 69, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 106, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 70, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 107, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 67, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 107, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 68, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 107, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 69, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 107, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 70, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 108, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 67, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 108, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 68, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 108, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 69, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 108, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 70, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 109, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 67, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 109, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 68, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 109, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 69, and the second light chain variable region comprises a sequence that is at least 80%, 85%, 90%, or 95% identical to SEQ ID NO: 61.

In some embodiments, the first heavy chain variable region comprises a sequence that is at least 80%, 85%, 90%, or 95% identical to SEQ ID NO: 109, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 70, and the second light chain variable region comprises a sequence that is at least 80%, 85%, 90%, or 95% identical to SEQ ID NO: 61.

In some embodiments, the antigen-binding protein construct is a multi-specific antibody (e.g., a bispecific antibody). In some embodiments, the first antigen-binding domain is a single-chain variable fragment (scFV); and/or the second antigen-binding domain is a scFv. In some embodiments, the first light chain variable region and the second light chain variable region are identical.

In one aspect, the disclosure is related to a vector comprising one or more of the nucleic acids as described herein, a nucleic acid encoding the antibody or antigen-binding fragment thereof as described herein, or a nucleic acid encoding the antigen-binding protein construct as described herein.

In one aspect, the disclosure is related to a cell comprising the vector as described herein. In some embodiments, the cell is a CHO cell.

In one aspect, the disclosure is related to a cell comprising one or more of the nucleic acids as described herein, a nucleic acid encoding the antibody or antigen-binding fragment thereof as described herein, or a nucleic acid encoding the antigen-binding protein construct as described herein.

In one aspect, the disclosure is related to a method of producing an antibody or antigen-binding fragment thereof, or an antigen-binding protein construct, the method comprising (a) culturing the cell as described herein under conditions sufficient for the cell to produce the antibody or the antigen-binding fragment thereof, or the antigen-binding protein construct; and (b) collecting the antibody or the antigen-binding fragment thereof, or the antigen-binding protein construct produced by the cell.

In one aspect, the disclosure is related to an antibody-drug conjugate (ADC) comprising a therapeutic agent covalently bound to: (a) the antibody or antigen-binding fragment thereof as described herein; or (b) the antigen-binding protein construct as described herein. In some embodiments, the therapeutic agent is a cytotoxic or cytostatic agent. In some embodiments, the therapeutic agent is MMAE or MMAF.

In some embodiments, the therapeutic agent is selected from

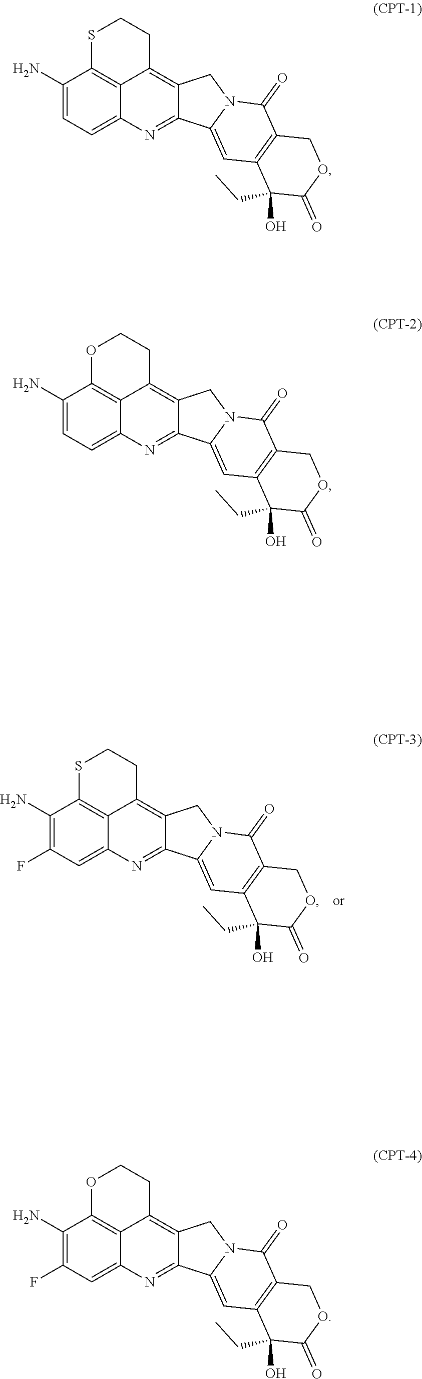 [SMILES: CC[C@@]1(O)C(=O)OCc2c1cc1n(c2=O)Cc2c-1nc1ccc(N)c3c1c2CCS3 ; CC[C@@]1(O)C(=O)OCc2c1cc1n(c2=O)Cc2c-1nc1ccc(N)c3c1c2CCO3 ; CC[C@@]1(O)C(=O)OCc2c1cc1n(c2=O)Cc2c-1nc1cc(F)c(N)c3c1c2CCS3 ; CC[C@@]1(O)C(=O)OCc2c1cc1n(c2=O)Cc2c-1nc1cc(F)c(N)c3c1c2CCO3]

In some embodiments, the therapeutic agent is linked to the antibody or antigen-binding fragment thereof, or the antigen-binding protein construct via a linker. In some embodiments, the linker has a structure of:

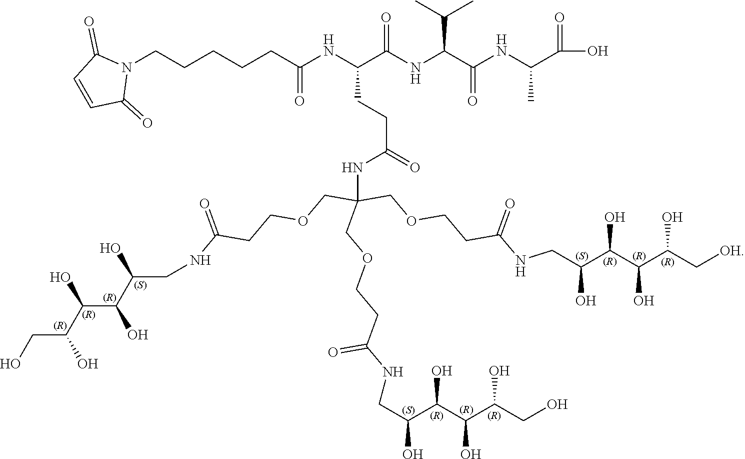

In some embodiments, the antibody-drug conjugate has a structure of:

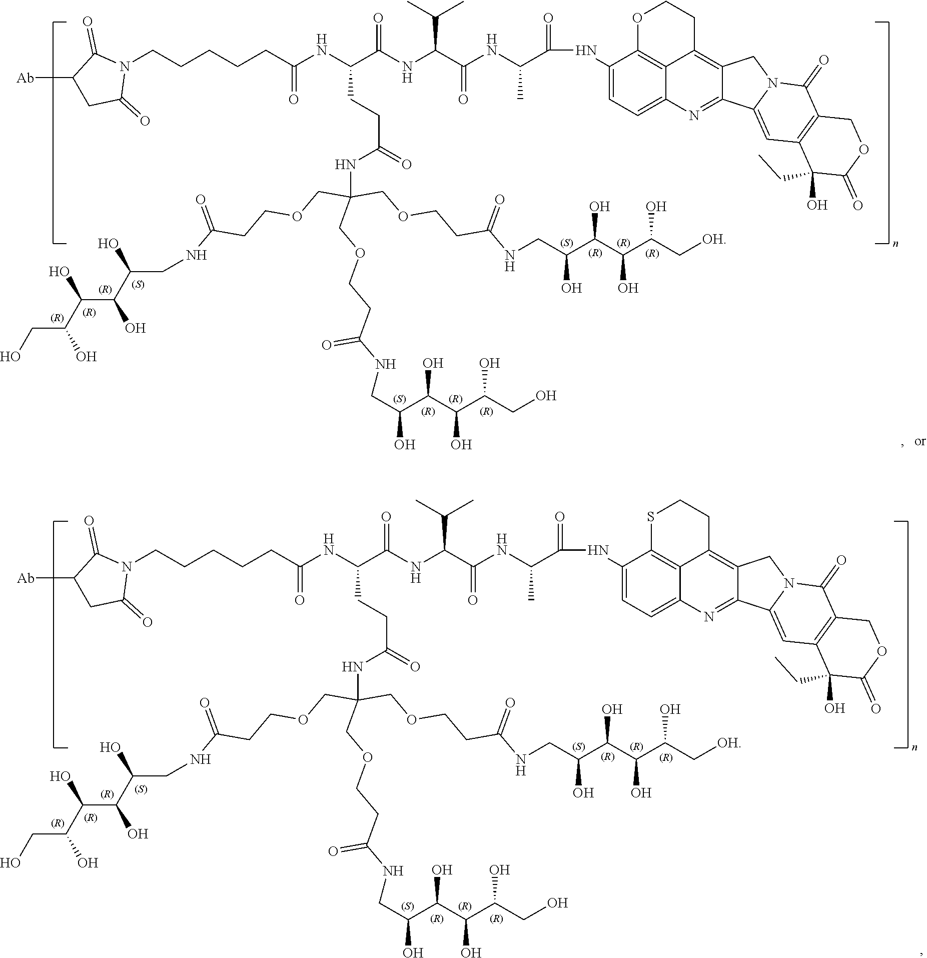

in some embodiments, n=1, 2, 3, 4, 5, 6, 7, or 8; in some embodiments, “Ab” represents the antibody or antigen-binding fragment thereof, or the antigen-binding protein construct described herein.

In one aspect, the disclosure is related to a method of treating a subject having cancer, the method comprising administering a therapeutically effective amount of a composition comprising the antibody or antigen-binding fragment thereof as described herein, the antigen-binding protein construct as described herein, or the antibody-drug conjugate as described herein, to the subject. In some embodiments, the subject has a cancer expressing EGFR and/or MET. In some embodiments, the cancer is a solid tumor, lung cancer (e.g., non-small cell lung cancer, lung adenocarcinoma, or lung carcinoma), gastric cancer (e.g., gastric carcinoma), skin cancer (e.g., skin carcinoma), colorectal cancer, breast cancer, head and neck cancer, ovarian cancer, prostate cancer, thyroid cancer, pancreatic cancer, CNS cancer, liver cancer, nasopharynx cancer, or brain cancer. In some embodiments, the subject is a human. In some embodiments, the method further comprises administering an anti-PD1 antibody to the subject. In some embodiments, the method further comprises administering a chemotherapy to the subject.

In one aspect, the disclosure is related to a method of decreasing the rate of tumor growth, the method comprising contacting a tumor cell with an effective amount of a composition comprising the antibody or antigen-binding fragment thereof as described herein, the antigen-binding protein construct as described herein, or the antibody-drug conjugate as described herein.

In one aspect, the disclosure is related to a method of killing a tumor cell, the method comprising contacting a tumor cell with an effective amount of a composition comprising the antibody or antigen-binding fragment thereof as described herein, the antigen-binding protein construct as described herein, or the antibody-drug conjugate as described herein.

In one aspect, the disclosure is related to a pharmaceutical composition comprising a pharmaceutically acceptable carrier and (a) the antibody or antigen-binding fragment thereof as described herein, (b) the antigen-binding protein construct as described herein, and/or (c) the antibody-drug conjugate as described herein.

In one aspect, the disclosure is related to an antibody-drug conjugate (ADC) comprising a therapeutic agent covalently bound to a bispecific antibody or antigen-binding fragment thereof comprising: a first antigen-binding domain that specifically binds to EGFR; and a second antigen-binding domain that specially binds to MET. In some embodiments, the drug-to-antibody ratio (DAR) is about 4 or 8.

In one aspect, the disclosure provides a nucleic acid comprising a polynucleotide encoding any polypeptide as described herein. In some embodiments, the nucleic acid encodes a bispecific antibody. In some embodiments, the nucleic acid is cDNA.

In one aspect, the disclosure provides a vector comprising one or more of the nucleic acids described herein.

In one aspect, the disclosure provides a cell comprising the vector described herein. In some embodiments, the cell is a CHO cell. In one aspect, the disclosure provides a cell comprising one or more of the nucleic acids described herein.

As used herein, the term “antigen-binding protein construct” is (i) a single polypeptide that includes at least two different antigen-binding domains or (ii) a complex of two or more polypeptides (e.g., the same or different polypeptides) that together form at least two different antigen-binding domains. Non-limiting examples and aspects of antigen-binding protein constructs are described herein. Additional examples and aspects of antigen-binding protein constructs are known in the art.

As used herein, the term “antigen-binding domain” refers to one or more protein domain(s) (e.g., formed from amino acids from a single polypeptide or formed from amino acids from two or more polypeptides (e.g., the same or different polypeptides) that is capable of specifically binding to one or more different antigen(s) (e.g., an effector antigen or control antigen). In some examples, an antigen-binding domain can bind to an antigen or epitope with specificity and affinity similar to that of naturally-occurring antibodies. In some embodiments, the antigen-binding domain can be an antibody or a fragment thereof. One example of an antigen-binding domain is an antigen-binding domain formed by a VH-VL dimer. In some embodiments, an antigen-binding domain can include an alternative scaffold. In some embodiments, the antigen-binding domain is a VHH. Non-limiting examples of antigen-binding domains are described herein. Additional examples of antigen-binding domains are known in the art. In some examples, an antigen-binding domain can bind to a single antigen (e.g., one of an effector antigen and a control antigen). In other examples, an antigen-binding domain can bind to two different antigens (e.g., an effector antigen and a control antigen).

The term “antibody” is used herein in its broadest sense and includes certain types of immunoglobulin molecules that include one or more antigen-binding domains that specifically bind to an antigen or epitope. An antibody specifically includes, e.g., intact antibodies (e.g., intact immunoglobulins), antibody fragments, bispecific antibodies, and multi-specific antibodies. One example of an antibody is a protein complex that includes two heavy chains and two light chains. Additional examples of an antibody are described herein.

As used herein, the term “multispecific antigen-binding protein construct” is an antigen-binding protein construct that includes two or more different antigen-binding domains that collectively specifically bind two or more different epitopes. The two or more different epitopes may be epitopes on the same antigen (e.g., a single polypeptide present on the surface of a cell) or on different antigens (e.g., different proteins present on the surface of the same cell or present on the surface of different cells). In some aspects, a multi-specific antigen-binding protein construct binds two different epitopes (i.e., a “bispecific antigen-binding protein construct”). In some aspects, a multi-specific antigen-binding protein construct binds three different epitopes (i.e., a “trispecific antigen-binding protein construct”). In some aspects, a multi-specific antigen-binding protein construct binds four different epitopes (i.e., a “quadspecific antigen-binding protein construct”). In some aspects, a multi-specific antigen-binding protein construct binds five different epitopes (i.e., a “quintspecific antigen-binding protein construct”). Each binding specificity may be present in any suitable valency. Non-limiting examples of multispecific antigen-binding protein constructs are described herein.

As used herein, the term “bispecific antibody” refers to an antibody that binds to two different epitopes. The epitopes can be on the same antigen or on different antigens.

As used herein, the term “common light chain” refers to a light chain that can interact with two or more different heavy chains, forming different antigen-binding sites, wherein these different antigen-binding sites can specifically bind to different antigens or epitopes. Similarly, the term “common light chain variable region” refers to a light chain variable region that can interact with two or more different heavy chain variable regions, forming different antigen-binding sites, wherein these different antigen-binding sites can specifically bind to different antigens or epitopes. In some embodiments, the antigen-binding construct can have a common light chain. In some embodiments, the antigen-binding construct can have a common light chain variable region.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram showing a bispecific antibody having the knobs-into-holes structure with common light chain.

FIG. 2 shows the cancer cell killing activity of E-6C4-M-2F11 and E-6C4-M-2F11-ADC at 10 μg/mL for 72 hours. ISO-ADC is a human IgG1 isotype control coupled with MMAE. Amivantamab analog is a bispecific antibody control for E-6C4-M-2F11. Medium is a blank control.

FIG. 3 shows the average tumor volume in different groups of B-NDG mice that were injected with NCI-H1975 cells, and were treated with 10 mg/kg E-6C4-M-2F11-ADC (G2), 3 mg/kg E-6C4-M-2F11-ADC (G3), 10 mg/kg E-6C4-M-2F11 (G4), or 3 mg/kg E-6C4-M-2F11 (G5). PBS was injected as a control (G1).

FIG. 4 shows the average tumor volume in different groups of B-NDG mice that were injected with NCI-H292 cells, and were treated with 10 mg/kg E-6C4-M-2F11-ADC (G2), 3 mg/kg E-6C4-M-2F11-ADC (G3), 1 mg/kg E-6C4-M-2F11-ADC (G4), 10 mg/kg Amivantamab analog (G5), 3 mg/kg Amivantamab analog (G6), or 1 mg/kg Amivantamab analog (G7). PBS was injected as a control (G1).

FIG. 5 shows the average tumor volume in different groups of B-NDG mice that were injected with NCI-H292 cells, and were treated with ISO-ADC (G2), E-6C4-M-2F11-ADC (G3), E-6C4-M-2G10-ADC (G4), E-6C4-ADC (G5), M-2G10-ADC (G6), M-2F11-ADC (G7), CD28-E-6C4-ADC (G8), CD28-M-2F11-ADC (G9), or M-2G10-CD28-ADC (G10) at dose level of 3 mg/kg. PBS was injected as a control (GI).

FIG. 6 shows the average tumor volume in different groups of B-NDG mice that were injected with SNU-5 cells, and were treated with 10 mg/kg Amivantamab analog (G2), 3 mg/kg Amivantamab analog (G3), 10 mg/kg E-6C4-M-2F11-ADC (G4), 3 mg/kg E-6C4-M-2F11-ADC (G5), 10 mg/kg E-6C4-M-2G10-ADC (G6), 3 mg/kg E-6C4-M-2G10-ADC (G7), 10 mg/kg E-6C4-M-2F11 (G8), 3 mg/kg E-6C4-M-2F11 (G9), 10 mg/kg E-6C4-M-2G10 (G10), or 3 mg/kg E-6C4-M-2G10 (G11). PBS was injected as a control (G1).

FIG. 7 shows the average tumor volume in different groups of B-NDG mice that were injected with A431 cells, and were treated with ISO-ADC (G2), E-6C4-M-2F11-ADC (G3), E-6C4-M-2F11 (G4), or Amivantamab analog (G5) at a dose level of 3 mg/kg. PBS was injected as a control (G1).

FIG. 8 lists heavy chain variable region CDR sequences of anti-EGFR antibodies E-1G11, E-6C4, E-2B7, E-3A8, E-3D8, E-9A6, E-9D2, E-9F3, E-9H2 and anti-MET antibodies M-2F11, M-2G10, M-1E9, M-2F12 as defined by Kabat numbering.

FIG. 9 lists heavy chain variable region CDR sequences of anti-EGFR antibodies E-1G11, E-6C4, E-2B7, E-3A8, E-3D8, E-9A6, E-9D2, E-9F3, E-9H2 and anti-MET antibodies M-2F11, M-2G10, M-1E9, M-2F12 as defined by Chothia numbering.

FIG. 10 lists CDR sequences for the common light chain as defined by Kabat and Chothia numbering.

FIG. 11 lists antibody heavy chain and light chain variable region sequences discussed in the disclosure.

FIG. 12 lists additional amino acid sequences discussed in the disclosure.

FIG. 13 shows the average tumor volume in different groups of B-NDG mice that were injected with NCI-H1975 cells, and were treated with M-2F11-ADC (G2 and G4), E-6C4-ADC (G3 and G5), E-6C4-M-2F11 (G9 and G10), E-6C4-M-2F11-ADC (G11 and G12) or combination of E-6C4-ADC and M-2F11-ADC (G6, G7 and G8). PBS was injected as a control (G1).

FIG. 14 shows the average tumor volume in different groups of B-NDG mice that were injected with NCI-H1975 cells, and were treated with 3 mg/kg Cetuximab analog-ADC (G2), 3 mg/kg E-9A6-ADC (G3), 3 mg/kg E-9F3-ADC (G4), or 3 mg/kg E-9D2-ADC (G5). PBS was injected as a control (G1).

FIG. 15 shows the average tumor volume in different groups of Balb/c nude mice that were injected with NCI-H292 cells, and were treated with E-6C4-M-2F11-ADC (G2-G4), Telisotuzumab analog-ADC (GS), MRG003 analog-ADC (G6), or Amivantamab (G7). PBS was injected as a control (GI).

FIG. 16 shows the average tumor volume in different groups of B-NDG mice that were engrafted with patient-derived ampullary tumor fragments, and were treated with E-6C4-M-2F11-ADC (G2-G4), E-6C4-ADC (GS), Cetuximab analog-ADC (G6-G7), or Amivantamab (G8). PBS was injected as a control (G1).

FIG. 17 shows the average tumor volume in different groups of B-NDG mice that were engrafted with patient-derived gastric tumor fragments, and were treated with E-6C4-M-2F11-ADC (G2-G4), or Amivantamab (G5). PBS was injected as a control (G1).

FIG. 18A shows the average tumor volume in different groups of B-NDG mice that were injected with NUGC-4 cells, and were treated with anti-MET antibody ADCs (G3-G9). PBS (G1) or ISO-ADC (G2) was injected as a control.

FIG. 18B is a partially enlarged view of FIG. 18A, which shows the average tumor volume in different groups of B-NDG mice that were injected with NUGC-4 cells, and were treated with anti-MET antibody ADCs (G3-G9).

FIG. 19 shows exemplary anti-EGFR/MET bispecific antibodies with antigen binding arms derived from different antibodies.

### DETAILED DESCRIPTION

A bispecific antibody or antigen-binding fragment thereof is an artificial protein that can simultaneously bind to two different epitopes (e.g., on two different antigens). In some embodiments, a bispecific antibody or antigen-binding fragment thereof can have two arms. Each arm can have one heavy chain variable region and one light chain variable region, forming an antigen-binding domain (or an antigen-binding region). In some embodiments, the bispecific antibody has a common light chain.

The present disclosure relates to anti-EGFR antibodies or antigen binding fragments thereof, anti-MET antibodies or antigen binding fragments thereof, antigen-binding protein constructs (e.g., bispecific antibodies or antigen-binding fragments thereof) that specifically bind to two different antigens (e.g., EGFR and MET), and antibody drug conjugates.

### Anti-EGFR/MET Antigen-Binding Protein Construct

Epidermal growth factor receptor (EGFR, ErbBI or HER1) is a Type 1 transmembrane glycoprotein of 170 kDa that is encoded by the c-erbB1 proto-oncogene. The epidermal growth factor receptor is a member of the ErbB family of receptors, a subfamily of four closely related receptor tyrosine kinases: EGFR (ErbB-1), HER2/neu (ErbB-2), Her3 (ErbB-3) and Her4 (ErbB-4). In many cancer types, mutations affecting EGFR expression or activity could result in cancer. EGFR signaling is initiated by ligand binding followed by induction of conformational change, homodimerization or heterodimerization of the receptor with other ErbB family members, and trans-autophosphorylation of the receptor, which initiates signal transduction cascades that ultimately affect a wide variety of cellular functions, including cell proliferation and survival, increases in expression or kinase activity of EGFR have been linked with a range of human cancers, making EGFR an attractive target for therapeutic intervention. Increases in both the EGFR gene copy number and protein expression have been associated with favorable responses to the EGFR tyrosine kinase inhibitor, IRESSA™ (gefitinib), in non-small cell lung cancer.

MET, also called c-Met, tyrosine-protein kinase Met, or hepatocyte growth factor receptor (HGFR), is a protein that in humans is encoded by the MET gene. The protein possesses tyrosine kinase activity. The primary single chain precursor protein is post-translationally cleaved to produce the alpha and beta subunits, which are disulfide linked to form the mature receptor. Activation of MET by its ligand hepatocyte growth factor (HGF) stimulates a plethora of cell processes including growth, motility, invasion, metastasis, epithelial-rnesenchynial transition, angiogenesis/wound healing, and tissue regeneration. The exact stoichiometry of HGF:MET binding is unclear, but it is generally believed that two HGF molecules bind to two MET molecules leading to receptor dimerization and autophosphorylation at tyrosines 1230, 1234, and 1235. Ligand-independent MET autophospliorylation can also occur due to gene amplification, mutation or receptor over-expression.

MET is frequently amplified, mutated or over-expressed in many types of cancer including gastric, lung, colon, breast, bladder, head and neck, ovarian, prostate, thyroid, pancreatic, and CNS cancers. Missense mutations typically localized to the kinase domain are commonly found in hereditary papillary renal cell carcinomas (PRCC) and in 13% of sporadic PRCCs (Schmidt et al, Oncogene 18: 2343-2350, 1999), MET mutations localized to the semaphorin or juxtamembrane domains of MET are frequently found in gastric, head and neck, liver, ovarian, NSCLC and thyroid cancers. MET amplification has been detected in brain, colorectal, gastric, and lung cancers, often correlating with disease progression. Up to 4% and 20% of non-small cell lung cancer (NSCLC) and gastric cancers, respectively, exhibit MET amplification. MET overexpression is also frequently observed in lung cancer. Moreover, in clinical samples, nearly half of lung adenocarcinomas exhibited high levels of MET and HGF, both of which correlated with enhanced tumor growth rate, metastasis and poor prognosis.

Nearly 60% of all tumors that become resistant to EGF tyrosine kinase inhibitors increase MET expression, amplify MET, or increase MET only known ligand, HGF, suggesting the existence of a compensatory pathway for EGFR through MET. MET amplification was first identified in cultured cells that became resistant to gefitinib, an EGFR kinase inhibitor, and exhibited enhanced survival through the Her3 pathway. This was further validated in clinical samples where nine of 43 patients with acquired resistance to either erlotinib or gefitinib exhibited MET amplification.

Aberrant MET signaling has been implicated in the development/progression of many human cancers. This results from the overexpression of MET, activating mutations in MET, transactivation, autocrine or paracrine signaling, or MET gene amplification. A significant relationship between EGFR and MET signaling was recognized through the studies on cancer therapy outcomes. MET is a critical player in developing resistance to targeted therapies, including therapies directed at EGFR. Similarly, EGFR and downstream gene mutations such as KRAS, histologic transformation, and the activation of alternative pathways, which includes the MET signaling pathway, have been identified as mechanisms of resistance to EGFR-targeted therapies. Consequently, blocking one receptor tends to upregulate the other, leading to resistance to single-agent treatment. Amplification of MET and/or high levels of HGF ligand expression have been observed in NSCLC patients with intrinsic or acquired resistance to tyrosine kinase inhibitors of EGFR, including erlotinib and gefitinib. Conversely, MET-amplified lung cancer cells exposed to MET-inhibiting agents for a prolonged period develop resistance via the EGFR pathway. Because of the signaling crosstalk between EGFR and Met, inhibition of both receptors in combination may lead to improved outcomes for patients with MET- and EGFR-driven cancers. Additionally, concurrent inhibition may overcome or delay therapeutic resistance compared to the blockade of just one pathway.

Binding of a ligand such as EGF to EGFR stimulates receptor dimerization, autophosphorylation, activation of the receptor's internal, cytoplasmic tyrosine kinase domain, and initiation of multiple signal transduction and transactivation pathways involved in regulation of DNA synthesis (gene activation) and cell cycle progression or division. Inhibition of EGFR signaling may result in inhibition in one or more EGFR. In some embodiments, the EGFR ligands include EGF, TGFα, heparin binding EGF (HB-EGF), amphiregulin (AR), and epiregulm (EPI).

Binding of HGF to MET stimulates receptor dimerization, autophosphorylation, activation of the receptor's internal, cytoplasmic tyrosine kinase domain, and initiation of multiple signal transduction and transactivation pathways involved in regulation of DNA synthesis (gene activation) and cell cycle progression or division, inhibition of MET signaling may result in inhibition of one or more MET downstream signaling pathways and therefore neutralizing MET may have various effects, including inhibition of cell proliferation and differentiation, angiogenesis, cell motility and metastasis.

The roles of EGFR and MET in cancer are described, e.g., WO2014081954A1, WO2008/127710, WO2009/111691, WO2009/126834, WO2010/039248, WO2010/115551 and US2009/0042906; Engelman et al. “MET amplification leads to gefitinib resistance in lung cancer by activating ERBB3 signaling.” science 316.5827 (2007): 1039-1043; Bean et al. “MET amplification occurs with or without T790M mutations in EGFR mutant lung tumors with acquired resistance to gefitinib or erlotinib.” Proceedings of the National Academy of Sciences 104.52 (2007): 20932-20937, which are incorporated herein by reference in the entirety.

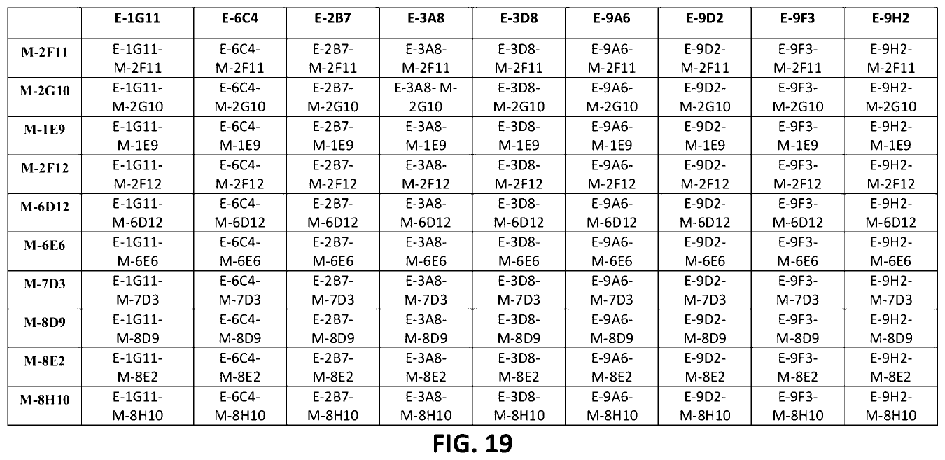

The anti-EGFR antibodies (e.g., E-1C12 (“1C12”), E-1G11 (“1G1”), E-6C4 (“6C4”), E-2B7 (“2B7”), E-3A8 (“3A8”), E-3D8 (“3D8”), E-9A6 (“9A6”), E-9D2 (“9D2”), E-9F3 (“9F3”) and E-9H2 (“9H2”)) and anti-MET antibodies (e.g., M-1C5 (“1C5”), M-2F11 (“2F11”), M-2G10 (“2G10”), M-1E9 (“1E9”), M-2F12 (“2F12”), M-6D12 (“6D12”), M-6E6 (“6E6”), M-7D3 (“7D3”), M-8D9 (“8D9”), M-8E2 (“8E2”), M-8H10 (“8H10”)) in the present disclosure are human antibodies produced in RenLite® mice. Because these antibodies have an identical fully-humanized common light chain, anti-EGFR/MET bispecific antibodies were generated having a heavy chain variable region targeting EGFR (e.g., any one of the VH targeting EGFR described herein), a heavy chain variable region targeting MET (e.g., any one of the VH targeting MET described herein), and two identical common light chain variable regions. Exemplary bispecific antibodies obtained are shown in FIG. 19. In some embodiments, the anti-EGFR antigen-binding domain comprising the CDRs of an anti-EGFR antibody as indicated in the first row of FIG. 19. In some embodiments, the anti-MET antigen-binding domain comprising the CDRs of an anti-MET antibody as indicated in the first column of FIG. 19. In some embodiments, the anti-EGFR antigen-binding domain comprises the VH and VL of an anti-EGFR antibody as indicated in the first row of FIG. 19. In some embodiments, the anti-MET antigen-binding domain comprises the VH and VL of an anti-MET antibody as indicated in the first column of FIG. 19. For example, E-1G11-M-2F11 refers to a bispecific anti-EGFR/MET antibody that contains an anti-EGFR antigen-binding domain that is derived from E-1G11 and an anti-MET antigen-binding domain that is derived from M-2F11. In some embodiments, the anti-EGFR antigen-binding domain comprises the CDRs of E-1G11. In some embodiments, the anti-MET antigen-binding domain comprises the CDRs of M-2F11. In some embodiments, the anti-EGFR antigen-binding domain comprises the VH and VL of E-1G11. In some embodiments, the anti-MET antigen-binding domain comprises the VH and VL of M-2F11.

The bispecific antibody described herein can be designed to have an IgG1 subtype structure with knobs-into-holes (KIH) mutations, which can promote heterodimerization and avoid wrong pairing between the two heavy chains.

In some embodiments, the bispecific antibody has a higher endocytosis rate than the corresponding monoclonal antibodies or the control bispecific antibodies.

In some embodiments, the bispecific antibody described herein can be conjugated with a therapeutic agent, forming an antibody drug conjugate (ADC). In some embodiments, the DAR of the ADCs described herein is about 3.8, about 3.9, about 4.0, about 4.1, about 4.2, about 4.3, about 4.4, about 4.5, about 4.6, about 4.7, about 5.0, about 5.5, about 6.0, about 6.5, about 7.0, about 7.5, about 8.0, about 8.5, or about 9.0. In some embodiments, the DAR of the ADCs described herein is about 3.5 to about 4.5, about 3.6 about 4.5, about 3.7 to about 4.5, about 3.8 to about 4.5, about 3.9 to about 4.5, about 4.0 to about 4.5, about 4.1 to about 4.5, about 4.2 to about 4.5, about 4.3 to about 4.5, about 4.4 to about 4.5, about 3.5 to about 4.4, about 3.6 to about 4.4, about 3.7 to about 4.4, about 3.8 to about 4.4, about 3.9 to about 4.4, about 4.0 to about 4.4, about 4.1 to about 4.4, about 4.2 to about 4.4, about 4.3 to about 4.4, about 3.5 to about 4.3, about 3.6 to about 4.3, about 3.7 to about 4.3, about 3.8 to about 4.3, about 3.9 to about 4.3, about 4.0 to about 4.3, about 4.1 to about 4.3, about 4.2 to about 4.3, about 3.5 to about 4.2, about 3.6 to about 4.2, about 3.7 to about 4.2, about 3.8 to about 4.2, about 3.9 to about 4.2, about 4.0 to about 4.2, about 4.1 to about 4.2, about 3.5 to about 4.1, about 3.6 to about 4.1, about 3.7 to about 4.1, about 3.8 to about 4.1, about 3.9 to about 4.1, about 4.0 to about 4.1, about 3.5 to about 4.0, about 3.6 to about 4.0, about 3.7 to about 4.0, about 3.8 to about 4.0, about 3.9 to about 4.0, about 3.5 to about 3.9, about 3.6 to about 3.9, about 3.7 to about 3.9, about 3.8 to about 3.9, about 3.5 to about 3.8, about 3.6 to about 3.8, about 3.7 to about 3.8, about 3.5 to about 3.7, about 3.6 to about 3.7, or about 3.5 to about 3.6.

In some embodiments, the DAR of the ADCs described herein is about 7.5 to about 8.5, about 7.6 to about 8.5, about 7.7 to about 8.5, about 7.8 to about 8.5, about 7.9 to about 8.5, about 8.0 to about 8.5, about 8.1 to about 8.5, about 8.2 to about 8.5, about 8.3 to about 8.5, about 8.4 to about 8.5, about 7.5 to about 8.4, about 7.6 to about 8.4, about 7.7 to about 8.4, about 7.8 to about 8.4, about 7.9 to about 8.4, about 8.0 to about 8.4, about 8.1 to about 8.4, about 8.2 to about 8.4, about 8.3 to about 8.4, about 7.5 to about 8.3, about 7.6 to about 8.3, about 7.7 to about 8.3, about 7.8 to about 8.3, about 7.9 to about 8.3, about 8.0 to about 8.3, about 8.1 to about 8.3, about 8.2 to about 8.3, about 7.5 to about 8.2, about 7.6 to about 8.2, about 7.7 to about 8.2, about 7.8 to about 8.2, about 7.9 to about 8.2, about 8.0 to about 8.2, about 8.1 to about 8.2, about 7.5 to about 8.1, about 7.6 to about 8.1, about 7.7 to about 8.1, about 7.8 to about 8.1, about 7.9 to about 8.1, about 8.0 to about 8.1, about 7.5 to about 8.0, about 7.6 to about 8.0, about 7.7 to about 8.0, about 7.8 to about 8.0, about 7.9 to about 8.0, about 7.5 to about 7.9, about 7.6 to about 7.9, about 7.7 to about 7.9, about 7.8 to about 7.9, about 7.5 to about 7.8, about 7.6 to about 7.8, about 7.7 to about 7.8, about 7.5 to about 7.7, about 7.6 to about 7.7, or about 7.5 to about 7.6.

In some embodiments, the anti-EGFR/MET ADC described herein can effectively inhibit in vitro cancer cell growth at a concentration of less than 10 μg/ml, less than 3.33 μg/ml, less than 1.11 μg/ml, less than 0.37 sg/ml, less than 0.12 μg/ml, less than 0.04 sg/ml, or less than 0.01 μg/ml. In some embodiments, the anti-EGFR/MET ADC described herein can inhibit in vivo cancer cell growth (e.g., lung cancer, gastric cancer, or skin cancer) in a xenograft mouse model at a dose level of less than 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg.

In some embodiments, the bispecific antibody or antigen-binding fragment thereof described herein has a common light chain.

### Anti-MET Antibodies and Antigen-Binding Fragments

The disclosure provides antibodies and antigen-binding fragments thereof that specifically bind to MET. The anti-EGFR/MET antigen-binding protein construct (e.g., bispecific antibodies) can include an antigen binding region that is derived from these antibodies.

The antibodies and antigen-binding fragments described herein are capable of binding to MET. The disclosure provides anti-MET antibodies M-1C5 (“1C5”), M-2F11 (“2F11”), M-2G10 (“2G10”), M-1E9 (“1E9”), M-2F12 (“2F12”), M-6D12 (“6D12”), M-6E6 (“6E6”), M-7D3 (“7D3”), M-8D9 (“8D9”), M-8E2 (“8E2”), and M-8H10 (“8H10”) and the antibodies derived therefrom.

The CDR sequences for 2F11, and 2F11 derived antibodies (e.g., human antibodies) include CDRs of the heavy chain variable domain, SEQ ID NOs: 19-21, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 49-51, and CDR sequences of the light chain variable domain are set forth in SEQ ID NOs: 1-3. The human light chain variable region and human heavy chain variable region for 2F11 are shown in SEQ ID NO: 61 and SEQ ID NO: 67, respectively.

The CDR sequences for 2G10, and 2G10 derived antibodies include CDRs of the heavy chain variable domain, SEQ ID NOs: 22-24, and CDRs of the light chain variable domain, SEQ ID NOs: 1-3, as defined by Kabat numbering. Under Chothia numbering, the CDR sequences of the heavy chain variable domain are set forth in SEQ ID NOs: 52-54, and CDRs of the light chain variable domain are set forth in SEQ ID NOs: 1-3. The human light chain variable region and human heavy chain variable region for 2G10 are shown in SEQ ID NO: 61 and SEQ ID NO: 68, respectively.

The CDR sequences for 1E9, and 1E9 derived antibodies include CDRs of the heavy chain variable domain, SEQ ID NOs: 25-27, and CDRs of the light chain variable domain, SEQ ID NOs: 1-3, as defined by Kabat numbering. Under Chothia numbering, the CDR sequences of the heavy chain variable domain are set forth in SEQ ID NOs: 55-57, and CDRs of the light chain variable domain are set forth in SEQ ID NOs: 1-3. The human light chain variable region and human heavy chain variable region for 1E9 are shown in SEQ ID NO: 61 and SEQ ID NO: 69, respectively.

The CDR sequences for 2F12, and 2F12 derived antibodies include CDRs of the heavy chain variable domain, SEQ ID NOs: 28-30, and CDRs of the light chain variable domain, SEQ ID NOs: 1-3, as defined by Kabat numbering. Under Chothia numbering, the CDR sequences of the heavy chain variable domain are set forth in SEQ ID NOs: 58-60, and CDRs of the light chain variable domain are set forth in SEQ ID NOs: 1-3. The human light chain variable region and human heavy chain variable region for 2F12 are shown in SEQ ID NO: 61 and SEQ ID NO: 70, respectively.

The CDR sequences for 6D12, and 6D12 derived antibodies (e.g., human antibodies) include CDRs of the heavy chain variable domain, SEQ ID NOs: 112-114, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 130-132, and CDR sequences of the light chain variable domain are set forth in SEQ ID NOs: 1-3. The human light chain variable region and human heavy chain variable region for 6D12 are shown in SEQ ID NO: 61 and SEQ ID NO: 148, respectively.

The CDR sequences for 6E6, and 6E6 derived antibodies (e.g., human antibodies) include CDRs of the heavy chain variable domain, SEQ ID NOs: 115-117, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 133-135, and CDR sequences of the light chain variable domain are set forth in SEQ ID NOs: 1-3. The human light chain variable region and human heavy chain variable region for 6E6 are shown in SEQ ID NO: 61 and SEQ ID NO: 149, respectively.

The CDR sequences for 7D3, and 7D3 derived antibodies (e.g., human antibodies) include CDRs of the heavy chain variable domain, SEQ ID NOs: 118-120, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 136-138, and CDR sequences of the light chain variable domain are set forth in SEQ ID NOs: 1-3. The human light chain variable region and human heavy chain variable region for 7D3 are shown in SEQ ID NO: 61 and SEQ ID NO: 150, respectively.

The CDR sequences for 8D9, and 8D9 derived antibodies (e.g., human antibodies) include CDRs of the heavy chain variable domain, SEQ ID NOs: 121-123, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 139-141, and CDR sequences of the light chain variable domain are set forth in SEQ ID NOs: 1-3. The human light chain variable region and human heavy chain variable region for 8D9 are shown in SEQ ID NO: 61 and SEQ ID NO: 151, respectively.

The CDR sequences for 8E2, and 8E2 derived antibodies (e.g., human antibodies) include CDRs of the heavy chain variable domain, SEQ ID NOs: 124-126, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 142-144, and CDR sequences of the light chain variable domain are set forth in SEQ ID NOs: 1-3. The human light chain variable region and human heavy chain variable region for 8D9 are shown in SEQ ID NO: 61 and SEQ ID NO: 152, respectively.

The CDR sequences for 8H10, and 8H10 derived antibodies (e.g., human antibodies) include CDRs of the heavy chain variable domain, SEQ ID NOs: 127-129, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 145-147, and CDR sequences of the light chain variable domain are set forth in SEQ ID NOs: 1-3. The human light chain variable region and human heavy chain variable region for 8H10 are shown in SEQ ID NO: 61 and SEQ ID NO: 153, respectively.

Furthermore, in some embodiments, the antibodies or antigen-binding fragments thereof described herein can also contain one, two, or three heavy chain variable region CDRs selected from the group of SEQ ID NOs: 19-21, SEQ ID NOs: 22-24, SEQ ID NOs: 25-27, SEQ ID NOs: 28-30, SEQ ID NOs: 49-51, SEQ ID NOs: 52-54, SEQ ID NOs: 55-57, SEQ ID NOs: 58-60, SEQ ID NOs: 112-114, SEQ ID NOs: 115-117, SEQ ID NOs: 118-120, SEQ ID NOs: 121-123, SEQ ID NOs: 124-126, SEQ ID NOs: 127-129, SEQ ID NOs: 130-132, SEQ ID NOs: 133-135, SEQ ID NOs: 136-138, SEQ ID NOs: 139-141, SEQ ID NOs: 142-144, and SEQ ID NOs: 145-147; and/or one, two, or three light chain variable region CDRs selected from the group of SEQ ID NOs: 1-3.

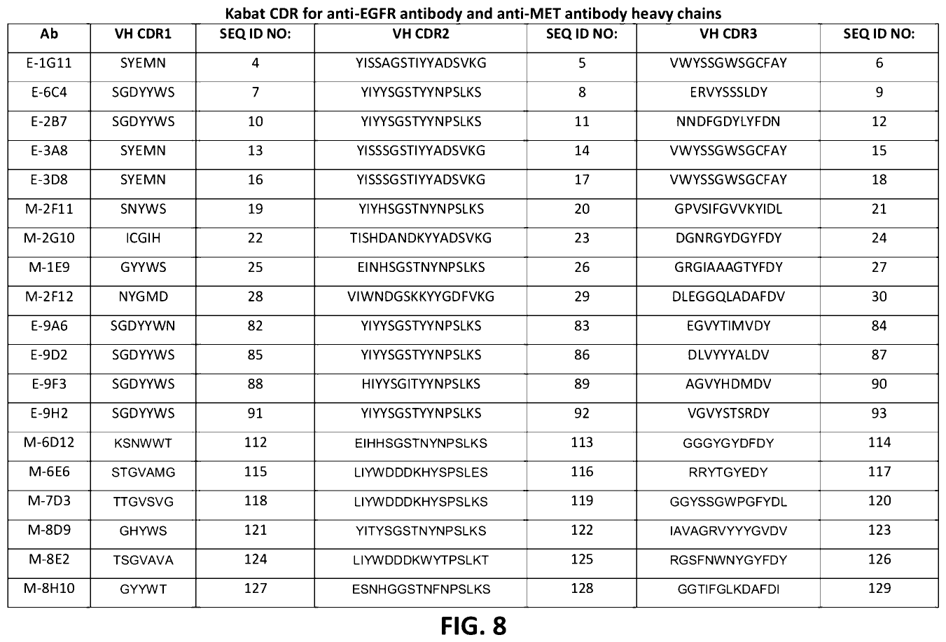

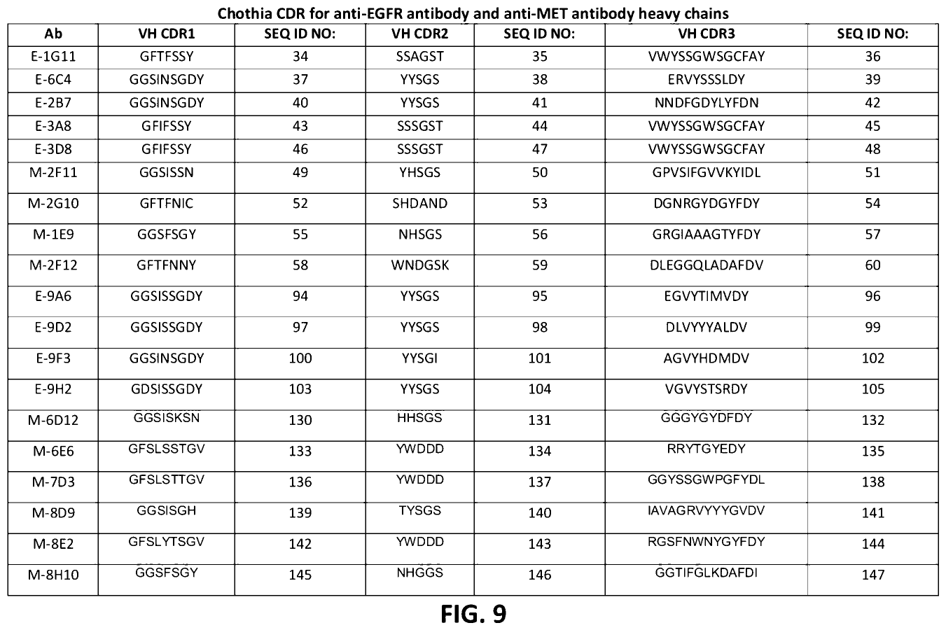

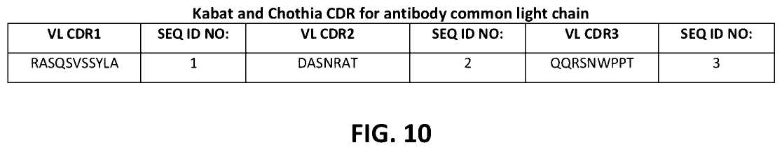

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIGS. 8 and 10 (Kabat CDR) and FIGS. 9 and 10 (Chothia CDR).

In some embodiments, the antibody or an antigen-binding fragment described herein can contain a heavy chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 49 with zero, one or two amino acid insertions, deletions, or substitutions; SEQ ID NO: 50 with zero, one or two amino acid insertions, deletions, or substitutions; SEQ ID NO: 51 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 52 with zero, one or two amino acid insertions, deletions, or substitutions; SEQ ID NO: 53 with zero, one or two amino acid insertions, deletions, or substitutions; SEQ ID NO: 54 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 55 with zero, one or two amino acid insertions, deletions, or substitutions; SEQ ID NO: 56 with zero, one or two amino acid insertions, deletions, or substitutions; SEQ ID NO: 57 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 58 with zero, one or two amino acid insertions, deletions, or substitutions; SEQ ID NO: 59 with zero, one or two amino acid insertions, deletions, or substitutions: SEQ ID NO: 60 with zero, one or two amino acid insertions, deletions, or substitutions.

In some embodiments, the antibody or an antigen-binding fragment described herein can contain a light chain variable domain containing one, two, or three of the CDRs of SEQ ID NO: 112 with zero, one or two amino acid insertions, deletions, or substitutions; SEQ ID NO: 113 with zero, one or two amino acid insertions, deletions, or substitutions; SEQ ID NO: 114 with zero, one or two amino acid insertions, deletions, or substitutions.

In some embodiments, the antibody or an antigen-binding fragment described herein can contain a light chain variable domain containing one, two, or three of the CDRs of SEQ ID NO: 115 with zero, one or two amino acid insertions, deletions, or substitutions; SEQ ID NO: 116 with zero, one or two amino acid insertions, deletions, or substitutions; SEQ ID NO: 117 with zero, one or two amino acid insertions, deletions, or substitutions.

In some embodiments, the antibody or an antigen-binding fragment described herein can contain a light chain variable domain containing one, two, or three of the CDRs of SEQ ID NO: 118 with zero, one or two amino acid insertions, deletions, or substitutions; SEQ ID NO: 119 with zero, one or two amino acid insertions, deletions, or substitutions; SEQ ID NO: 120 with zero, one or two amino acid insertions, deletions, or substitutions.

In some embodiments, the antibody or an antigen-binding fragment described herein can contain a light chain variable domain containing one, two, or three of the CDRs of SEQ ID NO: 121 with zero, one or two amino acid insertions, deletions, or substitutions; SEQ ID NO: 122 with zero, one or two amino acid insertions, deletions, or substitutions; SEQ ID NO: 123 with zero, one or two amino acid insertions, deletions, or substitutions.

In some embodiments, the antibody or an antigen-binding fragment described herein can contain a light chain variable domain containing one, two, or three of the CDRs of SEQ ID NO: 124 with zero, one or two amino acid insertions, deletions, or substitutions; SEQ ID NO: 125 with zero, one or two amino acid insertions, deletions, or substitutions; SEQ ID NO: 126 with zero, one or two amino acid insertions, deletions, or substitutions.

In some embodiments, the antibody or an antigen-binding fragment described herein can contain a light chain variable domain containing one, two, or three of the CDRs of SEQ ID NO: 127 with zero, one or two amino acid insertions, deletions, or substitutions; SEQ ID NO: 128 with zero, one or two amino acid insertions, deletions, or substitutions; SEQ ID NO: 129 with zero, one or two amino acid insertions, deletions, or substitutions.

In some embodiments, the antibody or an antigen-binding fragment described herein can contain a light chain variable domain containing one, two, or three of the CDRs of SEQ ID NO: 130 with zero, one or two amino acid insertions, deletions, or substitutions; SEQ ID NO: 131 with zero, one or two amino acid insertions, deletions, or substitutions; SEQ ID NO: 132 with zero, one or two amino acid insertions, deletions, or substitutions.

In some embodiments, the antibody or an antigen-binding fragment described herein can contain a light chain variable domain containing one, two, or three of the CDRs of SEQ ID NO: 133 with zero, one or two amino acid insertions, deletions, or substitutions; SEQ ID NO: 134 with zero, one or two amino acid insertions, deletions, or substitutions; SEQ ID NO: 135 with zero, one or two amino acid insertions, deletions, or substitutions.

In some embodiments, the antibody or an antigen-binding fragment described herein can contain a light chain variable domain containing one, two, or three of the CDRs of SEQ ID NO: 136 with zero, one or two amino acid insertions, deletions, or substitutions; SEQ ID NO: 137 with zero, one or two amino acid insertions, deletions, or substitutions; SEQ ID NO: 138 with zero, one or two amino acid insertions, deletions, or substitutions.

In some embodiments, the antibody or an antigen-binding fragment described herein can contain a light chain variable domain containing one, two, or three of the CDRs of SEQ ID NO: 139 with zero, one or two amino acid insertions, deletions, or substitutions; SEQ ID NO: 140 with zero, one or two amino acid insertions, deletions, or substitutions: SEQ ID NO: 141 with zero, one or two amino acid insertions, deletions, or substitutions.

In some embodiments, the antibody or an antigen-binding fragment described herein can contain a light chain variable domain containing one, two, or three of the CDRs of SEQ ID NO: 142 with zero, one or two amino acid insertions, deletions, or substitutions; SEQ ID NO: 143 with zero, one or two amino acid insertions, deletions, or substitutions; SEQ ID NO: 144 with zero, one or two amino acid insertions, deletions, or substitutions.

In some embodiments, the antibody or an antigen-binding fragment described herein can contain a light chain variable domain containing one, two, or three of the CDRs of SEQ ID NO: 145 with zero, one or two amino acid insertions, deletions, or substitutions; SEQ ID NO: 146 with zero, one or two amino acid insertions, deletions, or substitutions; SEQ ID NO: 147 with zero, one or two amino acid insertions, deletions, or substitutions.

In some embodiments, the antibody or an antigen-binding fragment described herein can contain a light chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

The insertions, deletions, and substitutions can be within the CDR sequence, or at one or both terminal ends of the CDR sequence.

The disclosure also provides antibodies or antigen-binding fragments thereof that binds to MET. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 67, 68, 69, 70, 148, 149, 150, 151, 152 or 153, and the selected VL sequence is SEQ ID NO: 61.

In some embodiments, the antibody or antigen binding fragments thereof can have 3 VH CDRs that are identical to the CDRs of any VH sequences as described herein. In some embodiments, the antibody or antigen binding fragments thereof can have 3 VL CDRs that are identical to the CDRs of any VL sequences as described herein.

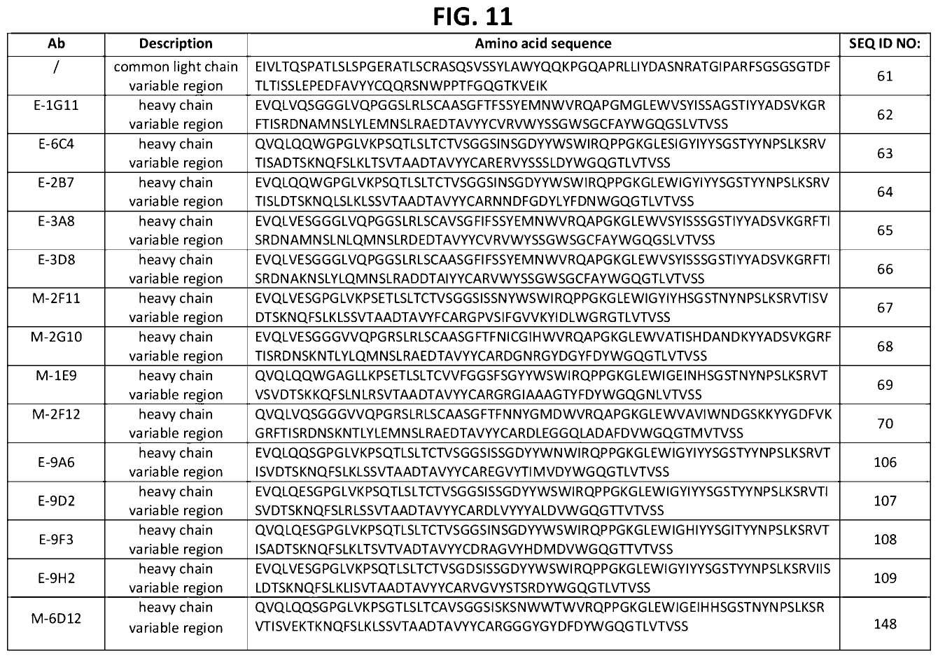

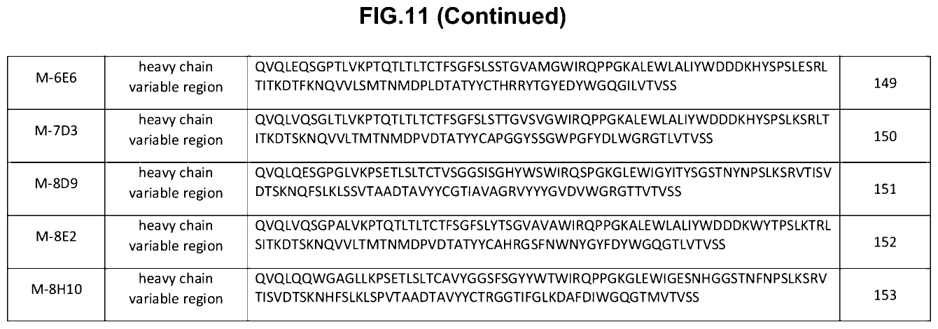

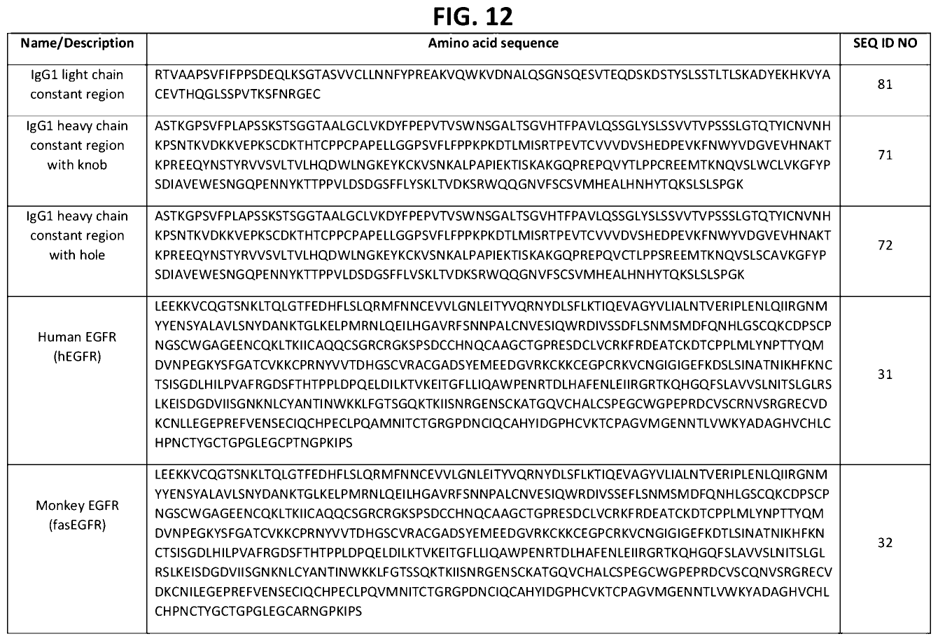

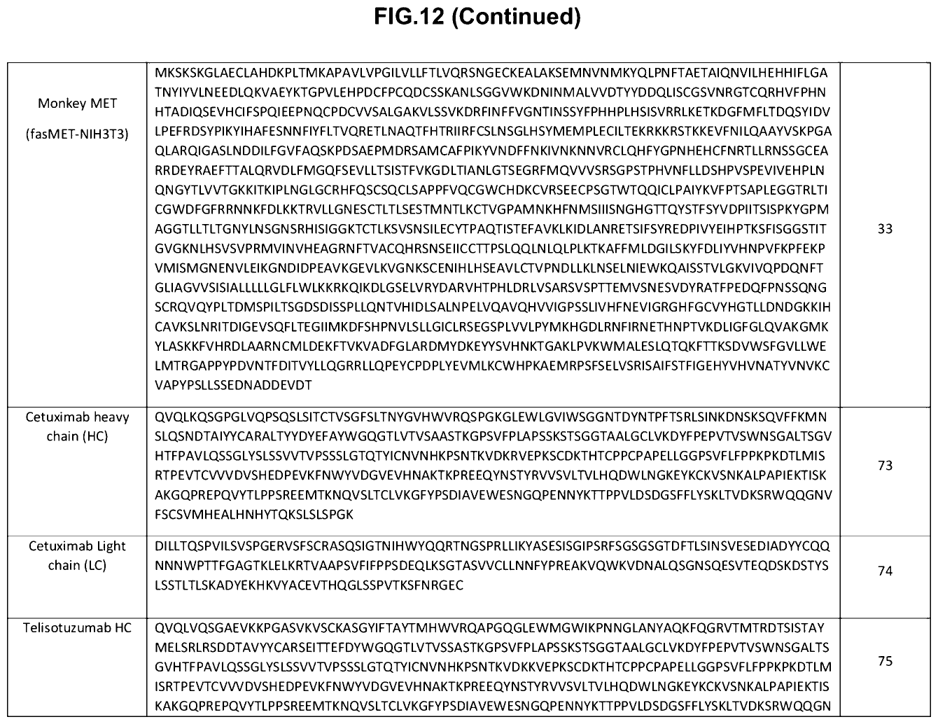

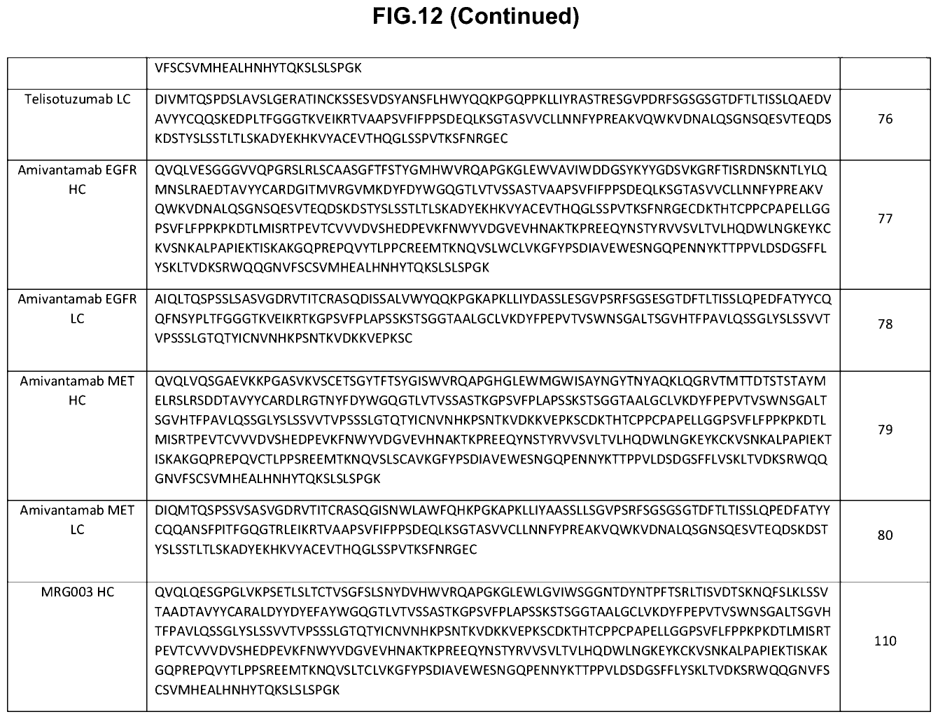

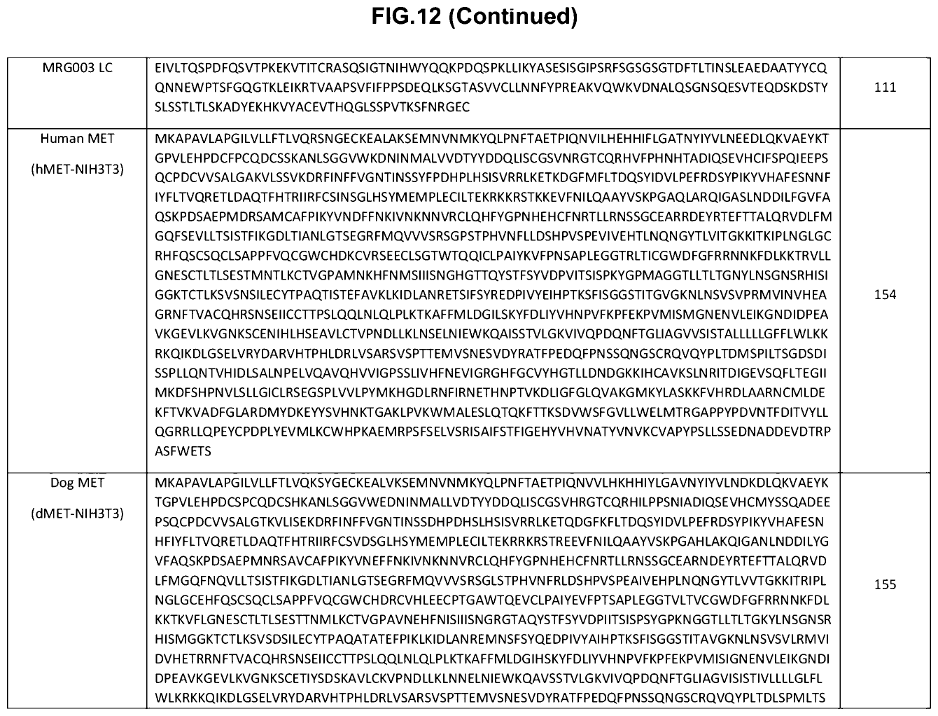

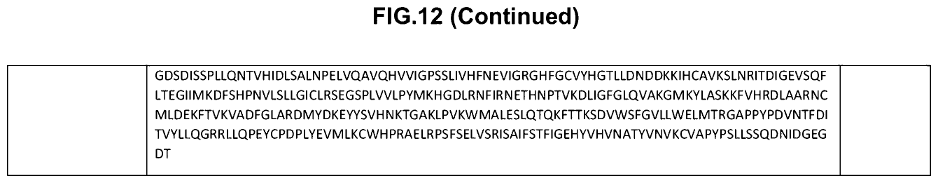

The disclosure also provides nucleic acid comprising a polynucleotide encoding a polypeptide comprising an immunoglobulin heavy chain or an immunoglobulin heavy chain. The immunoglobulin heavy chain or immunoglobulin light chain comprises CDRs as shown in FIG. 8, FIG. 9, or FIG. 10, or have sequences as shown in FIG. 11. When the polypeptides are paired with corresponding polypeptide (e.g., a corresponding heavy chain variable region or a corresponding light chain variable region), the paired polypeptides bind to MET.

The anti-MET antibodies and antigen-binding fragments can also be antibody variants (including derivatives and conjugates) of antibodies or antibody fragments and multi-specific (e.g., bispecific) antibodies or antibody fragments. Additional antibodies provided herein are polyclonal, monoclonal, multi-specific (multimeric, e.g., bispecific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG (e.g., IgG1) antibody or antigen-binding fragment thereof.

Fragments of antibodies are suitable for use in the methods provided so long as they retain the desired affinity and specificity of the full-length antibody. Thus, a fragment of an antibody that binds to MET will retain an ability to bind to MET. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

### Anti-EGFR Antibodies and Antigen-Binding Fragments

The disclosure provides several antibodies and antigen-binding fragments thereof that specifically bind to EGFR. In some embodiments, the anti-EGFR/MET antigen-binding protein constructs (e.g., bispecific antibodies) can include an antigen binding region that is derived from these antibodies.

The antibodies and antigen-binding fragments described herein are capable of binding to EGFR. The disclosure provides e.g., anti-EGFR antibodies E-1C12 (“1C12”), E-1G11 (“IG 1”), E-6C4 (“6C4”), E-2B7 (“2B7”), E-3A8 (“3A8”), E-3D8 (“3D8”), E-9A6 (“9A6”), E-9D2 (“9D2”), E-9F3 (“9F3”) and E-9H2 (“9H2”) and the antibodies derived therefrom.

The CDR sequences for 1G11, and 1G11 derived antibodies (e.g., human antibodies) include CDRs of the heavy chain variable domain, SEQ ID NOs: 4-6, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 34-36, and CDR sequences of the light chain variable domain are set forth in SEQ ID NOs: 1-3. The human light chain variable region and human heavy chain variable region for 1G11 are shown in SEQ ID NO: 61 and SEQ ID NO: 62, respectively.

The CDR sequences for 6C4, and 6C4 derived antibodies (e.g., human antibodies) include CDRs of the heavy chain variable domain, SEQ ID NOs: 7-9, and CDRs of the light chain variable domain, SEQ ID NOs: 1-3, as defined by Kabat numbering. Under Chothia numbering, the CDR sequences of the heavy chain variable domain are set forth in SEQ ID NOs: 37-38, and CDRs of the light chain variable domain are set forth in SEQ ID NOs: 1-3. The human light chain variable region and human heavy chain variable region for 6C4 are shown in SEQ ID NO: 61 and SEQ ID NO: 63, respectively.

The CDR sequences for 2B7, and 2B7 derived antibodies (e.g., human antibodies) include CDRs of the heavy chain variable domain, SEQ ID NOs: 10-12, and CDRs of the light chain variable domain, SEQ ID NOs: 1-3, as defined by Kabat numbering. Under Chothia numbering, the CDR sequences of the heavy chain variable domain are set forth in SEQ ID NOs: 40-42, and CDRS of the light chain variable domain are set forth in SEQ ID NOs: 1-3. The human light chain variable region and human heavy chain variable region for 2B8 are shown in SEQ ID NO: 61 and SEQ ID NO: 64, respectively.

The CDR sequences for 3A8, and 3A8 derived antibodies (e.g., human antibodies) include CDRs of the heavy chain variable domain, SEQ ID NOs: 13-15, and CDRs of the light chain variable domain, SEQ ID NOs: 1-3, as defined by Kabat numbering. Under Chothia numbering, the CDR sequences of the heavy chain variable domain are set forth in SEQ ID NOs: 43-45, and CDRS of the light chain variable domain are set forth in SEQ ID NOs: 1-3. The human light chain variable region and human heavy chain variable region for 3A8 are shown in SEQ ID NO: 61 and SEQ ID NO: 65, respectively.

The CDR sequences for 3D8, and 3D8 derived antibodies (e.g., human antibodies) include CDRs of the heavy chain variable domain, SEQ ID NOs: 16-18, and CDRs of the light chain variable domain, SEQ ID NOs: 1-3, as defined by Kabat numbering. Under Chothia numbering, the CDR sequences of the heavy chain variable domain are set forth in SEQ ID NOs: 46-48, and CDRs of the light chain variable domain are set forth in SEQ ID NOs: 1-3. The human light chain variable region and human heavy chain variable region for 3D8 are shown in SEQ ID NO: 61 and SEQ ID NO: 66, respectively.

The CDR sequences for 9A6, and 9A6 derived antibodies (e.g., human antibodies) include CDRs of the heavy chain variable domain, SEQ ID NOs: 82-84, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 94-96, and CDR sequences of the light chain variable domain are set forth in SEQ ID NOs: 1-3. The human light chain variable region and human heavy chain variable region for 9A6 are shown in SEQ ID NO: 61 and SEQ ID NO: 106, respectively.

The CDR sequences for 9D2, and 9D2 derived antibodies (e.g., human antibodies) include CDRs of the heavy chain variable domain, SEQ ID NOs: 85-87, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 97-99, and CDR sequences of the light chain variable domain are set forth in SEQ ID NOs: 1-3. The human light chain variable region and human heavy chain variable region for 9D2 are shown in SEQ ID NO: 61 and SEQ ID NO: 107, respectively.

The CDR sequences for 9F3, and 9F3 derived antibodies (e.g., human antibodies) include CDRs of the heavy chain variable domain, SEQ ID NOs: 88-90, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 100-102, and CDR sequences of the light chain variable domain are set forth in SEQ ID NOs: 1-3. The human light chain variable region and human heavy chain variable region for 9F3 are shown in SEQ ID NO: 61 and SEQ ID NO: 108, respectively.

The CDR sequences for 9H2, and 9H2 derived antibodies (e.g., human antibodies) include CDRs of the heavy chain variable domain, SEQ ID NOs: 91-93, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 103-105, and CDR sequences of the light chain variable domain are set forth in SEQ ID NOs: 1-3. The human light chain variable region and human heavy chain variable region for 9H2 are shown in SEQ ID NO: 61 and SEQ ID NO: 109, respectively.

Furthermore, in some embodiments, the antibodies or antigen-binding fragments thereof described herein can also contain one, two, or three heavy chain variable region CDRs selected from the group of SEQ ID NOs: 4-6, SEQ ID NOs: 7-9, SEQ ID NOs: 10-12, SEQ ID NOs: 13-15, SEQ ID NOs: 16-18, SEQ ID NOs: 34-36, SEQ ID NOs: 37-39, SEQ ID NOs: 40-42, SEQ ID NOs: 43-45, SEQ ID NOs: 46-48, SEQ ID NOs: 82-84, SEQ ID NOs: 85-87, SEQ ID NOs: 88-90, SEQ ID NOs: 91-93, SEQ ID NOs: 94-96, SEQ ID NOs: 97-99, SEQ ID NOs: 100-102 and SEQ ID NOs: 103-105; and/or one, two, or three light chain variable region CDRs selected from the group of SEQ ID NOs: 1-3.

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIGS. 8 and 10 (Kabat CDR) and FIGS. 9 and 10 (Chothia CDR).

In some embodiments, the antibody or an antigen-binding fragment described herein can contain a heavy chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions: SEQ ID NO: 6 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions: SEQ ID NO: 9 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions: SEQ ID NO: 15 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 37 with zero, one or two amino acid insertions, deletions, or substitutions; SEQ ID NO: 38 with zero, one or two amino acid insertions, deletions, or substitutions; SEQ ID NO: 39 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 40 with zero, one or two amino acid insertions, deletions, or substitutions; SEQ ID NO: 41 with zero, one or two amino acid insertions, deletions, or substitutions; SEQ ID NO: 42 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 43 with zero, one or two amino acid insertions, deletions, or substitutions; SEQ ID NO: 44 with zero, one or two amino acid insertions, deletions, or substitutions; SEQ ID NO: 45 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 46 with zero, one or two amino acid insertions, deletions, or substitutions; SEQ ID NO: 47 with zero, one or two amino acid insertions, deletions, or substitutions; SEQ ID NO: 48 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 82 with zero, one or two amino acid insertions, deletions, or substitutions; SEQ ID NO: 83 with zero, one or two amino acid insertions, deletions, or substitutions; SEQ ID NO: 84 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 85 with zero, one or two amino acid insertions, deletions, or substitutions; SEQ ID NO: 86 with zero, one or two amino acid insertions, deletions, or substitutions; SEQ ID NO: 87 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 88 with zero, one or two amino acid insertions, deletions, or substitutions; SEQ ID NO: 89 with zero, one or two amino acid insertions, deletions, or substitutions: SEQ ID NO: 90 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 91 with zero, one or two amino acid insertions, deletions, or substitutions; SEQ ID NO: 92 with zero, one or two amino acid insertions, deletions, or substitutions; SEQ ID NO: 93 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 94 with zero, one or two amino acid insertions, deletions, or substitutions; SEQ ID NO: 95 with zero, one or two amino acid insertions, deletions, or substitutions; SEQ ID NO: 96 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 97 with zero, one or two amino acid insertions, deletions, or substitutions; SEQ ID NO: 98 with zero, one or two amino acid insertions, deletions, or substitutions; SEQ ID NO: 99 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 100 with zero, one or two amino acid insertions, deletions, or substitutions; SEQ ID NO: 101 with zero, one or two amino acid insertions, deletions, or substitutions; SEQ ID NO: 102 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 103 with zero, one or two amino acid insertions, deletions, or substitutions; SEQ ID NO: 104 with zero, one or two amino acid insertions, deletions, or substitutions; SEQ ID NO: 105 with zero, one or two amino acid insertions, deletions, or substitutions.

In some embodiments, the antibody or an antigen-binding fragment described herein can contain a light chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

The insertions, deletions, and substitutions can be within the CDR sequence, or at one or both terminal ends of the CDR sequence.

The disclosure also provides antibodies or antigen-binding fragments thereof that bind to EGFR. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NOs: 62, 63, 64, 65, 66, 106, 107, 108, or 109, and the selected VL sequence is SEQ ID NO: 61.

In some embodiments, the antibody or antigen binding fragment thereof can have 3 VH CDRs that are identical to the CDRs of any VH sequences as described herein. In some embodiments, the antibody or antigen binding fragment thereof can have 3 VL CDRs that are identical to the CDRs of any VL sequences as described herein.

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 8, FIG. 9, or FIG. 10, or have sequences as shown in FIG. 11. When the polypeptides are paired with corresponding polypeptide (e.g., a corresponding heavy chain variable region or a corresponding light chain variable region), the paired polypeptides bind to EGFR (e.g., human EGFR).

The anti-EGFR antibodies and antigen-binding fragments can also be antibody variants (including derivatives and conjugates) of antibodies or antibody fragments and multi-specific (e.g., bispecific) antibodies or antibody fragments. Additional antibodies provided herein are polyclonal, monoclonal, multi-specific (multimeric, e.g., bispecific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG (e.g., IgG1) antibody or antigen-binding fragment thereof.

Fragments of antibodies are suitable for use in the methods provided so long as they retain the desired affinity and specificity of the full-length antibody. Thus, a fragment of an antibody that binds to EGFR will retain an ability to bind to EGFR. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

### Antibodies, Antigen Binding Fragments and Antigen Binding Protein Constructs

The present disclosure provides antibodies, the antigen-binding fragments thereof, or the antigen-binding protein constructs (e.g., bispecific antibodies). The antigen-binding protein construct (e.g., bispecific antibody) can comprise an anti-EGFR antibody or antigen-binding fragment thereof, and anti-MET antibody or antigen-binding fragment thereof. These antigen-binding protein constructs (e.g., bispecific antibody), anti-EGFR antibodies, anti-MET antibodies, and antigen-binding fragments thereof can have various forms.

In general, antibodies (also called immunoglobulins) can be made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and/or two identical copies of a heavy chain. The heavy chains, which each contain one variable domain (or variable region. VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The antigen binding domain or antigen binding fragment is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFv has two heavy chain variable domains, and two light chain variable domains. In some embodiments, the scFv has two antigen binding regions (Antigen binding regions: A and B), and the two antigen binding regions can bind to the respective target antigens with different affinities.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the antibodies, the antigen-binding fragments thereof, or the antigen-binding protein constructs (e.g., bispecific antibodies) can bind to two different antigens or two different epitopes.

In some embodiments, the antibodies, the antigen-binding fragments thereof, or the antigen-binding protein constructs (e.g., bispecific antibodies) can comprises one, two, or three heavy chain variable region CDRs selected from FIGS. 8 and 9. In some embodiments, the antibodies, the antigen-binding fragments thereof, or the antigen-binding protein constructs (e.g., bispecific antibodies) can comprises one, two, or three light chain variable region CDRs selected from FIG. 10.

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

In some embodiments, the multi-specific antibody is a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Any of the antibodies, the antigen-binding fragments thereof, or the antigen-binding protein constructs (e.g., bispecific antibodies) described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

The antibodies, the antigen-binding fragments thereof, or the antigen-binding protein constructs (e.g., bispecific antibodies) can also have various forms. Many different formats of antigen binding constructs are known in the art, and are described e.g., in Suurs, et al. “A review of bispecific antibodies and antibody constructs in oncology and clinical challenges,” Pharmacology & therapeutics (2019), which is incorporated herein by reference in the entirety.

In some embodiments, the antigen-binding protein construct is a BiTe, a (scFv)₂, a nanobody, a nanobody-HSA, a DART, a TandAb, a scDiabody, a scDiabody-CH3, scFv-CH-CL-scFv, a HSAbody, scDiabody-HAS, or a tandem-scFv. In some embodiments, the antigen-binding protein construct is a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, an Intrabody, a dock and lock, a lmmTAC, an IgG-IgG conjugate, a Cov-X-Body, or a scFv1-PEG-scFv2.

In some embodiments, the antigen-binding protein construct can be a TrioMab. In a TrioMab, the two heavy chains are from different species, wherein different sequences restrict the heavy-light chain pairing.

In some embodiments, the antigen-binding protein construct has two different heavy chains and one common light chain. Heterodimerization of heavy chains can be based on the knobs-into-holes or some other heavy chain pairing technique.

In some embodiments, CrossMAb technique can be used produce bispecific antibodies. CrossMAb technique can be used enforce correct light chain association in bispecific heterodimeric IgG antibodies, this technique allows the generation of various bispecific antibody formats, including bi-(1+1), tri-(2+1) and tetra-(2+2) valent bispecific antibodies, as well as non-Fc tandem antigen-binding fragment (Fab)-based antibodies. These formats can be derived from any existing antibody pair using domain crossover, without the need for the identification of common light chains, post-translational processing/in vitro chemical assembly or the introduction of a set of mutations enforcing correct light chain association. The method is described in Klein et al., “The use of CrossMAb technology for the generation of bi- and multispecific antibodies.” MAbs. Vol. 8. No. 6. Taylor & Francis, 2016, which is incorporated by reference in its entirety. In some embodiments, the CHI in the heavy chain and the CL domain in the light chain are swapped.

The antigen-binding protein construct can be a Duobody. The Fab-exchange mechanism naturally occurring in IgG4 antibodies is mimicked in a controlled matter in IgG1 antibodies, a mechanism called controlled Fab exchange. This format can ensure specific pairing between the heavy-light chains.

In Dual-variable-domain antibody (DVD-Ig), additional VH and variable light chain (VL) domain are added to each N-terminus for bispecific targeting. This format resembles the IgG-scFv, but the added binding domains are bound individually to their respective N-termini instead of a scFv to each heavy chain N-terminus.

In scFv-IgG, the two scFv are connected to the C-terminus of the heavy chain (CH3). The scFv-IgG format has two different bivalent binding sites and is consequently also called tetravalent. There are no heavy-chain and light-chain pairing problem in the scFv-IgG.

In some embodiments, the antigen-binding protein construct can be have a IgG-IgG format. Two intact IgG antibodies are conjugated by chemically linking the C-terminals of the heavy chains.

The antigen-binding protein construct can also have a Fab-scFv-Fc format. In Fab-scFv-Fc format, a light chain, heavy chain and a third chain containing the Fc region and the scFv are assembled. It can ensure efficient manufacturing and purification.

In some embodiments, antigen-binding protein construct can be a TF. Three Fab fragments are linked by disulfide bridges. Two fragments target the tumor associated antigen (TAA) and one fragment targets a hapten. The TF format does not have an Fc region.

ADAPTIR has two scFvs bound to each side of an Fc region. It abandons the intact IgG as a basis for its construct, but conserves the Fc region to extend the half-life and facilitate purification.

Bispecific T cell Engager (“BiTE”) consists of two scFvs, VLA VHA and VHB VLB on one peptide chain. It has only binding domains, no Fc region.

In BiTE-Fc, an Fc region is fused to the BiTE construct. The addition of Fc region enhances half-life leading to longer effective concentrations, avoiding continuous IV.

Dual affinity retargeting (DART) has two peptide chains connecting the opposite fragments, thus VLA with VHB and VLB with VHA, and a sulfur bond at their C-termini fusing them together. In DART, the sulfur bond can improve stability over BiTEs.

In DART-Fc, an Fc region is attached to the DART structure. It can be generated by assembling three chains, two via a disulfide bond, as with the DART. One chain contains half of the Fc region which will dimerize with the third chain, only expressing the Fc region. The addition of Fc region enhances half-life leading to longer effective concentrations, avoiding continuous IV.

In tetravalent DART, four peptide chains are assembled. Basically, two DART molecules are created with half an Fc region and will dimerize. This format has bivalent binding to both targets, thus it is a tetravalent molecule.

Tandem diabody (TandAb) comprises two diabodies. Each diabody consists of an VHA and VLB fragment and a VHA and VLB fragment that are covalently associated. The two diabodies are linked with a peptide chain. It can improve stability over the diabody consisting of two scFvs. It has two bivalent binding sites.

The ScFv-scFv-toxin includes toxin and two scFv with a stabilizing linker. It can be used for specific delivery of payload.

In modular scFv-scFv-scFv, one scFv directed against the TAA is tagged with a short recognizable peptide is assembled to a bsAb consisting of two scFvs, one directed against CD3 and one against the recognizable peptide.

In ImmTAC, a stabilized and soluble T cell receptor is fused to a scFv recognizing CD3. By using a TCR, the ImmTAC is suitable to target processed, e.g. intracellular, proteins.

Tri-specific nanobody has two single variable domains (nanobodies) with an additional module for half-life extension. The extra module is added to enhance half-life.

In Trispecific Killer Engager (TriKE), two scFvs are connected via polypeptide linkers incorporating human IL-15. The linker to IL-15 is added to increase survival and proliferation of NKs.

In some embodiments, the antigen-binding protein construct is a bispecific antibody. In some embodiments, the bispecific antibody in present disclosure is designed to be 1+1 (monovalent for each target) and has an IgG1 subtype structure. This can reduce the avidity to cells with low expression levels of EGFR and MET, and increase the avidity to cells that co-express EGFR and MET, to achieve enhanced targeting function.

In some embodiments, the antibodies, the antigen-binding fragments thereof, the antigen-binding protein constructs (e.g., the anti-MET antibody, the anti-EGFR antibody, or the bispecific antibody) have a light chain constant region that is at least 80%, 85%, 86%, 87%, 88%, 89%, 90%, 91%, 92%, 93%, 94%, 95%, 96%, 97%, 98%, 99% or 100% identical to SEQ ID NO: 81, and a heavy chain constant region that is at least 80%, 85%, 86%, 87%, 88%, 89%, 90%, 91%, 92%, 93%, 94%, 95%, 96%, 97%, 98%, 99% or 100% identical to any one of SEQ ID NOs: 71 and 72.

In some embodiments, the anti-EGFR/MET antigen-binding protein construct (e.g., antibodies, bispecific antibodies, or antibody fragments thereof) include KIH mutations. In some embodiments, the antigen-binding protein construct includes a first antigen-binding domain that specifically binds to EGFR, and a second antigen-binding domain that specially binds to MET. In some embodiments, the first antigen-binding domain includes a heavy chain that including one or more knob mutations (a knob heavy chain), and the second antigen-binding domain includes a heavy chain including one or more hole mutations (a hole heavy chain). In some embodiments, the first antigen-binding domain includes a heavy chain that including one or more hole mutations (a hole heavy chain), and the second antigen-binding domain includes a heavy chain including one or more knob mutations (a knob heavy chain). In some embodiments, the anti-EGFR/MET antigen-binding protein construct includes a knob heavy chain comprising a constant region that is at least 80%, 85%, 86%, 87%, 88%, 89%, 90%, 91%, 92%, 93%, 94%, 95%, 96%, 97%, 98%, 99% or 100% identical to SEQ ID NO: 71. In some embodiments, the anti-EGFR/MET antigen-binding protein construct includes a hole heavy chain comprising a constant region that is at least 80%, 85%, 86%, 87%, 88%, 89%, 90%, 91%, 92%, 93%, 94%, 95%, 96%, 97%, 98%, 99% or 100% identical to SEQ ID NO: 72.

In some embodiments, the bispecific antibody or antigen-binding fragment thereof described herein has a common light chain.

### Antibody Drug Conjugates (ADCs)

In some embodiments, the antibodies, the antigen-binding fragments thereof, or the antigen-binding protein constructs (e.g., bispecific antibodies) described herein can be conjugated to a therapeutic agent, optionally with a linker, to form an antibody-drug conjugate. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., monomethyl auristatin E, monomethyl auristatin F, camptothecin, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, I-dehydrotestosterone, glucocorticoids, procaine, tetracaine, lidocaine, propranolol, puromycin, epirubicin, and cyclophosphamide and analogs). In some embodiments, the therapeutic agent is MMAE or MMAF.

Definitions of specific functional groups and chemical terms are described in more detail below. For purpose of this invention, the chemical elements are identified in accordance with the Periodic Table of the Elements, CAS version, Handbook of Chemistry and Physics, 75^{th} Edition, inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3P Edition, Cambridge University Press, Cambridge, 1987.

All ranges cited herein are inclusive, unless expressly stated to the contrary. When a range of values is listed, it is intended to encompass each value and sub-range within the range. For example, “C₁₋₄” is intended to encompass, C₁, C₂. C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₃₋₄, C₄₋₅, and C₅₋₆.

The compounds or any formula depicting and describing the compounds of the present disclosure may have one or more chiral (asymmetric) centers. The present invention encompasses all stereoisomeric forms of the compounds or any formula depicting and describing the compounds of the present invention. Centers of asymmetry that are present in the compounds or any formula depicting and describing the compounds of the present invention can all independently of one another have (R) or (S) configuration. When bonds to a chiral carbon are depicted as straight lines in the structural formulas, or when a compound name is recited without an (R) or (S) chiral designation for a chiral carbon, it is understood that both the (R) and (S) configurations of each such chiral carbon, and hence each enantiomer or diastereomer and mixtures thereof, are embraced within the formula or by the name.

The disclosure includes all possible enantiomers and diastereomers and mixtures of two or more stereoisomers, for example mixtures of enantiomers and/or diastereomers, in all ratios. Thus, enantiomers are a subject of the disclosure in enantiomerically pure form, both as levorotatory and as dextrorotatory antipodes, in the form of racemates and in the form of mixtures of the two enantiomers in all ratios. In the case of a cis/trans isomerism the disclosure includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis.

Unless otherwise stated, the structures depicted herein are also meant to include the compounds that differ only in the presence of one or more isotopically enriched atoms, in other words, the compounds wherein one or more atoms are replaced by atoms having the same atomic number, but an atomic mass or mass number different from the atomic mass or mass number which predominates in nature. Such compounds are referred to as a “isotopic variant”. The present disclosure is intended to include all pharmaceutically acceptable isotopic variants of the compounds or any formula depicting and describing the compounds of the present invention. Examples of isotopes suitable for inclusion in the compounds of the present invention include, but not limited to, isotopes of hydrogen, such as ²H (i.e., D) and ³H; carbon, such as ¹¹C, ¹³C, and ¹⁴C; chlorine, such as ³⁶Cl; fluorine, such as ¹⁸F; iodine, such as ¹²³I and ¹²⁵I; nitrogen, such as ¹³N and ¹⁵N; oxygen, such as ¹⁵O, ¹⁷O, and ISO; phosphorus, such as ³²P; and sulfur, such as ³⁵S. Certain isotopic variants of the compounds or any formula depicting and describing the compounds of the present disclosure, for example those incorporating a radioactive isotope, may be useful in drug and/or substrate tissue distribution studies. Particularly, compounds having the depicted structures that differ only in the replacement with heavier isotopes, such as the replacement of hydrogen by deuterium (²H, or D), can afford certain therapeutic advantages, for example, resulting from greater metabolic stability, increased in vivo half-life, or reduced dosage requirements and, hence, may be utilized in some particular circumstances. Isotopic variants of compounds or any formula depicting and describing the compounds of the present disclosure can generally be prepared by techniques known to those skilled in the art or by processes analogous to those described in the accompanying examples and synthesis using an appropriate isotopically-labeled reagent in place of the non-labeled reagent previously employed.

The compounds as provided herein are described with reference to both generic formulas and specific compounds. In addition, the compounds of the present disclosure may exist in a number of different forms or derivatives, all within the scope of the disclosure. These include, for example, pharmaceutically acceptable salts, tautomers, stereoisomers, racemic mixtures, regioisomers, prodrugs, solvated forms, different crystal forms or polymorphs, and active metabolites, etc.

As used herein, the term “pharmaceutically acceptable salt”, unless otherwise stated, includes salts that retain the biological effectiveness of the free acid/base form of the specified compound and that are not biologically or otherwise undesirable. Pharmaceutically acceptable salts may include salts formed with inorganic bases or acids and organic bases or acids. In cases where the compounds of the present disclosure contain one or more acidic or basic groups, the disclosure also comprises their corresponding pharmaceutically acceptable salts. Thus, the compounds of the present invention which contain acidic groups, such as carboxyl groups, can be present in salt form, and can be used according to the invention, for example, as alkali metal salts, alkaline earth metal salts, aluminum salts or as ammonium salts. More non-limiting examples of such salts include lithium salts, sodium salts, potassium salts, calcium salts, magnesium salts, barium salts, or salts with ammonia or organic amines such as ethylamine, ethanolamine, diethanolamine, triethanolamine, piperidine, N-methylglutamine, or amino acids. These salts are readily available, for instance, by reacting the compound having an acidic group with a suitable base, e.g., lithium hydroxide, sodium hydroxide, sodium propoxide, potassium hydroxide, potassium ethoxide, magnesium hydroxide, calcium hydroxide, or barium hydroxide. Other base salts of compounds of the present disclosure include but are not limited to copper (I), copper (II), iron (II), iron (III), manganese (II), and zinc salts. Compounds of the present disclosure which contain one or more basic groups, e.g., groups which can be protonated, can be present in salt form, and can be used according to the disclosure in the form of their addition salts with inorganic or organic acids. Examples of suitable acids include hydrogen chloride, hydrogen bromide, hydrogen iodide, phosphoric acid, sulfuric acid, nitric acid, methanesulfonic acid, p-toluenesulfonic acid, naphthalenedisulfonic acid, sulfoacetic acid, trifluoroacetic acid, oxalic acid, acetic acid, tartaric acid, lactic acid, salicylic acid, benzoic acid, carbonic acid, formic acid, propionic acid, pivalic acid, diethylacetic acid, malonic acid, succinic acid, pimelic acid, fumaric acid, malonic acid, maleic acid, malic acid, embonic acid, mandelic acid, sulfaminic acid, phenylpropionic acid, gluconic acid, ascorbic acid, isonicotinic acid, citric acid, adipic acid, taurocholic acid, glutaric acid, stearic acid, glutamic acid, or aspartic acid, and other acids known to those skilled in the art. The salts which are formed are, inter alia, hydrochlorides, chlorides, hydrobromides, bromides, iodides, sulfates, phosphates, methanesulfonates (mesylates), tosylates, carbonates, bicarbonates, formates, acetates, sulfoacetates, triflates, oxalates, malonates, maleates, succinates, tartrates, malates, embonates, mandelates, fumarates, lactates, citrates, glutarates, stearates, aspartates, and glutamates. The stoichiometry of the salts formed from the compounds of the disclosure may moreover be an integral or non-integral multiple of one.

Compounds of the present disclosure which contain basic nitrogen-containing groups can be quaternized using agents such as C₁₋₄alkyl halides, for example, methyl, ethyl, isopropyl, and tert-butyl chloride, bromide, and iodide; diC₁₋₄alkyl sulfates, for example, dimethyl, diethyl, and diamyl sulfate; C₁₀₋₁₈alkyl halides, for example, decyl, dodecyl, lauryl, myristyl, and stearyl chloride, bromide, and iodide; and arylC₁₋₄alkyl halides, for example, benzyl chloride and phenethyl bromide.

If the compounds of the present disclosure simultaneously contain acidic and basic groups in the molecule, the disclosure also includes, in addition to the salt forms mentioned, inner salts or betaines (zwitterions). The respective salts can be obtained by customary methods which are known to those skilled in the art, for example by contacting these with an organic or inorganic acid or base in a solvent or dispersant, or by anion exchange or cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts. For a review on more suitable salts, see Stahl and Wermuth, Handbook of Pharmaceutical Salts: Properties, Selection, and Use (Wiley-VCH, 2002).

The compound or any formula depicting and describing the compounds of the present disclosure and pharmaceutically acceptable salts thereof may exist in unsolvated and solvated forms. As used herein, the term “solvate” refers to a molecular complex comprising the compound of Formula (I), or a pharmaceutically acceptable salt thereof, and one or more pharmaceutically acceptable solvent molecules. For example, the term “hydrate” is employed when the solvent is water.

Pharmaceutically acceptable solvates in accordance with the present disclosure may include those wherein the solvent of crystallization may be isotopically substituted, e.g., D₂O, d₆-acetone, d₆-DMSO.

### Linker (Linking Agent Compound)

In some embodiments, the therapeutic agent is conjugated via a linker (or a linking agent compound). As used herein, the term “linker” or “linking agent compound” refers to a compound that can connect a ligand (e.g., the antibodies, the antigen-binding fragments thereof, or the antigen-binding protein constructs (e.g., bispecific antibodies) described herein) and a therapeutic agent (e.g., any of the therapeutic agents described herein) together to form a ligand-drug conjugate by reacting with a group of the ligand compound and the therapeutic agent compound respectively by, for example, a coupling reaction.

In some embodiments, the linker described herein is a compound having the following formula:

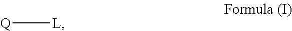

or a pharmaceutically acceptable salt, solvate, stereoisomer, or isotopic variant thereof, wherein Q denotes to a junction moiety capable of being coupled to a ligand via a bond selected from the group consisting of carbonyl, thioether, amide, disulfide and hydrazone bond; L denotes to a linker moiety capable of connecting Q to a therapeutic agent.

In some embodiments, the junction moiety (Q in Formula (I)) has the following structure:

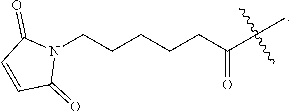 [SMILES: *C(=O)CCCCCN1C(=O)C=CC1=O]

In some embodiments, the linker moiety (L in Formula (I)) has the following formula:

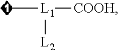

where L₁ is a polypeptide residue consisting of three to eight amino acid residues which comprises at least one amino acid residue with a side chain carboxyl group, for example, glutamic acid residue or aspartic acid residue, where “—COOH” denotes carboxyl group of an amino acid residue at C-terminal of the polypeptide residue;
L₂ is absent or a monodentate, bidentate or tridentate hydrophilic group attached to the side chain carboxyl group on the amino acid residue of the polypeptide residue L₁, and L₂ has a structure of —NHC(R^{L2a})(R^{L2b})(R^{L2c}), where R^{L2a}, R^{L2b}, and R^{L2c} are each independently selected from the group consisting of H, —(CH₂O)(CH₂CH₂O)ₘ(CH₂)ₚC(O)OH, and —(CH₂O) (CH₂CH₂O)ₘ(CH₂)ₚC(O)NHR^{L2d}, R^{L2d} is H or C₁₋₆ alkyl optionally substituted with 1 to 6 hydroxy groups, each m is independently an integer from 0 to 10, preferably 0 to 4, for example 0, 1, 2, 3, or 4, especially preferably m is 0, and each p is independent an integer from 1 to 4, for example, 1, 2, 3, or 4; and
[symbol] denotes to the N-terminal side of the polypeptide residue covalently attached to the junction moiety Q.

In some embodiments, the polypeptide residue L₁ is ^{NH}-Glu-Val-Ala-^{COOH}. In some embodiments, the hydrophilic group L₂ has the following structure:

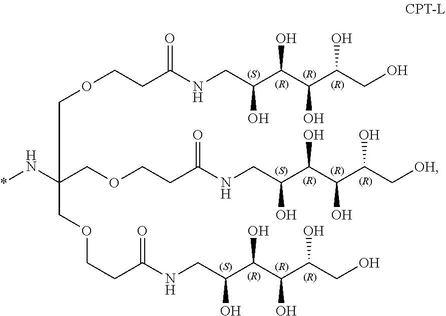

wherein “*” denotes the site covalently attached to polypeptide residue Li, e.g., side chain of the Glu residue in ^{NH}-Glu-Val-Ala-^{COOH}.

In some embodiments, the linker described herein is a compound having the following structure:

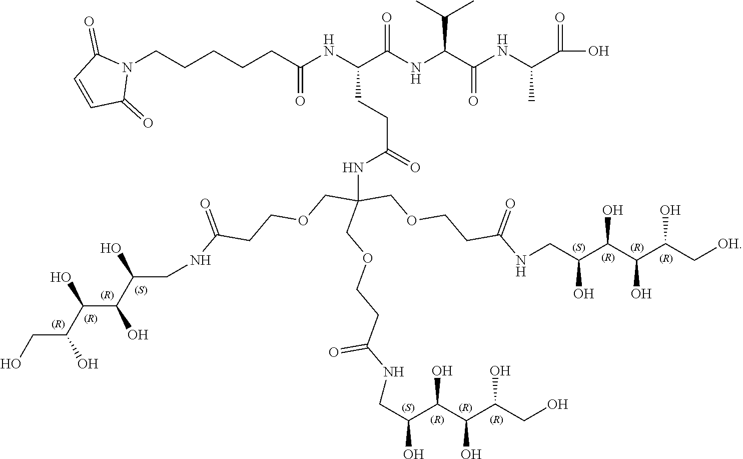

In some embodiments, the linker is a VC linker. Details of the linkers used for ADCs can be found, e.g., in Su, Z. et al. “Antibody-drug conjugates: Recent advances in linker chemistry.” Acta Pharmaceutica Sinica B (2021), which is incorporated herein by reference in its entirety.

### Therapeutic Agent

In some embodiments, the therapeutic agent that is conjugated to the antibodies, the antigen-binding fragments thereof, or the antigen-binding protein constructs (e.g., bispecific antibodies) described herein is discussed as follows.

In some embodiments, the therapeutic agent described herein is a cytotoxic agent. In some embodiments, the cytotoxic agent is a camptothecin compound, an analogue or a derivative thereof. In some preferred embodiments, the camptothecin compound is a compound having the following structure:

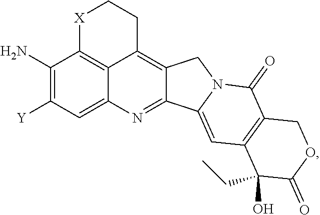

wherein X is selected from the group consisting of —CH2-, O and S; Y is selected from the group consisting of H, D, and F.

In some embodiments, the therapeutic agent is (S)-4-amino-9-ethyl-9-hydroxy-1,9,12,15-tetrahydro-13H-pyrano[3′,4′:6,7]indolizino[1,2-b]thiopyrano[4,3,2-de]quinoline-10,13(2H)-dione) (CPT-1). The structure of CPT-1 is shown below:

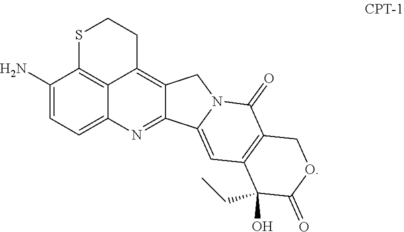 [SMILES: CC[C@@]1(O)C(=O)OCc2c1cc1n(c2=O)Cc2c-1nc1ccc(N)c3c1c2CCS3]

In some embodiments, the therapeutic agent is (S)-4-amino-9-ethyl-9-hydroxy-1,9,12,15-tetrahydro-13H-pyrano[4,3,2-de]pyrano[3′,4′:6,7]indolizino[1,2-b]quinoline-10,13(2H)-dione (CPT-2). The structure of CPT-2 is shown below:

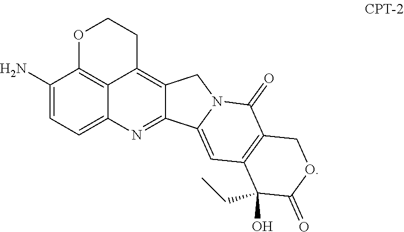 [SMILES: CC[C@@]1(O)C(=O)OCc2c1cc1n(c2=O)Cc2c-1nc1ccc(N)c3c1c2CCO3]

In some embodiments, the therapeutic agent is CPT3. The structure of CPT-3 is shown below:

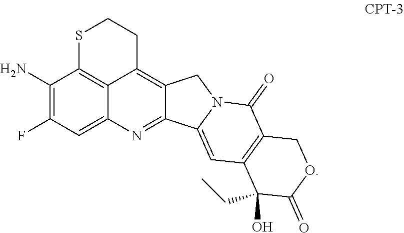 [SMILES: CC[C@@]1(O)C(=O)OCc2c1cc1n(c2=O)Cc2c-1nc1cc(F)c(N)c3c1c2CCS3]

In some embodiments, the therapeutic agent is (S)-4-amino-9-ethyl-5-fluoro-9-hydroxy-1,9,12,15-tetrahydro-13H-pyrano[4,3,2-de]pyrano[3′,4′:6,7]indolizino[1,2-b]quinoline-10,13(2H)-dione (CPT-4). The structure of CPT-4 is shown below:

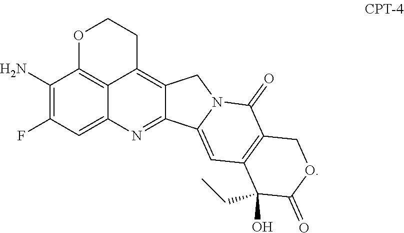 [SMILES: CC[C@@]1(O)C(=O)OCc2c1cc1n(c2=O)Cc2c-1nc1cc(F)c(N)c3c1c2CCO3]

In some embodiments, the therapeutic agent is an auristatin, such as auristatin E (also known in the art as a derivative of dolastatin-10) or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include AFP, MMAF, and MMAE. The synthesis and structure of exemplary auristatins are described in U.S. Patent Application Publication No. 2003-0083263; International Patent Publication No. WO 04/010957, International Patent Publication No. WO 02/088172, and U.S. Pat. Nos. 7,498,298, 6,884,869, 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4.879,278; 4,816,444; and 4,486,414, each of which is incorporated by reference herein in its entirety and for all purposes.

Auristatins have been shown to interfere with microtubule dynamics and nuclear and cellular division and have anticancer activity. Auristatins bind tubulin and can exert a cytotoxic or cytostatic effect on cancer cell. There are a number of different assays, known in the art, which can be used for determining whether an auristatin or resultant antibody-drug conjugate exerts a cytostatic or cytotoxic effect on a desired cell.

In some embodiments, the therapeutic agent is a chemotherapeutic agent. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK7; razoxane; sizofiran; spirogermanium; tenuazonic acid; triaziquone; 2′,2′,2′-trichlorotriethylamine; urethan; vindesine; dacarbazine; mannomustine; mitobronitol; mitolactol; pipobroman; gacytosine; arabinoside (“Ara-C”); cyclophosphamide; taxanes, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. A detailed description of the chemotherapeutic agents can be found in, e.g., US20180193477A1, which is incorporated by reference in its entirety.

### Linker-Therapeutic Agent Compound

In some embodiments, a linker (e.g., any of the linkers described herein) and a therapeutic agent (e.g., any of the therapeutic agents described herein) can be linked to form a “linker-therapeutic agent” compound.

In some embodiments, the linker-therapeutic agent compound has the following structure:

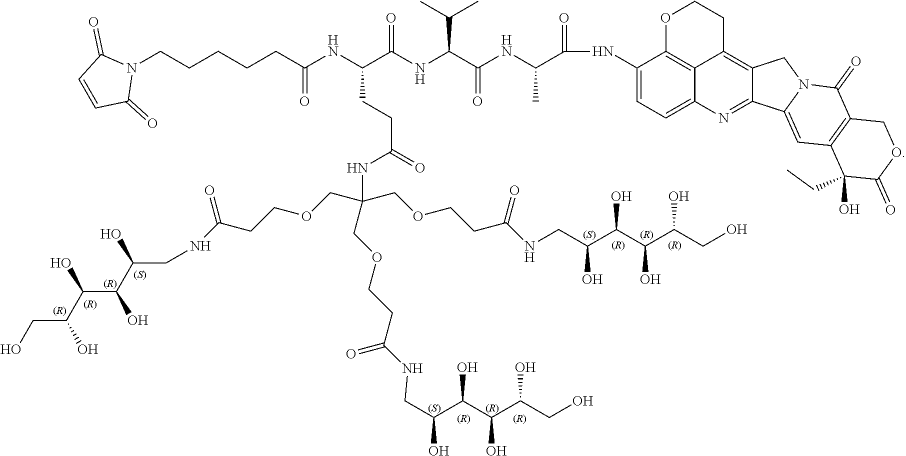

In some embodiments, the linker-therapeutic agent compound has the following structure:

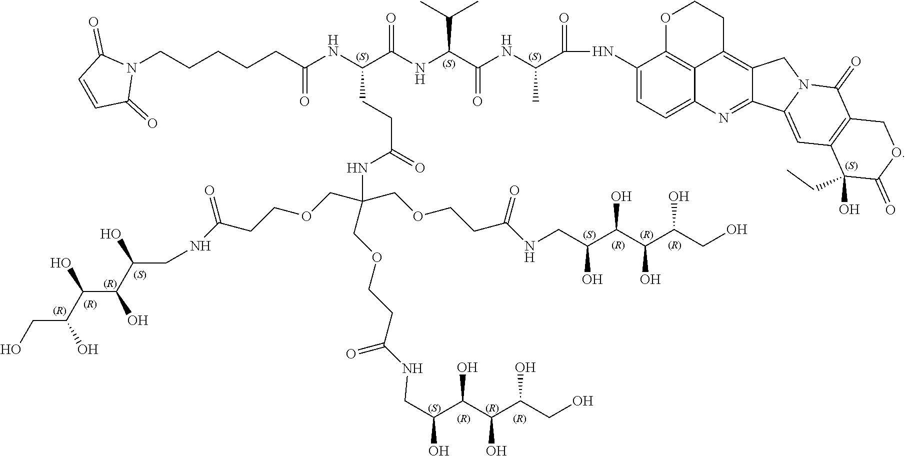

In some embodiments, an antibody (“Ab”), e.g., any of the antibodies, the antigen-binding fragments thereof, or the antigen-binding protein constructs (e.g., bispecific antibodies) described herein, can be linked to a linker-therapeutic agent compound (e.g., any of the linker-therapeutic agent compounds described herein) to generate an antibody-drug conjugate. In some embodiments, the antibody-drug conjugate has the following structure:

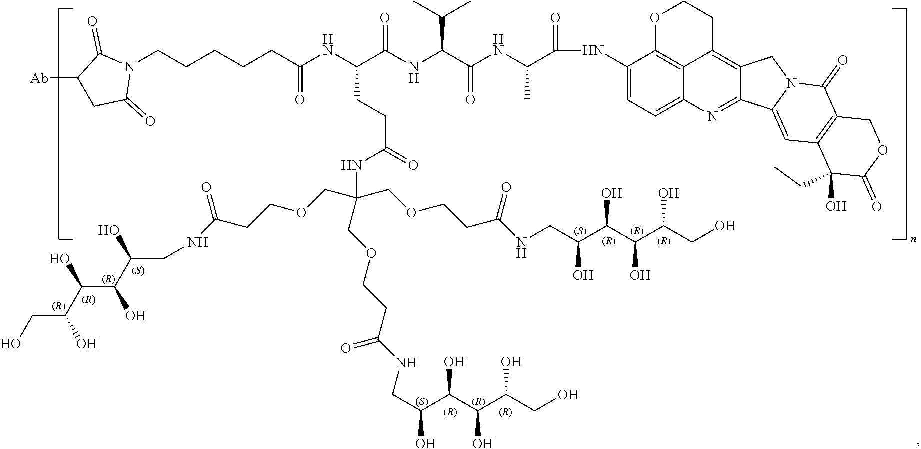

wherein n=1, 2, 3, 4, 5, 6, 7, or 8.

### Antibody and ADC Characteristics

The anti-EGFR/MET antigen-binding protein construct (e.g., antibodies, bispecific antibodies, or antibody fragments thereof) can include an antigen-binding region that is derived from any anti-EGFR antibody or any antigen-binding fragment thereof as described herein.

The disclosure provides antibodies and antigen-binding fragments thereof that specifically bind to EGFR. The antibodies and antigen-binding fragments described herein are capable of binding to EGFR. These antibodies can be agonists or antagonists. The anti-EGFR antibodies, or antigen-binding fragments thereof described herein can bind to EGFR, and block the binding between EGFR and EGF, and/or the binding between EGFR and TGFα. By blocking the binding between EGFR and EGF, and/or the binding between EGFR and TGFα, the anti-EGFR antibodies can inhibit the EGFR-associated signaling pathway and thus treating cancer (e.g., NSCLC). In some embodiments, the anti-EGFR antibodies, or antigen-binding fragments thereof can initiate CDC or ADCC.

General techniques can be used to measure the affinity of an antibody for an antigen include, e.g., ELISA, RIA, and surface plasmon resonance (SPR). Affinities can be deduced from the quotient of the kinetic rate constants (KD=koff/kon). In some implementations, the antibodies, the antigen-binding fragments thereof, or the antigen-binding protein construct (e.g., bispecific antibody), can bind to EGFR (e.g., human EGFR, monkey EGFR, mouse EGFR, and/or chimeric EGFR) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

In some embodiments, the antibodies, the antigen-binding fragments thereof, or the antigen-binding protein construct (e.g., bispecific antibody) can bind to EGFR (e.g., human EGFR, monkey EGFR, mouse EGFR, and/or chimeric EGFR) with a KD of less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 50 nM, 40 nM, 30 nM, 20 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, or greater than 1×10⁻¹⁰ M.

The anti-EGFR/MET antigen-binding protein construct (e.g., bispecific antibodies) can also include an antigen-binding region that is derived from any anti-MET antibody or antigen-binding fragment thereof as described herein. The anti-MET antibodies or antigen-binding fragments thereof described herein can block the binding between MET and HGF. In some embodiments, by binding to MET, the antibody can also inhibit MET-associated signaling pathways, thereby inhibiting cell proliferation, differentiation, and/or metastasis. Thus, in some embodiments, the antibodies or antigen-binding fragments thereof as described herein are MET agonist. In some embodiments, the antibodies or antigen-binding fragments thereof are MET antagonist.

In some implementations, the antibodies, the antigen-binding fragments thereof, or the antigen-binding protein constructs (e.g., bispecific antibody) can bind to MET (e.g., human MET, monkey MET, mouse MET, and/or chimeric MET) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷ M, less than 1×10⁻⁹ M, less than 1×10⁻⁹ M, or less than 1×10⁻¹⁰ M. In some embodiments, the KD is less than 50 nM, 40 nM, 30 nM, 20 nM, 15 nM, 10 nM, 9 nM, 8 nM, 7 nM, 6 nM, 5 nM, 4 nM, 3 nM, 2 nM, or 1 nM.

In some embodiments, KD is greater than 1×10⁻⁷ M, greater than 1×10⁻⁸ M, greater than 1×10⁻⁹ M. or greater than 1×10⁻¹⁰ M.

Because the antigen-binding protein construct (e.g., bispecific antibody) binds to both MET and EGFR, for cells that express both MET and EGFR, the antigen-binding protein construct has a higher binding affinity to these cells. Avidity can be used to measure the binding affinity of an antigen-binding protein construct to these cells. Avidity is the accumulated strength of multiple affinities of individual non-covalent binding interactions.

Thermal stabilities can also be determined. The antibodies, the antigen-binding fragments thereof, or the antigen-binding protein constructs (e.g., bispecific antibody) as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. As IgG can be described as a multi-domain protein, the melting curve sometimes shows two transitions, with a first denaturation temperature, Tm D1, and a second denaturation temperature Tm D2. The presence of these two peaks often indicate the denaturation of the Fc domains (Tm D1) and Fab domains (Tm D2), respectively. When there are two peaks, Tm usually refers to Tm D2. Thus, in some embodiments, the antibodies or antigen binding fragments as described herein has a Tm D1 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, the antibodies or antigen binding fragments as described herein has a Tm D2 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm, Tm D1, Tm D2 are less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibodies, the antigen-binding fragments thereof, the antigen-binding protein constructs (e.g., bispecific antibody), can bind to human EGFR or monkey EGFR. In some embodiments, the antibodies, the antigen-binding fragments thereof, the antigen-binding protein constructs (e.g., bispecific antibody), cannot bind to human EGFR or monkey EGFR. In some embodiments, the antibodies, the antigen-binding fragments thereof, the antigen-binding protein constructs (e.g., bispecific antibody), can bind to human MET or monkey MET. In some embodiments, the antibodies, the antigen-binding fragments thereof, the antigen-binding protein constructs (e.g., bispecific antibody), cannot bind to human MET or monkey MET.

In some embodiments, the antibodies, the antigen-binding fragments thereof, the antigen-binding protein constructs (e.g., the anti-MET antibody, the anti-EGFR antibody, or the bispecific antibody) has a purity that is greater than 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by HPLC. In some embodiments, the purity is less than 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by HPLC.

In some embodiments, the antibodies, the antigen-binding fragments thereof, or the antigen-binding protein constructs (e.g., the anti-MET antibody, the anti-EGFR antibody, or the bispecific antibody) has a tumor growth inhibition rate or percentage (TGI %) that is greater than 10%, 20%, 30%, 40%, 50%, 60%, 70%, 800%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, or 150%. The TGI (%) can be determined, e.g., at 3, 4, 5, 6, 7, 8, 9, 10, 11, 12, 13, 14, 15, 16, 17, 18, 19, 20, 21, 22, 23, or 24 days after the treatment starts. As used herein, the tumor growth inhibition rate or percentage (TGI %) is calculated using the following formula:

$$TGI ( \% ) = [ 1 - ( Ti - T 0 ) / ( Vi - V 0 ) ] \times 100 \%$$

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibody, the antigen-binding fragment thereof, or the antigen-binding protein construct (e.g., bispecific antibody) has a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4.

In some embodiments, the antibody, the antigen-binding fragment thereof, or the antigen-binding protein construct (e.g., bispecific antibody) does not have a functional Fc region. For example, the antibodies or antigen binding fragments are Fab, Fab′, F(ab′)₂, and Fv fragments.

In some embodiments, the protein constructs as described herein have an Fc region without effector function. In some embodiments, the Fc is a human IgG4 Fc. In some embodiments, the Fc does not have a functional Fc region. For example, the Fc region has LALA mutations (L234A and L235A mutations in EU numbering), or LALA-PG mutations (L234A, L235A, P329G mutations in EU numbering).

Some other modifications to the Fc region can be made. For example, a cysteine residue(s) can be introduced into the Fc region, thereby allowing interchain disulfide bond formation in this region. The homodimeric fusion protein thus generated may have any increased half-life in vitro and/or in vivo.

In some embodiments, the IgG4 has S228P mutation (EU numbering). The S228P mutation prevents in vivo and in vitro IgG4 Fab-arm exchange.

In some embodiments, Fc regions are provided having a carbohydrate structure that lacks fucose attached (directly or indirectly) to an Fc region. For example, the amount of fucose in such Fc region composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in Fc region sequences. Such fucosylation variants may have improved ADCC function. In some embodiments, to reduce glycan heterogeneity, the Fc region can be further engineered to replace the Asparagine at position 297 with Alanine (N297A).

In some embodiments, the main peak of HPLC-SEC accounts for at least 80%, 85%, 90%, 95%, 96%, 97%, 98%, 99%, or 99.5% of the protein complex described herein after purification by protein A-based affinity chromatography and/or size-exclusive chromatography.

In some embodiments, the ADC described herein has an IC50 for in vitro killing of cancer cells (e.g., lung cancer cell line NCI-H1975) of less than 2 μg/ml, less than 1.5 μg/ml, less than 1 μg/ml, less than 0.9 μg/ml, less than 0.8 μg/ml, less than 0.7 μg/ml, less than 0.6 μg/ml, less than 0.5 μg/ml, less than 0.4 μg/ml, less than 0.3 μg/ml, less than 0.2 μg/ml, or less than 0.1 μg/ml.

In some embodiments, the bispecific antibody described herein has a higher endocytosis rate than the corresponding monoclonal antibodies and/or control bispecific antibodies described herein. In some embodiments, the anti-EGFR antibody described herein has a higher endocytosis rate than Cetuximab analog. In some embodiments, the anti-MET antibody described herein has a higher endocytosis rate than Telisotuzumab analog. In some embodiments, the bispecific antibody described herein has a higher endocytosis rate than Amivantamab analog.

### Methods of Making Antigen-Binding Protein Constructs

An isolated fragment of human protein can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of the protein and encompasses an epitope of the protein such that an antibody raised against the peptide forms a specific immune complex with the protein.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of the protein) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Phage display (panning) can be used to optimize antibody sequences with desired binding affinities. In this technique, a gene encoding single chain Fv (comprising VH or VL) can be inserted into a phage coat protein gene, causing the phage to “display” the scFv on its outside while containing the gene for the protein on its inside, resulting in a connection between genotype and phenotype. These displaying phages can then be screened against target antigens, in order to detect interaction between the displayed antigen binding sites and the target antigen. Thus, large libraries of proteins can be screened and amplified in a process called in vitro selection, and antibodies sequences with desired binding affinities can be obtained.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

In some embodiments, a mouse (e.g., RenMab™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode the light chains of antibodies (kappa chain). The kappa chain immunoglobulin locus can include e.g., human IGKV (variable) genes, human IGKJ (joining) genes, and mouse light chain constant domain genes. A detailed description regarding RenMab™ mice can be found in PCT/CN2020/075698 or US20200390073A1, which is incorporated herein by reference in its entirety.

In some embodiments, a mouse (e.g., RenLite™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode a common light chain. The kappa chain immunoglobulin locus can include e.g., a human IGKV (variable) gene, a human IGKJ (joining) gene, and mouse light chain constant domain genes. A detailed description regarding RenLite™ mice can be found in PCT/CN2021/097652, which is incorporated herein by reference in its entirety.

Identity or homology with respect to an original sequence is usually the percentage of amino acid residues present within the candidate sequence that are identical with a sequence present within the human, humanized, or chimeric antibody or fragment, after aligning the sequences and introducing gaps, if necessary, to achieve the maximum percent sequence identity, and not considering any conservative substitutions as part of the sequence identity.

In some embodiments, a covalent modification can be made to the antibodies, the antigen-binding fragments thereof, or the antigen-binding protein constructs (e.g., bispecific antibodies). These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the methods described here are designed to make a bispecific antibody. Bispecific antibodies can be made by engineering the interface between a pair of antibody molecules to maximize the percentage of heterodimers that are recovered from recombinant cell culture. For example, the interface can contain at least a part of the CH₃ domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, knobs-into-holes (KIH) technology can be used, which involves engineering CH3 domains to create either a “knob” or a “hole” in each heavy chain to promote heterodimerization. The KIH technique is described e.g., in Xu, Yiren, et al. “Production of bispecific antibodies in ‘knobs-into-holes’ using a cell-free expression system.” MAbs. Vol. 7. No. 1. Taylor & Francis, 2015, which is incorporated by reference in its entirety. In some embodiments, one heavy chain has a T366W, and/or S354C (knob) substitution (EU numbering), and the other heavy chain has an Y349C, T366S, L368A, and/or Y407V (hole) substitution (EU numbering). In some embodiments, one heavy chain has one or more of the following substitutions Y349C and T366W (EU numbering). The other heavy chain can have one or more the following substitutions E356C, T366S, L368A, and Y407V (EU numbering). Furthermore, a substitution (-ppcpScp->-ppcpPcp-) can also be introduced at the hinge regions of both substituted IgG.

Bispecific antibodies can also include e.g., cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bispecific antibodies from antibody fragments are also known in the art. For example, bispecific antibodies can be prepared using chemical linkage. Brennan et al. (Science 229:81, 1985) describes a procedure where intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bispecific antibody.

### Recombinant Vectors

The present disclosure also provides recombinant vectors (e.g., expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors. Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of sequence homology (e.g., amino acid sequence homology or nucleic acid homology) can also be determined. How to determine percentage of sequence homology is known in the art. In some embodiments, amino acid residues conserved with similar physicochemical properties (percent homology), e.g. leucine and isoleucine, can be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

The disclosure provides one or more nucleic acid encoding any of the polypeptides as described herein. In some embodiments, the nucleic acid (e.g., cDNA) includes a polynucleotide encoding a polypeptide of a heavy chain as described herein. In some embodiments, the nucleic acid includes a polynucleotide encoding a polypeptide of a light chain as described herein. In some embodiments, the nucleic acid includes a polynucleotide encoding a scFv polypeptide as described herein.

In some embodiments, the vector can have two of the nucleic acids as described herein, wherein the vector encodes the VL region and the VH region that together bind to EGFR. In some embodiments, a pair of vectors is provided, wherein each vector comprises one of the nucleic acids as described herein, wherein together the pair of vectors encodes the VL region and the VH region that together bind to EGFR.

In some embodiments, the vector includes two of the nucleic acids as described herein, wherein the vector encodes the VL region and the VH region that together bind to MET. In some embodiments, a pair of vectors is provided, wherein each vector comprises one of the nucleic acids as described herein, wherein together the pair of vectors encodes the VL region and the VH region that together bind to MET.

Vectors can also be constructed to express specific antibodies or polypeptides. In some embodiments, a vector can be constructed to co-express anti-EGFR antibody light chain (EGFR-K) and heavy chain (EGFR-H). In some embodiments, a vector can contain sequences of, from 5′ end to 3′ end, cytomegalovirus promotor (CMV), EGFR-K, polyadenylation (PolyA), CMV, EGFR-H, PolyA, simian vacuolating virus 40 terminator (SV40) and glutamine synthetase marker (GS). In some embodiments, a vector can be constructed to co-express anti-MET antibody light chain (MET-K) and anti-MET antibody heavy chain (MET-H). In some embodiments, a vector can contain sequences of, from 5′ end to 3′ end, CMV, MET-K, PolyA, CMV. MET-H, SV40 and GS. In some embodiments, a vector can be constructed to express anti-MET antibody scFv polypeptide chain. In some embodiments, a first vector expressing antibody heavy chains (e.g., any of the heavy chains described herein) and a second vector expressing antibody light chains (e.g., any of the light chains described herein) are used to co-transfect cells (e.g., CHO cells) to produce the monoclonal antibody or antigen-binding fragment thereof described herein. In some embodiments, a first vector expressing an anti-EGFR antibody heavy chain (e.g., any of the anti-EGFR antibody heavy chains described herein), a second vector expressing an anti-MET antibody heavy chain (e.g., any of the anti-MET antibody heavy chains described herein), and a third vector expressing a common light chain (e.g., any of the common light chains described herein) are used to co-transfect cells (e.g., CHO cells) to produce the antigen-binding protein construct described herein (e.g., any of the anti-EGFR/MET bispecific antibodies described herein).

### Methods of Treatment

The methods described herein include methods for the treatment of disorders associated with cancer. Generally, the methods include administering a therapeutically effective amount of engineered antibodies, the antigen-binding fragments thereof, or the antigen-binding protein constructs (e.g., bispecific antibodies) as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the cancer is a chemotherapy resistant cancer.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of antibodies, the antigen-binding fragments thereof, or the antigen-binding protein constructs (e.g., bispecific antibodies), or an antibody drug conjugates disclosed herein to a subject in need thereof, e.g., a subject having, or identified or diagnosed as having, a cancer, e.g., solid tumor, lung cancer (e.g., non-small cell lung cancer, lung adenocarcinoma, or lung carcinoma), gastric cancer (e.g., gastric carcinoma), skin cancer (e.g., skin carcinoma), colorectal cancer, breast cancer, head and neck cancer, ovarian cancer, prostate cancer, thyroid cancer, pancreatic cancer, CNS cancer, liver cancer, nasopharynx cancer, ampullary carcinoma or brain cancer.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-drug conjugates, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody, an antigen binding fragment, or an antibody-drug conjugate is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody, antigen binding fragment, or antibody-drug conjugate may vary, depending on, inter alia, patient history as well as other factors such as the type (and/or dosage) of antibody used.

Effective amounts and schedules for administering the antibodies, antibody-encoding polynucleotides, antibody-drug conjugates, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, antibody-drug conjugates, and/or compositions disclosed herein, the route of administration, the particular type of antibodies, antibody-encoding polynucleotides, antigen binding fragments, antibody-drug conjugates, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody, the antigen-binding fragment thereof, or the antigen-binding protein construct (e.g., a bispecific antibody) is 0.01 mg/kg to 100 mg/kg. In some embodiments, the dosage can be less than 100 mg/kg, 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about or at least 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, the antigen-binding fragment thereof, or the antigen-binding protein construct (e.g., a bispecific antibody), antibody-drug conjugates, or pharmaceutical composition (e.g., any of the antibodies, antigen-binding fragments, antibody-drug conjugates, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody, the antigen-binding fragment thereof, the antigen-binding protein construct (e.g., a bispecific antibody), or antibody-drug conjugate, and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, antibody-drug conjugate, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, antibody-drug conjugate, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, antibody-drug conjugate, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments, antibody-drug conjugates (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-PD-1 antibody, an anti-PD-L1 antibody, anti-PD-L2 antibody, an anti-LAG-3 antibody, an anti-TIGIT antibody, an anti-BTLA antibody, an anti-CTLA4 antibody, an anti-CD40 antibody, an anti-OX40 antibody, an anti-4-1BB antibody, an anti-TIM3 antibody, or an anti-GITR antibody.

### Pharmaceutical Compositions and Routes of Administration

Also provided herein are pharmaceutical compositions that contain at least one (e.g., one, two, three, or four) of the antigen-binding protein constructs, antibodies (e.g., bispecific antibodies), antigen-binding fragments, or antibody-drug conjugates described herein. Two or more (e.g., two, three, or four) of any of the antigen-binding protein constructs, antibodies, antigen-binding fragments, or antibody-drug conjugates described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradennal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antigen-binding protein constructs, antibodies, antigen-binding fragments, antibody-drug conjugates described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antigen-binding protein constructs, antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antigen-binding protein constructs, antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antigen-binding protein constructs, antibodies or antigen-binding fragments, or antibody-drug conjugates described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 0.1 mg/kg to about 0.5 mg/kg). While these doses cover a broad range, one of ordinary skill in the art will understand that therapeutic agents, including antigen-binding protein constructs, antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof, or antibody-drug conjugates for various uses as described herein.

### EXAMPLES

The invention is further described in the following examples, which do not limit the scope of the invention described in the claims.

### Example 1. Preparation and Analysis of Anti-EGFR Antibody

### Generation of Anti-EGFR Antibodies

Human EGFR protein (hEGFR-Fc, ACRO Biosystems Inc., Cat #: EGR-H5252) or DNA encoding this protein was emulsified with adjuvants, and was used to immunize RenLite™ mice (Biocytogen, complete human heavy chain variable domain combined with a common light chain substitution in situ). The mice are described, e.g., in PCT/CN2021/097652, which is incorporated herein by reference in its entirety. Before immunization, retro-orbital blood was collected as a negative control.

Freund's complete adjuvant (CFA) was used for the first immunization and Freund's incomplete adjuvant (IFA) was used for the second, third, and fourth immunizations. A total of four immunizations were performed. The first and second immunizations were separated by two weeks, and the remaining immunizations were separated by one week. One week after the fourth immunization, retro-orbital blood was collected, and the antibody titer of serum was detected by fluorescence activated cell sorting (FACS). One week later, mice with high titer were further injected with hEGFR-Fc protein and CHO-S cells expressing human EGFR antigen through the tail vein for impulse immunization.

Antigen-specific immune cells were isolated from the immunized mice to further obtain anti-EGFR antibodies or to obtain the light chain and heavy chain variable region sequences of the anti-EGFR antibodies. For example, single cell technology (e.g., Beacon® Optofluidic System, Berkeley Lights Inc.) was used to screen and find plasma cells secreting antigen-specific monoclonal antibodies, and then reverse transcription and PCR sequencing were used to obtain antibody variable region sequences. The obtained variable region sequences were cloned into a vector containing a sequence encoding the human IgG1 constant region for antibody expression. Binding of the expressed antibodies to EGFR was verified by FACS. Exemplary antibodies obtained by this method included: E-1C12, E-1G11, E-2B7, E-3A8, E-3D8, E-6C4, E-9A6, E-9D2, E-9F3 and E-9H2.

These antibodies contained substantially the same light chain, and their VH and VL CDR 1-3 sequences are shown in FIG. 8, FIG. 9 or FIG. 10. The VH and VL regions of E-1G11, E-2B7, E-3A8, E-3D8, E-6C4, E-9A6, E-9D2, E-9F3 and E-9H2 are shown in FIG. 11.

### Cross-Species Binding Analysis of Anti-EGFR Antibodies

CHO-S-hEGFR cells and CHO-S-fasEGFR cells were obtained by transfecting CHO-S cells with vectors encoding amino acids 25-645 of human EGFR protein (SEQ ID NO: 31) and amino acids 25-645 of monkey (crab-eating macaque; or Macaca fascicularis) EGFR protein (SEQ ID NO: 32), respectively. The CHO-S-hEGFR cells or CHO-S-fasEGFR cells were transferred to 96-well plates respectively, at a density of 5×10⁴ cells/well. Serially diluted anti-EGFR antibodies were added to the 96-well plate, and incubated at 4° C. for 30 minutes. Afterwards, the cells were incubated with a secondary antibody Anti-hIgG-Fc-Alex Flour 647 (RL1-H) (Jackson ImmunoResearch Laboratories, Inc., Cat #: 109-606-170) at 4° C. in the dark for 15 minutes, followed by flow cytometry analysis. As shown in the table below, all six anti-EGFR antibodies can bind to human EGFR and monkey EGFR.

TABLE 1

| Antibody | EGFR Protein | Percentage of Positive Cells | Evaluation |
|---|---|---|---|
| NC | hEGFR | 0 | No binding |
| | fasEGFR | 0.2% | No binding |
| E-1C12 | hEGFR | 29.9% | Binding |
| | fasEGFR | 28.2% | Binding |
| E-1G11 | hEGFR | 34.2% | Binding |
| | fasEGFR | 31.9% | Binding |
| E-2B7 | hEGFR | 26.1% | Binding |
| | fasEGFR | 28.6% | Binding |
| E-3A8 | hEGFR | 31.2% | Binding |
| | fasEGFR | 23.8% | Binding |
| E-3D8 | hEGFR | 28.8% | Binding |
| | fasEGFR | 24.1% | Binding |
| E-6C4 | hEGFR | 26.4% | Binding |
| | fasEGFR | 25.4% | Binding |

In another similar experiment, the cross-species binding activities of anti-EGFR antibodies (E-9A6, E-9D2, E-9F3 and E-9H2) to CHO-S-hEGFR cells or CHO-S-fasEGFR were verified by flow cytometry. The results are shown in the table below.

TABLE 2

| Antibody | EGFR Protein | Percentage of Positive Cells | Evaluation |
|---|---|---|---|
| Negative | hEGFR | 0.46% | No binding |
| control | fasEGFR | 0.65% | No binding |
| E-9A6 | hEGFR | 56.9% | Binding |
| | fasEGFR | 55.4% | Binding |
| E-9D2 | hEGFR | 38.7% | Binding |
| | fasEGFR | 45.9% | Binding |
| E-9F3 | hEGFR | 53.1% | Binding |
| | fasEGFR | 53.7% | Binding |
| E-9H2 | hEGFR | 44.3% | Binding |
| | fasEGFR | 41.5% | Binding |

The results showed that anti-EGFR antibodies E-9A6, E-9D2, E-9F3 and E-9H2 can bind to human EGFR and monkey EGFR.

### Binding Affinity of Anti-EGFR Antibodies

The binding affinity of the anti-EGFR antibodies to His-tagged human EGFR protein (hEGFR-His, ACROBiosystems Inc., Cat #: EGR-H5222) and His-tagged monkey (crab-eating macaque; or Macaca fascicularis) EGFR protein (fasEGFR-His, ACROBiosystems Inc., Cat #: EGR-C52H1) was measured by surface plasmon resonance (SPR) using Biacore™ (Biacore, Inc., Piscataway N.J.) 8K biosensor equipped with pre-immobilized Protein A sensor chips.

Each purified anti-EGFR antibody was captured on the Protein A chip (Series S Sensor Chip Protein A) for the detection. Specifically, 1 μg/mL purified anti-EGFR antibody was loaded at 10 μL/min to bind to a gradient concentration of recombinant hEGFR-His or fasEGFR-His (200 nM). The flow rate was 30 μL/min. The binding and dissociation times were set to 180 seconds and 400 seconds, respectively. The chip was regenerated after the last injection of each titration with a glycine solution (pH 2.0) at 30 μL/min for 30 seconds.

Data analysis was performed in Biacore™ 8K Evaluation Software 3.0 using a standard 1:1 binding model. Affinity values were deduced from the quotient of the kinetic rate constants (KD=koff/kon).

As a person of ordinary skill in the art would understand, the same method with appropriate adjustments for parameters (e.g., antibody concentration) was performed for each tested anti-EGFR antibody. The results for the tested antibodies are summarized in the table below.

TABLE 3

| Antibody | EGFR protein | kon (1/Ms) | koff (1/s) | KD (M) |
|---|---|---|---|---|
| E-1C12 | hEGFR-His | 4.63E+04 | 5.21E−04 | 1.12E−08 |
| | fasEGFR-His | 7.53E+04 | 4.77E−06 | 6.34E−11 |
| E-1G11 | hEGFR-His | 4.23E+04 | 1.23E−03 | 2.90E−08 |
| | fasEGFR-His | 3.67E+04 | 9.12E−04 | 2.49E−08 |
| E-2B7 | hEGFR-His | 6.10E+04 | 2.86E−04 | 4.69E−09 |
| | fasEGFR-His | 6.43E+04 | 3.42E−04 | 5.32E−09 |
| E-3A8 | hEGFR-His | 4.35E+04 | 1.01E−03 | 2.32E−08 |
| | fasEGFR-His | 4.87E+04 | 1.04E−03 | 2.13E−08 |
| E-3D8 | hEGFR-His | 3.39E+04 | 1.32E−03 | 3.90E−08 |
| | fasEGFR-His | 3.87E+04 | 1.32E−03 | 3.40E−08 |
| E-6C4 | hEGFR-His | 1.38E+05 | 8.65E−03 | 6.27E−08 |
| | fasEGFR-His | 1.90E+05 | 1.72E−02 | 9.03E−08 |

The results showed that all six anti-EGFR antibodies can bind to both human and monkey EGFR with a good binding affinity.

In another similar experiment, the binding affinity of anti-EGFR antibodies (E-9A6, E-9D2, E-9F3 and E-9H2) to hEGFR-His or fasEGFR-His was verified. As shown in the table below, all four antibodies E-9A6, E-92, E-9F3 and E-9H2 can bind to human EGFR and monkey EGFR.

TABLE 4

| Antibody | EGFR protein | kon (1/Ms) | koff (1/s) | KD (M) |
|---|---|---|---|---|
| E-9A6 | hEGFR-His | 1.86E+05 | 1.76E−03 | 9.44E−09 |
| | fasEGFR-His | 1.38E+05 | 1.73E−03 | 1.26E−08 |
| E-9D2 | hEGFR-His | 2.52E+05 | 1.52E−02 | 6.03E−08 |
| | fasEGFR-His | 2.03E+05 | 2.03E−02 | 9.98E−08 |
| E-9F3 | hEGFR-His | 6.49E+04 | 1.77E−03 | 2.72E−08 |
| | fasEGFR-His | 4.76E+04 | 1.90E−03 | 3.99E−08 |
| E-9H2 | hEGFR-His | 1.54E+05 | 1.51E−02 | 9.83E−08 |
| | fasEGFR-His | 1.17E+05 | 1.77E−02 | 1.52E−07 |

### Internalization of Anti-EGFR Antibodies

Anti-EGFR antibodies (2.5 μg/ml) and pHAb-Goat Anti-Human IgG secondary antibody (Promega (Beijing) Biotech Co., Ltd., Cat #: G9845) were added to SNU-5 cells (ATCC, Reference No.: CRL-5973). After incubating at 37° C. and 5% CO₂ for 1 hour, the cells were centrifuged and washed with FACS buffer. The mean fluorescence intensity (MFI) values were measured using a flow cytometer, and the endocytosis rates of the anti-EGFR antibodies were calculated. The results are shown in the table below.

TABLE 5

| Antibody | MFI | Percentage of Positive |
|---|---|---|
| NC | 11418 | 0.51% |
| NC + Secondary antibody | 11472 | 0.65% |
| Cetuximab analog | 14719 | 1.54% |
| E-1C12 | 13696 | 2.13% |
| E-1G11 | 20158 | 32.90% |
| E-2B7 | 14556 | 3.92% |
| E-3A8 | 16120 | 7.98% |
| E-3D8 | 16937 | 10.20% |
| E-6C4 | 15005 | 4.72% |

Cetuximab is an EGFR-targeting chimeric monoclonal IgG1 antibody originally developed by ImClone Systems and first launched in Switzerland in 2003 as Erbitux™ by Merck KGaA as a monotherapy and in combination with irinotecan for the treatment of irinotecan-refractory metastatic colorectal cancer, and its heavy chain and light chain sequences are shown in SEQ ID NO: 73 and SEQ ID NO: 74, respectively.

In another similar experiment, the internalization of anti-EGFR antibodies (E-9A6, E-9D2, E-9F3 and E-9H2) in A431 cells (ATCC, Reference No.: CRL-1555) for 1 hour were verified by flow cytometry. The secondary antibody used in the experiment was: pHAb-AffiniPure Fab Fragment Goat Anti-Human IgG (Jackson Immuno Research, Cat #: 109-007-008). The results are shown in the table below.

TABLE 6

| Antibody | Percentage of Positive |
|---|---|
| NC | 0.48% |
| NC + Secondary antibody | 0.82% |
| E-9A6 | 98.2% |
| E-9D2 | 99.6% |
| E-9F3 | 98.3% |
| E-9H2 | 95.6% |

### Example 2. Preparation and Analysis of Anti-MET Antibodies

### Generation of Anti-MET Antibodies

hMET-Fc (ACROBiosystems Inc., Cat #: MET-H5256) was used to immunize RenLite™ mice. Before immunization, retro-orbital blood was collected from the mice as a negative control. Freund's complete adjuvant (CFA) was used for the first immunization, and Freund's incomplete adjuvant (IFA) was used for the second, third, and fourth immunizations. A total of four immunizations were conducted. One week after the fourth immunization, retro-orbital blood was collected, and the serum titer of anti-human MET antibodies was detected by FACS. Two weeks after the fourth immunization, hMET-Fc and CHO-S cells expressing human MET antigen were used for impulse immunization by tail vein injection.

Antigen-specific immune cells were isolated (from immune organs of the immunized mice) to further obtain anti-MET antibodies or to obtain the light chain and heavy chain variable region sequences of the anti-MET antibodies. Single-cell technology (e.g., Beacon® Optofluidic System, Berkeley Lights Inc.) was used to screen and find plasma cells secreting antigen-specific monoclonal antibodies, and then reverse transcription and PCR sequencing were used to obtain the antibody variable region sequences. The obtained variable region sequences were cloned into a vector containing a sequence encoding the human IgG1 constant region for antibody expression. Binding of the expressed antibodies to MET was verified by FACS. Exemplary fully human antibodies obtained by this method included: M-1C5, M-1E9, M-2F11, M-2F12, M-2G10, M-6D12, M-6E6, M-7D3, M-8D9, M-8E2 and M-81H10. These antibodies have the same light chain, whose sequence is consistent with the common light chain sequence of the anti-EGFR antibodies, and their VH CDR 1-3 sequences are shown in FIG. 8 and FIG. 9. The VH regions of M-1E9, M-2F11, M-2F12, M-2G10, M-6D12, M-6E6, M-7D3, M-8D9, M-8E2 and M-8H10 are shown in FIG. 11.

### Cross-Species Binding Analysis of Anti-MET Antibodies

SNU-5 cells or fasMET-NIH3T3 cells were transferred to a 96-well plate at a density of 5×10⁴ cells/well. Serially diluted anti-MET antibodies were added into the 96-well plate, and incubated at 4° C. for 30 minutes. Afterwards, the cells were incubated with Anti-hIgG-Fc-Alex Flour 647 (RL1-H) (Jackson ImmunoResearch Laboratories, Inc., Cat #:109-606-170) at 4° C. in the dark for 15 minutes, followed by flow cytometry analysis. As shown in the table below, all anti-MET antibodies can bind to human and monkey MET except M-2F12.

TABLE 7

| Antibody | Cell | Percentage of Positive Cells | Evaluation |
|---|---|---|---|
| NC | SNU-5 | 2.5% | No binding |
| | fasMET-NIH3T3 | 0.8% | No binding |
| M-1C5 | SNU-5 | 94.6% | Binding |
| | fasMET-NIH3T3 | 20.4% | Binding |
| M-1E9 | SNU-5 | 99.3% | Binding |
| | fasMET-NIH3T3 | 48.8% | Binding |
| M-2F11 | SNU-5 | 97.1% | Binding |
| | fasMET-NIH3T3 | 71.1% | Binding |
| M-2F12 | SNU-5 | 95.6% | Binding |
| | fasMET-NIH3T3 | 0.18% | No binding |
| M-2G10 | SNU-5 | 98.7% | Binding |
| | fasMET-NIH3T3 | 21.0% | Binding |

SNU-5 is human gastric carcinoma cell line with high MET expression. fasMET-NIH3T3 cells were obtained by transfecting NIH3T3 cells to express monkey (Macaca fascicularis) MET protein (SEQ ID NO: 33).

In another similar experiment, the cross-species binding activities of anti-MET antibodies M-6D12, M-6E6, M-7D3, M-8D9, M-8E2 and M-8H10 to hMET-NIH3T3 cells, fasMET-NIH3T3 cells or dMET-NIH3T3 cells were verified by flow cytometry. The results are shown in the table below. As shown in the table below, all anti-MET antibodies can bind to human and monkey MET.

TABLE 8

| Antibody | Cell | Percentage of Positive Cells | Evaluation |
|---|---|---|---|
| NC | hMET-NIH3T3 | 1.01% | No binding |
| | fasMET-NIH3T3 | 1.29% | No binding |
| | dMET-NIH3T3 | 0.29% | No binding |
| M-6D12 | hMET-NIH3T3 | 99.8% | Binding |
| | fasMET-NIH3T3 | 71.4% | Binding |
| | dMET-NIH3T3 | 5.33% | No binding |
| M-6E6 | hMET-NIH3T3 | 99.2% | Binding |
| | fasMET-NIH3T3 | 49.2% | Binding |
| | dMET-NIH3T3 | 73.2% | Binding |
| M-7D3 | hMET-NIH3T3 | 99.6% | Binding |
| | fasMET-NIH3T3 | 63.4% | Binding |
| | dMET-NIH3T3 | 74.7% | Binding |
| M-8D9 | hMET-NIH3T3 | 99.8% | Binding |
| | fasMET-NIH3T3 | 65.3% | Binding |
| | dMET-NIH3T3 | 58.2% | Binding |
| M-8E2 | hMET-NIH3T3 | 99.6% | Binding |
| | fasMET-NIH3T3 | 62.2% | Binding |
| | dMET-NIH3T3 | 78.8% | Binding |
| M-8H10 | hMET-NIH3T3 | 99.8% | Binding |
| | fasMET-NIH3T3 | 67.8% | Binding |
| | dMET-NIH3T3 | 1.44% | No binding |

hMET-NIH3T3 cells were obtained by transfecting NIH-3T3 cells to express human MET protein (SEQ ID NO: 154). dMET-NIH13T3 cells were obtained by transfecting NIH3T3 cells to express dog (Cans lupus) MET protein (SEQ ID NO:155).

### Binding Affinity of Anti-MET Antibodies

The binding affinity of the anti-MET antibodies to His-tagged human MET protein (hMET-His, ACROBiosystems Inc., Cat #: MET-4-H5227) and His-tagged monkey MET (fasMET-His, Sino Biological Inc., Cat #: 70008-E048H) was measured using Biacore-3 (Biacore, Inc., Piscataway N.J.) 8K biosensor equipped with pre-immobilized Protein A sensor chips. The results for the tested antibodies are summarized in the table below.

TABLE 9

| Antibody | MET protein | kon (1/Ms) | koff (1/s) | KD (M) |
|---|---|---|---|---|
| M-1E9 | hMET-His | 2.32E+04 | 6.98E−04 | 3.01E−08 |
| | fasMET-His | 2.74E+04 | 2.99E−04 | 1.09E−08 |
| M-2F11 | hMET-His | 5.77E+04 | 3.13E−03 | 5.42E−08 |
| | fasMET-His | 9.09E+04 | 4.68E−03 | 5.16E−08 |
| M-2G10 | hMET-His | 1.89E+05 | 6.05E−03 | 3.19E−08 |
| | fasMET-His | 3.32E+05 | 1.68E−02 | 5.06E−08 |

The results showed that M-1E9, M-2F11 and M-2G10 can bind to both human and monkey MET with a good binding affinity.

In another similar experiment, the binding affinity of anti-MET antibodies M-6D12, M-6E6, M-7D3, M-819, M-8E2 and M-8H10 to hMET-His, fasMET-His or dMET-His (His-tagged dog MET, Sino, Cat #:70008-D08H) was verified. Telisotuzumab analog was used as a positive control. As shown in the table below, all six antibodies can bind to human MET and monkey MET, and M-6E6, M-7D3 and M-8E2 can also bind to dog MET with a good binding affinity. However, Telisotuzumab analog only showed binding affinity to human MET and monkey MET, 0 but no binding affinity to dog MET (data not shown).

Telisotuzumab is a humanized IgG1 monoclonal antibody targeting MET, which is in early clinical development at AbbVie for the treatment of advanced solid tumors with MET gene amplification, and its heavy chain and light chain sequences are shown in SEQ ID) NO: 75 and SEQ ID NO: 76, respectively.

TABLE 10

| Antibody | EGFR protein | kon (1/Ms) | koff (1/s) | KD (M) |
|---|---|---|---|---|
| M-6D12 | hMET-His | 3.91E+04 | 2.37E−03 | 6.06E−08 |
| | fasMET-His | 3.94E+04 | 4.16E−03 | 1.06E−07 |
| | dMET-His | — | — | — |
| M-6E6 | hMET-His | 6.25E+04 | 6.34E−04 | 1.01E−08 |
| | fasMET-His | 3.34E+04 | 6.08E−04 | 1.82E−08 |
| | dMET-His | 6.90E+04 | 1.75E−02 | 2.53E−07 |
| M-7D3 | hMET-His | 5.91E+04 | 6.84E−04 | 1.16E−08 |
| | fasMET-His | 4.83E+04 | 7.42E−04 | 1.54E−08 |
| | dMET-His | 5.50E+04 | 3.43E−03 | 6.23E−08 |
| M-8D9 | hMET-His | 2.71E+04 | 5.05E−04 | 1.86E−08 |
| | fasMET-His | 2.38E+04 | 9.90E−04 | 4.17E−08 |
| | dMET-His | — | — | — |
| M-8E2 | hMET-His | 5.22E+04 | 6.83E−04 | 1.31E−08 |
| | fasMET-His | 3.61E+04 | 1.25E−03 | 3.47E−08 |
| | dMET-His | 7.69E+04 | 3.84E−03 | 5.00E−08 |
| M-8H10 | hMET-His | 6.97E+04 | 1.38E−03 | 1.99E−08 |
| | fasMET-His | 8.35E+04 | 3.21E−03 | 3.85E−08 |
| | dMET-His | — | — | — |

(“—” means no binding)

The above results showed that all anti-MET antibodies can bind to human and monkey MET. M-6E6, M-7D3 and M-8E2 can also bind to dog MET.

### Internalization of Anti-MET Antibodies

Anti-MET antibodies (2.5% g/ml) and pHAb-GoatAnti-Human IgG Antibody (Promega (Beijing) Biotech Co., Ltd., Cat #: G9845) were added to SUN-5 cells. After incubating for 1 hour, the cells were centrifuged and washed with FACS buffer. The MFI values were measured using a flow cytometer, and the endocytosis rates of the anti-MET antibodies were calculated. The results are shown in the table below.

TABLE 11

| Antibody | MFI | Percentage of Positive |
|---|---|---|
| NC | 11418 | 0.51% |
| NC + Secondary antibody | 11473 | 0.65% |
| Telisotuzumab analog | 21700 | 36.9% |
| M-1C5 | 13681 | 2.32% |
| M-1E9 | 17123 | 14.9% |
| M-2F11 | 25321 | 62.6% |
| M-2F12 | 17123 | 15.7% |
| M-2G10 | 27024 | 69.2% |

The data showed that M-2F11 and M-2G10 had higher cell endocytosis rates compared with positive control Telisotuzunmab analog.

In another similar experiment, the internalization of anti-MET antibodies M-61D12, M-6E6, M-7D3, M-8D9, M-8E2 and M-8H10 in SNU-5 cells for 2.5 hour were verified by flow cytometry. The secondary antibody used in the experiment was: pHAb-AffiniPure Fab Fragment Goat Anti-Human IgG (Jackson Immuno Research, Cat #: 109-007-008). The results are shown in the table below, which showed that the anti-MET antibodies M-6D312, M-6E6, M-7D33, M-8D39, M-8E2 and M-8H10 showed high endocytosis rates at 2.5h in SNU-5 cells.

TABLE 12

| Antibody | MFI | Percentage of Positive |
|---|---|---|
| NC | 13177 | 1.00% |
| NC + Secondary antibody | 13528 | 1.14% |
| ISO | 13420 | 0.81% |
| M-6D12 | 42494 | 88.1% |
| M-6E6 | 38090 | 82.3% |
| M-7D3 | 39419 | 83.3% |
| M-8D9 | 35655 | 83.1% |
| M-8E2 | 38813 | 81.7% |
| M-8H10 | 43755 | 86.2% |

### Example 3. Preparation and Analysis of Anti-EGFR/MET Bispecific Antibodies

Provided herein are bispecific antigen-binding molecules targeting EGFR and MET. These antigen-binding molecules are referred to as anti-EGFR/MET bispecific antibody below.

### Preparation of Anti-EGFR/MET Bispecific Antibodies

Anti-EGFR antibodies (E-IC12, E-1G11, E-2B7, E-3A8, E-3D8, E-6C4, E-9A6, E-9D2, E-9F3 and E-9H2) and anti-MET antibodies (M-1C5, M-1E9, M-2F11, M-2F12, M-2G10, M-6D12, M-6E6, M-7D3, M-8D9, M-8E2 and M-8H10) can be paired to form various bispecific antibodies. Vectors encoding the light chain and heavy chain of the antibodies were constructed. CHO-S cells were co-transfected with three vectors, including a first vector encoding the heavy chain of an anti-EGFR antibody, a second vector encoding the heavy chain of an anti-MET antibody, and a third vector encoding the common light chain. After 14 days of culture, the cell supernatant was collected and purified by Protein A affinity chromatography.

Various methods can be used to reduce the chance of wrong pairing between the two heavy chains. For example, knobs-into-holes mutations were introduced in the Fc regions of the anti-EGFR arm heavy chain and the anti-MET arm heavy chain. Exemplary bispecific antibodies obtained include: E-1G11-M-1C5, E-1G11-M-1E9, E-1G11-M-2F11, E-1G11-M-2F12, E-1G11-M-2G10, E-2B7-M-1C5, E-2B7-M-1E9, E-2B7-M-2F11, E-2B7-M-2F12, E-2B7-M-2G10, E-3A8-M-1C5, E-3A8-M-1E9, E-3A8-M-2F11, E-3A8-M-2F12, E-3A8-M-2G10, E-3D8-M-1C5, E-3D8-M-1E9, E-3D8-M-2F11, E-3D8-M-2F12, E-3D8-M-2G10, E-6C4-M-1C5, E-6C4-M-1E9, E-6C4-M-2F11, E-6C4-M-2F12, E-6C4-M-2G10, E-9A6-M-1C5, E-9A6-M-1E9, E-9A6-M-2F11, E-9A6-M-2F12, E-9A6-M-2G10, E-9D2-M-1C5, E-9D2-M-1E9, E-9D2-M-2F11, E-9D2-M-2F12, E-9D2-M-2G10, E-9F3-M-1C5, E-9F3-M-1E9, E-9F3-M-2F11, E-9F3-M-2F12, E-9F3-M-2G10, E-9H2-M-1C5, E-9H2-M-1E9, E-9H2-M-2F11, E-9H2-M-2F12, E-9H2-M-2G10, E-1G11-M-6D12, E-1G11-M-6E6, E-1G11-M-7D3, E-1G11-M-8D9, E-1G11-M-8E2, E-1G11-M-8H10, E-2B7-M-6D12, E-2B7-M-6E6, E-2B7-M-7D3, E-2B7-M-8D9, E-2B7-M-8E2 and E-2B7-M-8H10. To verify the binding affinity of bispecific antibodies, anti-EGFR or anti-MET control bispecific antibodies were also generated, in which one arm of the control bispecific antibody recognizes EGFR or MET, and the other arm recognizes CD28. Similar methods were used to generated these control bispecific antibodies, e.g., obtaining VH sequences by immunizing RenLiter™ mice. Exemplary control bispecific antibodies are named as E-1G11-CD28 and M-2F12-CD28.

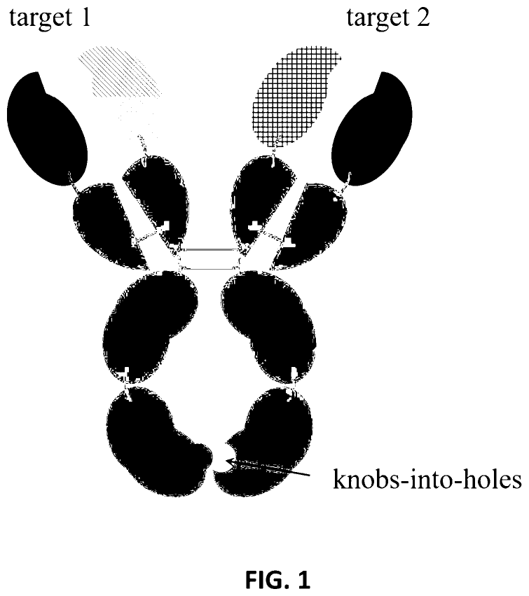

Knobs-into-holes mutations were introduced to all the bispecific antibodies. For example, in E-1G11-M-2F12, the heavy chain constant region of E-1G11 includes knob mutations, and the heavy chain constant region of M-2F12 includes hole mutations. In E-1G11-CD28, the heavy chain constant region of E-1G11 includes knob mutations, and the heavy chain constant region of CD28 includes hole mutations. An exemplary antibody structure is shown in FIG. 1, where target 1 and target 2 can be EGFR and MET; respectively; MET and EGFR, respectively; EGFR and CD28, respectively; or CD28 and MET, respectively.

The sequences of the light chain constant region, the heavy chain constant region with knob mutations, and the heavy chain constant region with hole mutations are shown in SEQ ID NO: 81, SEQ ID NO: 71 and SEQ ID NO: 72, respectively.

Internalization of Antibodies Targeting EGFR and/or MET

Anti-EGFR antibodies, anti-MET antibodies, or anti-EGFR/MET bispecific antibodies, together with the pHAb-Goat anti-human IgG secondary antibody were added to NCI-H1975 cells (ATCC, Reference No.: CRL-5908), HCC827 cells (ATCC, Reference No.: CRL-2868) and NCI-H292 (ATCC, Reference No.: CRL-1848) cells, respectively, and incubated for 1 hour. The cells were centrifuged and washed with FACS buffer. MFI was measured using a flow cytometer. Endocytosis rates of antibodies were calculated. For isotype control (ISO), an antibody targeting an irrelevant target protein was used. The results are shown in the following table.

TABLE 13

| | NCI-H1975 | | HCC827 | | NCI-H292 | |
|---|---|---|---|---|---|---|
| | | Positive | | Positive | | Positive |
| Antibodies | MFI | Population | MFI | Population | MFI | Population |
| ISO | 10667 | 4.79% | 9332 | 0.50% | 5607 | 1.01% |
| NC | 10016 | 1.96% | 8892 | 0.58% | 5657 | 0.95% |
| Telisotuzumab | Not detected | Not detected | 10344 | 0.63% | 6969 | 2.32% |
| analog | | | | | | |
| Amivantamab | Not detected | Not detected | 30401 | 88.90% | 13815 | 67.00% |
| analog | | | | | | |
| E-6C4 | 24191 | 66.70% | 27250 | 85.40% | 10858 | 30.50% |
| M-2F11 | 22321 | 55.00% | 10344 | 0.75% | 6867 | 2.48% |
| M-2G10 | 22321 | 57.20% | 11139 | 1.48% | 7113 | 3.07% |
| CD28-E-6C4 | 17034 | 25.10% | 11689 | 1.40% | 8866 | 9.22% |
| CD28-M-2F11 | 14880 | 24.80% | 8778 | 0.74% | 6142 | 1.90% |
| M-2G10-CD28 | 15667 | 24.30% | 9183 | 0.75% | 6288 | 1.65% |
| E-6C4-M-2F11 | 34467 | 83.50% | 16601 | 32.30% | 10179 | 19.80% |
| E-6C4-M-2G10 | 25388 | 67.50% | 14134 | 16.60% | 9458 | 12.40% |

Amivantamab is a fully human bispecific antibody targeting EGFR and MET developed by Janssen. It was approved in the U.S. and in the E.U, in 2021 under the name of Rybrevant for the treatment of adult patients with locally advanced or metastatic non-small cell lung cancer (NSCLC) with EGFR exon 20 insertion mutations whose disease has progressed on or after platinum-based chemotherapy. The heavy chain and light chain sequences of Amivantamab are shown in SEQ ID NOs: 77-80.

The results showed that the bispecific antibody E-6C4-M-2F11 had a higher endocytosis rate than E-6C4-M-2G10. These two bispecific antibodies also showed higher endocytosis rates than the corresponding monoclonal antibody M-2F II or M-2G10. In addition, the control bispecific antibodies CD28-E-6C4, CD28-M-2F11 and M-2G10-CD28 showed significantly lower endocytosis rates than the corresponding bispecific antibodies or monoclonal antibodies.

### Purity Analysis of Antibodies

Purified anti-EGFR/MET bispecific antibodies were analyzed by a non-reducing SDS-PAGE (sodium dodecyl sulfate-polyacrylamide gel electrophoresis) and SEC-UPLC (size exclusion chromatography-ultra performance liquid chromatography).

Non-reducing SDS-PAGE was performed using a 4-12% acrylamide gel. The protein samples were prepared as follows. First. 2.4 μL of the protein sample was mixed with 6 μL Tris-Glycine SDS Sample Buffer (2×) (Invitrogen, Cat #: LC2676) and 3.6 μL distilled water. The mixture was then boiled for 2 minutes and instantly centrifuged before loading. 4 g of each sample was loaded to the gel.

In the SEC-UPLC method, the antibody samples were diluted to 1 mg/mL with purified water and an Agilent 1290 chromatography system (connected with XBridge™ Protein BEH SEC column (200 Å, Waters Corporation)) was used. The following parameters were used: mobile phase: 100 mmol/L phosphate buffer (PB) (pH 7.4)+0.2 mol/L NaCl+10% acetonitrile; flow rate: 1.8 mU/min; column temperature: 25° C.; detection wavelength: 280 nm; injection volume: 10 μL; sample tray temperature: 6° C.; and running time: 7 minutes. Results are summarized in the table below.

TABLE 14

| Sample | Yield (mg/mL) | SEC-UPLC |
|---|---|---|
| Cetuximab analog | 5.10 | 100% |
| Telisotuzumab analog | 8.20 | 96.55% |
| E-1G11 | 10.10 | 100% |
| E-2B7 | 3.90 | 97.78% |
| E-3A8 | 6.40 | 97.99% |
| E-3D8 | 10.30 | 98.47% |
| E-6C4 | 6.00 | 95.64% |
| M-2F11 | 11.40 | 100% |
| M-2G10 | 11.60 | 96.42% |
| E-1G11-CD28 | 12.70 | 88.09% |
| E-3A8-CD28 | 6.50 | 71.37% |
| CD28-E-6C4 | 8.10 | 74.52% |
| CD28-M-2F11 | 5.80 | 98.62% |
| M-2G10-CD28 | 8.00 | 84.95% |
| E-1G11-M-2F11 | 10.40 | 97.81% |
| E-1G11-M-2G10 | 5.90 | 84.64% |
| E-3A8-M-2F11 | 3.90 | 95.76% |
| E-3D8-M-2G10 | 6.70 | 83.26% |
| E-6C4-M-2F11 | 7.10 | 100% |
| E-6C4-M-2G10 | 13.70 | 87.48% |

### Binding Activity of Anti-EGFR/MET Bispecific Antibodies

The binding activity of anti-EGFR/MET bispecific antibodies to human EGFR, human MET, monkey EGFR, and monkey MET were verified by surface plasmon resonance (SPR) using Biacore™ (Biacore, Inc., Piscataway N.J.) 8K biosensor equipped with pre-immobilized Protein A sensor chips.

Specifically, hEGFR-His (ACROBiosystems Inc., Cat #: EGR-H5222), hMET-His (ACROBiosystems Inc., Cat #: MET-H5227), fasEGFR-His (ACROBiosystems Inc., Cat #: EGR-C52H5), and fasMET-His (Sino Biological Inc., Cat #: 90304-C08H) were diluted to 400 nM, 200 nM, 100 nM, 50 nM, 25 nM, 6.25 nM, 3.125 nM, and 1.5626 nM, with 1×HBS-EP+ buffer (PH 7.4) and then injected into the Biacore™ 8K biosensor at 10 μL/min for about 50 seconds to achieve a desired protein density (e.g., about 100 response units (RU)). Purified antibodies at concentrations of 1 μg/ml with 1×HBS-EP+ buffer (PH 7.4) were then injected at 10 μL/min for 50 seconds. Dissociation was monitored for 400 seconds. The chip was regenerated after the last injection of each titration with a glycine solution (pH 1.5) at 30 μL/min for 30 seconds.

Kinetic association rates (kon) and dissociation rates (koff) were obtained simultaneously by fitting the data globally to a 1:1 Langmuir binding model (Karlsson, R. Roos. H. Fagerstam, L. Petersson, B., 1994. Methods Enzymology 6. 99-110) using Biacore™ 8K Evaluation Software 3.0. Affinities were deduced from the quotient of the kinetic rate constants (KD=koff/kon).

As a person of ordinary skill in the art would understand, the same method with appropriate adjustments for parameters (e.g., antibody concentration) was performed for each tested antibody. The results for the tested antibodies are summarized in the table below.

TABLE 15

| | Analyte 1 | | | |
|---|---|---|---|---|
| | Solution | kon (1/Ms) | koff (1/s) | KD (M) |
| Cetuximab | hEGFR-His | 1.40E+06 | 1.46E−03 | 1.05E−09 |
| analog | fasEGFR-His | 9.67E+05 | 1.74E−03 | 1.80E−09 |
| | hMET-His | — | — | No binding |
| | fasMET-His | — | — | No binding |
| Telisotuzumab | hEGFR-His | — | — | No binding |
| analog | fasEGFR-His | — | — | No binding |
| | hMET-His | 1.03E+05 | 1.00E−04 | 9.72E−10 |
| | fasMET-His | 6.26E+04 | 9.41E−05 | 1.50E−09 |
| E-2B7 | hEGFR-His | 7.15E+04 | 3.01E−04 | 4.22E−09 |
| | fasEGFR-His | 4.56E+04 | 2.96E−04 | 6.49E−09 |
| | hMET-His | — | — | No binding |
| | fasMET-His | — | — | No binding |
| E-6C4 | hEGFR-His | 2.40E+05 | 2.22E−02 | 9.24E−08 |
| | fasEGFR-His | 1.49E+05 | 2.35E−02 | 1.58E−07 |
| | hMET-His | — | — | No binding |
| | fasMET-His | — | — | No binding |
| M-2F11 | hEGFR-His | — | — | No binding |
| | fasEGFR-His | — | — | No binding |
| | hMET-His | 3.96E+04 | 2.20E−03 | 5.56E−08 |
| | fasMET-His | 3.50E+04 | 3.78E−03 | 1.08E−07 |
| M-2G10 | hEGFR-His | — | — | No binding |
| | fasEGFR-His | — | — | No binding |
| | hMET-His | 5.70E+04 | 3.62E−03 | 6.36E−08 |
| | fasMET-His | 7.72E+04 | 9.69E−03 | 1.26E−07 |
| E-2B7-M-2F11 | hEGFR-His | 7.38E+04 | 5.03E−04 | 6.82E−09 |
| | fasEGFR-His | 4.43E+04 | 5.22E−04 | 1.18E−08 |
| | hMET-His | 3.87E+04 | 1.09E−03 | 2.81E−08 |
| | fasMET-His | 4.30E+04 | 3.85E−03 | 8.94E−08 |
| E-6C4-M-2F11 | hEGFR-His | 3.04E+05 | 2.15E−02 | 7.07E−08 |
| | fasEGFR-His | 1.72E+05 | 1.98E−02 | 1.15E−07 |
| | hMET-His | 3.70E+04 | 1.87E−03 | 5.05E−08 |
| | fasMET-His | 3.63E+04 | 3.91E−03 | 1.08E−07 |
| E-6C4-M-2G10 | hEGFR-His | 3.28E+05 | 1.74E−02 | 5.31E−08 |
| | fasEGFR-His | 1.36E+05 | 1.10E−02 | 8.09E−08 |
| | hMET-His | 3.95E+04 | 1.65E−03 | 4.17E−08 |
| | fasMET-His | 5.59E+04 | 7.00E−03 | 1.25E−07 |

The results showed that the anti-EGFR/MET bispecific antibodies E-2B7-M-2F11, E-6C4-M-2F11, and E-6C4-M-2G10 can all bind to human EGFR, human MET, monkey EGFR, and monkey MET.

The binding activity of anti-EGFR/MET bispecific antibody E-6C4-M-2F11 to other human EGF family proteins was also verified, the result showed that E-6C4-M-2F11 can not bind human HER2, HER3 and HER4 (Data not shown).

Stability of anti-EGFR/MET bispecific antibodies

Six anti-EGFR/MET bispecific antibodies E-1G11-M-2F11, E-3 Å8-M-2F11, E-3 Å8-M-2G10, E-3D8-M-2F11, E-6C4-M-2F11 and E-6C4-M-2G10 were diluted to 5 mg/ml using a buffer at pH 6.0 (3 mg/ml histidine, 80 mg/ml sucrose, and 0.2 mg/ml Tween® 80). The diluted antibodies were kept in sealed Eppendorf tubes at 4±3° C. (hereinafter referred to as 4° C.) for 7 days; or at 40±3° C. (hereinafter referred to as 40° C.) for 7 days, and their thermal stability was evaluated. Alternatively, the bispecific antibodies were frozen at −80° C. then thawed at room temperature. The freeze-thaw experiment was repeated 10 times (in 5 days) and the antibody samples were detected after the last thaw at room temperature. The bispecific antibodies were also incubated at low pH conditions. Specifically, the antibodies were incubated in 1 mol/L acetic acid at pH 3.5 for 0 hour or 6 hours.

After the above treatments, the following analyses were performed: (1) observing the solution appearance and presence of visible non-soluble objects; (2) detecting the purity changes of antibodies by Size-Exclusion Ultra Performance Liquid Chromatography (SEC-UPLC) (indicated as the percentage of the main peak area to the sum of all peak areas (Purity, %)); (3) detecting changes in the apparent hydrophobicity of the antibodies using the Hydrophobic Interaction Chromatography-High Performance Liquid Chromatography (HIC-HPLC) method (indicated as the retention time of the main peak (HIC, min)); (4) detecting the purity changes of antibodies by capillary electrophoresis-sodium dodecyl sulphate (CE-SDS) under non-reducing (CE-SDS(NR)) conditions (indicated as the percentage of the main peak area to the sum of all peak areas (Purity, %)); (5) detecting charge variants in the antibodies by the Capillary Isoelectric Focusing (cIEF) method (indicated as the percentages of the main component, acidic component, and alkaline component).

In the SEC-UPLC experiments, the antibody samples were diluted to 1 mg/mL with purified water and an Agilent 1290 chromatograph system (connected with XBridge™ Protein BEH SEC column (200 Å, Waters Corporation)) was used. The following parameters were used: mobile phase: 100 mmol/L phosphate buffer (pH 7.4)+0.2 mol/L NaCl+10% acetonitrile; flow rate: 1.8 mL/min; column temperature: 25° C.; detection wavelength: 280 nm; injection volume: 10 μL; sample tray temperature: about 6° C.; and running time: 7 minutes.

In the HIC-HPLC experiments, Agilent 1260 chromatograph system (connected with ProPac™ HIC-10 column (4.6×250 mm, Thermo Scientific)) was used, and samples were diluted using mobile phase A to 0.5 mg/mL. The following parameters were used: mobile phase A: 0.9 M ammonium sulfate, 0.1 M phosphate buffer (PB), 10% acetonitrile pH 6.5; mobile phase B: 0.1 M PB, 10% acetonitrile pH 6.5; flow rate: 0.8 mL/min; gradient: 0 min 100% A, 2 min 100% A, 32 min 100% B, 34 min 100% B, 35 min 100% A, and 45 min 100% A; column temperature: 30° C.; detection wavelength: 280 nm; injection volume: 10 μL; sample tray temperature: about 6° C.; and running time: 45 minutes.

In the cIEF experiments, a Maurice cIEF Method Development Kit (Protein Simple, Cat #: PS-MDK01-C) was used for sample preparation. Specifically, 40 μg protein sample was mixed with the following reagents in the kit: 1 μL Maurice cIEF pl Marker-4.05, 1 μL Maurice cIEF pl Marker-9.99, 35 μL 1% Methyl Cellulose Solution, 2 μL Maurice cIEF 500 mM Arginine, 4 μL Ampholytes (Pharmalyte pH ranges 3-10), and water (added to make a final volume of 100 μL). On the Maurice analyzer (Protein Simple, Santa Clara, CA), Maurice cIEF Cartridges (PS-MC02-C) were used to generate imaging capillary isoelectric focusing spectra. The sample was focused for a total of 10 minutes. The analysis software installed on the instrument was used to integrate the absorbance of the 280 nm-focused protein.

In the CE-SDS(NR) experiments, Maurice (Protein simple, Maurice™) and Maurice CE-SDS Size Application Kit (Protein simple, Cat #: PS-MAK02-S) were used. 54 μL Sample Buffer, 6 μL antibody sample, 2.4 μL 25× internal standard, 3 μL 250 nM Iodoacetamide (SIGMA, Cat #: 16125) were add to a microcentrifuge tube, followed by centrifugation at 3000 rpm for 1 minute and heating in a 70° C. water bath for 10 min. The samples were then cooled to room temperature followed by centrifugation at 10000 rpm for 3 minutes. Supernatant sample preparations were then transferred to a 96-well plate and tested in Maurice. The following parameters were used: injection voltage 4.6 kV, injection time 20 seconds, separation voltage 5.75 kV, and separation time 40 minutes.

Detailed results of anti-EGFR/MET bispecific antibodies are shown in the table below. The results showed that E-6C4-M-2F11 and E-6C4-M-2G10 had better stability as well as physical and chemical properties than other tested antibodies.

TABLE 16

| | | | | | | | cIEF | | | |
|---|---|---|---|---|---|---|---|---|---|---|
| | | | | CE-SDS (NR) | | | Acidic | Main | Basic | |
| | | SEC | | Purity | HL | | peak | peak | peak | |
| Antibody | Treatment | Purity(%) | HIC (min) | (%) | (%) | HHL(%) | (%) | (%) | (%) | PI |
| E-1G11- | 0 d | 90.64 | 2.61/5.11 | 90.1 | 5.6 | 2.5 | 21.4 | 73.6 | 4.9 | 8.455 |
| M-2F11 | 40° C. 7 d | 92.28 | 2.63/5.09 | 89.0 | 5.0 | 4.3 | 26.7 | 66.7 | 6.6 | 8.546 |
| | freeze 10 | 90.65 | 2.60/5.11 | 89.0 | 5.5 | 3.7 | 22.2 | 72.8 | 5.0 | 8.527 |
| | pH 3.5 0 h | 94.45 | 2.57/5.18 | 96.8 | 3.2 | — | 32.7 | 54.7 | 12.6 | 8.753 |
| | pH 3.5 6 h | 95.61 | 2.47/5.17 | 91.1 | 4.0 | 1.4 | 25.7 | 68.4 | 5.9 | 8.732 |
| E-3D8- | 0 d | 95.10 | 4.69 | 68.3 | 16.7 | 5.5 | 8.0 | 85.5 | 6.5 | 8.590 |
| M-2G10 | 40° C. 7 d | 95.41 | 4.69 | 75.1 | 14.4 | 7.0 | — | 100 | — | 8.702 |
| | freeze 10 | 93.64 | 4.69 | 6.0 | 16.2 | 5.6 | 0.8 | 93.0 | 6.2 | 8.606 |
| | pH 3.5 0 h | 97.90 | 4.74 | 77.8 | 10.7 | 5.3 | 1.0 | 99.0 | — | 8.945 |
| | pH 3.5 6 h | 98.54 | 4.75 | 78.5 | 10.5 | 5.2 | 0.3 | 99.7 | — | 8.885 |
| E-3D8- | 0 d | 80.79 | 2.56 | 97.2 | — | 2.1 | 52.1 | 47.9 | | 8.836 |
| M-2F11 | 40° C. 7 d | 84.32 | 2.57 | 97.9 | — | 2.1 | 54.8 | 41.8 | 3.4 | 8.713 |
| | freeze 10 | 84.01 | 2.56 | 96.4 | — | 2.1 | 53.8 | 46.2 | — | 8.717 |
| | pH 3.5 0 h | 84.74 | 2.58 | 96.6 | — | 1.7 | 48.7 | 51.3 | — | 9.041 |
| | pH 3.5 6 h | 84.67 | 2.59 | 98.2 | — | 1.8 | 54.5 | 41.1 | 4.5 | 9.053 |
| E-6C4-M- | 0 d | 90.82 | 7.41 | 88.8 | 6.0 | 2.9 | 33.6 | 62.5 | 3.9 | 8.766 |
| 2F11 | 40° C. 7 d | 92.82 | 7.41 | 87.3 | 6.4 | 4.1 | 40.0 | 55.1 | 4.9 | 8.736 |
| | freeze 10 | 92.38 | 7.42 | 88.4 | 6.0 | 3.1 | 31.3 | 65.0 | 3.7 | 8.675 |
| | pH 3.5 0 h | 96.48 | 4.00/7.45 | 84.0 | 4.7 | 2.2 | 22.1 | 77.5 | 0.4 | 9.028 |
| | pH 3.5 6 h | 95.87 | 7.46 | 88.3 | 3.7 | 4.0 | 22.3 | 74.8 | 2.9 | 9.027 |
| E-604-M- | 0 d | 86.72 | 4.01 | 79.1 | 4.9 | 4.6 | 50.4 | 44.9 | 4.7 | 8.544 |
| 2G10 | 40° C. 7 d | 87.70 | 4.06 | 83.2 | 3.9 | 5.9 | 56.5 | 37.3 | 6.2 | 8.561 |
| | freeze 10 | 87.32 | 4.01 | 87.3 | 5.4 | 4.9 | 54.7 | 45.3 | — | 8.613 |
| | pH 3.5 0 h | 90.47 | 4.05 | 89.1 | 3.9 | — | 53.2 | 40.5 | 6.3 | 9.014 |
| | pH 3.5 6 h | 90.74 | 4.06 | 85.5 | 4.0 | 3.3 | 55.9 | 44.1 | — | 8.948 |
| | 0 d | 86.72 | 4.01 | 79.1 | 4.9 | 4.6 | 50.4 | 44.9 | 4.7 | 8.544 |

### Example 4. Antibody Drug Conjugates

After Protein A purification, bispecific antibodies E-1 Gil-M-2F11, E-1 G11-M-2G10, E-2B7-M-2F11, E-3 Å8-M-2F11, E-3 Å8-M-2G10, E-31D8-M-2F11, E-3D8-M-2G10, E-6C4-M-2F11 and E-6C4-M-2G10 were dialyzed and concentrated in PBS buffer by ultrafiltration. The concentration was determined by UV absorption. These antibodies were used for the subsequent antibody drug coupling reactions.

Coupling of Antibodies with Drug Molecules

The purified antibodies were coupled with MMAE (monomethyl auristatin E) or MMAF (monomethyl auristatin F) through a maleimidocaproyl-valine-citrulline-p-aminobenzyloxycarbonyl (VC) linker.

For the names of antibody-drug conjugates, “ADC” is added directly after the antibody name. For example, if E-1G11-M-2F11 is coupled to MMAE, it is named as E-1G11-M-2F11-ADC.

HIC-HPLC were used to detect the coupling of antibodies with drug molecules. In the HIC-HPLC experiments, an Agilent 1260 chromatography system (connected with ProPac™ HIC-10 column (4.6×250 mm, Thermo Scientific)) was used, and samples were diluted using mobile phase A to 0.5 mg/mL. The following parameters were used: mobile phase A: 0.9 M ammonium sulfate, 0.1 M phosphate buffer (PB), 10% acetonitrile pH 6.5; mobile phase B: 0.1 M PB, 10% acetonitrile pH 6.5; flow rate: 0.8 mL/min; gradient: 0 min 100% A, 2 min 100% A, 32 min 100% B, 34 min 100% B, 35 min 100% A, and 45 min 100% A; column temperature: 30° C.; detection wavelength: 280 nm; injection volume: 10 μL; sample tray temperature: about 6° C.; and running time: 45 minutes.

For isotype control, a human IgG1 isotype control was coupled to MMAE to form isotype-ADC (ISO-ADC). The HIC-HPLC detection results are shown in the table below. The results show that the drug-to-antibody ratio (DAR) of ADC is about 4. Wherein the average DAR is determined by multiplying PA % (PA % is the peak area percentage as measured by the area under the 280 nm peak) multiplied by the corresponding drug load of 0, 2, 4, 6, or 8 and divided by 100. For example, the average DAR of E-1G11-M-2F11-ADC can be calculated as: [(6.69×0)+(23.45×2)+(41.60×4)+(18.60×6)+(9.67×8)]/100=4.0.

TABLE 17

| | PA % at each DAR Peak | | | | | Average |
|---|---|---|---|---|---|---|
| | D0 | D2 | D4 | D6 | D8 | DAR |
| E-3A8-ADC | 5.92 | 27.59 | 23.51 | 35.96 | 7.03 | 4.2 |
| E-6C4-ADC | 4.10 | 29.48 | 43.39 | 17.40 | 5.64 | 3.8 |
| E-3D8-ADC | 1.29 | 27.51 | 39.21 | 22.28 | 9.71 | 4.2 |
| M-2G10-ADC | 1.80 | 22.70 | 42.65 | 22.01 | 10.83 | 4.3 |
| M-2F11-ADC | 2.58 | 26.21 | 47.04 | 17.71 | 6.46 | 4.0 |
| CD28-E-6C4-ADC | 4.23 | 27.85 | 29.95 | 25.35 | 13.07 | 4.3 |
| CD28-M-2F11-ADC | 1.76 | 21.83 | 44.04 | 22.18 | 10.19 | 4.3 |
| M-2G10-CD28-ADC | 2.68 | 26.06 | 39.50 | 21.14 | 10.61 | 4.2 |
| E-1G11-M-2F11-ADC | 6.69 | 23.45 | 41.60 | 18.60 | 9.67 | 4.0 |
| E-1G11-M-2G10-ADC | 6.29 | 20.76 | 33.71 | 26.52 | 12.72 | 4.4 |
| E-2B7-M-2F11-ADC | 8.28 | 26.40 | 27.54 | 26.80 | 10.98 | 4.1 |
| E-3A8-M-2F11-ADC | 4.94 | 25.87 | 46.26 | 16.68 | 6.26 | 3.9 |
| E-3A8-M-2G10-ADC | 6.97 | 24.68 | 25.84 | 32.43 | 10.08 | 4.3 |
| E-3D8-M-2F11-ADC | 1.86 | 19.36 | 39.66 | 22.28 | 16.84 | 4.7 |
| E-3D8-M-2G10-ADC | 3.05 | 22.24 | 32.81 | 23.38 | 18.52 | 4.6 |
| E-6C4-M-2F11-ADC | 5.34 | 30.04 | 39.23 | 18.46 | 6.92 | 3.8 |
| E-6C4-M-2G10-ADC | 5.97 | 30.57 | 36.78 | 19.60 | 7.08 | 3.8 |

### In Vitro Killing Activity

Different concentrations of purified antibodies (10 μg/mL, 3.33 μg/mL, 1.11 μg/mL, 0.37 μg/mL, 0.123 μg/mL, 0.041 μg/mL, 0.013 μg/mL, and 0.004 μg/mL) and corresponding ADCs were used to treat human lung cancer cell line NCI-H1975 (5×10³) cultured in a cell culture plate, and the killing activity was detected after 72 hours of incubation in IncuCyte (Sartorius AG, IncuCyte® S3). The results are shown in the table below.

TABLE 18

| Antibodies | IC50 (μg/mL) |
|---|---|
| Amivantamab analog | Not detected |
| E-6C4 | Not detected |
| M-2F11 | Not detected |
| E-6C4-ADC | 0.064 |
| M-2F11-ADC | 0.163 |
| CD28-E-6C4 | Not detected |
| CD28-M-2F11 | Not detected |
| CD28-E-6C4-ADC | 1.550 |
| CD28-M-2F11-ADC | 0.889 |
| E-6C4-M-2F11 | Not detected |
| E-6C4-M-2F11-ADC | 0.212 |

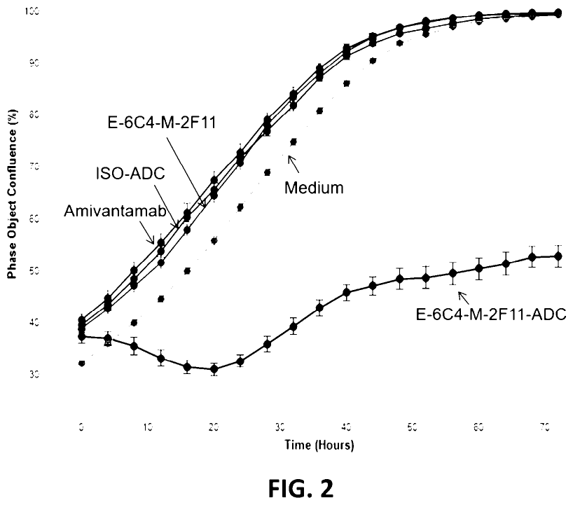

Some of the data was further analyzed, for example, the cancer cell killing results of the bispecific antibody E-6C4-M-2F11, and corresponding ADC E-6C4-M-2F11-ADC at a concentration of 10 μg/mL are shown in FIG. 2. Detailed killing results of E-6C4-M-2F11-ADC at different concentrations and different times are shown in the table below.

TABLE 19

| | 10 μg/mL | 3.33 μg/mL | 1.11 μg/mL | 0.37 μg/mL | 0.12 μg/mL | 0.04 μg/mL | 0.01 μg/mL |
|---|---|---|---|---|---|---|---|
| 0 h | 37.36 | 39.91 | 37.09 | 39.32 | 40.52 | 42.14 | 41.63 |
| 8 h | 35.54 | 38.55 | 37.19 | 42.39 | 45.58 | 49.02 | 49.97 |
| 16 h | 31.49 | 33.57 | 34.14 | 39.14 | 46.69 | 56.13 | 60.43 |
| 24 h | 32.63 | 35.03 | 35.64 | 40.68 | 48.16 | 63.21 | 72.08 |
| 32 h | 39.29 | 42.54 | 43.09 | 49.66 | 57.32 | 71.08 | 82.00 |
| 40 h | 45.83 | 48.88 | 49.60 | 56.70 | 65.99 | 79.45 | 90.99 |
| 48 h | 48.38 | 51.54 | 51.50 | 61.07 | 70.95 | 86.52 | 95.97 |
| 56 h | 49.51 | 53.29 | 54.17 | 64.16 | 75.85 | 91.12 | 98.27 |
| 64 h | 51.34 | 56.09 | 55.99 | 67.57 | 79.84 | 95.07 | 99.25 |
| 72 h | 52.86 | 57.44 | 57.58 | 70.57 | 83.68 | 97.12 | 99.71 |

Note:
the values represent phase object confluence (%).

The above results indicate that neither E-6C4-M-2F11 nor Amivantamab analog has the ability to kill NCI-H1975 at the highest concentration of 10 μg/m. However, E-6C4-M-2F11-ADC could effectively inhibit the growth of tumor cells at various concentrations in a dose-dependent manner.

### Example 5. Anti-Tumor-Activity in NCI-H1975 Xenograft Model

The antibodies were tested for their effect on tumor growth in vivo in a model of lung adenocarcinoma. Specifically, about 5×10⁶ NCI-H1975 cells were injected subcutaneously in B-NDG mice (Biocytogen Pharmaceuticals (Beijing) Co., Ltd., Cat #: B-CM-002). When the tumors in the mice reached a volume of about 300 mm³, the mice were randomly placed into different groups based on the volume of the tumor. The mice were then injected with phosphate buffer saline (PBS) or antibodies by intravenous (i.v.) administration. The frequency of administration was once a week (2 administrations in total). Details are shown in the table below.

The lengths of the long axis and the short axis of the tumor were measured and the volume of the tumor was calculated as 0.5×(long axis)×(short axis)². The tumor growth inhibition (TGI) was calculated using the following formula: TGI (%)=[1−(Ti−T0)/(Vi−V0)]×100. Ti is the average tumor volume in the treatment group on day i. T0 is the average tumor volume in the treatment group on day zero. Vi is the average tumor volume in the control group on day i. V0 is the average tumor volume in the control group on day zero. T-test was performed for statistical analysis. A TGI higher than 60% indicates clear suppression of tumor growth. P<0.05 is a threshold to indicate significant difference.

TABLE 20

| | No. of | | | | | | Total No. of |
|---|---|---|---|---|---|---|---|
| Group | mice | Antibodies | Dosage | | Route | Frequency | administration |
| G1 | 5 | PBS | — | | i.v. | QW | 2 |
| G2 | 5 | ISO-ADC | 3 | mg/kg | i.v. | QW | 2 |
| G3 | 5 | E-1G11-M-2F11-ADC | 3 | mg/kg | i.v. | QW | 2 |
| G4 | 5 | E-1G11-M-2G10-ADC | 3 | mg/kg | i.v. | QW | 2 |
| G5 | 5 | E-2B7-M-2F11-ADC | 3 | mg/kg | i.v. | QW | 2 |
| G6 | 5 | E-3A8-M-2G10-ADC | 3 | mg/kg | i.v. | QW | 2 |
| G7 | 5 | E-3D8-M-2F11-ADC | 3 | mg/kg | i.v. | QW | 2 |
| G8 | 5 | E-3D8-M-2G10-ADC | 3 | mg/kg | i.v. | QW | 2 |
| G9 | 5 | E-6C4-M-2F11-ADC | 3 | mg/kg | i.v. | QW | 2 |
| G10 | 5 | E-6C4-M-2G10-ADC | 3 | mg/kg | i.v. | QW | 2 |
| G11 | 5 | Amivantamab analog | 10 | mg/kg | i.v. | QW | 2 |

The body weight of the mice was also measured twice a week. On the day of grouping (Day 0), the average body weight of each group was in the range of 21.5 g-24.3 g. At the end of the experiment (Day 21), the average weight of each group was in the range of 20.4 g-24.9 g. Thus, the average weight change of each group was in the range of 91.1%-103.7%. The results showed that the tested antibodies were well tolerated and were not obviously toxic to the mice.

The table below summarizes the results for this experiment, including the tumor volumes on the day of grouping (Day 0), 11 days after grouping (Day 11) and at the end of the experiment (Day 21); the survival rate of the mice; TGI (%); and the statistical differences (P value) of tumor volume between the treatment and control groups.

TABLE 21

| | Tumor volume (mm³) | | | | | |
|---|---|---|---|---|---|---|
| Group | Day 0 | Day 11 | Day 21 | Survival | TGI (%) | P value |
| G1 | 260 ± 14 | 987 ± 82 | 2359 ± 182 | 5/5 | NA | NA |
| G2 | 260 ± 20 | 594 ± 30 | 1340 ± 116 | 5/5 | 48.5 | 0.003 |
| G3 | 260 ± 19 | 145 ± 18 | 107 ± 4 | 5/5 | 107.3 | 1.7E−06 |
| G4 | 260 ± 19 | 226 ± 14 | 185 ± 33 | 5/5 | 103.6 | 2.5E−06 |
| G5 | 260 ± 18 | 197 ± 20 | 174 ± 14 | 5/5 | 104.1 | 2.2E−06 |
| G6 | 259 ± 23 | 234 ± 19 | 124 ± 25 | 5/5 | 106.4 | 1.9E−06 |
| G7 | 259 ± 23 | 177 ± 21 | 109 ± 19 | 5/5 | 107.2 | 1.8E−06 |
| G8 | 259 ± 23 | 152 ± 20 | 103 ± 17 | 5/5 | 107.5 | 1.7E−06 |
| G9 | 259 ± 18 | 79 ± 31 | 38 ± 7 | 5/5 | 110.5 | 1.4E−06 |
| G10 | 260 ± 19 | 107 ± 15 | 83 ± 10 | 5/5 | 108.4 | 1.6E−06 |
| G11 | 260 ± 19 | 143 ± 20 | 294 ± 40 | 5/5 | 98.4 | 3.9E−06 |

The tumor volumes in all treatment groups (G3-G11) were smaller than those in the control group (G1 and G2). The treatment groups had different tumor inhibitory effects. All eight anti-EGFR/MET bispecific antibody ADCs at a dose level of 3 mg/kg(G3-G10) showed sustained and potent tumor suppression effects with TGI exceeding 100%, especially E-6C4-M-2F11-ADC (G9), which had the highest TGI of 110.5%. The TGI values of all tested ADCs were higher than that of the positive control Amivantamab analog at 10 mg/kg (TGI: 98.4%).

In another experiment, about 5×10⁵ NCI-H1975 cells were injected subcutaneously in B-NDG mice to determine the anti-tumor activity of E-6C4-M-2F11 and E-6C4-M-2F11-ADC, and when the tumor reached to a volume about 400 mm³, the mice were randomly placed into a control group and different treatment group based on tumor size. Details of grouping and dosing are shown in the table below.

TABLE 22

| | No. of | | | | | | Total No. of |
|---|---|---|---|---|---|---|---|
| Group | mice | Antibodies | Dosage | | Route | Frequency | administration |
| G1 | 5 | PBS | — | | i.v. | QW | 2 |
| G2 | 5 | E-6C4-M-2F11-ADC | 10 | mg/kg | i.v. | QW | 2 |
| G3 | 5 | E-6C4-M-2F11-ADC | 3 | mg/kg | i.v. | QW | 2 |
| G4 | 5 | E-6C4-M-2F11 | 10 | mg/kg | i.v. | QW | 2 |
| G5 | 5 | E-6C4-M-2F11 | 3 | mg/kg | i.v. | QW | 2 |

During the experimental period, little difference was observed between the body weight of mice in each group. The tumor volume and body weight were measured twice a week. The table below summarizes the results of this experiment, including the tumor volumes on the day of grouping (Day 0), 12 days after grouping (Day 12) and at the end of the experiment (Day 22); the survival rate of the mice; TGI (%); and the statistical differences (P value) of tumor volume between the treatment and control groups.

TABLE 23

| | Tumor volume (mm³) | | | | | |
|---|---|---|---|---|---|---|
| Group | Day 0 | Day 12 | Day 22 | Survival | TGI (%) | P value |
| G1 | 384 ± 9 | 1243 ± 91 | 2016 ± 156 | 5/5 | NA | NA |
| G2 | 384 ± 14 | 183 ± 33 | 102 ± 35 | 5/5 | 117.3 | 2.2E−06 |
| G3 | 384 ± 10 | 340 ± 16 | 222 ± 14 | 5/5 | 109.9 | 3.1E−06 |
| G4 | 384 ± 11 | 378 ± 63 | 1009 ± 156 | 5/5 | 61.7 | 0.002 |
| G5 | 384 ± 14 | 1067 ± 115 | 1740 ± 110 | 5/5 | 16.9 | 0.187 |

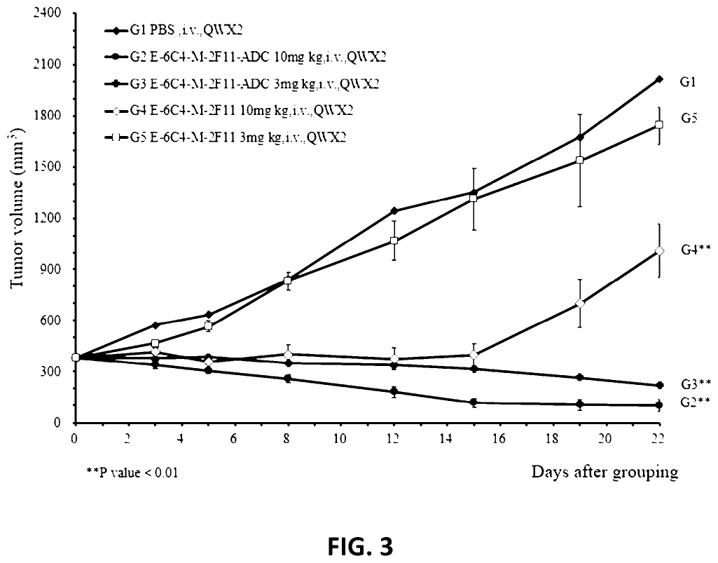

The tumor volume of mice in different groups treated with the antibodies, ADCs, or PBS are shown in FIG. 3, in which the anti-EGFR/MET bispecific antibody ADC E-6C4-M-2F11-ADC (G2 and G3) showed a better tumor inhibitory effect compared with PBS in the control groups (GI), and the corresponding anti-EGFR/MET bispecific antibody E-6C4-M-2F11 in groups G4 and G5.

In another experiment, about 1×10⁶ NCI-H1975 cells were injected subcutaneously in B-NDG mice to determine the anti-tumor activity of E-9A6-ADC, E-9F3-ADC and E-9D2-ADC, and when the tumor reached to a volume about 200 mm³, the mice were randomly placed into a control group and different treatment groups based on tumor size. Details of grouping and dosing are shown in the table below.

TABLE 24

| | No. of | | | | | Total No. of |
|---|---|---|---|---|---|---|
| Group | mice | Antibodies | Dosage | Route | Frequency | administration |
| G1 | 5 | PBS | — | i.v. | QW | 2 |
| G2 | 5 | Cetuximab analog-ADC | 3 mg/kg | i.v. | QW | 2 |
| G3 | 5 | E-9A6-ADC | 3 mg/kg | i.v. | QW | 2 |
| G4 | 5 | E-9F3-ADC | 3 mg/kg | i.v. | QW | 2 |
| G5 | 5 | E-9D2-ADC | 3 mg/kg | i.v. | QW | 2 |

The tumor volume and body weight were measured twice a week. The table below summarizes the results of this experiment, including the tumor volumes on the day of grouping (Day 0), 17 days after grouping (Day 17) and at the end of the experiment (Day 31); TGI (%); and the statistical differences (P value) of tumor volume between the treatment and control groups.

TABLE 25

| | Tumor volume (mm³) | | | TGI | |
|---|---|---|---|---|---|
| Group | Day 0 | Day 17 | Day 31 | (%) | P value |
| G1 | 192 ± 5 | 1938 ± 168 | 3341 ± 67 | NA | NA |
| G2 | 193 ± 11 | 352 ± 116 | 1712 ± 262 | 51.7 | 0.014 |
| G3 | 193 ± 9 | 116 ± 10 | 847 ± 80 | 79.2 | 9.830E−06 |
| G4 | 193 ± 9 | 87 ± 14 | 408 ± 71 | 93.2 | 2.520E−06 |
| G5 | 193 ± 15 | 159 ± 62 | 701 ± 296 | 83.9 | 0.003 |

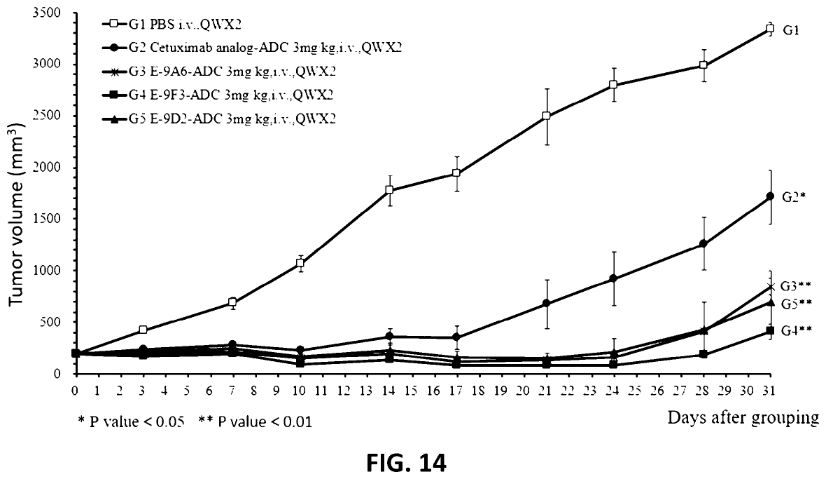

The tumor volume of mice in different groups treated with the ADCs or PBS are shown in FIG. 14, in which the anti-EGFR antibodies E-9 Å6-ADC, E-9F3-ADC and E-9D2-ADC showed a better tumor inhibitory effect compared with Cetuximab analog-ADC in the positive control group (G2).

In another experiment, NCI-H1975 cells were injected subcutaneously in Balb/c nude mice to determine the anti-tumor activity of ADCs, and when the tumor reached to a volume of about 400 mm³, the mice were randomly placed into a control group and different treatment groups based on tumor size. The mice were then injected with PBS, E-6C4-M-2F II-ADC (1.5 mg/kg, 3 mg/kg or 6 mg/kg, QW, 2 administrations in total) or Amivantamab (Janssen, Reference No.: LFS0L03) (10 mg/kg, BIW, 4 administrations in total) by intravenous (i.v.) administration. The results showed that the anti-tumor activities of all the three treatment groups of E-6C4-M-2F11-ADC (with TGI of 97.5%, 110.3% and 110.8% respectively) is stronger than that of Amivantamab at a dosage of 10 mg/kg (with a TGI of 12.9%).

### Example 6. Anti-Tumor Activity in NCI-H292 Xenograft Model

The antibodies were tested for their effect on tumor growth in vivo in a model of lung carcinoma. About 1×10⁷ NCI-H292 cells were injected subcutaneously in B-NDG mice. When the tumor in mice reached a volume of about 300 mm³, the mice were randomly placed into different groups based on the volume of the tumor. The mice were then injected with PBS or antibodies by intravenous (i.v.) administration. The frequency of administration was once a week (2 administrations in total). Details are shown in the table below.

TABLE 26

| | No. of | | | | | | Total No. of |
|---|---|---|---|---|---|---|---|
| Group | mice | Antibodies | Dosage | | Route | Frequency | administration |
| G1 | 5 | PBS | — | | i.v. | QW | 2 |
| G2 | 5 | E-6C4-M-2F11-ADC | 10 | mg/kg | i.v. | QW | 2 |
| G3 | 5 | E-6C4-M-2F11-ADC | 3 | mg/kg | i.v. | QW | 2 |
| G4 | 5 | E-6C4-M-2F11-ADC | 1 | mg/kg | i.v. | QW | 2 |
| G5 | 5 | Amivantamab analog | 10 | mg/kg | i.v. | QW | 2 |
| G6 | 5 | Amivantamab analog | 3 | mg/kg | i.v. | QW | 2 |
| G7 | 5 | Amivantamab analog | 1 | mg/kg | i.v. | QW | 2 |

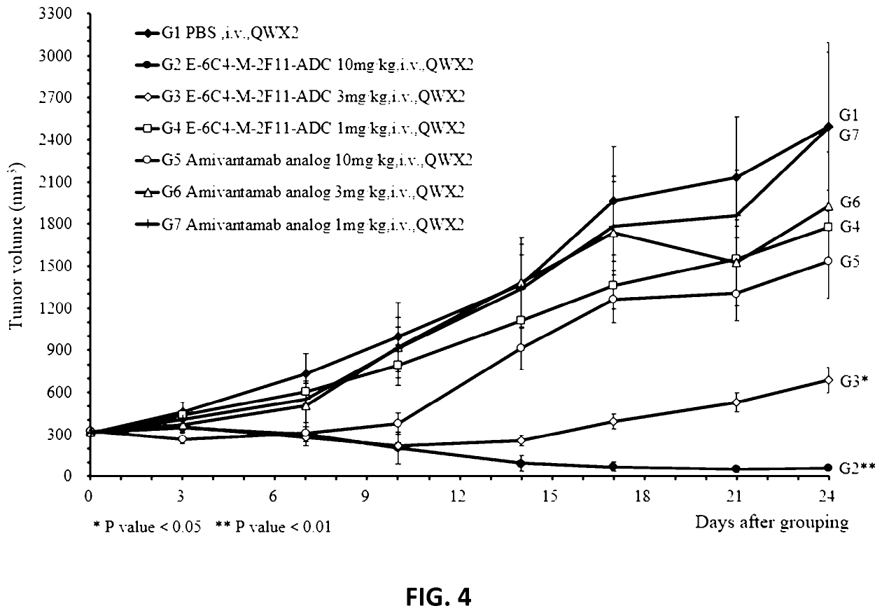

During the experimental period, little difference was observed between the body weight of mice in each group. The tumor size in groups treated with the antibodies are shown in FIG. 4. The table below summarizes the results for this experiment, including the tumor volumes on the day of grouping (Day 0), 14 days after grouping (Day 14), and at the end of the experiment (Day 24); the survival rate of the mice; TGI (%); and the statistical differences (P value) of tumor volume between the treatment and control groups.

TABLE 27

| | Tumor volume (mm³) | | | | | |
|---|---|---|---|---|---|---|
| Group | Day 0 | Day 14 | Day 24 | Survival | TGI (%) | P value |
| G1 | 310 ± 8 | 1371 ± 285 | 2493 ± 602 | 4/5 | NA | NA |
| G2 | 316 ± 14 | 94 ± 60 | 59 ± 22 | 5/5 | 111.8 | 0.002 |
| G3 | 316 ± 14 | 256 ± 40 | 687 ± 90 | 5/5 | 83.0 | 0.012 |
| G4 | 310 ± 15 | 1113 ± 215 | 1780 ± 263 | 5/5 | 32.7 | 0.279 |
| G5 | 327 ± 11 | 915 ± 150 | 1538 ± 267 | 4/5 | 44.5 | 0.197 |
| G6 | 314 ± 11 | 1380 ± 320 | 1925 ± 389 | 4/5 | 26.2 | 0.458 |
| G7 | 311 ± 14 | 1338 ± 243 | 2485 ± 536 | 4/5 | 0.4 | 0.993 |

During the experimental period, the body weight of mice in each group was maintained. The body weight of mice treated with 10 mg/kg of E-6C4-M-2F1-ADC (G2) showed an increasing trend, from 21.1 g on Day 0 to 21.8 g on Day 24, with an average weight change of 103.4%. Under the same dose levels, the anti-EGFR/MET bispecific antibody ADC showed a better anti-tumor activity than the positive control Amivantamab analog, in a dose-dependent manner.

In another experiment, about 5×10⁶ NCI-H292 cells were injected subcutaneously in B-NDG mice to determine the anti-tumor activity of E-6C4-M-2F11 and E-6C4-M-2F11-ADC, and when the tumor reached to a volume of about 200 mm³, the mice were randomly placed into a control group and different treatment groups based on tumor size. Details of grouping and dosing are shown in the table below.

TABLE 28

| | No. of | | | | | Total No. of |
|---|---|---|---|---|---|---|
| Group | mice | Antibodies | Dosage | Route | Frequency | administration |
| G1 | 5 | PBS | — | i.v. | QW | 2 |
| G2 | 5 | ISO-ADC | 3 mg/kg | i.v. | QW | 2 |
| G3 | 5 | E-6C4-M-2F11-ADC | 3 mg/kg | i.v. | QW | 2 |
| G4 | 5 | E-6C4-M-2G10-ADC | 3 mg/kg | i.v. | QW | 2 |
| G5 | 5 | E-6C4-ADC | 3 mg/kg | i.v. | QW | 2 |
| G6 | 5 | M-2G10-ADC | 3 mg/kg | i.v. | QW | 2 |
| G7 | 5 | M-2F11-ADC | 3 mg/kg | i.v. | QW | 2 |
| G8 | 5 | CD28-E-6C4-ADC | 3 mg/kg | i.v. | QW | 2 |
| G9 | 5 | CD28-M-2F11-ADC | 3 mg/kg | i.v. | QW | 2 |
| G10 | 5 | M-2G10-CD28-ADC | 3 mg/kg | i.v. | QW | 2 |

During the experimental period, little difference was observed between the body weight of mice in each group. The tumor volume and bodyweight were measured twice a week. The table below summarizes the results for this experiment, including the tumor volumes on the day of grouping (Day 0), 11 days after grouping (Day 11) and at the end of the experiment (Day 21); the survival rate of the mice; TGI (%); and the statistical differences (P value) of tumor volume between the treatment and control groups.

TABLE 29

| | Tumor volume (mm³) | | | | | |
|---|---|---|---|---|---|---|
| Group | Day 0 | Day 11 | Day 21 | Survival | TGI (%) | P value |
| G1 | 264 ± 24 | 1136 ± 192 | 2421 ± 303 | 2/5 | NA | NA |
| G2 | 264 ± 29 | 1006 ± 318 | 1950 ± 559 | 3/5 | 21.8 | 0.578 |
| G3 | 265 ± 27 | 264 ± 60 | 606 ± 151 | 5/5 | 84.2 | 0.002 |
| G4 | 265 ± 26 | 172 ± 63 | 431 ± 195 | 5/5 | 92.3 | 0.003 |
| G5 | 265 ± 28 | 130 ± 23 | 275 ± 50 | 5/5 | 99.5 | 7.5E−05 |
| G6 | 265 ± 33 | 623 ± 177 | 1225 ± 535 | 5/5 | 55.4 | 0.246 |
| G7 | 264 ± 34 | 545 ± 111 | 959 ± 218 | 5/5 | 67.8 | 0.015 |
| G8 | 265 ± 28 | 389 ± 30 | 710 ± 123 | 5/5 | 79.4 | 0.001 |
| G9 | 265 ± 32 | 675 ± 114 | 1409 ± 385 | 5/5 | 46.9 | 0.171 |
| G10 | 265 ± 31 | 785 ± 229 | 1510 ± 508 | 5/5 | 42.4 | 0.340 |

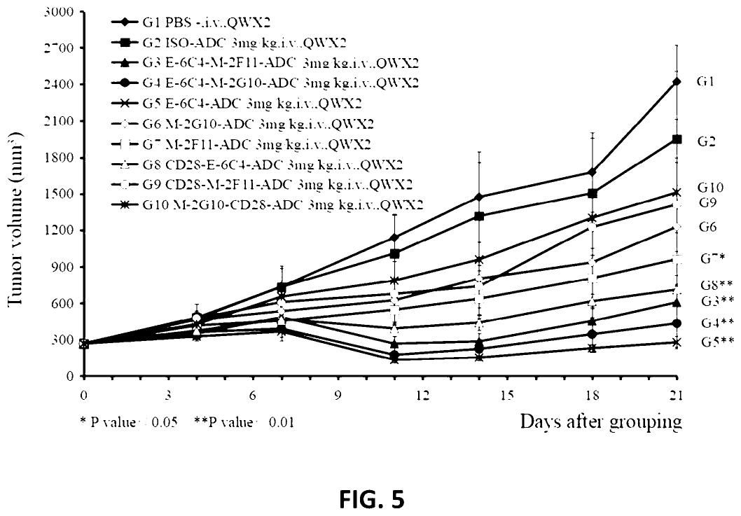

The tumor size in groups treated with the antibodies are shown in FIG. 5. The treatment groups showed different tumor inhibitory effects. Overall, the anti-tumor activity of the bispecific antibody ADCs is stronger than that of monoclonal antibody ADCs, and stronger than that of control bispecific antibody ADCs.

In another experiment, about 2×10⁶ NCI-H292 cells were injected subcutaneously in Balb/c nude mice to determine the anti-tumor activity of ADCs, and when the tumor reached to a volume of about 300 mm³, the mice were randomly placed into a control group and different treatment groups based on tumor size. The mice were then injected with PBS, ADCs or Amivantamab (Janssen, Reference No.: LFS0L03) by intravenous (i.v.) administration. Details of grouping and dosing are shown in the table below.

TABLE 30

| | No. of | | | | | | Total No. of |
|---|---|---|---|---|---|---|---|
| Group | mice | Antibodies | Dosage | | Route | Frequency | administration |
| G1 | 6 | PBS | — | | i.v. | QW | 2 |
| G2 | 6 | E-6C4-M-2F11-ADC | 6 | mg/kg | i.v. | QW | 2 |
| G3 | 6 | E-6C4-M-2F11-ADC | 3 | mg/kg | i.v. | QW | 2 |
| G4 | 6 | E-6C4-M-2F11-ADC | 1.5 | mg/kg | i.v. | QW | 2 |
| G5 | 6 | Telisotuzumab analog- | 3 | mg/kg | i.v. | QW | 2 |
| | | ADC | | | | | |
| G6 | 6 | MRG003 analog-ADC | 3 | mg/kg | i.v. | QW | 2 |
| G7 | 6 | Amivantamab | 10 | mg/kg | i.v. | BIW | 4 |

MRG003 is an antibody-drug conjugate consisting of fully human IgG1 monoclonal antibody targeting EGFR conjugated to monomethyl auristatin E (MMAE) for the treatment of solid tumors, which is in early clinical development at Shanghai Miracogen, and its heavy chain and light chain sequences are shown in SEQ ID NO: 110 and SEQ 2 NO: 1l, respectively.

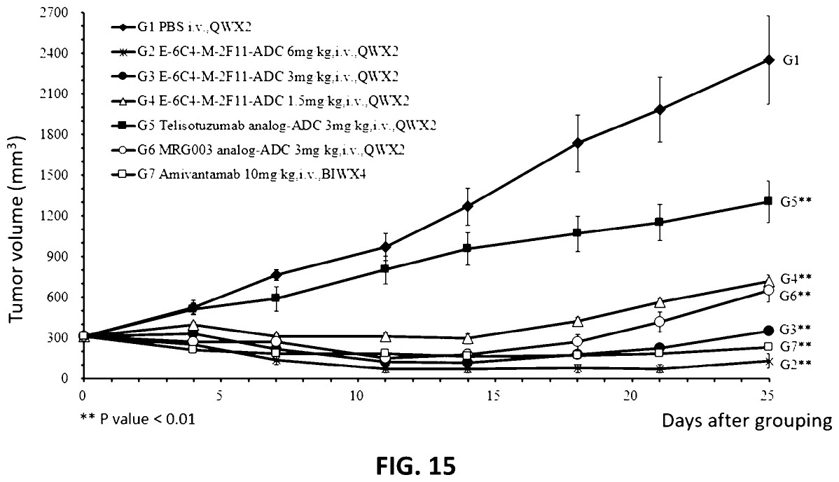

The tumor volume and body weight were measured twice a week. The tumor size in groups treated with the ADCs are shown in FIG. 15. The results showed that the anti-tumor activity of E-6C4-M-2F11-ADC (G3) is stronger than that of the positive control groups (G5-G6) at a dosage of 3 mg/kg, and the anti-tumor activity E-6C4-M-2F11-ADC at 6 mg/kg (G2) is stronger than that of Amivantamab at a dosage of 10 mg/kg. In addition, the bispecific antibody ADC E-6C4-M-2F11-ADC showed dose-dependent tumor-inhibiting effects.

### Example 7. Anti-Tumor Activity in SNU-5 Xenograft Model

The antibodies were tested for their effect on tumor growth in vivo in a model of gastric carcinoma. About 1×10⁷ SNU-5 cells were injected subcutaneously in B-NDG mice. When the tumors in the mice reached a volume of about 200 mm³, the mice were randomly placed into different groups based on the volume of the tumor. The mice were then injected with PBS or antibodies by intravenous (i.v.) administration. The frequency of administration was once a week (2 administrations in total). Details are shown in the table below.

TABLE 31

| | No. of | | | | | | Total No. of |
|---|---|---|---|---|---|---|---|
| Group | mice | Antibodies | Dosage | | Route | Frequency | administration |
| G1 | 5 | PBS | — | | i.v. | QW | 2 |
| G2 | 5 | Amivantamab analog | 10 | mg/kg | i.v. | QW | 2 |
| G3 | 5 | Amivantamab analog | 3 | mg/kg | i.v. | QW | 2 |
| G4 | 5 | E-6C4-M-2F11-ADC | 10 | mg/kg | i.v. | QW | 2 |
| G5 | 5 | E-6C4-M-2F11-ADC | 3 | mg/kg | i.v. | QW | 2 |
| G6 | 5 | E-6C4-M-2G10-ADC | 10 | mg/kg | i.v. | QW | 2 |
| G7 | 5 | E-6C4-M-2G10-ADC | 3 | mg/kg | i.v. | QW | 2 |
| G8 | 5 | E-6C4-M-2F11 | 10 | mg/kg | i.v. | QW | 2 |
| G9 | 5 | E-6C4-M-2F11 | 3 | mg/kg | i.v. | QW | 2 |
| G10 | 5 | E-6C4-M-2G10 | 10 | mg/kg | i.v. | QW | 2 |
| G11 | 5 | E-6C4-M-2G10 | 3 | mg/kg | i.v. | QW | 2 |

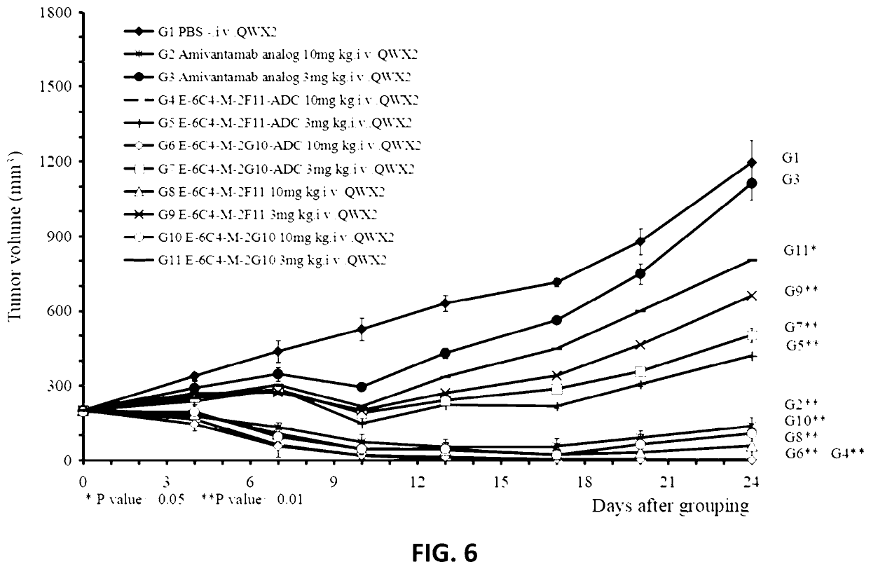

During the experimental period, little difference was observed between the body weight of mice in each group. The tumor size in groups treated with the ADCs are shown in FIG. 6. The table below summarizes the results for this experiment, including the tumor volumes on the day of grouping (Day 0), 10 days after grouping (Day 10), and at the end of the experiment (Day 24); the survival rate of the mice; TOI (%); and the statistical differences (P value) of tumor volume between the treatment and control groups.

TABLE 32

| | Tumor volume (mm³) | | | | | |
|---|---|---|---|---|---|---|
| Group | Day 0 | Day 10 | Day 24 | Survival | TGI (%) | P value |
| G1 | 200 ± 6 | 525 ± 45 | 1194 ± 91 | 5/5 | NA | NA |
| G2 | 200 ± 9 | 72 ± 9 | 136 ± 35 | 5/5 | 106.4 | 4.5E−06 |
| G3 | 200 ± 5 | 294 ± 10 | 1111 ± 64 | 5/5 | 8.4 | 0.475 |
| G4 | 200 ± 8 | 17 ± 2 | 0 | 5/5 | 120.1 | 1.0E−06 |
| G5 | 200 ± 8 | 147 ± 17 | 419 ± 96 | 5/5 | 78.0 | 3.7E−04 |
| G6 | 200 ± 7 | 19 ± 3 | 0 | 5/5 | 120.1 | 1.0E−06 |
| G7 | 200 ± 6 | 189 ± 22 | 502 ± 78 | 5/5 | 69.7 | 4.1E−04 |
| G8 | 200 ± 6 | 43 ± 3 | 57 ± 6 | 5/5 | 114.4 | 1.5E−06 |
| G9 | 200 ± 7 | 200 ± 23 | 661 ± 74 | 5/5 | 53.7 | 0.002 |
| G10 | 194 ± 8 | 44 ± 6 | 106 ± 29 | 5/5 | 108.8 | 3.1E−06 |
| G11 | 200 ± 6 | 216 ± 28 | 803 ± 94 | 5/5 | 39.4 | 0.017 |

In the SNU-5 gastric carcinoma model, ADCs E-6C4-M-2F11-ADC and E-6C4-M-2G10-ADC both showed strong anti-tumor activities at a dose level of 10 mg/kg or 3 mg/kg. Their tumor-inhibiting effects were greater than those of the corresponding bispecific antibodies E-6C4-M-2F and E-6C4-M-2G10, and greater than that of the positive control Amivantamab analog.

### Example 8. Anti-Tumor Activity in A431 Xenograft Model

The antibodies were tested for their effect on tumor growth in vivo in a model of skin carcinoma. About 5×10⁶ A431 cells (ATCC, Reference No.: CRL-1555) were injected subcutaneously in B-NDG mice. When the tumors in the mice reached a volume of about 300 mm³, the mice were randomly placed into different groups based on the volume of the tumor. The mice were then injected with PBS or antibodies by intravenous (i.v.) administration. The frequency of administration was once a week (2 administrations in total). Details are shown in the table below.

TABLE 33

| | No. of | | | | | Total No. of |
|---|---|---|---|---|---|---|
| Group | mice | Antibodies | Dosage | Route | Frequency | administration |
| G1 | 5 | PBS | 3 mg/kg | i.v. | QW | 2 |
| G2 | 5 | ISO-ADC | 3 mg/kg | i.v. | QW | 2 |
| G3 | 5 | E-6C4-M-2F11-ADC | 3 mg/kg | i.v. | QW | 2 |
| G4 | 5 | E-6C4-M-2F11 | 3 mg/kg | i.v. | QW | 2 |
| G5 | 5 | Amivantamab analog | 3 mg/kg | i.v. | QW | 2 |

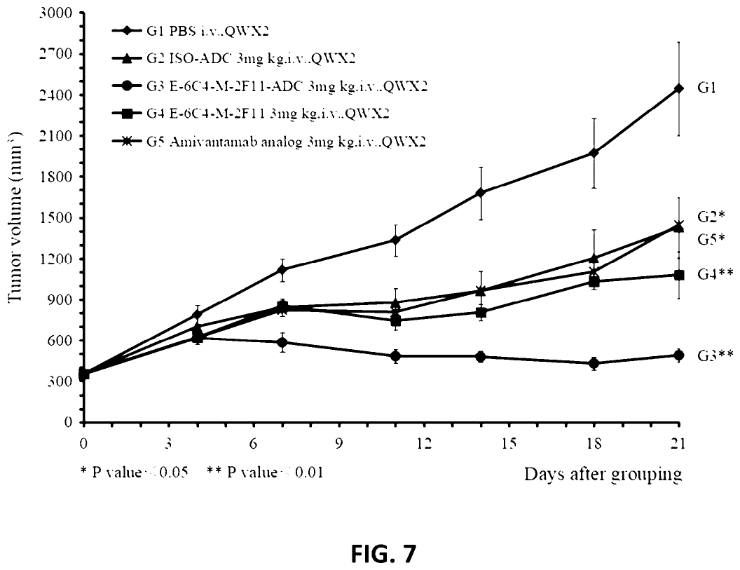

During the experimental period, little difference was observed between the body weight of mice in each group. The tumor size in groups treated with the antibodies are shown in FIG. 7. Similar to the above experimental results, in the A431 skin carcinoma in vivo model, E-6C4-M-2F11-ADC exhibited a greater tumor-inhibiting effect than the corresponding bispecific antibody E-6C4-M-2F11, and both had a greater tumor-inhibiting effect than the positive control Amivantamab analog.

### Example 9. In Vivo Efficacy in Human Lung Patient-Derived Xenograft (PDX) Model

The antibodies were tested for their effect in two human lung PDX (PDX001 and PDX002) models. Immunofluorescence staining of patient-derived lung tumor fragments was performed and the images were analyzed via HALO 3.2 version. The results showed that EGFR positive cell and MET positive cell in PDX001 were 24.28% and 25.71% respectively. In PDX002, EGFR positive cell and MET positive cell were 12.88% and 105.93% respectively.

In PDX001 model, B-NDG mice were engrafted in the right flank with patient-derived lung tumor fragments (2 mm×2 mm×2 mm). When the tumors in the mice reached a volume of about 250-300 mm³, the mice were randomly placed into different groups based on the volume of the tumor. The mice were then injected with PBS, Amivantamab analog or E-6C4-M-2F11-ADC by intravenous (i.v.) administration. Details of the administration scheme are shown in the table below.

TABLE 34

| | No. of | | Dosage | | | | Total No. of |
|---|---|---|---|---|---|---|---|
| Group | mice | Antibodies | (mg/kg) | | Route | Frequency | administration |
| G1 | 6 | PBS | — | | i.v. | QW | 2 |
| G2 | 6 | E-6C4-M-2F11-ADC | 10 | mg/kg | i.v. | QW | 2 |
| G3 | 6 | E-6C4-M-2F11-ADC | 3 | mg/kg | i.v. | QW | 2 |
| G4 | 6 | E-6C4-M-2F11-ADC | 1 | mg/kg | i.v. | QW | 2 |
| G5 | 6 | Amivantamab analog | 10 | mg/kg | i.v. | QW | 2 |
| G6 | 6 | Amivantamab analog | 3 | mg/kg | i.v. | QW | 2 |
| G7 | 6 | Amivantamab analog | 1 | mg/kg | i.v. | QW | 2 |

The tumor volume was measured twice a week and the results are shown in Table 26, which show that, compared with the control group (G1) and Amivantamab analog treatment groups (G5, G6 and G7), the treatment with E-6C4-M-2F11-ADC at 10 mg/kg (G2) and 3 mg/kg (G3) resulted in robust tumor growth inhibition in EGFR/MET co-expressing human lung PDX model, with a TGI % of 109.3% and 38.3% respectively on Day 23 (23 days after grouping).

TABLE 35

| | Tumor volume (mm³) | | | | | |
|---|---|---|---|---|---|---|
| Group | Day 0 | Day 12 | Day 23 | Survival | TGI (%) | P value |
| G1 | 223 ± 15 | 1241 ± 122 | 2511 ± 349 | 5/6 | NA | NA |
| G2 | 223 ± 17 | 47 ± 12 | 10 ± 2 | 6/6 | 109.3 | 2.37E−05 |
| G3 | 223 ± 17 | 423 ± 160 | 1635 ± 863 | 6/6 | 38.3 | 0.406 |
| G4 | 223 ± 16 | 1340 ± 119 | 2791 ± 204 | 5/6 | −12.2 | 0.510 |
| G5 | 223 ± 22 | 1132 ± 77 | 2666 ± 212 | 6/6 | −6.8 | 0.703 |
| G6 | 223 ± 18 | 1448 ± 195 | 2465 ± 145 | 3/6 | 2.0 | 0.926 |
| G7 | 223 ± 18 | 1144 ± 86 | 3077 ± 235 | 4/6 | −24.7 | 0.246 |

In PDX002 model, when the tumors in the mice reached a volume of about 250-300 mm³, the B-NDG mice were divided to a control group and different treatment groups based on tumor size (6 mice per group). The treatment groups were randomly selected for E-6C4-M-2F11-ADC treatment at 3 mg/kg (G2) and 1 mg/kg (G3), or Amivantanmab analog treatment at 3 mg/kg (G4) and 1 mg/kg (G5). The control group mice were injected with PBS (G1). The frequency of administration was once a week (two times of administrations in total).

The tumor size in each group are shown in Table 36. Compared with the control group (G1) and Amivantamab analog treatment groups (G4 and G5), E-6C4-M-2F11-ADC treatment at 3 mg/kg (G2) exhibited better tumor-suppressing effect in MET high-expressing human lung PDX model.

TABLE 36

| | Tumor volume (mm³) | | | | | | |
|---|---|---|---|---|---|---|---|
| Group | Day 0 | Day 10 | Day 21 | Day 35 | Survival | TGI (%) | P value |
| G1 | 267 ± 10 | 618 ± 41 | 1681 ± 206 | 3294 ± 154 | 2/6 | NA | NA |
| G2 | 267 ± 11 | 381 ± 38 | 308 ± 38 | 538 ± 80 | 6/6 | 91.1 | 2.78E−06 |
| G3 | 267 ± 12 | 479 ± 35 | 900 ± 121 | 2619 ± 224 | 6/6 | 22.3 | 0.155 |
| G4 | 267 ± 13 | 686 ± 55 | 1791 ± 214 | 3509 ± 239 | 3/6 | −7.1 | 0.884 |
| G5 | 267 ± 12 | 805 ± 99 | 2279 ± 253 | — | 0/6 | — | — |

### Example 10. In Vivo Efficacy in Human Pancreatic PDX Model

The antibodies were tested for their effect in two human pancreatic PDX (PDX003 and PDX004) models. Immunofluorescence staining of patient-derived pancreatic tumor fragments was performed and the images were analyzed via HALO 3.2 version. The results showed that EGFR positive cell and MET positive cell in PDX003 were 7.98% and 32.44% respectively. In PDX004, EGFR positive cell and MET positive cell were 54.65% and 36.63% respectively.

In PDX003 model, B-NDG mice were engrafted in the right flank with patient-derived pancreatic tumor fragments (2 mm×2 mm×2 mm). When the tumors in the mice reached a volume of about 300-400 mm³, the mice were randomly placed into different groups based on the volume of the tumor. The mice were then injected with PBS, Amivantamab analog or E-6C4-M-2F11-ADC by intravenous (i.v.) administration. Details of the administration scheme are shown in the table below.

TABLE 37

| | No. of | | Dosage | | | Total No. of |
|---|---|---|---|---|---|---|
| Group | mice | Antibodies | (mg/kg) | Route | Frequency | administration |
| G1 | 6 | PBS | — | i.v. | QW | 2 |
| G2 | 6 | E-6C4-M-2F11-ADC | 10 mg/kg | i.v. | QW | 2 |
| G3 | 6 | E-6C4-M-2F11-ADC | 3 mg/kg | i.v. | QW | 2 |
| G4 | 6 | E-6C4-M-2F11-ADC | 1 mg/kg | i.v. | QW | 2 |
| G5 | 6 | Amivantamab analog | 10 mg/kg | i.v. | QW | 2 |
| G6 | 6 | Amivantamab analog | 3 mg/kg | i.v. | QW | 2 |
| G7 | 6 | Amivantamab analog | 1 mg/kg | i.v. | QW | 2 |

The tumor volume was measured twice a week and the results are shown in Table 39, which show that compared with the control group, treatment with E-6C4-M-2F11-ADC resulted in significant tumor growth inhibition in human pancreatic PDX model at 10 mg/kg, 3 mg/kg and 1 mg/kg, with a TGI % of 129.2%, 127.7% and 45.8% respectively on Day 21.

TABLE 38

| | Tumor volume (mm³) | | | | | |
|---|---|---|---|---|---|---|
| Group | Day 0 | Day 11 | Day 21 | Survival | TGI (%) | P value |
| G1 | 340 ± 13 | 837 ± 75 | 1389 ± 114 | 5/6 | NA | NA |
| G2 | 340 ± 15 | 70 ± 15 | 33 ± 3 | 6/6 | 129.2 | 3.25E−07 |
| G3 | 339 ± 13 | 70 ± 5 | 48 ± 3 | 6/6 | 127.7 | 3.61E07 |
| G4 | 340 ± 17 | 493 ± 45 | 909 ± 85 | 6/6 | 45.8 | 0.007 |
| G5 | 340 ± 16 | 666 ± 60 | 1146 ± 123 | 6/6 | 23.2 | 0.179 |
| G6 | 340 ± 18 | 705 ± 62 | 1216 ± 96 | 6/6 | 16.5 | 0.273 |
| G7 | 340 ± 18 | 811 ± 65 | 1410 ± 130 | 6/6 | −2.0 | 0.905 |

In PDX004 model, when the tumors in the mice reached a volume of about 250-300 mm³, the mice were divided to a control group and different treatment groups based on tumor size (6 mice per group). The tested antibody and the frequency of administration was similar to Table 28.

The results showed that E-6C4-M-2F11-ADC treatment groups (including 1 mg/kg, 3 mg/kg and 10 mg/kg dosage) all resulted in a substantial antitumor activity in human pancreatic PDX model, with a TGI % of 46.9%, 109.4% and 110.9% respectively on Day 35 (35 days after grouping), which were higher than that of Amivantanmab analog treatment groups (−13.3%, −0.6% and 4.1% respectively).

### Example 11. Pharmacokinetic Profiles

The pharmacokinetic clearance rates of the anti-EGFR/MET bispecific antibody ADC were determined in C57BL/6 mice. Specifically, the mice were placed into four groups (5 mice per group), and administered with E-6C4-M-2F11-ADC (G1, 3 mg/kg; G2, 10 mg/kg) or E-6C4-M-2F11 (G3, 3 mg/kg; G4, 10 mg/kg) by intravenous injection. Blood samples were collected 3 days before administration and 15 minutes. 6 hours, 1 day, 2 day, 5 days, 10 days, 14 days and 21 days after administration.

The serum levels of antibody and ADC were determined by sandwich ELISA. Briefly, Goat Anti-Human IgG (H+L) (Jackson ImmunoResearch Inc., Cat #: 109-005-088) or anti-MMAE mIgG (ACRO Biosystems Inc., Cat #: MME-M5252) was diluted to a final concentration of 2000 ng/mL, added to a 96-well plate (ELISA plate) at 100 μL/well, and then incubated overnight at 2-8° C. After the incubation, the plate was washed with PBS-T buffer (PBS supplemented with Tween™ 20) 4 times. Antibody-unbound areas were blocked with 2% BSA (bovine serum albumin) for 2 hours at 37° C. Afterwards, the plate was washed with PBS-T buffer 4 times. After washing, 100 μL of blocking buffer (2% BSA) was added to each well. The wells were sealed and incubated at 37° C. for 1 hour. After washing the plate using a plate washer, Peroxidase AffiniPure F(ab′)₂ Fragment Goat Anti-Human IgG, Fcγ fragment specific (Jackson ImmunoResearch Inc., Cat #: 109-036-098) was added at 100 PL/well to each well of the plate, and incubated at 37° C. for 1 hour. After washing the plate, tetramethylbenzidine (TMB) solution was added at 100 μL/well to the 96-well plate as the substrate. After incubating at room temperature in the dark, 100 μL stop solution (Beyotime, Cat #: P0215) was added to each well. Luminescent signals of the plate was measured at 450 nm and 630 nm to calculate the concentrations. The absorbance value and corresponding concentration of the calibration sample prepared by each test product was used to create a standard curve with four parameters (i.e., T_{1/2}, Cₘₐₓ, AUC_{0-21 day}, and CL). The standard curve was used to calculate the antibody or ADC concentration of each serum sample. A drug concentration-time curve was created using the calculated sample concentration at each time point. Phoenix™ WinNolin 8.3 was used to calculate the pharmacokinetic parameters.

The results are shown in the table below, which show that the conjugation of the drug molecules dose not affect in vivo pharmacokinetic clearance rates of the bispecific antibodies.

TABLE 39

| | | | | | | CL |
|---|---|---|---|---|---|---|
| | Dose | | T_{1/2} | Cₘₐₓ | AUC_{0-21 day} | (mL/ |
| Group | (mg) | LBA | (hr) | (μg/mL) | (hr*μg/mL) | hr/kg) |
| G1 | 3 | ADC | 124.426 | 55.578 | 2799.082 | 1.025 |
| | 3 | Antibody | 302.356 | 55.592 | 5634.357 | 0.380 |
| G2 | 10 | ADC | 100.935 | 174.629 | 10520.343 | 0.918 |
| | 10 | Antibody | 256.849 | 172.604 | 19540.211 | 0.393 |
| G3 | 3 | Antibody | 303.726 | 45.621 | 6370.19 | 0.340 |
| G4 | 10 | Antibody | 350.753 | 188.259 | 25367.833 | 0.256 |

T_{1/2}: Terminal Half Life;
Cₘₐₓ: Max Concentration;
AUC_{0-21 day}: Area under Blood Concentration-time Curve 0-21 day
CL: Clearance

### Example 12. In Vivo Efficacy of Combination of the ADCs

In another experiment, about 5×10⁵ NCI-H1975 cells were injected subcutaneously in B-NDG mice to determine the in vivo efficacy of combination of E-6C4-ADC and M-2FC11-AD. When the tumor reached to a volume about 300 mm³, the mice were randomly placed into a control group and different treatment group based on tumor size. Details of grouping and dosing are shown in the table below.

TABLE 40

| | No. of | | | | | | Total No. of |
|---|---|---|---|---|---|---|---|
| Group | mice | Antibodies | Dosage | | Route | Frequency | administration |
| G1 | 6 | PBS | — | | i.v. | QW | 2 |
| G2 | 6 | M-2F11-ADC | 3 | mg/kg | i.v. | QW | 2 |
| G3 | 6 | E-6C4-ADC | 3 | mg/kg | i.v. | QW | 2 |
| G4 | 6 | M-2F11-ADC | 1.5 | mg/kg | i.v. | QW | 2 |
| G5 | 6 | E-6C4-ADC | 1.5 | mg/kg | i.v. | QW | 2 |
| G6 | 6 | E-6C4-ADC | 3 | mg/kg | i.v. | QW | 2 |
| | | M-2F11-ADC | 3 | mg/kg | | | |
| G7 | 6 | E-6C4-ADC | 1.5 | mg/kg | i.v. | QW | 2 |
| | | M-2F11-ADC | 1.5 | mg/kg | | | |
| G8 | 6 | E-6C4-ADC | 0.75 | mg/kg | i.v. | QW | 2 |
| | | M-2F11-ADC | 0.75 | mg/kg | | | |
| G9 | 6 | E-6C4-M-2F11 | 1.5 | mg/kg | i.v. | QW | 2 |
| G10 | 6 | E-6C4-M-2F11 | 3 | mg/kg | i.v. | QW | 2 |
| G11 | 6 | E-6C4-M-2F11-ADC | 1.5 | mg/kg | i.v. | QW | 2 |
| G12 | 6 | E-6C4-M-2F11-ADC | 3 | mg/kg | i.v. | QW | 2 |

During the experimental period, little difference was observed between the body weight of mice in each group. The tumor volume and body weight were measured twice a week. The table below summarizes the results of this experiment, including the tumor volumes on the day of grouping (Day 0), 14 days after grouping (Day 14), 21 days after grouping (Day 21), and 39 days after grouping (Day 39, when applicable); the survival rate of the mice; TGI (%); and P value of tumor volume between the treatment and control groups at Day 21.

TABLE 41

| | Tumor volume (mm³) | | | | | | |
|---|---|---|---|---|---|---|---|
| Group | Day 0 | Day 14 | Day 21 | Day 39 | Survival | TGI (%) | P value |
| G1 | 287 ± 6 | 2074 ± 165 | 2940 ± 185 | NA | 5/6 | NA | NA |
| G2 | 287 ± 6 | 220 ± 18 | 104 ± 5 | 1963 ± 111 | 6/6 | 106.9% | 3.86E−08 |
| G3 | 287 ± 6 | 150 ± 14 | 117 ± 18 | 2146 ± 190 | 6/6 | 106.4% | 4.25E−08 |
| G4 | 287 ± 7 | 575 ± 73 | 965 ± 149 | NA | 6/6 | 74.4% | 1.46E−05 |
| G5 | 287 ± 7 | 828 ± 84 | 1521 ± 174 | NA | 6/6 | 53.5% | 3.46E−04 |
| G6 | 287 ± 7 | 63 ± 16 | 42 ± 11 | 901 ± 159 | 6/6 | 109.2% | 3.26E−08 |
| G7 | 287 ± 8 | 214 ± 30 | 107 ± 33 | 2187 ± 211 | 6/6 | 106.8% | 4.76E−08 |
| G8 | 287 ± 9 | 716 ± 56 | 1358 ± 108 | NA | 6/6 | 59.7% | 2.96E−05 |
| G9 | 287 ± 8 | 1641 ± 62 | 2593 ± 122 | NA | 6/6 | 13.1% | 0.141 |
| G10 | 287 ± 9 | 1209 ± 112 | 2114 ± 153 | NA | 6/6 | 31.1% | 0.007 |
| G11 | 287 ± 10 | 216 ± 16 | 217 ± 10 | 2458 ± 142 | 6/6 | 102.6% | 5.60E−08 |
| G12 | 287 ± 9 | 57 ± 23 | 40 ± 13 | 1121 ± 306 | 6/6 | 109.3% | 3.27E−08 |

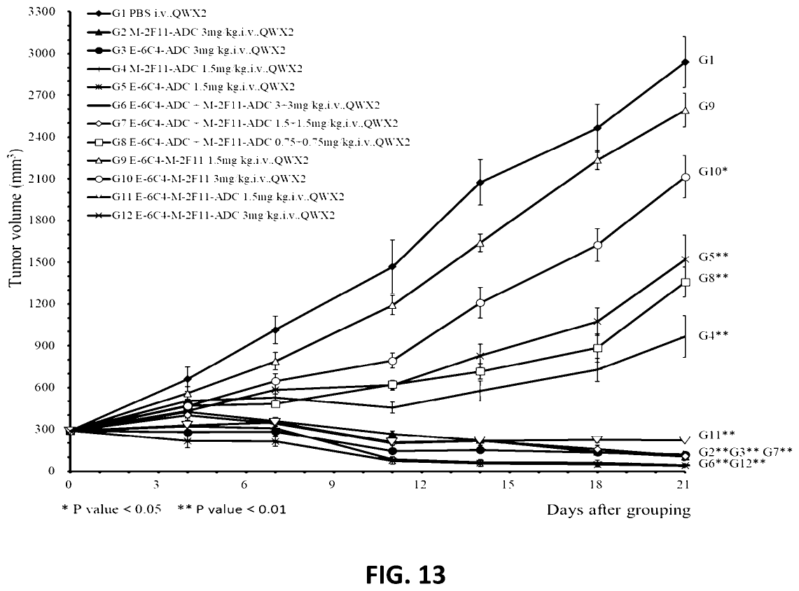

The tumor volume of mice in different groups treated with the ADCs or PBS are shown in FIG. 13. Compared with the PBS group (G1), the monoclonal antibody ADC groups E-6C4-ADC (G3 and G5) and M-2F11-ADC (G2 and G4), the bispecific antibody ADC groups (G11 and G12) and the combination groups of the monoclonal antibody ADC (G6, G7 and G8) all significantly exhibited antitumor activity at Day 21.

In particular, the TGI % of M-2F11-ADC (G4) and E-6C4-ADC (G5) at 1.5 mg/kg were comparable to that of the combination of M-2F11-ADC and E-6C4-ADC (G8) at 0.75 mg/kg, but was lower than that of E-6C4-M-2F11-ADC (G11) at 1.5 mg/kg.

Similar to the above results, the TGI % of M-2F11-ADC (G2) and E-6C4-ADC (G3) at 3 mg/kg were similar to that of the combination of M-2F11-ADC and E-6C4-ADC (G7) at 1.5 mg/kg, but was lower than that of E-6C4-M-2F11-ADC (G12) at 3 mg/kg.

The mouse tumor volume and survival were continued to be monitored after Day 21. At the end of the experiment on Day 39, all mice died in groups Gi, G4, G5, G8, G9 and G10, in contrast, all mice survived in groups G2, G3, G6, G7, G11 and G12, the tumor volume results at Day 39 showed that at a dose level of 3 mg/kg, E-6C4-M-2F11-ADC (G12) treatment had a better anti-tumor effect as compared to M-2F11-ADC (G2), E-6C4-ADC (G3) or their combination (G7).

The results also indicate that the anti-EGFR/MET bispecific antibody has a synergistic effect on tumor suppression.

### Example 13. In Vivo Efficacy in Human Ampullary Carcinoma PDX Model

The ADCs were tested for their effect in human ampullary carcinoma PDX model. Immunofluorescence staining of patient-derived ampullary tumor fragments was performed and the images were analyzed via HALO 3.2 version. The results showed that EGFR positive cell and MET positive cell were 94.03% and 0.25% respectively.

B-NDG mice were engrafted in the right flank with patient-derived ampullary tumor fragments (2 mm×2 mm×2 mm). When the tumors in the mice reached a volume of about 250-300 mm³, the mice were randomly placed into different groups based on the volume of the tumor. The mice were then injected with PBS, ADCs or Amivantamab (Janssen, Cat #: LFS0L03) by intravenous (i.v.) administration. Details of the administration scheme are shown in the table below.

TABLE 42

| | No. of | | Dosage | | | | Total No. of |
|---|---|---|---|---|---|---|---|
| Group | mice | Antibodies | (mg/kg) | | Route | Frequency | administration |
| G1 | 6 | PBS | — | | i.v. | QW | 2 |
| G2 | 6 | E-6C4-M-2F11-ADC | 6 | mg/kg | i.v. | QW | 2 |
| G3 | 6 | E-6C4-M-2F11-ADC | 3 | mg/kg | i.v. | QW | 2 |
| G4 | 6 | E-6C4-M-2F11-ADC | 1.5 | mg/kg | i.v. | QW | 2 |
| G5 | 6 | E-6C4-ADC | 3 | mg/kg | i.v. | QW | 2 |
| G6 | 6 | Cetuximab analog-ADC | 6 | mg/kg | i.v. | QW | 2 |
| G7 | 6 | Cetuximab analog-ADC | 3 | mg/kg | i.v. | QW | 2 |
| G8 | 6 | Amivantamab | 10 | mg/kg | i.v. | BIW | 4 |

The table below summarizes the results for this experiment, including the tumor volumes on the day of grouping (Day 0), 14 days after grouping (Day 14) and 21 days after grouping (Day 21): TGI (%); and the statistical differences (P value) of tumor volume between the treatment and control groups.

TABLE 43

| | Tumor volume (mm³) | | | TGI | |
|---|---|---|---|---|---|
| Group | Day 0 | Day 14 | Day 21 | (%) | P value |
| G1 | 273 ± 10 | 780 ± 82 | 1114 ± 148 | NA | NA |
| G2 | 273 ± 11 | 152 ± 20 | 65 ± 10 | 124.8% | 3.378E−05 |
| G3 | 273 ± 12 | 310 ± 77 | 244 ± 98 | 103.5% | 0.001 |
| G4 | 273 ± 12 | 538 ± 106 | 522 ± 127 | 70.4% | 0.016 |
| G5 | 273 ± 11 | 373 ± 48 | 299 ± 87 | 96.9% | 0.001 |
| G6 | 273 ± 14 | 290 ± 40 | 234 ± 71 | 104.7% | 3.174E−04 |
| G7 | 273 ± 11 | 413 ± 72 | 440 ± 102 | 80.2% | 0.004 |
| G8 | 273 ± 13 | 756 ± 78 | 1047 ± 104 | 8% | 0.719 |

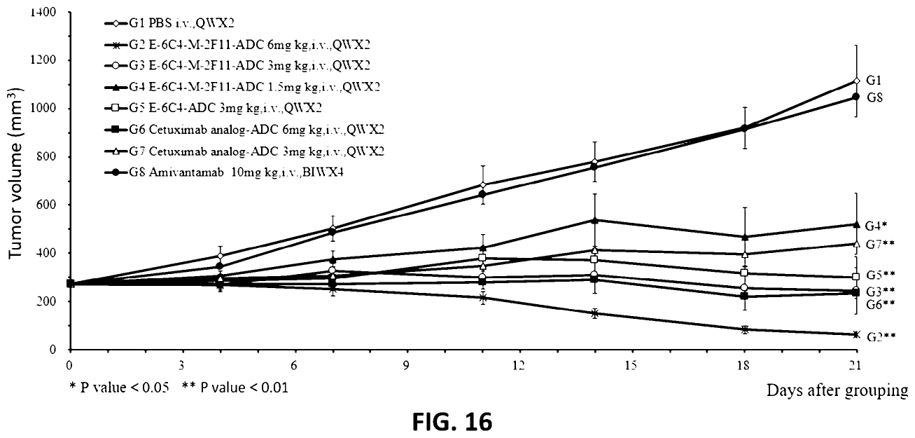

The tumor volume of mice in different groups are shown in FIG. 16. The results showed that compared with the control group G1, the treatment groups showed different tumor inhibition effects. Among the treatment groups, at the same dosage, E-6C4-M-2F11-ADC (G2 and G3) showed the best tumor inhibitory effect, followed by the corresponding anti-EGFR antibody ADC E-6C4-ADC, and finally the positive control Cetuximab analog-ADC. E-6C4-M-2F11-ADC showed dose-dependent tumor-inhibiting effects. In addition, the experiment was then continued until 39 days after grouping (Day 39) and the E-6C4-M-2F11-ADC (G2-G3) still showed better tumor inhibitory effect with TGI of 108.6% and 83.7% respectively than E-6C4-ADC (G5) with TGI of 71.0% and Cetuximab analog-ADC (G6-G7) with TGI of 78.7% and 57.5% respectively.

### Example 14. In Vivo Efficacy in Human Gastric Cancer PDX Model

Immunofluorescence staining of patient-derived gastric tumor fragments was performed and the images were analyzed via HALO 3.2 version. The results showed that EGFR positive cell and MET positive cell in the gastric PDX model were 80.53% and 3.10% respectively.

B-NDG mice were engrafted in the right flank with patient-derived gastric tumor fragments (2 mm×2 mm×2 mm). When the tumors in the mice reached a volume of about 250-300 mm³, the mice were randomly placed into different groups based on the volume of the tumor. The mice were then injected with PBS, ADCs or Amivantamab (Janssen, Reference No.: LFS01L03) by intravenous (i.v.) administration. Details of the administration scheme are shown in the table below.

TABLE 44

| | No. of | | Dosage | | | | Total No. of |
|---|---|---|---|---|---|---|---|
| Group | mice | Antibodies | (mg/kg) | | Route | Frequency | administration |
| G1 | 5 | PBS | — | | i.v. | QW | 2 |
| G2 | 5 | E-6C4-M-2F11-ADC | 6 | mg/kg | i.v. | QW | 2 |
| G3 | 5 | E-6C4-M-2F11-ADC | 3 | mg/kg | i.v. | QW | 2 |
| G4 | 5 | E-6C4-M-2F11-ADC | 1.5 | mg/kg | i.v. | QW | 2 |
| G5 | 5 | Amivantamab | 10 | mg/kg | i.v. | BIW | 6 |

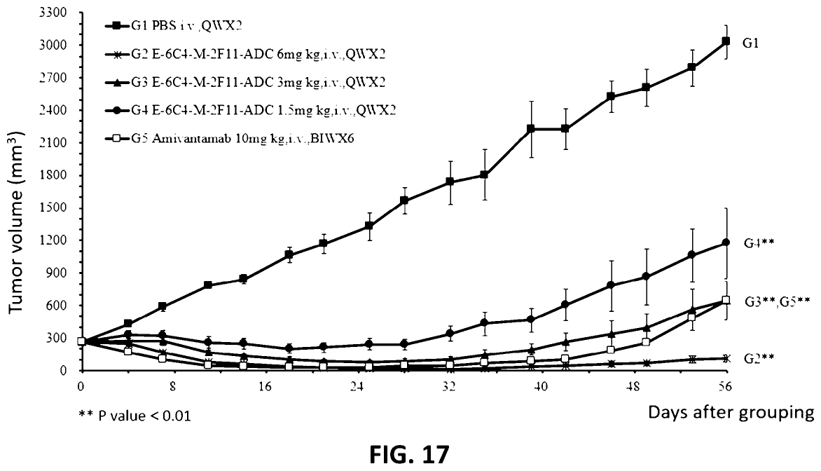

The tumor volume was measured twice a week and the results are shown in table below and FIG. 17, which show that the treatment group with E-6C4-M-2F11-ADC (G2-G4) resulted in sustained robust tumor growth inhibition in human gastric PDX model compared with the control group (G1), and the E-6C4-M-2F11-ADC of 6 mg/kg showed a better tumor inhibitory effect compared with Amivantamab of 10 mg/kg. The tumor volume was continued to be monitored until 77 days after grouping (Day 77), and E-6C4-M-2F1 l-ADC (G2-G4) still showed better tumor inhibitory effects compared with Amivantamab (10 mg/kg).

TABLE 45

| | Tumor volume (mm³) | | | | | |
|---|---|---|---|---|---|---|
| Group | Day 0 | Day 28 | Day 56 | Survival | TGI (%) | P value |
| G1 | 262 ± 23 | 1563 ± 123 | 3033 ± 157 | 3/5 | NA | NA |
| G2 | 262 ± 29 | 19 ± 3 | 109 ± 34 | 5/5 | 105.5% | 3.672E−07 |
| G3 | 262 ± 26 | 86 ± 23 | 648 ± 180 | 5/5 | 86.1% | 1.070E−04 |
| G4 | 262 ± 27 | 240 ± 48 | 1176 ± 325 | 5/5 | 67.0% | 0.006 |
| G5 | 262 ± 24 | 40 ± 6 | 643 ± 391 | 5/5 | 86.3% | 0.004 |

### Example 15. Anti-Tumor Activity in NUGC-4 Xenograft Model

The ADCs were tested for their effects on tumor growth in vivo in a model of gastric cancer. About 5×10⁶ NUGC-4 cells were injected subcutaneously in B-NDG mice. When the tumor size reached about 200 mm³, the mice were randomly placed into treatment or control groups based on tumor size. The mice were then injected with PBS or ADCs by intravenous (i.v.) administration. The frequency of administration was once a week (2 administrations in total). Details are shown in the table below.

TABLE 46

| | | | | | | Total |
|---|---|---|---|---|---|---|
| | | | | | | No. of |
| | No. of | | | | Fre- | adminis- |
| Group | mice | Antibodies | Dosage | Route | quency | tration |
| G1 | 5 | PBS | — | i.v. | QW | 2 |
| G2 | 5 | ISO-ADC | 3 mg/kg | i.v. | QW | 2 |
| G3 | 5 | M-2F11-ADC | 3 mg/kg | i.v. | QW | 2 |
| G4 | 5 | M-8H10-ADC | 3 mg/kg | i.v. | QW | 2 |
| G5 | 5 | M-8D9-ADC | 3 mg/kg | i.v. | QW | 2 |
| G6 | 5 | M-8E2-ADC | 3 mg/kg | i.v. | QW | 2 |
| G7 | 5 | M-6D12-ADC | 3 mg/kg | i.v. | QW | 2 |
| G8 | 5 | M-7D3-ADC | 3 mg/kg | i.v. | QW | 2 |
| G9 | 5 | Telisotuzumab | 3 mg/kg | i.v. | QW | 2 |
| | | analog-ADC | | | | |

The weights of mice in different groups increased except G1 and G2, which showed that the ADCs of G3-G9 were well tolerated and were not obviously toxic to the mice.

The table below summarizes the results for this experiment, including the tumor volumes on the day of grouping (Day 0), 14 days after grouping (Day 14) and at the end of the experiment (Day 28); TGI (%); the statistical differences (P value) of tumor volumes between the treatment and control groups; and the number of tumor-free mice.

TABLE 47

| | | | | | P value | |
|---|---|---|---|---|---|---|
| | Tumor volume (mm³) | | | | (tumor | Tumor-free |
| Group | Day 0 | Day 14 | Day 28 | TGI (%) | volume) | (Day 28) |
| G1 | 198 ± 12 | 699 ± 82 | 1560 ± 134 | NA | NA | 0/5 |
| G2 | 198 ± 13 | 689 ± 32 | 1755 ± 142 | −14.3 | 0.347 | 0/5 |
| G3 | 198 ± 14 | 45 ± 3 | 37 ± 7 | 111.8 | 3.29E−06 | 0/5 |
| G4 | 198 ± 13 | 46 ± 6 | 51 ± 21 | 110.8 | 3.84E−06 | 0/5 |
| G5 | 198 ± 13 | 41 ± 5 | 8 ± 4 | 113.9 | 2.83E−06 | 2/5 |
| G6 | 198 ± 11 | 50 ± 9 | 59 ± 27 | 110.2 | 4.22E−06 | 0/5 |
| G7 | 198 ± 15 | 47 ± 3 | 20 ± 2 | 113.1 | 2.99E−06 | 0/5 |
| G8 | 197 ± 13 | 47 ± 6 | 19 ± 2 | 113.1 | 2.98E−06 | 0/5 |
| G9 | 198 ± 14 | 60 ± 10 | 88 ± 67 | 108.1 | 9.68E−06 | 0/5 |

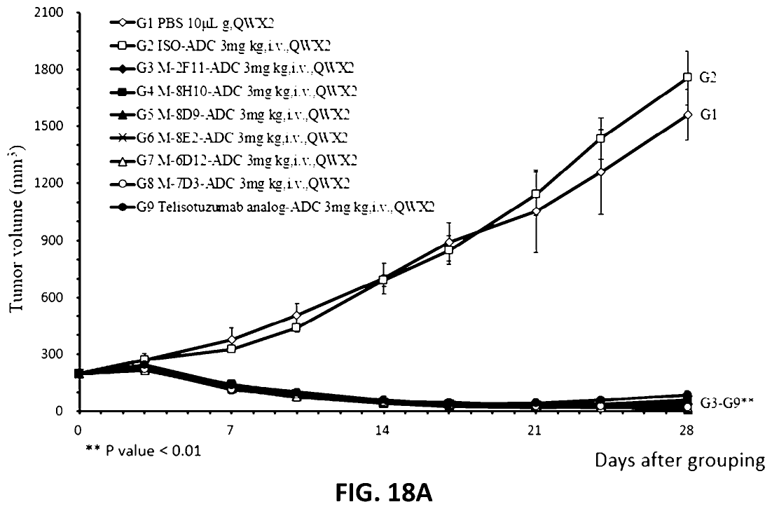

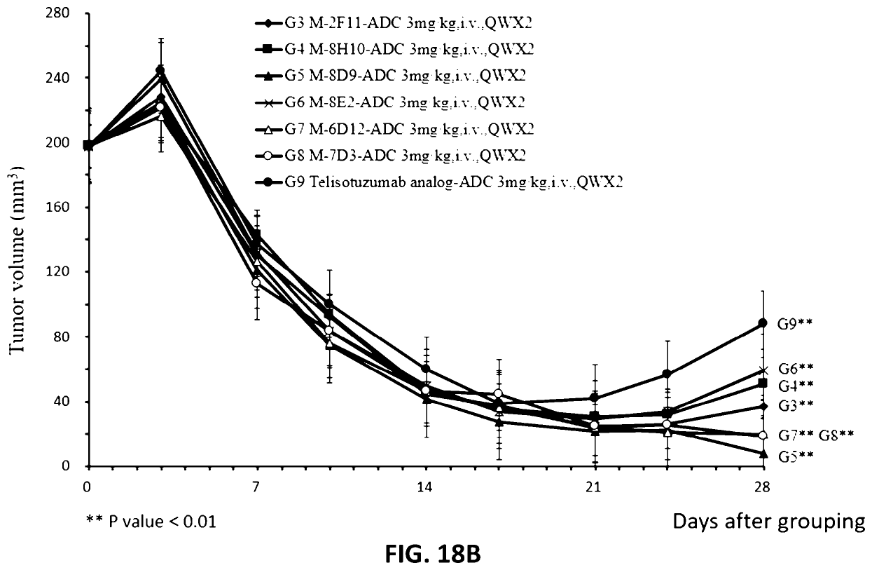

The tumor volume was measured twice a week, and the results are shown in Table 47 and FIGS. 18A-18B, which showed that the treatment groups with anti-MET antibody ADCs (G3-G19) resulted in robust tumor growth inhibition in human NUGC-4 xenograft model compared with the control group (G11 and G2). The tumor volume of Telisotuzumab analog-ADC (G9) was higher than that of G3-G8 from Day 21 to Day 28, suggesting that the anti-MET ADCs (G3-G8) had better tumor inhibitory effect compared with Telisotuzumab analog-ADC at a dosage of 3 mg/kg. Specifically, on Day 28 post grouping, the survived number of mice was 3 in M-8D9-ADC treatment group (G5) including 2 tumor-free mice.

### Example 16. Antibody Drug Conjugates

The purified antibodies were coupled with CPT-1, CPT-2, CPT-3, or CPT-4, through a CPT-L linker. For the names of antibody-drug conjugates, CPTx (x=1, 2, 3, or 4) is added directly after the antibody name. For example, if E-6C4-M-2F11 is coupled to CPT-1, it is named as E-6C4-M-2F11-CPT1. As another example, if E-6C4-M-2F11 is coupled to CPT-2, it is named as E-6C4-M-2F11-CPT2. Exemplary ADCs obtained by this method included: E-1G11-CPT2, E-2B7-CPT2, E-3A8-CPT2, E-3D8-CPT1, E-6C4-CPT2, E-9 Å6-CPT1, M-1E9-CPT1, M-2F11-CPT2, M-2F12-CPT1, M-2G10-CPT2, M-8D9-CPT1, E-1G11-M-2F11-CPT2, E-1G11-M-2G10-CPT2, E-6C4-M-1C5-CPT1, E-6C4-M-2F11-CPT2, E-6C4-M-2G10-CPT2, E-9H2-M-2G10-CPT1, E-1G11-M-8D9-CPT2, and E-2B7-M-8H10-CPT1.

HIC-HPLC was used to detect the coupling of antibodies with drug molecules. A human IgG1 molecule was coupled to CPT-2 to form isotype-CPT2 (ISO-CPT2), as an isotype control. The HIC-HPLC detection results showed that the drug-to-antibody ratio (DAR) of the ADCs was about 4 or 8. With regard to the ADC names, if the DAR of E-6C4-M-2F11-CPT1 is 4, the ADC is named E-6C4-M-2F11-CPT1 (DAR4). If the DAR of E-6C4-M-2F11-CPT1 is 8, the ADC is named E-6C4-M-2F11-CPT1 (DAR8).

### Example 17. Anti-Tumor Activity of ADCs in Lung Cancer Model

The ADCs were tested for their inhibitory effects of tumor growth in vivo in a model of lung carcinoma. Specifically, about 2×10⁶ NCI-H292 cells were injected subcutaneously in Balb/c nude mice. When the tumor in mice reached a volume of about 300 mm³, the mice were randomly placed into different groups based on the tumor volume. The mice were then injected with PBS, ADCs, or Amivantamab by intravenous (i.v.) administration. The frequency of ADC administration was once a week (1 administration in total). Details of the dosing schedule, route, and frequency are shown in the table below.

TABLE 48

| | No. of | | | | | | Total No. of |
|---|---|---|---|---|---|---|---|
| Group | mice | Antibodies | Dosage | | Route | Frequency | administration |
| G1 | 5 | PBS | — | | i.v. | QW | 1 |
| G2 | 5 | ISO-CPT2 (DAR4) | 10 | mg/kg | i.v. | QW | 1 |
| G3 | 5 | E-6C4-M-2F11-CPT2 (DAR4) | 3 | mg/kg | i.v. | QW | 1 |
| G4 | 5 | E-6C4-M-2F11-CPT2 (DAR4) | 6 | mg/kg | i.v. | QW | 1 |
| G5 | 5 | E-6C4-M-2F11-CPT2 (DAR4) | 10 | mg/kg | i.v. | QW | 1 |
| G6 | 5 | ISO-CPT2 (DAR8) | 10 | mg/kg | i.v. | QW | 1 |
| G7 | 5 | E-6C4-M-2F11-CPT2 (DAR8) | 3 | mg/kg | i.v. | QW | 1 |
| G8 | 5 | E-6C4-M-2F11-CPT2 (DAR8) | 6 | mg/kg | i.v. | QW | 1 |
| G9 | 5 | E-6C4-M-2F11-CPT2 (DAR8) | 10 | mg/kg | i.v. | QW | 1 |
| G10 | 5 | Amivantamab | 10 | mg/kg | i.v. | BIW | 4 |

The results showed that E-6C4-M-2F1-CPT2 with DAR4 and DAR8 both exhibited good tumor inhibitory effects.

In a similar experiment, about 2×10⁶ NCI-H1975 cells were injected subcutaneously in Balb/c nude mice. When the tumor in mice reached a volume of about 300 mm³, the mice were randomly placed into different groups based on the tumor volume. The mice were then injected with PBS, ADCs, or Amivantamab by intravenous (i.v.) administration. Details of the dosing schedule, route, and frequency are shown in the table below.

TABLE 49

| | No. of | | | | | | Total No. of |
|---|---|---|---|---|---|---|---|
| Group | mice | Antibodies | Dosage | | Route | Frequency | administration |
| G1 | 5 | PBS | — | | i.v. | QW | 1 |
| G2 | 5 | ISO-CPT2 (DAR4) | 6 | mg/kg | i.v. | QW | 1 |
| G3 | 5 | E-6C4-M-2F11-CPT2 (DAR4) | 1.5 | mg/kg | i.v. | QW | 1 |
| G4 | 5 | E-6C4-M-2F11-CPT2 (DAR4) | 3 | mg/kg | i.v. | QW | 1 |
| G5 | 5 | E-6C4-M-2F11-CPT2 (DAR4) | 6 | mg/kg | i.v. | QW | 1 |
| G6 | 5 | ISO-CPT2 (DAR8) | 6 | mg/kg | i.v. | QW | 1 |
| G7 | 5 | E-6C4-M-2F11-CPT2 (DAR8) | 1.5 | mg/kg | i.v. | QW | 1 |
| G8 | 5 | E-6C4-M-2F11-CPT2 (DAR8) | 3 | mg/kg | i.v. | QW | 1 |
| G9 | 5 | E-6C4-M-2F11-CPT2 (DAR8) | 6 | mg/kg | i.v. | QW | 1 |
| G10 | 5 | Amivantamab | 10 | mg/kg | i.v. | BIW | 4 |

Similar to the above results, E-6C4-M-2F11-CPT2 with DAR4 and DAR8 both obtained good inhibitory effects of tumor growth.

### Example 18. Anti-Tumor Activity of ADCs in Pancreatic Cancer Mode

B-NDG mice were engrafted in the right flank with pancreatic cancer patient-derived tumor tissue fragments (2 mm×2 mm×2 mm). When the tumors in the mice reached a volume of about 200-300 mm³, the mice were randomly placed into different groups based on the tumor volume. The mice were then injected with PBS, ADCs, or Amivantamab by i.v. administration. Details of the dosing schedule, route, and frequency are shown in the table below.

TABLE 50

| | No. of | | | | | Total No. of |
|---|---|---|---|---|---|---|
| Group | mice | Antibodies | Dosage | Route | Frequency | administration |
| G1 | 5 | PBS | — | i.v. | QW | 1 |
| G2 | 5 | Telisotuzumab analog-ADC | 3 mg/kg | i.v. | QW | 1 |
| G3 | 5 | MRG003 analog-ADC | 3 mg/kg | i.v. | QW | 1 |
| G4 | 5 | ISO-CPT2 (DAR4) | 6 mg/kg | i.v. | QW | 1 |
| G5 | 5 | E-6C4-M-2F11-CPT2 (DAR4) | 1 mg/kg | i.v. | QW | 1 |
| G6 | 5 | E-6C4-M-2F11-CPT2 (DAR4) | 3 mg/kg | i.v. | QW | 1 |
| G7 | 5 | E-6C4-M-2F11-CPT2 (DAR4) | 6 mg/kg | i.v. | QW | 1 |
| G8 | 5 | ISO-CPT2 (DAR8) | 6 mg/kg | i.v. | QW | 1 |
| G9 | 5 | E-6C4-M-2F11-CPT2 (DAR8) | 1 mg/kg | i.v. | QW | 1 |
| G10 | 5 | E-6C4-M-2F11-CPT2 (DAR8) | 3 mg/kg | i.v. | QW | 1 |
| G11 | 5 | E-6C4-M-2F11-CPT2 (DAR8) | 6 mg/kg | i.v. | QW | 1 |
| G12 | 5 | Amivantamab | 10 mg/kg | i.v. | BIW | 4 |

The results showed that E-6C4-M-2F11-CPT2 with DAR4 and DAR8 both exhibited a superior efficacy in treating pancreatic cancer.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to EGFR (epidermal growth factor receptor) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 4-6, respectively, and the selected VL CDRs 1, 2, 3 amino acid sequences are set forth in SEQ ID NOs: 1-3, respectively;
(2) the selected VH CDRs 1, 2, 3 amino acid sequences are set forth in SEQ ID NOs: 7-9, respectively, and the selected VL CDRs 1, 2, 3 amino acid sequences are set forth in SEQ ID NOs: 1-3, respectively;
(3) the selected VH CDRs 1, 2, 3 amino acid sequences are set forth in SEQ ID NOs: 10-12, respectively, and the selected VL CDRs 1, 2, 3 amino acid sequences are set forth in SEQ ID NOs: 1-3, respectively;
(4) the selected VH CDRs 1, 2, 3 amino acid sequences are set forth in SEQ ID NOs: 13-15, respectively, and the selected VL CDRs 1, 2, 3 amino acid sequences are set forth in SEQ ID NOs: 1-3, respectively;
(5) the selected VH CDRs 1, 2, 3 amino acid sequences are set forth in SEQ ID NOs: 16-18, respectively, and the selected VL CDRs 1, 2, 3 amino acid sequences are set forth in SEQ ID NOs: 1-3, respectively;
(6) the selected VH CDRs 1, 2, 3 amino acid sequences are set forth in SEQ ID NOs: 34-36, respectively, and the selected VL CDRs 1, 2, 3 amino acid sequences are set forth in SEQ ID NOs: 1-3, respectively;
(7) the selected VH CDRs 1, 2, 3 amino acid sequences are set forth in SEQ ID NOs: 37-39, respectively, and the selected VL CDRs 1, 2, 3 amino acid sequences are set forth in SEQ ID NOs: 1-3, respectively;
(8) the selected VH CDRs 1, 2, 3 amino acid sequences are set forth in SEQ ID NOs: 40-42, respectively, and the selected VL CDRs 1, 2, 3 amino acid sequences are set forth in SEQ ID NOs: 1-3, respectively;
(9) the selected VH CDRs 1, 2, 3 amino acid sequences are set forth in SEQ ID NOs: 43-45, respectively, and the selected VL CDRs 1, 2, 3 amino acid sequences are set forth in SEQ ID NOs: 1-3, respectively;
(10) the selected VH CDRs 1, 2, 3 amino acid sequences are set forth in SEQ ID NOs: 46-48, respectively, and the selected VL CDRs 1, 2, 3 amino acid sequences are set forth in SEQ ID NOs: 1-3, respectively;
(11) the selected VH CDRs 1, 2, 3 amino acid sequences are set forth in SEQ ID NOs: 82-84, respectively, and the selected VL CDRs 1, 2, 3 amino acid sequences are set forth in SEQ ID NOs: 1-3, respectively;
(12) the selected VH CDRs 1, 2, 3 amino acid sequences are set forth in SEQ ID NOs: 85-87, respectively, and the selected VL CDRs 1, 2, 3 amino acid sequences are set forth in SEQ ID NOs: 1-3, respectively;
(13) the selected VH CDRs 1, 2, 3 amino acid sequences are set forth in SEQ ID NOs: 88-90, respectively, and the selected VL CDRs 1, 2, 3 amino acid sequences are set forth in SEQ ID NOs: 1-3, respectively;
(14) the selected VH CDRs 1, 2, 3 amino acid sequences are set forth in SEQ ID NOs: 91-93, respectively, and the selected VL CDRs 1, 2, 3 amino acid sequences are set forth in SEQ ID NOs: 1-3, respectively;
(15) the selected VH CDRs 1, 2, 3 amino acid sequences are set forth in SEQ ID NOs: 94-96, respectively, and the selected VL CDRs 1, 2, 3 amino acid sequences are set forth in SEQ ID NOs: 1-3, respectively;
(16) the selected VH CDRs 1, 2, 3 amino acid sequences are set forth in SEQ ID NOs: 97-99, respectively, and the selected VL CDRs 1, 2, 3 amino acid sequences are set forth in SEQ ID NOs: 1-3, respectively;
(17) the selected VH CDRs 1, 2, 3 amino acid sequences are set forth in SEQ ID NOs: 100-102, respectively, and the selected VL CDRs 1, 2, 3 amino acid sequences are set forth in SEQ ID NOs: 1-3, respectively; and
(18) the selected VH CDRs 1, 2, 3 amino acid sequences are set forth in SEQ ID NOs: 103-105, respectively, and the selected VL CDRs 1, 2, 3 amino acid sequences are set forth in SEQ ID NOs: 1-3, respectively.

2. The antibody or antigen-binding fragment thereof of claim 1, wherein the VH comprises CDRs 1, 2, 3 with the amino acid sequences set forth in SEQ ID NOs: 4-6, respectively, and the VL comprises CDRs 1, 2, 3 with the amino acid sequences set forth in SEQ ID NOs: 1-3, respectively, according to the Kabat numbering scheme.

3. The antibody or antigen-binding fragment thereof of claim 1, wherein the VH comprises CDRs 1, 2, 3 with the amino acid sequences set forth in SEQ ID NOs: 7-9, respectively, and the VL comprises CDRs 1, 2, 3 with the amino acid sequences set forth in SEQ ID NOs: 1-3, respectively, according to the Kabat numbering scheme.

4. The antibody or antigen-binding fragment thereof of claim 1, wherein the VH comprises CDRs 1, 2, 3 with the amino acid sequences set forth in SEQ ID NOs: 10-12, respectively, and the VL comprises CDRs 1, 2, 3 with the amino acid sequences set forth in SEQ ID NOs: 1-3, respectively, according to the Kabat numbering scheme.

5. The antibody or antigen-binding fragment thereof of claim 1, wherein the VH comprises CDRs 1, 2, 3 with the amino acid sequences set forth in SEQ ID NOs: 13-15, respectively, and the VL comprises CDRs 1, 2, 3 with the amino acid sequences set forth in SEQ ID NOs: 1-3, respectively, according to the Kabat numbering scheme.

6. The antibody or antigen-binding fragment thereof of claim 1, wherein the VH comprises CDRs 1, 2, 3 with the amino acid sequences set forth in SEQ ID NOs: 16-18, respectively, and the VL comprises CDRs 1, 2, 3 with the amino acid sequences set forth in SEQ ID NOs: 1-3, respectively, according to the Kabat numbering scheme.

7. The antibody or antigen-binding fragment thereof of claim 1, wherein the VH comprises CDRs 1, 2, 3 with the amino acid sequences set forth in SEQ ID NOs: 82-84, respectively, and the VL comprises CDRs 1, 2, 3 with the amino acid sequences set forth in SEQ ID NOs: 1-3, respectively, according to the Kabat numbering scheme.

8. The antibody or antigen-binding fragment thereof of claim 1, wherein the VH comprises CDRs 1, 2, 3 with the amino acid sequences set forth in SEQ ID NOs: 85-87, respectively, and the VL comprises CDRs 1, 2, 3 with the amino acid sequences set forth in SEQ ID NOs: 1-3, respectively, according to the Kabat numbering scheme.

9. The antibody or antigen-binding fragment thereof of claim 1, wherein the VH comprises CDRs 1, 2, 3 with the amino acid sequences set forth in SEQ ID NOs: 88-90, respectively, and the VL comprises CDRs 1, 2, 3 with the amino acid sequences set forth in SEQ ID NOs: 1-3, respectively, according to the Kabat numbering scheme.

10. The antibody or antigen-binding fragment thereof of claim 1, wherein the VH comprises CDRs 1, 2, 3 with the amino acid sequences set forth in SEQ ID NOs: 91-93, respectively, and the VL comprises CDRs 1, 2, 3 with the amino acid sequences set forth in SEQ ID NOs: 1-3, respectively, according to the Kabat numbering scheme.

11. The antibody or antigen-binding fragment thereof of claim 1, wherein the VH comprises CDRs 1, 2, 3 with the amino acid sequences set forth in SEQ ID NOs: 34-36, respectively, and the VL comprises CDRs 1, 2, 3 with the amino acid sequences set forth in SEQ ID NOs: 1-3, respectively, according to the Chothia numbering scheme.

12. The antibody or antigen-binding fragment thereof of claim 1, wherein the VH comprises CDRs 1, 2, 3 with the amino acid sequences set forth in SEQ ID NOs: 37-39, respectively, and the VL comprises CDRs 1, 2, 3 with the amino acid sequences set forth in SEQ ID NOs: 1-3, respectively, according to the Chothia numbering scheme.

13. The antibody or antigen-binding fragment thereof of claim 1, wherein the VH comprises CDRs 1, 2, 3 with the amino acid sequences set forth in SEQ ID NOs: 40-42, respectively, and the VL comprises CDRs 1, 2, 3 with the amino acid sequences set forth in SEQ ID NOs: 1-3, respectively, according to the Chothia numbering scheme.

14. The antibody or antigen-binding fragment thereof of claim 1, wherein the VH comprises CDRs 1, 2, 3 with the amino acid sequences set forth in SEQ ID NOs: 43-45, respectively, and the VL comprises CDRs 1, 2, 3 with the amino acid sequences set forth in SEQ ID NOs: 1-3, respectively, according to the Chothia numbering scheme.

15. The antibody or antigen-binding fragment thereof of claim 1, wherein the VH comprises CDRs 1, 2, 3 with the amino acid sequences set forth in SEQ ID NOs: 46-48, respectively, and the VL comprises CDRs 1, 2, 3 with the amino acid sequences set forth in SEQ ID NOs: 1-3, respectively, according to the Chothia numbering scheme.

16. The antibody or antigen-binding fragment thereof of claim 1, wherein the VH comprises CDRs 1, 2, 3 with the amino acid sequences set forth in SEQ ID NOs: 94-96, respectively, and the VL comprises CDRs 1, 2, 3 with the amino acid sequences set forth in SEQ ID NOs: 1-3, respectively, according to the Chothia numbering scheme.

17. The antibody or antigen-binding fragment thereof of claim 1, wherein the VH comprises CDRs 1, 2, 3 with the amino acid sequences set forth in SEQ ID NOs: 97-99, respectively, and the VL comprises CDRs 1, 2, 3 with the amino acid sequences set forth in SEQ ID NOs: 1-3, respectively, according to the Chothia numbering scheme.

18. The antibody or antigen-binding fragment thereof of claim 1, wherein the VH comprises CDRs 1, 2, 3 with the amino acid sequences set forth in SEQ ID NOs: 100-102, respectively, and the VL comprises CDRs 1, 2, 3 with the amino acid sequences set forth in SEQ ID NOs: 1-3, respectively, according to the Chothia numbering scheme.

19. The antibody or antigen-binding fragment thereof of claim 1, wherein the VH comprises CDRs 1, 2, 3 with the amino acid sequences set forth in SEQ ID NOs: 103-105, respectively, and the VL comprises CDRs 1, 2, 3 with the amino acid sequences set forth in SEQ ID NOs: 1-3, respectively, according to the Chothia numbering scheme.

20. An antibody or antigen-binding fragment thereof that binds to EGFR comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 62, and the selected VL sequence is SEQ ID NO: 61;
(2) the selected VH sequence is SEQ ID NO: 63, and the selected VL sequence is SEQ ID NO: 61;
(3) the selected VH sequence is SEQ ID NO: 64, and the selected VL sequence is SEQ ID NO: 61;
(4) the selected VH sequence is SEQ ID NO: 65, and the selected VL sequence is SEQ ID NO: 61;
(5) the selected VH sequence is SEQ ID NO: 66, and the selected VL sequence is SEQ ID NO: 61;
(6) the selected VH sequence is SEQ ID NO: 106, and the selected VL sequence is SEQ ID NO: 61;
(7) the selected VH sequence is SEQ ID NO: 10⁷, and the selected VL sequence is SEQ ID NO: 61;
(8) the selected VH sequence is SEQ ID NO: 108, and the selected VL sequence is SEQ ID NO: 61; and
(9) the selected VH sequence is SEQ ID NO: 109, and the selected VL sequence is SEQ ID NO: 61.

21. The antibody or antigen-binding fragment thereof of claim 20, wherein the VH comprises the sequence of SEQ ID NO: 62 and the VL comprises the sequence of SEQ ID NO: 61.

22. The antibody or antigen-binding fragment thereof of claim 20, wherein the VH comprises the sequence of SEQ ID NO: 63 and the VL comprises the sequence of SEQ ID NO: 61.

23. The antibody or antigen-binding fragment thereof of claim 20, wherein the VH comprises the sequence of SEQ ID NO: 64 and the VL comprises the sequence of SEQ ID NO: 61.

24. The antibody or antigen-binding fragment thereof of claim 20, wherein the VH comprises the sequence of SEQ ID NO: 65 and the VL comprises the sequence of SEQ ID NO: 61.

25. The antibody or antigen-binding fragment thereof of claim 20, wherein the VH comprises the sequence of SEQ ID NO: 66 and the VL comprises the sequence of SEQ ID NO: 61.

26. The antibody or antigen-binding fragment thereof of claim 20, wherein the VH comprises the sequence of SEQ ID NO: 106 and the VL comprises the sequence of SEQ ID NO: 61.

27. The antibody or antigen-binding fragment thereof of claim 20, wherein the VH comprises the sequence of SEQ ID NO: 10⁷ and the VL comprises the sequence of SEQ ID NO: 61.

28. The antibody or antigen-binding fragment thereof of claim 20, wherein the VH comprises the sequence of SEQ ID NO: 108 and the VL comprises the sequence of SEQ ID NO: 61.

29. The antibody or antigen-binding fragment thereof of claim 20, wherein the VH comprises the sequence of SEQ ID NO: 109 and the VL comprises the sequence of SEQ ID NO: 61.

30. An antibody or antigen-binding fragment thereof that binds to EGFR comprising
a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 62, and the selected VL sequence is SEQ ID NO: 61;
(2) the selected VH sequence is SEQ ID NO: 63, and the selected VL sequence is SEQ ID NO: 61;
(3) the selected VH sequence is SEQ ID NO: 64, and the selected VL sequence is SEQ ID NO: 61;
(4) the selected VH sequence is SEQ ID NO: 65, and the selected VL sequence is SEQ ID NO: 61;
(5) the selected VH sequence is SEQ ID NO: 66, and the selected VL sequence is SEQ ID NO: 61;
(6) the selected VH sequence is SEQ ID NO: 106, and the selected VL sequence is SEQ ID NO: 61;
(7) the selected VH sequence is SEQ ID NO: 10⁷, and the selected VL sequence is SEQ ID NO: 61;
(8) the selected VH sequence is SEQ ID NO: 108, and the selected VL sequence is SEQ ID NO: 61; and
(9) the selected VH sequence is SEQ ID NO: 109, and the selected VL sequence is SEQ ID NO: 61.

31. The antibody or antigen-binding fragment thereof of any one of claims 1-30, wherein the antibody or antigen-binding fragment thereof specifically binds to human or monkey EGFR.

32. The antibody or antigen-binding fragment thereof of any one of claims 1-31, wherein the antibody or antigen-binding fragment thereof is a human or humanized antibody or antigen-binding fragment thereof.

33. The antibody or antigen-binding fragment thereof of any one of claims 1-32, wherein the antibody or antigen-binding fragment is a single-chain variable fragment (scFV).

34. The antibody or antigen-binding fragment thereof of any one of claims 1-33, wherein the antibody or antigen-binding fragment thereof is a multispecific antibody (e.g., a bispecific antibody) or an antigen-binding fragment thereof.

35. The antibody or antigen-binding fragment thereof of claim 34, wherein the antibody or antigen-binding fragment thereof further specifically binds to MET.

36. An antibody or antigen-binding fragment thereof that cross-competes with the antibody or antigen-binding fragment thereof of any one of claims 1-35.

37. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 4-6, respectively, and wherein the VH, when paired with a light chain variable region (VL) comprising the amino acid sequence set forth in SEQ ID NO: 61, binds to EGFR;
(2) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 62, binds to EGFR;
(3) an immunoglobulin heavy chain or a fragment thereof comprising a VH comprising CDRs 1, 2, and 3 comprising the amino acid sequences set forth in SEQ ID NOs: 7-9, respectively, and wherein the VH, when paired with a VL comprising the amino acid sequence set forth in SEQ ID NO: 61, binds to EGFR;
(4) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 63, binds to EGFR;
(5) an immunoglobulin heavy chain or a fragment thereof comprising a VH comprising CDRs 1, 2, and 3 comprising the amino acid sequences set forth in SEQ ID NOs: 10-12, respectively, and wherein the VH, when paired with a VL comprising the amino acid sequence set forth in SEQ ID NO: 61, binds to EGFR;
(6) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 64, binds to EGFR;
(7) an immunoglobulin heavy chain or a fragment thereof comprising a VH comprising CDRs 1, 2, and 3 comprising the amino acid sequences set forth in SEQ ID NOs: 13-15, respectively, and wherein the VH, when paired with a VL comprising the amino acid sequence set forth in SEQ ID NO: 61, binds to EGFR;
(8) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 65, binds to EGFR;
(9) an immunoglobulin heavy chain or a fragment thereof comprising a VH comprising CDRs 1, 2, and 3 comprising the amino acid sequences set forth in SEQ ID NOs: 16-18, respectively, and wherein the VH, when paired with a VL comprising the amino acid sequence set forth in SEQ ID NO: 61, binds to EGFR;
(10) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 66, binds to EGFR;
(11) an immunoglobulin heavy chain or a fragment thereof comprising a VH comprising CDRs 1, 2, and 3 comprising the amino acid sequences set forth in SEQ ID NOs: 34-36, respectively, and wherein the VH, when paired with a VL comprising the amino acid sequence set forth in SEQ ID NO: 61, binds to EGFR;
(12) an immunoglobulin heavy chain or a fragment thereof comprising a VH comprising CDRs 1, 2, and 3 comprising the amino acid sequences set forth in SEQ ID NOs: 37-39, respectively, and wherein the VH, when paired with a VL comprising the amino acid sequence set forth in SEQ ID NO: 61, binds to EGFR;
(13) an immunoglobulin heavy chain or a fragment thereof comprising a VH comprising CDRs 1, 2, and 3 comprising the amino acid sequences set forth in SEQ ID NOs: 40-42, respectively, and wherein the VH, when paired with a VL comprising the amino acid sequence set forth in SEQ ID NO: 61, binds to EGFR;
(14) an immunoglobulin heavy chain or a fragment thereof comprising a VH comprising CDRs 1, 2, and 3 comprising the amino acid sequences set forth in SEQ ID NOs: 43-45, respectively, and wherein the VH, when paired with a VL comprising the amino acid sequence set forth in SEQ ID NO: 61, binds to EGFR;
(15) an immunoglobulin heavy chain or a fragment thereof comprising a VH comprising CDRs 1, 2, and 3 comprising the amino acid sequences set forth in SEQ ID NOs: 46-48, respectively, and wherein the VH, when paired with a VL comprising the amino acid sequence set forth in SEQ ID NO: 61, binds to EGFR;
(16) an immunoglobulin heavy chain or a fragment thereof comprising a VH comprising CDRs 1, 2, and 3 comprising the amino acid sequences set forth in SEQ ID NOs: 82-84, respectively, and wherein the VH, when paired with a VL comprising the amino acid sequence set forth in SEQ ID NO: 61, binds to EGFR;
(17) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 106, binds to EGFR;
(18) an immunoglobulin heavy chain or a fragment thereof comprising a VH comprising CDRs 1, 2, and 3 comprising the amino acid sequences set forth in SEQ ID NOs: 85-87, respectively, and wherein the VH, when paired with a VL comprising the amino acid sequence set forth in SEQ ID NO: 61, binds to EGFR;
(19) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 10⁷, binds to EGFR;
(20) an immunoglobulin heavy chain or a fragment thereof comprising a VH comprising CDRs 1, 2, and 3 comprising the amino acid sequences set forth in SEQ ID NOs: 88-90, respectively, and wherein the VH, when paired with a VL comprising the amino acid sequence set forth in SEQ ID NO: 61, binds to EGFR;
(21) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 108, binds to EGFR;
(22) an immunoglobulin heavy chain or a fragment thereof comprising a VH comprising CDRs 1, 2, and 3 comprising the amino acid sequences set forth in SEQ ID NOs: 91-93, respectively, and wherein the VH, when paired with a VL comprising the amino acid sequence set forth in SEQ ID NO: 61, binds to EGFR;
(23) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 109, binds to EGFR;
(24) an immunoglobulin heavy chain or a fragment thereof comprising a VH comprising CDRs 1, 2, and 3 comprising the amino acid sequences set forth in SEQ ID NOs: 94-96, respectively, and wherein the VH, when paired with a VL comprising the amino acid sequence set forth in SEQ ID NO: 61, binds to EGFR;
(25) an immunoglobulin heavy chain or a fragment thereof comprising a VH comprising CDRs 1, 2, and 3 comprising the amino acid sequences set forth in SEQ ID NOs: 97-99, respectively, and wherein the VH, when paired with a VL comprising the amino acid sequence set forth in SEQ ID NO: 61, binds to EGFR;
(26) an immunoglobulin heavy chain or a fragment thereof comprising a VH comprising CDRs 1, 2, and 3 comprising the amino acid sequences set forth in SEQ ID NOs: 100-102, respectively, and wherein the VH, when paired with a VL comprising the amino acid sequence set forth in SEQ ID NO: 61, binds to EGFR; and
(27) an immunoglobulin heavy chain or a fragment thereof comprising a VH comprising CDRs 1, 2, and 3 comprising the amino acid sequences set forth in SEQ ID NOs: 103-105, respectively, and wherein the VH, when paired with a VL comprising the amino acid sequence set forth in SEQ ID NO: 61, binds to EGFR.

38. The nucleic acid of claim 37, wherein the VH when paired with a VL specifically binds to human or monkey EGFR.

39. The nucleic acid of claim 37 or 38, wherein the immunoglobulin heavy chain or the fragment thereof is a human or humanized immunoglobulin heavy chain or a fragment thereof.

40. The nucleic acid of any one of claims 37-39, wherein the nucleic acid encodes a single-chain variable fragment (scFv).

41. The nucleic acid of any one of claims 37-40, wherein the nucleic acid is cDNA.

42. An antibody or antigen-binding fragment thereof that binds to MET (tyrosine-protein kinase Met) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 19-21, respectively, and the selected VL CDRs 1, 2, 3 amino acid sequences are set forth in SEQ ID NOs: 1-3, respectively;
(2) the selected VH CDRs 1, 2, 3 amino acid sequences are set forth in SEQ ID NOs: 22-24, respectively, and the selected VL CDRs 1, 2, 3 amino acid sequences are set forth in SEQ ID NOs: 1-3, respectively;
(3) the selected VH CDRs 1, 2, 3 amino acid sequences are set forth in SEQ ID NOs: 25-27, respectively, and the selected VL CDRs 1, 2, 3 amino acid sequences are set forth in SEQ ID NOs: 1-3, respectively;
(4) the selected VH CDRs 1, 2, 3 amino acid sequences are set forth in SEQ ID NOs: 28-30, respectively, and the selected VL CDRs 1, 2, 3 amino acid sequences are set forth in SEQ ID NOs: 1-3, respectively;
(5) the selected VH CDRs 1, 2, 3 amino acid sequences are set forth in SEQ ID NOs: 49-51, respectively, and the selected VL CDRs 1, 2, 3 amino acid sequences are set forth in SEQ ID NOs: 1-3, respectively;
(6) the selected VH CDRs 1, 2, 3 amino acid sequences are set forth in SEQ ID NOs: 52-54, respectively, and the selected VL CDRs 1, 2, 3 amino acid sequences are set forth in SEQ ID NOs: 1-3, respectively;
(7) the selected VH CDRs 1, 2, 3 amino acid sequences are set forth in SEQ ID NOs: 55-57, respectively, and the selected VL CDRs 1, 2, 3 amino acid sequences are set forth in SEQ ID NOs: 1-3, respectively;
(8) the selected VH CDRs 1, 2, 3 amino acid sequences are set forth in SEQ ID NOs: 58-60, respectively, and the selected VL CDRs 1, 2, 3 amino acid sequences are set forth in SEQ ID NOs: 1-3, respectively;
(9) the selected VH CDRs 1, 2, 3 amino acid sequences are set forth in SEQ ID NOs: 112-114, respectively, and the selected VL CDRs 1, 2, 3 amino acid sequences are set forth in SEQ ID NOs: 1-3, respectively;
(10) the selected VH CDRs 1, 2, 3 amino acid sequences are set forth in SEQ ID NOs: 115-117, respectively, and the selected VL CDRs 1, 2, 3 amino acid sequences are set forth in SEQ ID NOs: 1-3, respectively;
(11) the selected VH CDRs 1, 2, 3 amino acid sequences are set forth in SEQ ID NOs: 118-120, respectively, and the selected VL CDRs 1, 2, 3 amino acid sequences are set forth in SEQ ID NOs: 1-3, respectively;
(12) the selected VH CDRs 1, 2, 3 amino acid sequences are set forth in SEQ ID NOs: 121-123, respectively, and the selected VL CDRs 1, 2, 3 amino acid sequences are set forth in SEQ ID NOs: 1-3, respectively;
(13) the selected VH CDRs 1, 2, 3 amino acid sequences are set forth in SEQ ID NOs: 124-126, respectively, and the selected VL CDRs 1, 2, 3 amino acid sequences are set forth in SEQ ID NOs: 1-3, respectively;
(14) the selected VH CDRs 1, 2, 3 amino acid sequences are set forth in SEQ ID NOs: 127-129, respectively, and the selected VL CDRs 1, 2, 3 amino acid sequences are set forth in SEQ ID NOs: 1-3, respectively;
(15) the selected VH CDRs 1, 2, 3 amino acid sequences are set forth in SEQ ID NOs: 130-132, respectively, and the selected VL CDRs 1, 2, 3 amino acid sequences are set forth in SEQ ID NOs: 1-3, respectively;
(16) the selected VH CDRs 1, 2, 3 amino acid sequences are set forth in SEQ ID NOs: 133-135, respectively, and the selected VL CDRs 1, 2, 3 amino acid sequences are set forth in SEQ ID NOs: 1-3, respectively;
(17) the selected VH CDRs 1, 2, 3 amino acid sequences are set forth in SEQ ID NOs: 136-138, respectively, and the selected VL CDRs 1, 2, 3 amino acid sequences are set forth in SEQ ID NOs: 1-3, respectively;
(18) the selected VH CDRs 1, 2, 3 amino acid sequences are set forth in SEQ ID NOs: 139-141, respectively, and the selected VL CDRs 1, 2, 3 amino acid sequences are set forth in SEQ ID NOs: 1-3, respectively;
(19) the selected VH CDRs 1, 2, 3 amino acid sequences are set forth in SEQ ID NOs: 142-144, respectively, and the selected VL CDRs 1, 2, 3 amino acid sequences are set forth in SEQ ID NOs: 1-3, respectively; and
(20) the selected VH CDRs 1, 2, 3 amino acid sequences are set forth in SEQ ID NOs: 145-147, respectively, and the selected VL CDRs 1, 2, 3 amino acid sequences are set forth in SEQ ID NOs: 1-3, respectively.

43. The antibody or antigen-binding fragment thereof of claim 42, wherein the VH comprises CDRs 1, 2, 3 with the amino acid sequences set forth in SEQ ID NOs: 19-21, respectively, and the VL comprises CDRs 1, 2, 3 with the amino acid sequences set forth in SEQ ID NOs: 1-3, respectively, according to the Kabat numbering scheme.

44. The antibody or antigen-binding fragment thereof of claim 42, wherein the VH comprises CDRs 1, 2, 3 with the amino acid sequences set forth in SEQ ID NOs: 22-24, respectively, and the VL comprises CDRs 1, 2, 3 with the amino acid sequences set forth in SEQ ID NOs: 1-3, respectively, according to the Kabat numbering scheme.

45. The antibody or antigen-binding fragment thereof of claim 42, wherein the VH comprises CDRs 1, 2, 3 with the amino acid sequences set forth in SEQ ID NOs: 25-27, respectively, and the VL comprises CDRs 1, 2, 3 with the amino acid sequences set forth in SEQ ID NOs: 1-3, respectively, according to the Kabat numbering scheme.

46. The antibody or antigen-binding fragment thereof of claim 42, wherein the VH comprises CDRs 1, 2, 3 with the amino acid sequences set forth in SEQ ID NOs: 28-30, respectively, and the VL comprises CDRs 1, 2, 3 with the amino acid sequences set forth in SEQ ID NOs: 1-3, respectively, according to the Kabat numbering scheme.

47. The antibody or antigen-binding fragment thereof of claim 42, wherein the VH comprises CDRs 1, 2, 3 with the amino acid sequences set forth in SEQ ID NOs: 49-51, respectively, and the VL comprises CDRs 1, 2, 3 with the amino acid sequences set forth in SEQ ID NOs: 1-3, respectively, according to the Chothia numbering scheme.

48. The antibody or antigen-binding fragment thereof of claim 42, wherein the VH comprises CDRs 1, 2, 3 with the amino acid sequences set forth in SEQ ID NOs: 52-54, respectively, and the VL comprises CDRs 1, 2, 3 with the amino acid sequences set forth in SEQ ID NOs: 1-3, respectively, according to the Chothia numbering scheme.

49. The antibody or antigen-binding fragment thereof of claim 42, wherein the VH comprises CDRs 1, 2, 3 with the amino acid sequences set forth in SEQ ID NOs: 55-57, respectively, and the VL comprises CDRs 1, 2, 3 with the amino acid sequences set forth in SEQ ID NOs: 1-3, respectively, according to the Chothia numbering scheme.

50. The antibody or antigen-binding fragment thereof of claim 42, wherein the VH comprises CDRs 1, 2, 3 with the amino acid sequences set forth in SEQ ID NOs: 58-60, respectively, and the VL comprises CDRs 1, 2, 3 with the amino acid sequences set forth in SEQ ID NOs: 1-3, respectively, according to the Chothia numbering scheme.

51. The antibody or antigen-binding fragment thereof of claim 42, wherein the VH comprises CDRs 1, 2, 3 with the amino acid sequences set forth in SEQ ID NOs: 112-114, respectively, and the VL comprises CDRs 1, 2, 3 with the amino acid sequences set forth in SEQ ID NOs: 1-3, respectively, according to the Kabat numbering scheme.

52. The antibody or antigen-binding fragment thereof of claim 42, wherein the VH comprises CDRs 1, 2, 3 with the amino acid sequences set forth in SEQ ID NOs: 115-117, respectively, and the VL comprises CDRs 1, 2, 3 with the amino acid sequences set forth in SEQ ID NOs: 1-3, respectively, according to the Kabat numbering scheme.

53. The antibody or antigen-binding fragment thereof of claim 42, wherein the VH comprises CDRs 1, 2, 3 with the amino acid sequences set forth in SEQ ID NOs: 118-120, respectively, and the VL comprises CDRs 1, 2, 3 with the amino acid sequences set forth in SEQ ID NOs: 1-3, respectively, according to the Kabat numbering scheme.

54. The antibody or antigen-binding fragment thereof of claim 42, wherein the VH comprises CDRs 1, 2, 3 with the amino acid sequences set forth in SEQ ID NOs: 121-123, respectively, and the VL comprises CDRs 1, 2, 3 with the amino acid sequences set forth in SEQ ID NOs: 1-3, respectively, according to the Kabat numbering scheme.

55. The antibody or antigen-binding fragment thereof of claim 42, wherein the VH comprises CDRs 1, 2, 3 with the amino acid sequences set forth in SEQ ID NOs: 124-126, respectively, and the VL comprises CDRs 1, 2, 3 with the amino acid sequences set forth in SEQ ID NOs: 1-3, respectively, according to the Kabat numbering scheme.

56. The antibody or antigen-binding fragment thereof of claim 42, wherein the VH comprises CDRs 1, 2, 3 with the amino acid sequences set forth in SEQ ID NOs: 127-129, respectively, and the VL comprises CDRs 1, 2, 3 with the amino acid sequences set forth in SEQ ID NOs: 1-3, respectively, according to the Kabat numbering scheme.

57. The antibody or antigen-binding fragment thereof of claim 42, wherein the VH comprises CDRs 1, 2, 3 with the amino acid sequences set forth in SEQ ID NOs: 130-132, respectively, and the VL comprises CDRs 1, 2, 3 with the amino acid sequences set forth in SEQ ID NOs: 1-3, respectively, according to the Chothia numbering scheme.

58. The antibody or antigen-binding fragment thereof of claim 42, wherein the VH comprises CDRs 1, 2, 3 with the amino acid sequences set forth in SEQ ID NOs: 133-135, respectively, and the VL comprises CDRs 1, 2, 3 with the amino acid sequences set forth in SEQ ID NOs: 1-3, respectively, according to the Chothia numbering scheme.

59. The antibody or antigen-binding fragment thereof of claim 42, wherein the VH comprises CDRs 1, 2, 3 with the amino acid sequences set forth in SEQ ID NOs: 136-138, respectively, and the VL comprises CDRs 1, 2, 3 with the amino acid sequences set forth in SEQ ID NOs: 1-3, respectively, according to the Chothia numbering scheme.

60. The antibody or antigen-binding fragment thereof of claim 42, wherein the VH comprises CDRs 1, 2, 3 with the amino acid sequences set forth in SEQ ID NOs: 139-141, respectively, and the VL comprises CDRs 1, 2, 3 with the amino acid sequences set forth in SEQ ID NOs: 1-3, respectively, according to the Chothia numbering scheme.

61. The antibody or antigen-binding fragment thereof of claim 42, wherein the VH comprises CDRs 1, 2, 3 with the amino acid sequences set forth in SEQ ID NOs: 142-144, respectively, and the VL comprises CDRs 1, 2, 3 with the amino acid sequences set forth in SEQ ID NOs: 1-3, respectively, according to the Chothia numbering scheme.

62. The antibody or antigen-binding fragment thereof of claim 42, wherein the VH comprises CDRs 1, 2, 3 with the amino acid sequences set forth in SEQ ID NOs: 145-147, respectively, and the VL comprises CDRs 1, 2, 3 with the amino acid sequences set forth in SEQ ID NOs: 1-3, respectively, according to the Chothia numbering scheme.

63. An antibody or antigen-binding fragment thereof that binds to MET comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 67, and the selected VL sequence is SEQ ID NO: 61;
(2) the selected VH sequence is SEQ ID NO: 68, and the selected VL sequence is SEQ ID NO: 61;
(3) the selected VH sequence is SEQ ID NO: 69, and the selected VL sequence is SEQ ID NO: 61;
(4) the selected VH sequence is SEQ ID NO: 70, and the selected VL sequence is SEQ ID NO: 61;
(5) the selected VH sequence is SEQ ID NO: 148, and the selected VL sequence is SEQ ID NO: 61;
(6) the selected VH sequence is SEQ ID NO: 149, and the selected VL sequence is SEQ ID NO: 61;
(7) the selected VH sequence is SEQ ID NO: 150, and the selected VL sequence is SEQ ID NO: 61;
(8) the selected VH sequence is SEQ ID NO: 151, and the selected VL sequence is SEQ ID NO: 61;
(9) the selected VH sequence is SEQ ID NO: 152, and the selected VL sequence is SEQ ID NO: 61; and
(10) the selected VH sequence is SEQ ID NO: 153, and the selected VL sequence is SEQ ID NO: 61.

64. The antibody or antigen-binding fragment thereof of claim 63, wherein the VH comprises the sequence of SEQ ID NO: 67 and the VL comprises the sequence of SEQ ID NO: 61.

65. The antibody or antigen-binding fragment thereof of claim 63, wherein the VH comprises the sequence of SEQ ID NO: 68 and the VL comprises the sequence of SEQ ID NO: 61.

66. The antibody or antigen-binding fragment thereof of claim 63, wherein the VH comprises the sequence of SEQ ID NO: 69 and the VL comprises the sequence of SEQ ID NO: 61.

67. The antibody or antigen-binding fragment thereof of claim 63, wherein the VH comprises the sequence of SEQ ID NO: 70 and the VL comprises the sequence of SEQ ID NO: 61.

68. The antibody or antigen-binding fragment thereof of claim 63, wherein the VH comprises the sequence of SEQ ID NO: 148 and the VL comprises the sequence of SEQ ID NO: 61.

69. The antibody or antigen-binding fragment thereof of claim 63, wherein the VH comprises the sequence of SEQ ID NO: 149 and the VL comprises the sequence of SEQ ID NO: 61.

70. The antibody or antigen-binding fragment thereof of claim 63, wherein the VH comprises the sequence of SEQ ID NO: 150 and the VL comprises the sequence of SEQ ID NO: 61.

71. The antibody or antigen-binding fragment thereof of claim 63, wherein the VH comprises the sequence of SEQ ID NO: 151 and the VL comprises the sequence of SEQ ID NO: 61.

72. The antibody or antigen-binding fragment thereof of claim 63, wherein the VH comprises the sequence of SEQ ID NO: 152 and the VL comprises the sequence of SEQ ID NO: 61.

73. The antibody or antigen-binding fragment thereof of claim 63, wherein the VH comprises the sequence of SEQ ID NO: 153 and the VL comprises the sequence of SEQ ID NO: 61.

74. An antibody or antigen-binding fragment thereof that binds to MET comprising
a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 67, and the selected VL sequence is SEQ ID NO: 61;
(2) the selected VH sequence is SEQ ID NO: 68, and the selected VL sequence is SEQ ID NO: 61;
(3) the selected VH sequence is SEQ ID NO: 69, and the selected VL sequence is SEQ ID NO: 61;
(4) the selected VH sequence is SEQ ID NO: 70, and the selected VL sequence is SEQ ID NO: 61;
(5) the selected VH sequence is SEQ ID NO: 148, and the selected VL sequence is SEQ ID NO: 61;
(6) the selected VH sequence is SEQ ID NO: 149, and the selected VL sequence is SEQ ID NO: 61;
(7) the selected VH sequence is SEQ ID NO: 150, and the selected VL sequence is SEQ ID NO: 61;
(8) the selected VH sequence is SEQ ID NO: 151, and the selected VL sequence is SEQ ID NO: 61;
(9) the selected VH sequence is SEQ ID NO: 152, and the selected VL sequence is SEQ ID NO: 61; and
(10) the selected VH sequence is SEQ ID NO: 153, and the selected VL sequence is SEQ ID NO: 61.

75. The antibody or antigen-binding fragment thereof of any one of claims 42-74, wherein the antibody or antigen-binding fragment thereof specifically binds to human, monkey or dog MET.

76. The antibody or antigen-binding fragment thereof of any one of claims 42-75, wherein the antibody or antigen-binding fragment is a human or humanized antibody or antigen-binding fragment thereof.

77. The antibody or antigen-binding fragment thereof of any one of claims 42-76, wherein the antibody or antigen-binding fragment thereof is a single-chain variable fragment (scFV).

78. The antibody or antigen-binding fragment thereof of any one of claims 42-77, wherein the antibody or antigen-binding fragment thereof is a multispecific antibody (e.g., a bispecific antibody) or an antigen-binding fragment thereof.

79. The antibody or antigen-binding fragment thereof of claim 78, wherein the antibody or antigen-binding fragment thereof further specifically binds to EGFR.

80. An antibody or antigen-binding fragment thereof that cross-competes with the antibody or antigen-binding fragment thereof of any one of claims 42-79.

81. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 19-21, respectively, and wherein the VH, when paired with a light chain variable region (VL) comprising the amino acid sequence set forth in SEQ ID NO: 61, binds to MET;
(2) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 67, binds to MET;
(3) an immunoglobulin heavy chain or a fragment thereof comprising a VH comprising CDRs 1, 2, and 3 comprising the amino acid sequences set forth in SEQ ID NOs: 22-24, respectively, and wherein the VH, when paired with a VL comprising the amino acid sequence set forth in SEQ ID NO: 61, binds to MET;
(4) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 68, binds to MET;
(5) an immunoglobulin heavy chain or a fragment thereof comprising a VH comprising CDRs 1, 2, and 3 comprising the amino acid sequences set forth in SEQ ID NOs: 25-27, respectively, and wherein the VH, when paired with a VL comprising the amino acid sequence set forth in SEQ ID NO: 61, binds to MET;
(6) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VII comprising the amino acid sequence set forth in SEQ ID NO: 69, binds to MET;
(7) an immunoglobulin heavy chain or a fragment thereof comprising a VH comprising CDRs 1, 2, and 3 comprising the amino acid sequences set forth in SEQ ID NOs: 28-30, respectively, and wherein the VH, when paired with a VL comprising the amino acid sequence set forth in SEQ ID NO: 61, binds to MET;
(8) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 70, binds to MET;
(9) an immunoglobulin heavy chain or a fragment thereof comprising a VH comprising CDRs 1, 2, and 3 comprising the amino acid sequences set forth in SEQ ID NOs: 49-51, respectively, and wherein the VH, when paired with a VL comprising the amino acid sequence set forth in SEQ ID NO: 61, binds to MET;
(10) an immunoglobulin heavy chain or a fragment thereof comprising a VH comprising CDRs 1, 2, and 3 comprising the amino acid sequences set forth in SEQ ID NOs: 52-54, respectively, and wherein the VH, when paired with a VL comprising the amino acid sequence set forth in SEQ ID NO: 61, binds to MET;
(11) an immunoglobulin heavy chain or a fragment thereof comprising a VH comprising CDRs 1, 2, and 3 comprising the amino acid sequences set forth in SEQ ID NOs: 55-57, respectively, and wherein the VH, when paired with a VL comprising the amino acid sequence set forth in SEQ ID NO: 61, binds to MET;
(12) an immunoglobulin heavy chain or a fragment thereof comprising a VH comprising CDRs 1, 2, and 3 comprising the amino acid sequences set forth in SEQ ID NOs: 58-60, respectively, and wherein the VH, when paired with a VL comprising the amino acid sequence set forth in SEQ ID NO: 61, binds to MET;
(13) an immunoglobulin heavy chain or a fragment thereof comprising a VH comprising CDRs 1, 2, and 3 comprising the amino acid sequences set forth in SEQ ID NOs: 112-114, respectively, and wherein the VH, when paired with a VL comprising the amino acid sequence set forth in SEQ ID NO: 61, binds to MET;
(14) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 148, binds to MET;
(15) an immunoglobulin heavy chain or a fragment thereof comprising a VH comprising CDRs 1, 2, and 3 comprising the amino acid sequences set forth in SEQ ID NOs: 115-117, respectively, and wherein the VH, when paired with a VL comprising the amino acid sequence set forth in SEQ ID NO: 61, binds to MET;
(16) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 149, binds to MET;
(17) an immunoglobulin heavy chain or a fragment thereof comprising a VH comprising CDRs 1, 2, and 3 comprising the amino acid sequences set forth in SEQ ID NOs: 118-120, respectively, and wherein the VH, when paired with a VL comprising the amino acid sequence set forth in SEQ ID NO: 61, binds to MET;
(18) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 150, binds to MET;
(19) an immunoglobulin heavy chain or a fragment thereof comprising a VH comprising CDRs 1, 2, and 3 comprising the amino acid sequences set forth in SEQ ID NOs: 121-123, respectively, and wherein the VH, when paired with a VL comprising the amino acid sequence set forth in SEQ ID NO: 61, binds to MET;
(20) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 151, binds to MET;
(21) an immunoglobulin heavy chain or a fragment thereof comprising a VH comprising CDRs 1, 2, and 3 comprising the amino acid sequences set forth in SEQ ID NOs: 124-126, respectively, and wherein the VH, when paired with a VL comprising the amino acid sequence set forth in SEQ ID NO: 61, binds to MET;
(22) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 152, binds to MET;
(23) an immunoglobulin heavy chain or a fragment thereof comprising a VH comprising CDRs 1, 2, and 3 comprising the amino acid sequences set forth in SEQ ID NOs: 127-129, respectively, and wherein the VH, when paired with a VL comprising the amino acid sequence set forth in SEQ ID NO: 61, binds to MET;
(24) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 153, binds to MET;
(25) an immunoglobulin heavy chain or a fragment thereof comprising a VH comprising CDRs 1, 2, and 3 comprising the amino acid sequences set forth in SEQ ID NOs: 130-132, respectively, and wherein the VH, when paired with a VL comprising the amino acid sequence set forth in SEQ ID NO: 61, binds to MET;
(26) an immunoglobulin heavy chain or a fragment thereof comprising a VH comprising CDRs 1, 2, and 3 comprising the amino acid sequences set forth in SEQ ID NOs: 133-135, respectively, and wherein the VH, when paired with a VL comprising the amino acid sequence set forth in SEQ ID NO: 61, binds to MET;
(27) an immunoglobulin heavy chain or a fragment thereof comprising a VH comprising CDRs 1, 2, and 3 comprising the amino acid sequences set forth in SEQ ID NOs: 136-138, respectively, and wherein the VH, when paired with a VL comprising the amino acid sequence set forth in SEQ ID NO: 61, binds to MET;
(28) an immunoglobulin heavy chain or a fragment thereof comprising a VH comprising CDRs 1, 2, and 3 comprising the amino acid sequences set forth in SEQ ID NOs: 139-141, respectively, and wherein the VH, when paired with a VL comprising the amino acid sequence set forth in SEQ ID NO: 61, binds to MET;
(29) an immunoglobulin heavy chain or a fragment thereof comprising a VH comprising CDRs 1, 2, and 3 comprising the amino acid sequences set forth in SEQ ID NOs: 142-144, respectively, and wherein the VH, when paired with a VL comprising the amino acid sequence set forth in SEQ ID NO: 61, binds to MET; and
(30) an immunoglobulin heavy chain or a fragment thereof comprising a VH comprising CDRs 1, 2, and 3 comprising the amino acid sequences set forth in SEQ ID NOs: 145-147, respectively, and wherein the VH, when paired with a VL comprising the amino acid sequence set forth in SEQ ID NO: 61, binds to MET.

82. The nucleic acid of claim 81, wherein the VH when paired with a VL specifically binds to human, monkey or dog MET.

83. The nucleic acid of claim 81 or 82, wherein the immunoglobulin heavy chain or the fragment thereof is a human or humanized immunoglobulin heavy chain or a fragment thereof.

84. The nucleic acid of any one of claims 81-83, wherein the nucleic acid encodes a single-chain variable fragment (scFv).

85. The nucleic acid of any one of claims 81-84, wherein the nucleic acid is cDNA.

86. An antigen-binding protein construct, comprising: a first antigen-binding domain that specifically binds to EGFR; and a second antigen-binding domain that specially binds to MET.

87. The antigen-binding protein construct of claim 86, wherein the first antigen-binding domain comprises a first heavy chain variable region (VH1) and a first light chain variable region (VL1); and the second antigen-binding domain comprises a second heavy chain variable region (VH2) and a second light chain variable region (VL2).

88. The antigen-binding protein construct of claim 87, wherein
the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively;
(7) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively;
(8) the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively;
(9) the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively;
(10) the selected VH1 CDRs 1, 2, 3 amino acid sequences are set forth in SEQ ID NOs: 46-48, respectively, and the selected VL1 CDRs 1, 2, 3 amino acid sequences are set forth in SEQ ID NOs: 1-3, respectively;
(11) the selected VH1 CDRs 1, 2, 3 amino acid sequences are set forth in SEQ ID NOs: 82-84, respectively, and the selected VL1 CDRs 1, 2, 3 amino acid sequences are set forth in SEQ ID NOs: 1-3, respectively;
(12) the selected VH1 CDRs 1, 2, 3 amino acid sequences are set forth in SEQ ID NOs: 85-87, respectively, and the selected VL1 CDRs 1, 2, 3 amino acid sequences are set forth in SEQ ID NOs: 1-3, respectively;
(13) the selected VH1 CDRs 1, 2, 3 amino acid sequences are set forth in SEQ ID NOs: 88-90, respectively, and the selected VL1 CDRs 1, 2, 3 amino acid sequences are set forth in SEQ ID NOs: 1-3, respectively;
(14) the selected VH1 CDRs 1, 2, 3 amino acid sequences are set forth in SEQ ID NOs: 91-93, respectively, and the selected VL1 CDRs 1, 2, 3 amino acid sequences are set forth in SEQ ID NOs: 1-3, respectively;
(15) the selected VH1 CDRs 1, 2, 3 amino acid sequences are set forth in SEQ ID NOs: 94-96, respectively, and the selected VL1 CDRs 1, 2, 3 amino acid sequences are set forth in SEQ ID NOs: 1-3, respectively;
(16) the selected VH1 CDRs 1, 2, 3 amino acid sequences are set forth in SEQ ID NOs: 97-99, respectively, and the selected VL1 CDRs 1, 2, 3 amino acid sequences are set forth in SEQ ID NOs: 1-3, respectively;
(17) the selected VH1 CDRs 1, 2, 3 amino acid sequences are set forth in SEQ ID NOs: 100-102, respectively, and the selected VL1 CDRs 1, 2, 3 amino acid sequences are set forth in SEQ ID NOs: 1-3, respectively; and
(18) the selected VH1 CDRs 1, 2, 3 amino acid sequences are set forth in SEQ ID NOs: 103-105, respectively, and the selected VL1 CDRs 1, 2, 3 amino acid sequences are set forth in SEQ ID NOs: 1-3, respectively.

89. The antigen-binding protein construct of claim 87 or 88, wherein
the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH12 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(2) the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(3) the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(4) the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(5) the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(6) the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(7) the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(8) the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(9) the selected VH2 CDRs 1, 2, 3 amino acid sequences are set forth in SEQ ID NOs: 112-114, respectively, and the selected VL2 CDRs 1, 2, 3 amino acid sequences are set forth in SEQ ID NOs: 1-3, respectively;
(10) the selected VH2 CDRs 1, 2, 3 amino acid sequences are set forth in SEQ ID NOs: 115-117, respectively, and the selected VL2 CDRs 1, 2, 3 amino acid sequences are set forth in SEQ ID NOs: 1-3, respectively;
(11) the selected VH2 CDRs 1, 2, 3 amino acid sequences are set forth in SEQ ID NOs: 118-120, respectively, and the selected VL2 CDRs 1, 2, 3 amino acid sequences are set forth in SEQ ID NOs: 1-3, respectively;
(12) the selected VH2 CDRs 1, 2, 3 amino acid sequences are set forth in SEQ ID NOs: 121-123, respectively, and the selected VL2 CDRs 1, 2, 3 amino acid sequences are set forth in SEQ ID NOs: 1-3, respectively;
(13) the selected VH2 CDRs 1, 2, 3 amino acid sequences are set forth in SEQ ID NOs: 124-126, respectively, and the selected VL2 CDRs 1, 2, 3 amino acid sequences are set forth in SEQ ID NOs: 1-3, respectively;
(14) the selected VH2 CDRs 1, 2, 3 amino acid sequences are set forth in SEQ ID NOs: 127-129, respectively, and the selected VL2 CDRs 1, 2, 3 amino acid sequences are set forth in SEQ ID NOs: 1-3, respectively;
(15) the selected VH2 CDRs 1, 2, 3 amino acid sequences are set forth in SEQ ID NOs: 130-132, respectively, and the selected VL2 CDRs 1, 2, 3 amino acid sequences are set forth in SEQ ID NOs: 1-3, respectively;
(16) the selected VH2 CDRs 1, 2, 3 amino acid sequences are set forth in SEQ ID NOs: 133-135, respectively, and the selected VL2 CDRs 1, 2, 3 amino acid sequences are set forth in SEQ ID NOs: 1-3, respectively;
(17) the selected VH2 CDRs 1, 2, 3 amino acid sequences are set forth in SEQ ID NOs: 136-138, respectively, and the selected VL2 CDRs 1, 2, 3 amino acid sequences are set forth in SEQ ID NOs: 1-3, respectively;
(18) the selected VH2 CDRs 1, 2, 3 amino acid sequences are set forth in SEQ ID NOs: 139-141, respectively, and the selected VL2 CDRs 1, 2, 3 amino acid sequences are set forth in SEQ ID NOs: 1-3, respectively;
(19) the selected VH2 CDRs 1, 2, 3 amino acid sequences are set forth in SEQ ID NOs: 142-144, respectively, and the selected VL2 CDRs 1, 2, 3 amino acid sequences are set forth in SEQ ID NOs: 1-3, respectively; and
(20) the selected VH2 CDRs 1, 2, 3 amino acid sequences are set forth in SEQ ID NOs: 145-147, respectively, and the selected VL2 CDRs 1, 2, 3 amino acid sequences are set forth in SEQ ID NOs: 1-3, respectively.

90. The antigen-binding protein construct of claim 89, wherein
(1) the selected VH1 CDRs 1, 2,3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(3) the selected VH1 CDRs 1, 2,3 amino acid sequences are set forth in SEQ ID NOs: 4-6, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(7) the selected VH1 CDRs 1,2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(8) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(9) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(10) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(11) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(12)(15) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(13) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(14) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(15) the selected VIII CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(16) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(17) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(18) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(19) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(20) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(21) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 31-33, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(22) the selected VH1 CDRs 1, 2, 3 amino acid sequences are set forth in SEQ ID NOs: 37-39, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(23) the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(24) the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(25) the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(26) the selected VH1 CDRs 1, 2, 3 amino acid sequences are set forth in SEQ ID NOs: 40-42, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(27) the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(28) the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(29) the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(30) the selected VH1 CDRs 1, 2, 3 amino acid sequences are set forth in SEQ ID NOs: 43-45, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(31) the selected VH1 CDRs 1, 2, 3 amino acid sequences are set forth in SEQ ID NOs: 46-48, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(32) the selected VH1 CDRs 1, 2, 3 amino acid sequences are set forth in SEQ ID NOs: 46-48, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(33) the selected VH1 CDRs 1, 2, 3 amino acid sequences are set forth in SEQ ID NOs: 46-48, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(34) the selected VH1 CDRs 1, 2, 3 amino acid sequences are set forth in SEQ ID NOs: 46-48, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(35) the selected VH1 CDRs 1, 2, 3 amino acid sequences are set forth in SEQ ID NOs: 82-84, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(36) the selected VH1 CDRs 1, 2, 3 amino acid sequences are set forth in SEQ ID NOs: 82-84, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(37) the selected VH1 CDRs 1, 2, 3 amino acid sequences are set forth in SEQ ID NOs: 82-84, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(38) the selected VH1 CDRs 1, 2, 3 amino acid sequences are set forth in SEQ ID NOs: 82-84, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(39) the selected VH1 CDRs 1, 2, 3 amino acid sequences are set forth in SEQ ID NOs: 85-87, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(40) the selected VH1 CDRs 1, 2, 3 amino acid sequences are set forth in SEQ ID NOs: 85-87, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(41) the selected VH1 CDRs 1, 2, 3 amino acid sequences are set forth in SEQ ID NOs: 85-87, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(42) the selected VH1 CDRs 1, 2, 3 amino acid sequences are set forth in SEQ ID NOs: 85-87, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(43) the selected VH1 CDRs 1, 2, 3 amino acid sequences are set forth in SEQ ID NOs: 88-90, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(44) the selected VH1 CDRs 1, 2, 3 amino acid sequences are set forth in SEQ ID NOs: 88-90, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(45) the selected VH1 CDRs 1, 2, 3 amino acid sequences are set forth in SEQ ID NOs: 88-90, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(46) the selected VH1 CDRs 1, 2, 3 amino acid sequences are set forth in SEQ ID NOs: 88-90, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(47) the selected VH1 CDRs 1, 2, 3 amino acid sequences are set forth in SEQ ID NOs: 91-93, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 19-21, respectively, and the selected VL2 CDRs 1, 2, 3 amino acid sequences are set forth in SEQ ID NOs: 1-3, respectively;
(48) the selected VH1 CDRs 1, 2, 3 amino acid sequences are set forth in SEQ ID NOs: 91-93, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 22-24, respectively, and the selected VL2 CDRs 1, 2, 3 amino acid sequences are set forth in SEQ ID NOs: 1-3, respectively;
(49) the selected VH1 CDRs 1, 2, 3 amino acid sequences are set forth in SEQ ID NOs: 91-93, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 25-27, respectively, and the selected VL2 CDRs 1, 2, 3 amino acid sequences are set forth in SEQ ID NOs: 1-3, respectively;
(50) the selected VH1 CDRs 1, 2, 3 amino acid sequences are set forth in SEQ ID NOs: 91-93, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 28-30, respectively, and the selected VL2 CDRs 1, 2, 3 amino acid sequences are set forth in SEQ ID NOs: 1-3, respectively;
(51) the selected VH1 CDRs 1, 2, 3 amino acid sequences are set forth in SEQ ID NOs: 94-96, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(52) the selected VH1 CDRs 1, 2, 3 amino acid sequences are set forth in SEQ ID NOs: 94-96, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(53) the selected VH1 CDRs 1, 2, 3 amino acid sequences are set forth in SEQ ID NOs: 94-96, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(54) the selected VH1 CDRs 1, 2, 3 amino acid sequences are set forth in SEQ ID NOs: 94-96, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(55) the selected VH1 CDRs 1, 2, 3 amino acid sequences are set forth in SEQ ID NOs: 97-99, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(56) the selected VH1 CDRs 1, 2, 3 amino acid sequences are set forth in SEQ ID NOs: 97-99, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(57) the selected VH1 CDRs 1, 2, 3 amino acid sequences are set forth in SEQ ID NOs: 97-99, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(58) the selected VH1 CDRs 1, 2, 3 amino acid sequences are set forth in SEQ ID NOs: 97-99, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(59)(65) the selected VH1 CDRs 1, 2, 3 amino acid sequences are set forth in SEQ ID NOs: 100-102, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(60) the selected VH1 CDRs 1, 2, 3 amino acid sequences are set forth in SEQ ID NOs: 100-102, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(61) the selected VH1 CDRs 1, 2, 3 amino acid sequences are set forth in SEQ ID NOs: 100-102, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively;
(62) the selected VH1 CDRs 1, 2, 3 amino acid sequences are set forth in SEQ ID NOs: 100-102, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively;
(63) the selected VH1 CDRs 1, 2, 3 amino acid sequences are set forth in SEQ ID NOs: 103-105, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(64) the selected VH1 CDRs 1, 2, 3 amino acid sequences are set forth in SEQ ID NOs: 103-105, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 1-3, respectively;
(65) the selected VH1 CDRs 1, 2, 3 amino acid sequences are set forth in SEQ ID NOs: 103-105, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 1-3, respectively; and
(66) the selected VH1 CDRs 1, 2, 3 amino acid sequences are set forth in SEQ ID NOs: 103-105, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 1-3, respectively.

91. The antigen-binding protein construct of any one of claims 87-90, wherein the first heavy chain variable region comprises a sequence that is at least 80%, 85%, 90%, or 95% identical to SEQ ID NO: 62, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 68, and the second light chain variable region comprises a sequence that is at least 80%, 85%, 90%, or 95% identical to SEQ ID NO: 61.

92. The antigen-binding protein construct of any one of claims 87-90, wherein the first heavy chain variable region comprises a sequence that is at least 80%, 85%, 90%, or 95% identical to SEQ ID NO: 62, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 69, and the second light chain variable region comprises a sequence that is at least 80%, 85%, 90%, or 95% identical to SEQ ID NO: 61.

93. The antigen-binding protein construct of any one of claims 87-90, wherein the first heavy chain variable region comprises a sequence that is at least 80%, 85%, 90%, or 95% identical to SEQ ID NO: 62, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 70, and the second light chain variable region comprises a sequence that is at least 80%, 85%, 90%, or 95% identical to SEQ ID NO: 61.

94. The antigen-binding protein construct of any one of claims 87-90, wherein the first heavy chain variable region comprises a sequence that is at least 80%, 85%, 90%, or 95% identical to SEQ ID NO: 63, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 69, and the second light chain variable region comprises a sequence that is at least 80%, 85%, 90%, or 95% identical to SEQ ID NO: 61.

95. The antigen-binding protein construct of any one of claims 87-90, wherein the first heavy chain variable region comprises a sequence that is at least 80%, 85%, 90%, or 95% identical to SEQ ID NO: 63, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 70, and the second light chain variable region comprises a sequence that is at least 80%, 85%, 90%, or 95% identical to SEQ ID NO: 61.

96. The antigen-binding protein construct of any one of claims 87-90, wherein the first heavy chain variable region comprises a sequence that is at least 80%, 85%, 90%, or 95% identical to SEQ ID NO: 64, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 67, and the second light chain variable region comprises a sequence that is at least 80%, 85%, 90%, or 95% identical to SEQ ID NO: 61.

97. The antigen-binding protein construct of any one of claims 87-90, wherein the first heavy chain variable region comprises a sequence that is at least 80%, 85%, 90%, or 95% identical to SEQ ID NO: 64, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 68, and the second light chain variable region comprises a sequence that is at least 80%, 85%, 90%, or 95% identical to SEQ ID NO: 61.

98. The antigen-binding protein construct of any one of claims 97-90, wherein the first heavy chain variable region comprises a sequence that is at least 80%, 85%, 90%, or 95% identical to SEQ ID NO: 64, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 69, and the second light chain variable region comprises a sequence that is at least 80%, 85%, 90%, or 95% identical to SEQ ID NO: 61.

99. The antigen-binding protein construct of any one of claims 87-90, wherein the first heavy chain variable region comprises a sequence that is at least 80%, 85%, 90%, or 95% identical to SEQ ID NO: 64, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 70, and the second light chain variable region comprises a sequence that is at least 80%, 85%, 90%, or 95% identical to SEQ ID NO: 61.

100. The antigen-binding protein construct of any one of claims 87-90, wherein the first heavy chain variable region comprises a sequence that is at least 80%, 85%, 90%, or 95% identical to SEQ ID NO: 65, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 67, and the second light chain variable region comprises a sequence that is at least 80%, 85%, 90%, or 95% identical to SEQ ID NO: 61.

101. The antigen-binding protein construct of any one of claims 87-90, wherein the first heavy chain variable region comprises a sequence that is at least 80%, 85%, 90%, or 95% identical to SEQ ID NO: 65, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 68, and the second light chain variable region comprises a sequence that is at least 80%, 85%, 90%, or 95% identical to SEQ ID NO: 61.

102. The antigen-binding protein construct of any one of claims 87-90, wherein the first heavy chain variable region comprises a sequence that is at least 80%, 85%, 90%, or 95% identical to SEQ ID NO: 65, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 69, and the second light chain variable region comprises a sequence that is at least 80%, 85%, 90%, or 95% identical to SEQ ID NO: 61.

103. The antigen-binding protein construct of any one of claims 87-90, wherein the first heavy chain variable region comprises a sequence that is at least 80%, 85%, 90%, or 95% identical to SEQ ID NO: 65, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 70, and the second light chain variable region comprises a sequence that is at least 80%, 85%, 90%, or 95% identical to SEQ ID NO: 61.

104. The antigen-binding protein construct of any one of claims 87-90, wherein the first heavy chain variable region comprises a sequence that is at least 80%, 85%, 90%, or 95% identical to SEQ ID NO: 66, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 67, and the second light chain variable region comprises a sequence that is at least 80%, 85%, 90%, or 95% identical to SEQ ID NO: 61.

105. The antigen-binding protein construct of any one of claims 87-90, wherein the first heavy chain variable region comprises a sequence that is at least 80%, 85%, 90%, or 95% identical to SEQ ID NO: 66, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 68, and the second light chain variable region comprises a sequence that is at least 80%, 85%, 90%, or 95% identical to SEQ ID NO: 61.

106. The antigen-binding protein construct of any one of claims 87-90, wherein the first heavy chain variable region comprises a sequence that is at least 80%, 85%, 90%, or 95% identical to SEQ ID NO: 66, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 69, and the second light chain variable region comprises a sequence that is at least 80%, 85%, 90%, or 95% identical to SEQ ID NO: 61.

107. The antigen-binding protein construct of any one of claims 87-90, wherein the first heavy chain variable region comprises a sequence that is at least 80%, 85%, 90%, or 95% identical to SEQ ID NO: 66, the first light chain variable region comprises a sequence that is at least 80%, 85%, 90%, or 95% identical to SEQ ID NO: 61, the second heavy chain variable region comprises a sequence that is at least 80%, 85%, 90%, or 95% identical to SEQ ID NO: 70, and the second light chain variable region comprises a sequence that is at least 80%, 85%, 90%, or 95% identical to SEQ ID NO: 61.

108. The antigen-binding protein construct of any one of claims 86-10⁷, wherein the antigen-binding protein construct is a multi-specific antibody (e.g., a bispecific antibody).

109. The antigen-binding protein construct of any one of claims 86-108, wherein the first antigen-binding domain is a single-chain variable fragment (scFv); and/or the second antigen-binding domain is a scFv.

110. The antigen-binding protein construct of any one of claims 87-109, wherein the first light chain variable region and the second light chain variable region are identical.

111. A vector comprising one or more of the nucleic acids of anyone of claims 37-41 and 81-85, a nucleic acid encoding the antibody or antigen-binding fragment thereof of any one of claims 1-36 and 42-80, or a nucleic acid encoding the antigen-binding protein construct of any one of claims 86-110.

112. A cell comprising the vector of claim 111.

113. The cell of claim 112, wherein the cell is a CHO cell.

114. A cell comprising one or more of the nucleic acids of any one of claims 37-41 and 81-85, a nucleic acid encoding the antibody or antigen-binding fragment thereof of any one of claims 1-36 and 42-80, or a nucleic acid encoding the antigen-binding protein construct of any one of claims 86-110.

115. A method of producing an antibody or antigen-binding fragment thereof, or an antigen-binding protein construct, the method comprising
(a) culturing the cell of any one of claims 112-114 under conditions sufficient for the cell to produce the antibody or the antigen-binding fragment thereof, or the antigen-binding protein construct; and
(b) collecting the antibody or the antigen-binding fragment thereof, or the antigen-binding protein construct produced by the cell.

116. An antibody-drug conjugate (ADC) comprising a therapeutic agent covalently bound to:
(a) the antibody or antigen-binding fragment thereof of any one of claims 1-36 and 42-80; or
(b) the antigen-binding protein construct of any one of claims 86-110.

117. The antibody-drug conjugate of claim 116, wherein the therapeutic agent is a cytotoxic or cytostatic agent.

118. The antibody-drug conjugate of claim 116 or 117, wherein the therapeutic agent is MMAE or MMAF.

119. The antibody-drug conjugate of claim 116, wherein the therapeutic agent is selected from

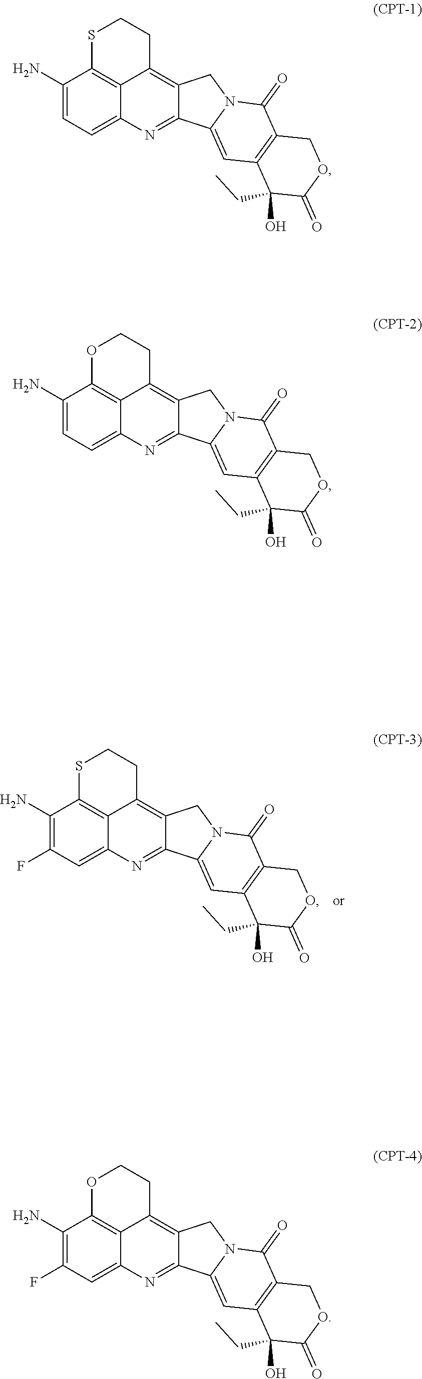 [SMILES: CC[C@@]1(O)C(=O)OCc2c1cc1n(c2=O)Cc2c-1nc1ccc(N)c3c1c2CCS3 ; CC[C@@]1(O)C(=O)OCc2c1cc1n(c2=O)Cc2c-1nc1ccc(N)c3c1c2CCO3 ; CC[C@@]1(O)C(=O)OCc2c1cc1n(c2=O)Cc2c-1nc1cc(F)c(N)c3c1c2CCS3 ; CC[C@@]1(O)C(=O)OCc2c1cc1n(c2=O)Cc2c-1nc1cc(F)c(N)c3c1c2CCO3]

120. The antibody-drug conjugate of claim 116 or 119, wherein the therapeutic agent is linked to the antibody or antigen-binding fragment thereof, or the antigen-binding protein construct via a linker.

121. The antibody-drug conjugate of any one of claims 116, 119, and 120, wherein the linker has a structure of:

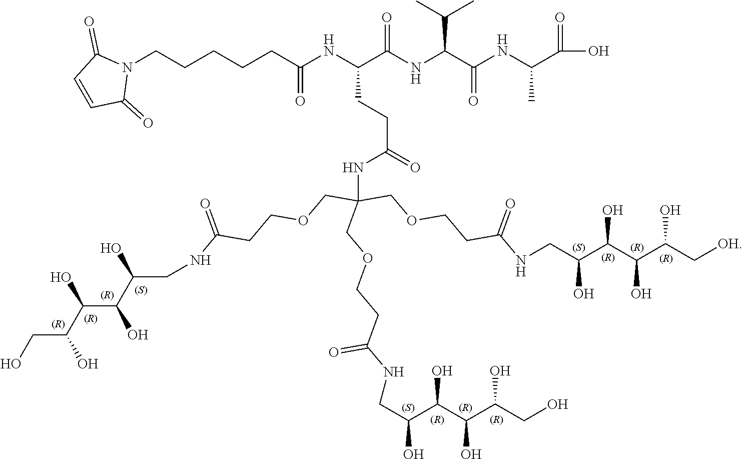

122. The antibody-drug conjugate of any one of claims 116 and 119-121, wherein the antibody-drug conjugate has a structure of: 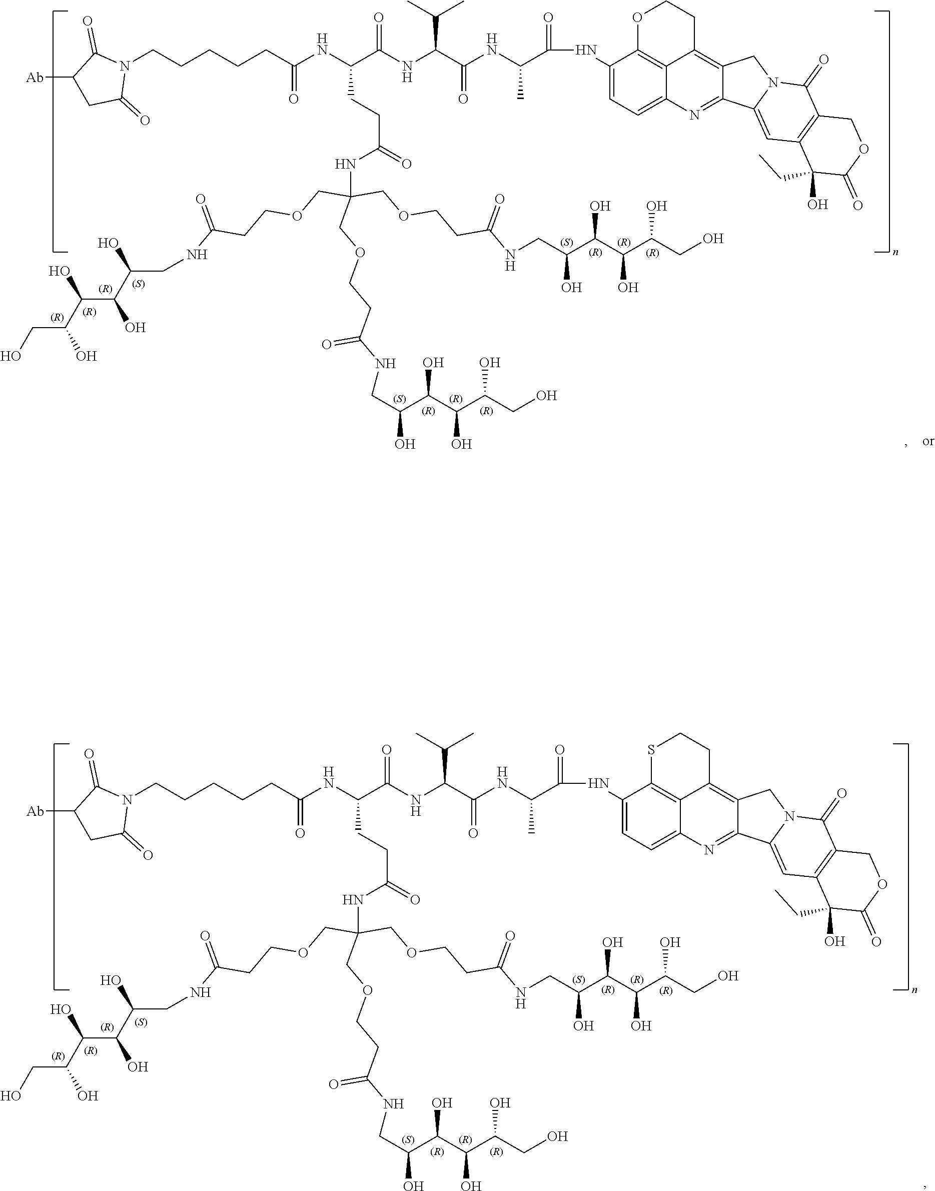
wherein n=1,2,3,4,5,6,7, or 8; wherein “Ab” represents the antibody or antigen-binding fragment thereof, or the antigen-binding protein construct.

123. A method of treating a subject having cancer, the method comprising administering a therapeutically effective amount of a composition comprising the antibody or antigen-binding fragment thereof of any one of claims 1-36 and 42-80, the antigen-binding protein construct of any one of claims 86-110, or the antibody-drug conjugate of any one of claims 116-122, to the subject.

124. The method of claim 123, wherein the subject has a cancer expressing EGFR and/or MET.

125. The method of claim 123, wherein the cancer is a solid tumor, lung cancer (e.g., non-small cell lung cancer, lung adenocarcinoma, or lung carcinoma), gastric cancer (e.g., gastric carcinoma), skin cancer (e.g., skin carcinoma), ampullary cancer (e.g., ampullary carcinoma) colorectal cancer, breast cancer, head and neck cancer, ovarian cancer, prostate cancer, thyroid cancer, pancreatic cancer, CNS cancer, liver cancer, nasopharynx cancer, or brain cancer.

126. The method of any one of claims 123-125, wherein the subject is a human.

127. The method of any one of claims 123-126, wherein the method further comprises administering an anti-PD1 antibody to the subject.

128. The method of any one of claims 123-127, wherein the method further comprises administering a chemotherapy to the subject.

129. A method of decreasing the rate of tumor growth, the method comprising contacting a tumor cell with an effective amount of a composition comprising the antibody or antigen-binding fragment thereof of any one of claims 1-36 and 42-80, the antigen-binding protein construct of any one of claims 86-110, or the antibody-drug conjugate of any one of claims 116-122.

130. A method of killing a tumor cell, the method comprising contacting a tumor cell with an effective amount of a composition comprising the antibody or antigen-binding fragment thereof of any one of claims 1-36 and 42-80, the antigen-binding protein construct of any one of claims 86-110, or the antibody-drug conjugate of any one of claims 116-122.

131. A pharmaceutical composition comprising a pharmaceutically acceptable carrier and
(a) the antibody or antigen-binding fragment thereof of any one of claims 1-36 and 42-80,
(b) the antigen-binding protein construct of any one of claims 86-110, and/or
(c) the antibody-drug conjugate of any one of claims 116-122.

132. An antibody-drug conjugate (ADC) comprising a therapeutic agent covalently bound to a bispecific antibody or antigen-binding fragment thereof comprising: a first antigen-binding domain that specifically binds to EGFR; and a second antigen-binding domain that specially binds to MET.

133. The antibody-drug conjugate (ADC) of claim 132, wherein the drug-to-antibody ratio (DAR) is about 4 or 8.


SEQUENCE LISTING (155 sequences)

SEQ ID NO: 1 - protein, 11 aa, synthetic construct
RASQSVSSYLA

SEQ ID NO: 2 - protein, 7 aa, synthetic construct
DASNRAT

SEQ ID NO: 3 - protein, 9 aa, synthetic construct
QQRSNWPPT

SEQ ID NO: 4 - protein, 5 aa, synthetic construct
SYEMN

SEQ ID NO: 5 - protein, 17 aa, synthetic construct
YISSAGSTIYYADSVKG

SEQ ID NO: 6 - protein, 13 aa, synthetic construct
VWYSSGWSGCFAY

SEQ ID NO: 7 - protein, 7 aa, synthetic construct
SGDYYWS

SEQ ID NO: 8 - protein, 16 aa, synthetic construct
YIYYSGSTYYNPSLKS

SEQ ID NO: 9 - protein, 10 aa, synthetic construct
ERVYSSSLDY

SEQ ID NO: 10 - protein, 7 aa, synthetic construct
SGDYYWS

SEQ ID NO: 11 - protein, 16 aa, synthetic construct
YIYYSGSTYYNPSLKS

SEQ ID NO: 12 - protein, 12 aa, synthetic construct
NNDFGDYLYFDN

SEQ ID NO: 13 - protein, 5 aa, synthetic construct
SYEMN

SEQ ID NO: 14 - protein, 17 aa, synthetic construct
YISSSGSTIYYADSVKG

SEQ ID NO: 15 - protein, 13 aa, synthetic construct
VWYSSGWSGCFAY

SEQ ID NO: 16 - protein, 5 aa, synthetic construct
SYEMN

SEQ ID NO: 17 - protein, 17 aa, synthetic construct
YISSSGSTIYYADSVKG

SEQ ID NO: 18 - protein, 13 aa, synthetic construct
VWYSSGWSGCFAY

SEQ ID NO: 19 - protein, 5 aa, synthetic construct
SNYWS

SEQ ID NO: 20 - protein, 16 aa, synthetic construct
YIYHSGSTNYNPSLKS

SEQ ID NO: 21 - protein, 14 aa, synthetic construct
GPVSIFGVVKYIDL

SEQ ID NO: 22 - protein, 5 aa, synthetic construct
ICGIH

SEQ ID NO: 23 - protein, 17 aa, synthetic construct
TISHDANDKYYADSVKG

SEQ ID NO: 24 - protein, 12 aa, synthetic construct
DGNRGYDGYFDY

SEQ ID NO: 25 - protein, 5 aa, synthetic construct
GYYWS

SEQ ID NO: 26 - protein, 16 aa, synthetic construct
EINHSGSTNYNPSLKS

SEQ ID NO: 27 - protein, 13 aa, synthetic construct
GRGIAAAGTYFDY

SEQ ID NO: 28 - protein, 5 aa, synthetic construct
NYGMD

SEQ ID NO: 29 - protein, 17 aa, synthetic construct
VIWNDGSKKYYGDFVKG

SEQ ID NO: 30 - protein, 13 aa, synthetic construct
DLEGGQLADAFDV

SEQ ID NO: 31 - protein, 621 aa, Homo sapiens [sequence omitted]

SEQ ID NO: 32 - protein, 621 aa, Macaca fascicularis
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSEFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDTLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSSQKTKIISNRGENSCKATGQVCHALCSPEGCWGPEPRDCVSCQNVSRGRECVDKCNILEGEPREFVENSECIQCHPECLPQVMNITCTGRGPDNCIQCAHYIDGPHCVKTCPAGVMGENNTLVWKYADAGHVCHLCHPNCTYGCTGPGLEGCARNGPKIPS

SEQ ID NO: 33 - protein, 1400 aa, Macaca fascicularis
MKSKSKGLAECLAHDKPLTMKAPAVLVPGILVLLFTLVQRSNGECKEALAKSEMNVNMKYQLPNFTAETAIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPNQCPDCVVSALGAKVLSSVKDRFINFFVGNTINSSYFPHHPLHSISVRRLKETKDGFMFLTDQSYIDVLPEFRDSYPIKYIHAFESNNFIYFLTVQRETLNAQTFHTRIIRFCSLNSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRAEFTTALQRVDLFMGQFSEVLLTSISTFVKGDLTIANLGTSEGRFMQVVVSRSGPSTPHVNFLLDSHPVSPEVIVEHPLNQNGYTLVVTGKKITKIPLNGLGCRHFQSCSQCLSAPPFVQCGWCHDKCVRSEECPSGTWTQQICLPAIYKVFPTSAPLEGGTRLTICGWDFGFRRNNKFDLKKTRVLLGNESCTLTLSESTMNTLKCTVGPAMNKHFNMSIIISNGHGTTQYSTFSYVDPIITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLHSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISIALLLLLGLFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDT

SEQ ID NO: 34 - protein, 7 aa, synthetic construct
GFTFSSY

SEQ ID NO: 35 - protein, 6 aa, synthetic construct
SSAGST

SEQ ID NO: 36 - protein, 13 aa, synthetic construct
VWYSSGWSGCFAY

SEQ ID NO: 37 - protein, 9 aa, synthetic construct
GGSINSGDY

SEQ ID NO: 38 - protein, 5 aa, synthetic construct
YYSGS

SEQ ID NO: 39 - protein, 10 aa, synthetic construct
ERVYSSSLDY

SEQ ID NO: 40 - protein, 9 aa, synthetic construct
GGSINSGDY

SEQ ID NO: 41 - protein, 5 aa, synthetic construct
YYSGS

SEQ ID NO: 42 - protein, 12 aa, synthetic construct
NNDFGDYLYFDN

SEQ ID NO: 43 - protein, 7 aa, synthetic construct
GFIFSSY

SEQ ID NO: 44 - protein, 6 aa, synthetic construct
SSSGST

SEQ ID NO: 45 - protein, 13 aa, synthetic construct
VWYSSGWSGCFAY

SEQ ID NO: 46 - protein, 7 aa, synthetic construct
GFIFSSY

SEQ ID NO: 47 - protein, 6 aa, synthetic construct
SSSGST

SEQ ID NO: 48 - protein, 13 aa, synthetic construct
VWYSSGWSGCFAY

SEQ ID NO: 49 - protein, 7 aa, synthetic construct
GGSISSN

SEQ ID NO: 50 - protein, 5 aa, synthetic construct
YHSGS

SEQ ID NO: 51 - protein, 14 aa, synthetic construct
GPVSIFGVVKYIDL

SEQ ID NO: 52 - protein, 7 aa, synthetic construct
GFTFNIC

SEQ ID NO: 53 - protein, 6 aa, synthetic construct
SHDAND

SEQ ID NO: 54 - protein, 12 aa, synthetic construct
DGNRGYDGYFDY

SEQ ID NO: 55 - protein, 7 aa, synthetic construct
GGSFSGY

SEQ ID NO: 56 - protein, 5 aa, synthetic construct
NHSGS

SEQ ID NO: 57 - protein, 13 aa, synthetic construct
GRGIAAAGTYFDY

SEQ ID NO: 58 - protein, 7 aa, synthetic construct
GFTFNNY

SEQ ID NO: 59 - protein, 6 aa, synthetic construct
WNDGSK

SEQ ID NO: 60 - protein, 13 aa, synthetic construct
DLEGGQLADAFDV

SEQ ID NO: 61 - protein, 107 aa, synthetic construct [sequence omitted]

SEQ ID NO: 62 - protein, 122 aa, synthetic construct
EVQLVQSGGGLVQPGGSLRLSCAASGFTFSSYEMNWVRQAPGMGLEWVSYISSAGSTIYYADSVKGRFTISRDNAMNSLYLEMNSLRAEDTAVYYCVRVWYSSGWSGCFAYWGQGSLVTVSS

SEQ ID NO: 63 - protein, 120 aa, synthetic construct
QVQLQQWGPGLVKPSQTLSLTCTVSGGSINSGDYYWSWIRQPPGKGLESIGYIYYSGSTYYNPSLKSRVTISADTSKNQFSLKLTSVTAADTAVYYCARERVYSSSLDYWGQGTLVTVSS

SEQ ID NO: 64 - protein, 122 aa, synthetic construct
EVQLQQWGPGLVKPSQTLSLTCTVSGGSINSGDYYWSWIRQPPGKGLEWIGYIYYSGSTYYNPSLKSRVTISLDTSKNQLSLKLSSVTAADTAVYYCARNNDFGDYLYFDNWGQGTLVTVSS

SEQ ID NO: 65 - protein, 122 aa, synthetic construct
EVQLVESGGGLVQPGGSLRLSCAVSGFIFSSYEMNWVRQAPGKGLEWVSYISSSGSTIYYADSVKGRFTISRDNAMNSLNLQMNSLRDEDTAVYYCVRVWYSSGWSGCFAYWGQGSLVTVSS

SEQ ID NO: 66 - protein, 122 aa, synthetic construct
EVQLVESGGGLVQPGGSLRLSCAASGFIFSSYEMNWVRQAPGKGLEWVSYISSSGSTIYYADSVKGRFTISRDNAKNSLYLQMNSLRADDTAIYYCARVWYSSGWSGCFAYWGQGTLVTVSS

SEQ ID NO: 67 - protein, 122 aa, synthetic construct
EVQLVESGPGLVKPSETLSLTCTVSGGSISSNYWSWIRQPPGKGLEWIGYIYHSGSTNYNPSLKSRVTISVDTSKNQFSLKLSSVTAADTAVYFCARGPVSIFGVVKYIDLWGRGTLVTVSS

SEQ ID NO: 68 - protein, 121 aa, synthetic construct
EVQLVESGGGVVQPGRSLRLSCAASGFTFNICGIHWVRQAPGKGLEWVATISHDANDKYYADSVKGRFTISRDNSKNTLYLQMNSLRAEDTAVYYCARDGNRGYDGYFDYWGQGTLVTVSS

SEQ ID NO: 69 - protein, 121 aa, synthetic construct
QVQLQQWGAGLLKPSETLSLTCVVFGGSFSGYYWSWIRQPPGKGLEWIGEINHSGSTNYNPSLKSRVTVSVDTSKKQFSLNLRSVTAADTAVYYCARGRGIAAAGTYFDYWGQGNLVTVSS

SEQ ID NO: 70 - protein, 122 aa, synthetic construct
QVQLVQSGGGVVQPGRSLRLSCAASGFTFNNYGMDWVRQAPGKGLEWVAVIWNDGSKKYYGDFVKGRFTISRDNSKNTLYLEMNSLRAEDTAVYYCARDLEGGQLADAFDVWGQGTMVTVSS

SEQ ID NO: 71 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 73 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 448 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 218 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 460 aa, synthetic construct
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 209 aa, synthetic construct
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 79 - protein, 449 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 80 - protein, 214 aa, synthetic construct
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 107 aa, synthetic construct [sequence omitted]

SEQ ID NO: 82 - protein, 7 aa, synthetic construct
SGDYYWN

SEQ ID NO: 83 - protein, 16 aa, synthetic construct
YIYYSGSTYYNPSLKS

SEQ ID NO: 84 - protein, 10 aa, synthetic construct
EGVYTIMVDY

SEQ ID NO: 85 - protein, 7 aa, synthetic construct
SGDYYWS

SEQ ID NO: 86 - protein, 16 aa, synthetic construct
YIYYSGSTYYNPSLKS

SEQ ID NO: 87 - protein, 10 aa, synthetic construct
DLVYYYALDV

SEQ ID NO: 88 - protein, 7 aa, synthetic construct
SGDYYWS

SEQ ID NO: 89 - protein, 16 aa, synthetic construct
HIYYSGITYYNPSLKS

SEQ ID NO: 90 - protein, 9 aa, synthetic construct
AGVYHDMDV

SEQ ID NO: 91 - protein, 7 aa, synthetic construct
SGDYYWS

SEQ ID NO: 92 - protein, 16 aa, synthetic construct
YIYYSGSTYYNPSLKS

SEQ ID NO: 93 - protein, 10 aa, synthetic construct
VGVYSTSRDY

SEQ ID NO: 94 - protein, 9 aa, synthetic construct
GGSISSGDY

SEQ ID NO: 95 - protein, 5 aa, synthetic construct
YYSGS

SEQ ID NO: 96 - protein, 10 aa, synthetic construct
EGVYTIMVDY

SEQ ID NO: 97 - protein, 9 aa, synthetic construct
GGSISSGDY

SEQ ID NO: 98 - protein, 5 aa, synthetic construct
YYSGS

SEQ ID NO: 99 - protein, 10 aa, synthetic construct
DLVYYYALDV

SEQ ID NO: 100 - protein, 9 aa, synthetic construct
GGSINSGDY

SEQ ID NO: 101 - protein, 5 aa, synthetic construct
YYSGI

SEQ ID NO: 102 - protein, 9 aa, synthetic construct
AGVYHDMDV

SEQ ID NO: 103 - protein, 9 aa, synthetic construct
GDSISSGDY

SEQ ID NO: 104 - protein, 5 aa, synthetic construct
YYSGS

SEQ ID NO: 105 - protein, 10 aa, synthetic construct
VGVYSTSRDY

SEQ ID NO: 106 - protein, 120 aa, synthetic construct
EVQLQQSGPGLVKPSQTLSLTCTVSGGSISSGDYYWNWIRQPPGKGLEWIGYIYYSGSTYYNPSLKSRVTISVDTSKNQFSLKLSSVTAADTAVYYCAREGVYTIMVDYWGQGTLVTVSS

SEQ ID NO: 107 - protein, 120 aa, synthetic construct
EVQLQESGPGLVKPSQTLSLTCTVSGGSISSGDYYWSWIRQPPGKGLEWIGYIYYSGSTYYNPSLKSRVTISVDTSKNQFSLRLSSVTAADTAVYYCARDLVYYYALDVWGQGTTVTVSS

SEQ ID NO: 108 - protein, 119 aa, synthetic construct
QVQLQESGPGLVKPSQTLSLTCTVSGGSINSGDYYWSWIRQPPGKGLEWIGHIYYSGITYYNPSLKSRVTISADTSKNQFSLKLTSVTVADTAVYYCDRAGVYHDMDVWGQGTTVTVSS

SEQ ID NO: 109 - protein, 120 aa, synthetic construct
EVQLVESGPGLVKPSQTLSLTCTVSGDSISSGDYYWSWIRQPPGKGLEWIGYIYYSGSTYYNPSLKSRVIISLDTSKNQFSLKLISVTAADTAVYYCARVGVYSTSRDYWGQGTLVTVSS

SEQ ID NO: 110 - protein, 449 aa, synthetic construct
QVQLQESGPGLVKPSETLSLTCTVSGFSLSNYDVHWVRQAPGKGLEWLGVIWSGGNTDYNTPFTSRLTISVDTSKNQFSLKLSSVTAADTAVYYCARALDYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 214 aa, synthetic construct
EIVLTQSPDFQSVTPKEKVTITCRASQSIGTNIHWYQQKPDQSPKLLIKYASESISGIPSRFSGSGSGTDFTLTINSLEAEDAATYYCQQNNEWPTSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 6 aa, synthetic construct
KSNWWT

SEQ ID NO: 113 - protein, 16 aa, synthetic construct
EIHHSGSTNYNPSLKS

SEQ ID NO: 114 - protein, 10 aa, synthetic construct
GGGYGYDFDY

SEQ ID NO: 115 - protein, 7 aa, synthetic construct
STGVAMG

SEQ ID NO: 116 - protein, 16 aa, synthetic construct
LIYWDDDKHYSPSLES

SEQ ID NO: 117 - protein, 9 aa, synthetic construct
RRYTGYEDY

SEQ ID NO: 118 - protein, 7 aa, synthetic construct
TTGVSVG

SEQ ID NO: 119 - protein, 16 aa, synthetic construct
LIYWDDDKHYSPSLKS

SEQ ID NO: 120 - protein, 13 aa, synthetic construct
GGYSSGWPGFYDL

SEQ ID NO: 121 - protein, 5 aa, synthetic construct
GHYWS

SEQ ID NO: 122 - protein, 16 aa, synthetic construct
YITYSGSTNYNPSLKS

SEQ ID NO: 123 - protein, 14 aa, synthetic construct
IAVAGRVYYYGVDV

SEQ ID NO: 124 - protein, 7 aa, synthetic construct
TSGVAVA

SEQ ID NO: 125 - protein, 16 aa, synthetic construct
LIYWDDDKWYTPSLKT

SEQ ID NO: 126 - protein, 13 aa, synthetic construct
RGSFNWNYGYFDY

SEQ ID NO: 127 - protein, 5 aa, synthetic construct
GYYWT

SEQ ID NO: 128 - protein, 16 aa, synthetic construct
ESNHGGSTNFNPSLKS

SEQ ID NO: 129 - protein, 13 aa, synthetic construct
GGTIFGLKDAFDI

SEQ ID NO: 130 - protein, 8 aa, synthetic construct
GGSISKSN

SEQ ID NO: 131 - protein, 5 aa, synthetic construct
HHSGS

SEQ ID NO: 132 - protein, 10 aa, synthetic construct
GGGYGYDFDY

SEQ ID NO: 133 - protein, 9 aa, synthetic construct
GFSLSSTGV

SEQ ID NO: 134 - protein, 5 aa, synthetic construct
YWDDD

SEQ ID NO: 135 - protein, 9 aa, synthetic construct
RRYTGYEDY

SEQ ID NO: 136 - protein, 9 aa, synthetic construct
GFSLSTTGV

SEQ ID NO: 137 - protein, 5 aa, synthetic construct
YWDDD

SEQ ID NO: 138 - protein, 13 aa, synthetic construct
GGYSSGWPGFYDL

SEQ ID NO: 139 - protein, 7 aa, synthetic construct
GGSISGH

SEQ ID NO: 140 - protein, 5 aa, synthetic construct
TYSGS

SEQ ID NO: 141 - protein, 14 aa, synthetic construct
IAVAGRVYYYGVDV

SEQ ID NO: 142 - protein, 9 aa, synthetic construct
GFSLYTSGV

SEQ ID NO: 143 - protein, 5 aa, synthetic construct
YWDDD

SEQ ID NO: 144 - protein, 13 aa, synthetic construct
RGSFNWNYGYFDY

SEQ ID NO: 145 - protein, 7 aa, synthetic construct
GGSFSGY

SEQ ID NO: 146 - protein, 5 aa, synthetic construct
NHGGS

SEQ ID NO: 147 - protein, 13 aa, synthetic construct
GGTIFGLKDAFDI

SEQ ID NO: 148 - protein, 119 aa, synthetic construct
QVQLQQSGPGLVKPSGTLSLTCAVSGGSISKSNWWTWVRQPPGKGLEWIGEIHHSGSTNYNPSLKSRVTISVEKTKNQFSLKLSSVTAADTAVYYCARGGGYGYDFDYWGQGTLVTVSS

SEQ ID NO: 149 - protein, 119 aa, synthetic construct
QVQLEQSGPTLVKPTQTLTLTCTFSGFSLSSTGVAMGWIRQPPGKALEWLALIYWDDDKHYSPSLESRLTITKDTFKNQVVLSMTNMDPLDTATYYCTHRRYTGYEDYWGQGILVTVSS

SEQ ID NO: 150 - protein, 123 aa, synthetic construct
QVQLVQSGLTLVKPTQTLTLTCTFSGFSLSTTGVSVGWIRQPPGKALEWLALIYWDDDKHYSPSLKSRLTITKDTSKNQVVLTMTNMDPVDTATYYCAPGGYSSGWPGFYDLWGRGTLVTVSS

SEQ ID NO: 151 - protein, 122 aa, synthetic construct
QVQLQESGPGLVKPSETLSLTCTVSGGSISGHYWSWIRQSPGKGLEWIGYITYSGSTNYNPSLKSRVTISVDTSKNQFSLKLSSVTAADTAVYYCGTIAVAGRVYYYGVDVWGRGTTVTVSS

SEQ ID NO: 152 - protein, 123 aa, synthetic construct
QVQLVQSGPALVKPTQTLTLTCTFSGFSLYTSGVAVAWIRQPPGKALEWLALIYWDDDKWYTPSLKTRLSITKDTSKNQVVLTMTNMDPVDTATYYCAHRGSFNWNYGYFDYWGQGTLVTVSS

SEQ ID NO: 153 - protein, 121 aa, synthetic construct
QVQLQQWGAGLLKPSETLSLTCAVYGGSFSGYYWTWIRQPPGKGLEWIGESNHGGSTNFNPSLKSRVTISVDTSKNHFSLKLSPVTAADTAVYYCTRGGTIFGLKDAFDIWGQGTMVTVSS

SEQ ID NO: 154 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 155 - protein, 1382 aa, Canis lupus
MKAPAVLAPGILVLLFTLVQKSYGECKEALVKSEMNVNMKYQLPNFTAETPIQNVVLHKHHIYLGAVNYIYVLNDKDLQKVAEYKTGPVLEHPDCSPCQDCSHKANLSGGVWEDNINMALLVDTYYDDQLISCGSVHRGTCQRHILPPSNIADIQSEVHCMYSSQADEEPSQCPDCVVSALGTKVLISEKDRFINFFVGNTINSSDHPDHSLHSISVRRLKETQDGFKFLTDQSYIDVLPEFRDSYPIKYVHAFESNHFIYFLTVQRETLDAQTFHTRIIRFCSVDSGLHSYMEMPLECILTEKRRKRSTREEVFNILQAAYVSKPGAHLAKQIGANLNDDILYGVFAQSKPDSAEPMNRSAVCAFPIKYVNEFFNKIVNKNNVRCLQHFYGPNHEHCFNRTLLRNSSGCEARNDEYRTEFTTALQRVDLFMGQFNQVLLTSISTFIKGDLTIANLGTSEGRFMQVVVSRSGLSTPHVNFRLDSHPVSPEAIVEHPLNQNGYTLVVTGKKITRIPLNGLGCEHFQSCSQCLSAPPFVQCGWCHDRCVHLEECPTGAWTQEVCLPAIYEVFPTSAPLEGGTVLTVCGWDFGFRRNNKFDLKKTKVFLGNESCTLTLSESTTNMLKCTVGPAVNEHFNISIIISNGRGTAQYSTFSYVDPIITSISPSYGPKNGGTLLTLTGKYLNSGNSRHISMGGKTCTLKSVSDSILECYTPAQATATEFPIKLKIDLANREMNSFSYQEDPIVYAIHPTKSFISGGSTITAVGKNLNSVSVLRMVIDVHETRRNFTVACQHRSNSEIICCTTPSLQQLNLQLPLKTKAFFMLDGIHSKYFDLIYVHNPVFKPFEKPVMISIGNENVLEIKGNDIDPEAVKGEVLKVGNKSCETIYSDSKAVLCKVPNDLLKLNNELNIEWKQAVSSTVLGKVIVQPDQNFTGLIAGVISISTIVLLLLGLFLWLKRKKQIKDLGSELVRYDARVHTPHLDRLVSARSVSPTTEMVSNESVDYRATFPEDQFPNSSQNGSCRQVQYPLTDLSPMLTSGDSDISSPLLQNTVHIDLSALNPELVQAVQHVVIGPSSLIVHFNEVIGRGHFGCVYHGTLLDNDDKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPRAELRPSFSELVSRISAIFSTFIGEHYVHVNATYVNVKCVAPYPSLLSSQDNIDGEGDT